(19) United States
(12) Patent Application Publication
(10) Pub. No.: US 2024/0245790 A1
(43) Pub. Date: Jul. 25, 2024

(54) CYCLIC CELL PENETRATING PEPTIDES

(71) Applicant: Entrada Therapeutics, Inc., Boston, MA (US)

(72) Inventors: Ziqing Qian, Wellesley, MA (US); Patrick Dougherty, Independence, OH (US); Mahboubeh Kheirabadi, Natick, MA (US); Haoming Liu, Canton, MA (US); Xiulong Shen, Waltham, MA (US)

(21) Appl. No.: 18/506,057

(22) Filed: Nov. 9, 2023

Related U.S. Application Data
(63) Continuation-in-part of application No. PCT/US2022/072217, filed on May 9, 2022.
(63) Continuation-in-part of application No. 18/553,379, filed on Jan. 1, 0001.
(60) Provisional application No. 63/186,664, filed on May 10, 2021.
(60) Provisional application No. 63/214,085, filed on Jun. 23, 2021.
(60) Provisional application No. 63/239,671, filed on Sep. 1, 2021.
(60) Provisional application No. 63/290,960, filed on Dec. 17, 2021.
(60) Provisional application No. 63/298,565, filed on Jan. 11, 2022.
(60) Provisional application No. 63/268,577, filed on Feb. 25, 2022.
(60) Provisional application No. 63/362,295, filed on Mar. 31, 2022.
(60) Provisional application No. 63/168,888, filed on Mar. 31, 2021.
(60) Provisional application No. 63/171,860, filed on Apr. 7, 2021.
(60) Provisional application No. 63/239,671, filed on Sep. 1, 2021.
(60) Provisional application No. 63/290,960, filed on Dec. 17, 2021.
(60) Provisional application No. 63/298,565, filed on Jan. 11, 2022.
(60) Provisional application No. 63/268,577, filed on Feb. 25, 2022.

(51) Int. Cl. A61K 47/64; A61K 47/54; A61K 47/60

(52) U.S. Cl. CPC A61K 47/64; A61K 47/545; A61K 47/60

(57) ABSTRACT

The present disclosure is directed to cell penetrating peptides, including cyclic cell penetrating peptides with high cytosolic delivery efficiency and reduced toxicity that are able to effectively deliver cargo inside a cell to treat a variety of conditions and diseases.

### CROSS-REFERENCE TO RELATED APPLICATIONS

This application is a continuation in part of International Application No. PCT/US2022/072217, filed on May 9, 2022, now published as WO 2022/241408, which claims priority to U.S. provisional patent application No. 63/186,664, which was filed on May 10, 2021, U.S. provisional patent application No. 63/214,085, which was filed on Jun. 23, 2021, U.S. provisional patent application No. 63/239,671, which was filed on Sep. 1, 2021, U.S. provisional patent application No. 63/290,960, which was filed on Dec. 17, 2021, U.S. provisional patent application No. 63/298,565, which was filed on Jan. 11, 2022, U.S. provisional patent application No. 63/268,577, which was filed on Feb. 25, 2022, U.S. provisional patent application No. 63/362,295, which was filed on Mar. 31, 2022, the disclosures of each of which are hereby incorporated by reference in their entireties. The present application is also a continuation in part of U.S. patent application Ser. No. 18/553,379, filed on Sep. 29, 2023, which is a U.S. National Stage Filing under 35 U.S.C. 371 from International Application No. PCT/US2022/071489, filed Mar. 31, 2022, now published as WO 2022/213118, which claims priority U.S. provisional patent application No. 63/168,888, which was filed on Mar. 31, 2021, U.S. provisional patent application No. 63/171,860, which was filed on Apr. 7, 2021, U.S. provisional patent application No. 63/239,671, which was filed on Sep. 1, 2021, U.S. provisional patent application No. 63/290,960, which was filed on Dec. 17, 2021, U.S. provisional patent application No. 63/298,565, which was filed on Jan. 11, 2022, U.S. provisional patent application No. 63/268,577 which was filed on Feb. 25, 2022, the disclosures of each of which are hereby incorporated by reference in their entireties.

### INCORPORATION BY REFERENCE OF SEQUENCE LISTING

This application contains a Sequence Listing which has been submitted electronically in ST26 format and is hereby incorporated by reference in its entirety. Said ST26 file, created on Feb. 29, 2024, is named “5892050US1.xml” and is 298,214 bytes in size.

### BACKGROUND

Nucleic acids and their synthetic analogs hold enormous potential as therapeutic agents, especially against targets that are challenging for conventional drug modalities (e.g., missing/defective proteins caused by genetic mutations).

However, a major problem in translating the potential of such therapies to the clinic is their limited ability to gain access to the intracellular compartment when administered systemically. Carrier systems, such as polymers, cationic liposomes or chemical modifications, for example, by the covalent attachment of cholesterol molecules, have been used to facilitate intracellular delivery. Still, intracellular delivery efficiency by these approaches is often low and improved delivery systems to increase efficacy of intracellular delivery have remained elusive.

In the late 1980s, it was discovered that the highly positively charged HIV Tat peptide could translocate across the mammalian cell membrane. Subsequently, other “cell penetrating peptides” (CPPs) have been discovered that are capable of penetrating the cell membrane at low micromolar concentrations without causing significant membrane damage. Qian et al. (2016) “Discovery and Mechanism of Highly Efficient Cyclic Cell-Penetrating Peptides.” Biochem. 55:2601-2612. However, effective cytosolic delivery by many of these CPPs is limited by poor endosomal escape efficiency.

Accordingly, new cell penetrating peptides and compositions comprising peptides with a suitable toxicity profile are needed.

The compositions and methods disclosed herein address these and other needs.

### SUMMARY

One potential strategy to subvert the membrane barrier and deliver drugs into cells is to attach them to “cell-penetrating peptides” (also referred to herein as CPPs or EEVs (endosomal escape vehicles)). Certain residues, such as arginine, that facilitate cytoplasmic delivery have been implicated as significant contributors to systemic organ toxicity.

New cell penetrating peptides and compositions comprising peptides with a suitable toxicity profile are provided.

The compositions and methods disclosed herein address these and other needs.

### BRIEF DESCRIPTION OF THE FIGURES

FIG. 1 shows non-limiting examples of cCPPs containing arginine surrogates for the delivery of cargo.

FIG. 2 shows the structure of conjugation product Compound 1b-PMO (EEV-PMO-MDX-1) formed between Compound 1b and a PMO.

FIG. 3 shows the change in cell viability in human fibroblast cell line WI38 upon treatment with various concentrations of EEV12 and Compound 1b.

FIG. 4 shows the quantification of lactate dehydrogenase (LDH) released from human fibroblast cell line WI38 upon treatment with various concentrations of EEV12 and Compound 1b.

FIG. 5 shows the change in cell viability in primary human renal proximal tubular epithelial cells (RPTECs) upon treatment with various concentrations of EEV12 and Compound 1b.

FIG. 6 shows the quantification of lactate dehydrogenase (LDH) release from primary human renal proximal tubular epithelial cells (RPTECs) upon treatment with various concentrations of EEV12 and Compound 1b.

FIG. 7 shows the change in cell viability in human umbilical vein endothelial cells (HUVECs) upon treatment with various concentrations of EEV12 and Compound 1b.

FIG. 8 shows the change in cell viability in human peripheral blood mononuclear cells (hPBMCs) upon treatment with various concentrations of EEV12 and Compound 1b.

FIG. 9 shows the quantification of serum histamine levels by LC/MS in male C57BL/6 mice following intravenous injection of various quantities of EEV12 and Compound 1b.

FIGS. 10A-10E show the quantification of exon 23 skipping as determined by RT-PCR in mdx mice 7 days after intravenous injection of various quantities of EEV-MDX-PMO-1 or EEV-MDX-PMO-2. FIG. 10A shows the quantification of exon 23 skipping in the transverse abdominis. FIG. 10B shows the quantification of exon 23 skipping in the heart. FIG. 10C shows the quantification of exon 23 skipping in the diaphragm. FIG. 10D shows the quantification of exon 23 skipping in the tibalis anterior. FIG. 10E shows the quantification of exon 23 skipping in the quadriceps.

FIG. 11 shows the Western Blot quantification of dystrophin production in mdx mice 7 days after intravenous injection of various quantities of EEV-MDX-PMO-1 or EEV-MDX-PMO-2 as described in Example 4. Values given are relative to dystrophin levels in the indicated tissue in wild-type C57BL/10 mice.

FIG. 12 shows the structure of conjugation product EEV-MDX-PMO-3 formed between Compound 4b and a PMO.

FIG. 13 shows the PCR agarose gel images for exon 23 skipping in mdx mice 3 days after intravenous injection of 40 mpk of EEV-MDX-PMO-2 and 40 mpk of EEV-MDX-PMO-3.

FIGS. 14A-14C show the quantification of exon 23 skipping as determined by RT-PCR in mdx mice 3 or 7 days, respectively, after intravenous injection of various quantities of EEV-MDX-PMO-2 or EEV-MDX-PMO-3 or R6-PMO. FIG. 14A shows the quantification of exon 23 skipping in the quadriceps. FIG. 14B shows the quantification of exon 23 skipping in the diaphragm. FIG. 14C shows the quantification of exon 23 skipping in the heart.

FIGS. 15A-15D show the Western Blot quantification of dystrophin production in mdx mice 3 days after intravenous injection of 40 mg/kg of EEV-MDX-PMO-2 or EEV-MDX-PMO-3. All values are given as dystrophin levels relative to dystrophin levels in wild-type C57BL/10 mice. FIG. 15A shows the relative quantification of dystrophin levels in the heart. FIG. 15B shows the relative quantification of dystrophin levels in the diaphragm. FIG. 15C shows the relative quantification of dystrophin levels in the tibalis anterior. FIG. 15D shows the relative quantification of dystrophin levels in the quadriceps.

FIGS. 16A and 16B show the conjugation chemistry for connecting a therapeutic moiety (TM) such as an antisense compound (AC) to a cell penetrating peptide (CPP). Other chemistries may be employed, for example thiol maleimide or copper catalyzed click chemistries

FIG. 17 shows the conjugation chemistry for connecting a sample oligonucleotide and a cCPP with additional linker modality containing a polyethylene glycol (PEG) moiety (SEQ ID NOS: 226-227).

FIGS. 18A-18C show the synthetic scheme for EEV-PMO-DMD44-1 (FIG. 18A), EEV-PMO-DMD44-2 (FIG. 18B), and EEV-PMO-DMD44-3 (FIG. 18C)

FIG. 19 shows restoration of dystrophin protein expression in DMDA45 muscle cells treated with EEV-PMO-DMD44-1, EEV-PMO-DMD44-2, or EEV-PMO-DMD44-3 as quantified by Western Blot.

FIGS. 20A-20B show exon skipping and drug concentration in tissues of hDMD mice treated with EEV-PMO-DMD44-1 (FIG. 20A) and EEV-PMO-DMD44-2 (FIG. 20B) via IV injection.

FIGS. 21A-21B depict exon skipping (FIG. 21A) and drug exposure (FIG. 21B) for EEV-PMO-DMD44-1 in a NHP model.

FIGS. 21C-21D depict exon skipping (FIG. 21C) and drug exposure (FIG. 21D) for EEV-PMO-DMD44-2 in a NHP model.

FIG. 22A-22F demonstrate RNA splicing measurements for Mbnl1 (for exon 5 inclusion;

FIG. 23A), Bin1 (for exon 11 inclusion; FIG. 22B), IR (for exon 11 inclusion; FIG. 22C), DMD (for exon 78 inclusion; FIG. 22D), LDB3 (for exon 11 inclusion; FIG. 22E) and Sos1 (for exon inclusion; FIG. 22F). T-test of treated versus untreated DM1 myotubes *p<0.05; **p<0.01; ***p<0.001.

FIGS. 23A-23B depict exon skipping and restoration of dystrophin in patient-derived muscle cells (FIG. 23A) and exon skipping in cardiac and skeletal muscles in a transgenic mouse carrying the full-length human DMD gene using a compound for exon 44 skipping (FIG. 23B).

FIGS. 24A-24C depict the tissue concentration and percent of exon skipping for a compound for exon 44 skipping in heart (FIG. 24A), tibialis anterior (FIG. 24B) and diaphragm (FIG. 25C) in a transgenic mouse carrying the full-length human DMD gene.

FIG. 25. depicts plasma levels over time after administration of a compound for exon 44 skipping to a NHP.

FIG. 26 demonstrates meaningful levels of exon skipping in both skeletal muscles and the heart of the NHP administered the compound for exon skipping.

FIG. 27 shows modified nucleotides used in antisense oligonucleotides described herein.

FIGS. 28A-28D provide structures for morpholino subunit monomers used in synthesizing phosphorodiamidate-linked morpholino oligomers. FIG. 28A provides the structure for adenine morpholino monomer. FIG. 28B provides the structure for cytosine morpholino monomer. FIG. 28C provides the structure for guanine morpholino monomer. FIG. 28D provides the structure for thymine morpholino monomer.

FIGS. 29A-29D illustrate conjugation chemistries for connecting AC to a cyclic cell penetrating peptide (cCPP). FIG. 29A shows the amide bond formation between peptides with carboxylic acid group or with TFP activated ester and primary amine residues at the 5′ end of AC. FIG. 29B shows the conjugation of secondary amine or primary amine modified AC at 3′ and peptide-TFP ester through amide bond formation. FIG. 29C shows the conjugation of peptide-azide to the 5′ cyclooctyne modified AC via copper-free azide-alkyne cycloaddition. FIG. 29D demonstrates another conjugation between 3′ modified cyclooctyne ACs or 3′ modified azide ACs and cCPP containing linker-azide or linker-alkyne/cyclooctyne moiety, via a copper-free azide-alkyne cycloaddition or cupper catalyzed azide-alkyne cycloaddition, respectively (click reaction).

FIG. 30 shows the conjugation chemistry for connecting AC and cCPP with an additional linker modality containing a polyethylene glycol (PEG) moiety. Other conjugation chemistries may be employed.

FIG. 31A shows a schematic for GYS1 knockdown via exon skipping and FIG. 31B shows a schematic for IRF-5 knockdown via exon skipping.

FIGS. 32A-32D show the level of GYS1 mRNA expression of untreated mice, mice treated with a PMO, and mice treated with various concentrations of an EEV-PMO in GAA knockout mouse model. FIG. 32A is a gel showing the level of GYS1 expression in the diaphragm of the mice and FIG. 32B is plot of the data in FIG. 32A. FIG. 32C is an SDS-PAGE gel showing the level of GYS1 expression in the cardiac muscle of the mice and FIG. 32D is plot of the data in FIG. 32C. (P>0.05=NS; P≤0.05=*; P≤0.01=**; P≤0.001=***)

FIGS. 33A-33D show plots of the level of GYS1 protein expression in the heart (FIG. 33A), diaphragm (FIG. 33B), quadriceps (FIG. 33C), and triceps (FIG. 33D) of untreated mice, mice treated with a PMO, and mice treated with an EEV-PMO at various time points after treatment. (P>0.05=NS; P≤0.05=*; P≤0.01=**; P≤0.001=***)

FIGS. 34A-34C are plots showing the level of IRF5 expression the liver (FIG. 34A), small intestine (FIG. 34B), and tibialis anterior (FIG. 34C) of mice treated with various concentrations of an EEV-PMO. (P>0.05=NS; P≤0.05=*; P≤0.01=**; P≤0.001=***)

FIG. 35 is a plot showing the level of IRF5 expression in an in vitro experiment where mouse macrophage cells were treated with various concentrations of EEV-PMO-IRF5-1. (P>0.05=NS; P≤0.05=*; P≤0.01=**; P≤0.001=***)

FIG. 36 is a plot showing the level of IRF5 expression in an in vitro experiment where mouse macrophage cells were treated with various EEV-PMO constructs then stimulated with R848. (P>0.05=NS; P≤0.05=*; P≤0.01=**; P≤0.001=***).

FIG. 37A-37E show the sequences (FIG. 37A; SEQ ID NOS: 228-229, 128-130, 234) and structures (FIG. 37B-37E) of various EEV-PMO compounds. FIG. 37B is the structure of EEV-PMO-IRF5-1. FIG. 37C is the structure of EEV-PMO-IRF5-3. FIG. 37D is the structure of EEV-PMO-IRF5-4. FIG. 37E is the structure of EEV-PMO-IRF5-2.

FIG. 38 is a bar graph showing the levels of IRF5 expression in RAW 264.7 Monocyte/Macrophage cells after treatment with compounds at various concentrations followed by R848 stimulation.

FIG. 39 is a bar graph showing the level of exon 4 skipping in RAW 264.7 Monocyte/Macrophage cells after treatment with compounds at various concentrations. NT=no treatment.

FIG. 40 is a bar graph showing the transcript levels in RAW 264.7 Monocyte/Macrophage cells after treatment with compounds at various concentrations.

FIG. 41A-41D show dose dependent correction of MBNL1 downstream genes in quadriceps 1 week after HSA-LR mice were injected with EEV-PMO-DM1-3; FIG. 41A: Atp2a1, FIG. 41B: Nfix, FIG. 41C: Clcn1, FIG. 41D: Mbnl1.

FIG. 42A-42D show dose dependent correction of MBNL1 downstream genes in gastrocnemius 1 week after HSA-LR mice were injected with EEV-PMO-DM1-3; FIG. 42A: Atp2a1, FIG. 42B: Nfix, FIG. 42C: Clcn1, FIG. 42D: Mbnl1.

FIG. 43A-43D show dose dependent correction of MBNL1 downstream genes in tibialis anterior 1 week after HSA-LR mice were injected with EEV-PMO-DM1-3; FIG. 43A: Atp2a1, FIG. 43B: Nfix, FIG. 43C: Clcn1, FIG. 43D: Mbnl1).

FIG. 44A-44D show dose dependent correction of MBNL1 downstream genes in triceps anterior 1 week after HSA-LR mice were injected with EEV-PMO-DM1-3; FIG. 44A: Atp2a1, FIG. 44B: Nfix, FIG. 44C: Clcn1, FIG. 44D: Mbnl1).

FIG. 45A-45D provide an overlay of the data shown in FIGS. 41A-D, 42A-D, 43A-D and 44A-D.

FIG. 46A-46D show that administration of EEV-PMO-DM1-3 resulted in ˜50-70% HSA mRNA knockdown in skeletal muscles of HSA-LR mice: FIG. 46A: quadriceps; FIG. 46B: Gastrocnemius; FIG. 46C: Triceps; and FIG. 46D: Tibialis Anterior. Statistical significance is calculated by one-way ANOVA relative to HSA-LR vehicle treated group (n=3). Dose is based on PMO.

FIG. 47A-47F are graphs showing a dose-dependent response for drug levels in various muscle tissues in mice administered EEV-PMO-DM1-3. FIG. 47A: quadriceps; FIG. 47B: triceps; FIG. 47C: heart; FIG. 47D: gastrocnemius; FIG. 47E: tibialis anterior; and FIG. 47F: diaphragm.

FIG. 48 shows PMO-DM1, the major metabolite detected in vivo.

FIG. 49A-49C depict EEV-PMO-DM1-3 exposure in Brain (FIG. 49A), Liver (FIG. 49B) and Kidney (FIG. 49C) after administration at 15, 30, 60 and 90 mpk.

FIG. 50 shows EEV-PMO-DM1-3 treatment reduces CUG foci in HSA-LR mouse TA muscle after 1 week.

FIG. 51 is a graph showing EEV-PMO-DM1-3 treatment reduces CUG foci in HSA-LR mouse TA muscle after 1 week.

FIG. 52 shows a dose dependent myotonia reduction in HSA-LR mice 7 days after treatment with EEV-PMO-DM1-3 at 15, 30, 60 and 90 mpk.

FIG. 53A-53C show the duration of effect of 80 mpk EEV-PMO-DM1-3 (60 mpk oligo) on Atp2a1 exon 22 inclusion in HSA-LR mice. Tibialis anterior (FIG. 53A); triceps (FIG. 53B); and quadriceps (FIG. 53C)

FIG. 54A-54C show the duration of effect of 80 mpk EEV-PMO-DM1-3 (60 mpk oligo) on Nfix exon 7 inclusion in HSA-LR mice. Tibialis anterior (FIG. 54A); triceps (FIG. 54B); and quadriceps (FIG. 54C).

FIG. 55A-55C show the duration of effect of 80 mpk EEV-PMO-DM1-3 (60 mpk oligo) on Mbnl1 exon 5 inclusion in HSA-LR mice. Tibialis anterior (FIG. 55A); triceps (FIG. 55B); and quadriceps (FIG. 55C).

FIG. 56A-56C show the duration of effect of 80 mpk EEV-PMO-DM1-3 (60 mpk oligo) on exon 22 inclusion in gastrocnemius of HSA-LR mice. Atp2a1 (FIG. 56A); Nfix (FIG. 56B); and Mbnl1 (FIG. 56C).

FIG. 57 show the duration of effect of 80 mpk EEV-PMO-DM1-3 (60 mpk oligo) in gastrocnemius, triceps, tibialis anterior and quadricep of HSA-LR mice.

FIG. 58A-58D show the duration of effect of 80 mpk EEV-PMO-DM1-3 (60 mpk oligo) in muscle tissue of HSA-LR mice. FIG. 58A: quadriceps, FIG. 58B: gastrocnemius, FIG. 58C: triceps; and FIG. 58D: tibialis anterior.

FIG. 59A-59D show the duration of effect of 80 mpk EEV-PMO-DM1-3 (60 mpk oligo) on Clcn1 exon 7a inclusion in HSA-LR mice. FIG. 59A: tibialis anterior; FIG. 59B: triceps; FIG. 59C: quadriceps; and FIG. 59D: gastrocnemius.

FIG. 60A-60D show EEV-PMO-DM1-3 showed HSA mRNA knockdown trend 1-week and 4-week post-injection. FIG. 60A: tibialis anterior; FIG. 60B: triceps; FIG. 60C: quadriceps; and FIG. 60D: gastrocnemius.

FIG. 61A-61D show a decrease in drug level with 80 mpk EEV-PMO-DM1-3 after 1 week to 4 weeks in muscle tissue. FIG. 61A: tibialis anterior; FIG. 61B: gastrocnemius; FIG. 61C: triceps; and FIG. 61D: gastrocnemius. EEV-PMO-DM1-3 (60 mpk oligo, 80 mpk whole drug) fully correct mis-splicing in gastrocnemius, triceps, tibialis anterior and quadricep post 1 week treatment.

FIG. 62A-62B show a decrease in drug levels was observed with 80 mpk dose of EEV-PMO-DM1-3 after 1 week to 4 week in liver and kidney.

FIG. 63A-63C show that EEV-PMO-DM1-3 promotes significant biomarker splicing correction in DM1 patient-derived muscle cells.

FIG. 64A-64C show that EEV-PMO-DM1-3 reduces nuclear foci in DM1 patient-derived muscle cells.

FIG. 65A-65B show that no issues with tolerability were observed with PMO-DM1 and EEV-PMO-DM1-3 up to about 800 micromolar.

FIG. 66A-66D show the level of exon 23 correction in muscle tissue of MDX mice five days after treatment with the indicated compounds. Results are shown for diaphragm (FIG. 66A), heart (FIG. 66B), tibialis anterior (FIG. 66C) and triceps (FIG. 66D).

FIG. 67A-67C show the level of dystrophin expression in muscle tissue of MDX mice five days after treatment with the indicated compounds. Results are shown for diaphragm (FIG. 67A), heart (FIG. 67B) and tibialis anterior (FIG. 67C).

### DETAILED DESCRIPTION

### Endosomal Escape Vehicles (EEVs)

An endosomal escape vehicle (EEV) is provided herein that can be used to transport a cargo across a cellular membrane, for example, to deliver the cargo to the cytosol or nucleus of a cell. Cargo can include a macromolecule, for example, a peptide or oligonucleotide, or a small molecule. The EEV can comprise a cell penetrating peptide (CPP), for example, a cyclic cell penetrating peptide (cCPP), which is conjugated to an exocyclic peptide (EP). The EP can be referred to interchangeably as a modulatory peptide (MP). The EP can comprise a sequence of a nuclear localization signal (NLS). The EP can be coupled to the cargo. The EP can be coupled to the cCPP. The EP can be coupled to the cargo and the cCPP. Coupling between the EP, cargo, cCPP, or combinations thereof, may be non-covalent or covalent. The EP can be attached through a peptide bond to the N-terminus of the cCPP. The EP can be attached through a peptide bond to the C-terminus of the cCPP. The EP can be attached to the cCPP through a side chain of an amino acid in the cCPP. The EP can be attached to the cCPP through a side chain of a lysine which can be conjugated to the side chain of a glutamine in the cCPP. The EP can be conjugated to the 5′ or 3′ end of an oligonucleotide cargo. The EP can be coupled to a linker. The exocyclic peptide can be conjugated to an amino group of the linker. The EP can be coupled to a linker via the C-terminus of an EP and a cCPP through a side chain on the cCPP and/or EP. For example, an EP may comprise a terminal lysine which can then be coupled to a cCPP containing a glutamine through an amide bond. When the EP contains a terminal lysine, and the side chain of the lysine can be used to attach the cCPP, the C- or N-terminus may be attached to a linker on the cargo.

### Exocyclic Peptides

The exocyclic peptide (EP) can comprise from 2 to 10 amino acid residues e.g., 2, 3, 4, 5, 6, 7, 8, 9, or 10 amino acid residues, inclusive of all ranges and values therebetween. The EP can comprise 6 to 9 amino acid residues. The EP can comprise from 4 to 8 amino acid residues.

Each amino acid in the exocyclic peptide may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein. For example, the amino acids can be A, G, P, K, R, V, F, H, Nal, or citrulline.

The EP can comprise at least one positively charged amino acid residue, e.g., at least one lysine residue and/or at least one amine acid residue comprising a side chain comprising a guanidine group, or a protonated form thereof. The EP can comprise 1 or 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four or more lysine residues. The EP can comprise 2, 3, or 4 lysine residues. The amino group on the side chain of each lysine residue can be substituted with a protecting group, including, for example, trifluoroacetyl (—COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF₃) group. The protecting group can be included to enable amide conjugation. The protecting group can be removed after the EP is conjugated to a cCPP.

The EP can comprise at least 2 amino acid residues with a hydrophobic side chain. The amino acid residue with a hydrophobic side chain can be selected from valine, proline, alanine, leucine, isoleucine, and methionine. The amino acid residue with a hydrophobic side chain can be valine or proline.

The EP can comprise at least one positively charged amino acid residue, e.g., at least one lysine residue and/or at least one arginine residue. The EP can comprise at least two, at least three or at least four or more lysine residues and/or arginine residues.

The EP can comprise KK, KR, RR, HH, HK, HR, RH, KKK, KGK, KBK, KBR, KRK, KRR, RKK, RRR, KKH, KHK, HKK, HRR, HRH, HHR, HBH, HHH, HHHH (SEQ ID NO: 58), KHKK (SEQ ID NO: 59), KKHK (SEQ ID NO: 60), KKKH (SEQ ID NO: 61), KHKH (SEQ ID NO. 62), HKHK (SEQ ID NO: 63), KKKK (SEQ ID NO: 64), KKRK (SEQ ID NO. 65), KRKK (SEQ ID NO: 66), KRRK (SEQ ID NO: 67), RKKR (SEQ ID NO: 68), RRRR (SEQ ID NO: 69), KGKK (SEQ ID NO: 70), KKGK (SEQ ID NO: 71), HBHBH (SEQ ID NO: 72), HBKBH (SEQ ID NO: 73), RRRRR (SEQ ID NO: 74), KKKKK (SEQ ID NO: 75), KKKRK (SEQ ID NO: 76), RKKKK (SEQ ID NO: 77), KRKKK (SEQ ID NO: 78), KKRKK (SEQ ID NO: 79), KKKKR (SEQ ID NO: 80), KBKBK (SEQ ID NO: 81), RKKKKG (SEQ ID NO: 82), KRKKKG (SEQ ID NO: 83), KKRKKG (SEQ ID NO: 84), KKKKRG (SEQ ID NO: 85), RKKKKB (SEQ ID NO: 86), KRKKKB (SEQ ID NO: 87), KKRKKB (SEQ ID NO: 88), KKKKRB (SEQ ID NO: 89), KKKRKV (SEQ ID NO: 90), RRRRRR (SEQ ID NO: 91), HHHHHH (SEQ ID NO: 92), RHRHRH (SEQ ID NO: 93), HRHRHR (SEQ ID NO: 94), KRKRKR (SEQ ID NO: 95), RKRKRK (SEQ ID NO: 96), RBRBRB (SEQ ID NO: 97), KBKBKB (SEQ ID NO: 98), PKKKRKV (SEQ ID NO: 103), PGKKRKV (SEQ ID NO: 104), PKGKRKV (SEQ ID NO: 105), PKKGRKV (SEQ ID NO: 106), PKKKGKV (SEQ ID NO: 107), PKKKRGV (SEQ ID NO: 108) or PKKKRKG (SEQ ID NO: 109), wherein B is beta-alanine. The amino acids in the EP can have D or L stereochemistry.

The EP can comprise KK, KR, RR, KKK, KGK, KBK, KBR, KRK, KRR, RKK, RRR, KKKK (SEQ ID NO: 64), KKRK (SEQ ID NO: 65), KRKK (SEQ ID NO: 66), KRRK (SEQ ID NO: 67), RKKR (SEQ ID NO: 68), RRRR (SEQ ID NO: 69), KGKK (SEQ ID NO: 70), KKGK (SEQ ID NO: 71), KKKKK (SEQ ID NO: 75), KKKRK (SEQ ID NO: 76), KBKBK (SEQ ID NO: 81), KKKRKV (SEQ ID NO: 90), PKKKRKV (SEQ ID NO: 103), PGKKRKV (SEQ ID NO: 104), PKGKRKV (SEQ ID NO: 105), PKKGRKV (SEQ ID NO: 106), PKKKGKV (SEQ ID NO: 107), PKKKRGV (SEQ ID NO: 108) or PKKKRKG (SEQ ID NO: 109). The EP can comprise PKKKRKV (SEQ ID NO: 103), RR, RRR, RHR, RBR, RBRBR (SEQ ID NO: 99), RBHBR (SEQ ID NO: 100), or HBRBH (SEQ ID NO: 101), wherein B is beta-alanine. The amino acids in the EP can have D or L stereochemistry.

The EP can consist of KK, KR, RR, KKK, KGK, KBK, KBR, KRK, KRR, RKK, RRR, KKKK (SEQ ID NO: 64), KKRK (SEQ ID NO: 65), KRKK (SEQ ID NO: 66), KRRK (SEQ ID NO: 67), RKKR (SEQ ID NO: 68), RRRR (SEQ ID NO: 69), KGKK (SEQ ID NO: 70), KKGK (SEQ ID NO: 71), KKKKK (SEQ ID NO: 75), KKKRK (SEQ ID NO: 76), KBKBK (SEQ ID NO: 81), KKKRKV (SEQ ID NO: 90), PKKKRKV (SEQ ID NO: 103), PGKKRKV (SEQ ID NO: 104), PKGKRKV (SEQ ID NO: 105), PKKGRKV (SEQ ID NO: 106), PKKKGKV (SEQ ID NO: 107), PKKKRGV (SEQ ID NO: 108) or PKKKRKG (SEQ ID NO: 109). The EP can consist of PKKKRKV (SEQ ID NO: 103), RR, RRR, RHR, RBR, RBRBR (SEQ ID NO: 99), RBHBR (SEQ ID NO: 100), or HBRBH (SEQ ID NO: 101), wherein B is beta-alanine. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV (SEQ ID NO: 103). The EP can consist of an NLS comprising the amino acid sequence PKKKRKV (SEQ ID NO: 103). The EP can comprise an NLS comprising an amino acid sequence selected from NLSKRPAAIKKAGQAKKKK (SEQ ID NO: 253), PAAKRVKLD (SEQ ID NO: 112), RQRRNELKRSF (SEQ ID NO: 113), RMRKFKNKGKDTAELRRRRVEVSVELR (SEQ ID NO: 114), KAKKDEQILKRRNV (SEQ ID NO: 115), VSRKRPRP (SEQ ID NO: 116), PPKKARED (SEQ ID NO: 117), PQPKKKPL (SEQ ID NO: 118), SALIKKKKKMAP (SEQ ID NO: 119), DRLRR (SEQ ID NO: 120), PKQKKRK (SEQ ID NO: 121), RKLKKKIKKL (SEQ ID NO: 122), REKKKFLKRR (SEQ ID NO: 123), KRKGDEVDGVDEVAKKKSKK (SEQ ID NO: 124) and RKCLQAGMNLEARKTKK (SEQ ID NO: 125). The EP can consist of an NLS comprising an amino acid sequence selected from NLSKRPAAIKKAGQAKKKK (SEQ ID NO: 253), PAAKRVKLD (SEQ ID NO: 112), RQRRNELKRSF (SEQ ID NO: 113), RMRKFKNKGKDTAELRRRRVEVSVELR (SEQ ID NO: 114), KAKKDEQILKRRNV (SEQ ID NO: 115), VSRKRPRP (SEQ ID NO: 116), PPKKARED (SEQ ID NO: 117), PQPKKKPL (SEQ ID NO: 118), SALIKKKKKMAP (SEQ ID NO: 119), DRLRR (SEQ ID NO: 120), PKQKKRK (SEQ ID NO: 121), RKLKKKIKKL (SEQ ID NO: 122), REKKKFLKRR (SEQ ID NO: 123), KRKGDEVDGVDEVAKKKSKK (SEQ ID NO: 124) and RKCLQAGMNLEARKTKK (SEQ ID NO: 125).

All exocyclic sequences can also contain an N-terminal acetyl group. Hence, for example, the EP can have the structure: Ac-PKKKRKV (SEQ ID NO: 103).

### Cell Penetrating Peptides (CPP)

The cell penetrating peptide (CPP) can comprise 6 to 20 amino acid residues. The cell penetrating peptide can be a cyclic cell penetrating peptide (cCPP). The cCPP is capable of penetrating a cell membrane. An exocyclic peptide (EP) can be conjugated to the cCPP, and the resulting construct can be referred to as an endosomal escape vehicle (EEV). The cCPP can direct a cargo (e.g., a therapeutic moiety (TM) such as an oligonucleotide, peptide or small molecule) to penetrate the membrane of a cell. The cCPP can deliver the cargo to the cytosol of the cell. The cCPP can deliver the cargo to a cellular location where a target (e.g., pre-mRNA) is located. To conjugate the cCPP to a cargo (e.g., peptide, oligonucleotide, or small molecule), at least one bond or lone pair of electrons on the cCPP can be replaced.

The total number of amino acid residues in the cCPP is in the range of from 6 to 20 amino acid residues, e.g., 6, 7, 8, 9, 10, 11, 12, 13, 14, 15, 16, 17, 18, 19, or 20 amino acid residues, inclusive of all ranges and subranges therebetween. The cCPP can comprise 6 to 13 amino acid residues. The cCPP disclosed herein can comprise 6 to 10 amino acids. By way of example, cCPP comprising 6-10 amino acid residues can have a structure according to any of Formula I-A to I-E:

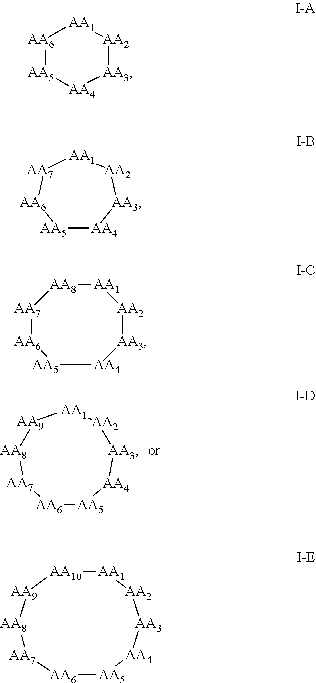

wherein AA₁, AA₂, AA₃, AA₄, AA₅, AA₆, AA₇, AA₈, AA₉, and AA₁₀ are amino acid residues.

The cCPP can comprise 6 to 8 amino acids. The cCPP can comprise 8 amino acids.

Each amino acid in the cCPP may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be a D-isomer of a natural amino acid. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein.

TABLE 1
Amino Acid Abbreviations

| | Abbreviations* | Abbreviations* |
|---|---|---|
| Amino Acid | L-amino acid | D-amino acid |
| 2-[2-[2- | AEEA, | NA |
| aminoethoxy]ethoxy]acetic acid | miniPEG, PEG2 | |
| Alanine | Ala (A) | ala (a) |
| Allo-isoleucine | Aile | Aile |
| Arginine | Arg (R) | arg (r) |
| Asparagine | Asn (N) | asn (n) |
| aspartic acid | Asp (D) | asp (d) |
| Cysteine | Cys (C) | cys (c) |
| Citrulline | Cit | Cit |
| Cyclohexylalanine | Cha | cha |
| 2,3-diaminopropionic acid | Dap | dap |
| 4-fluorophenylalanine | Fpa (Σ) | pfa |
| glutamic acid | Glu (E) | glu (e) |
| glutamine | Gln (Q) | gln (q) |
| glycine | Gly (G) | gly (g) |
| histidine | His (H) | his (h) |
| Homoproline (aka pipecolic acid) | Pip (Θ) | pip (θ) |
| isoleucine | Ile (I) | ile (i) |
| leucine | Leu (L) | leu (l) |
| lysine | Lys (K) | lys (k) |
| methionine | Met (M) | met (m) |
| 3-(2-naphthyl)-alanine | Nal (Φ) | nal (ϕ) |
| 3-(1-naphthyl)-alanine | 1-Nal | 1-nal |
| norleucine | Nle (Ω) | nle |
| phenylalanine | Phe (f) | phe (f) |
| phenylglycine | Phg (Ψ) | phg |
| 4- | F₂Pmp (Λ) | f₂pmp |
| (phosphonodifluoromethyl)phenylalanine | | |
| proline | Pro (P) | pro (p) |
| sarcosine | Sar (Ξ) | sar |
| selenocysteine | Sec (U) | sec (u) |
| serine | Ser (S) | ser (s) |
| threonine | Thr (T) | thr (y) |
| tyrosine | Tyr (Y) | tyr (y) |
| tryptophan | Trp (W) | trp (w) |
| valine | Val (V) | val (v) |
| Tert-butyl-alanine | Tle | tle |
| Penicillamine | Pen | Pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothienyl)-alanine | Bta | bta |

*single letter abbreviations: when shown in capital letters herein it indicates the L-amino acid form, when shown in lower case herein it indicates the D-amino acid form.

The cCPP can comprise 4 to 20 amino acids, wherein: (i) at least one amino acid has a side chain comprising a guanidine group, or a protonated form thereof; (ii) at least one amino acid has no side chain or a side chain comprising

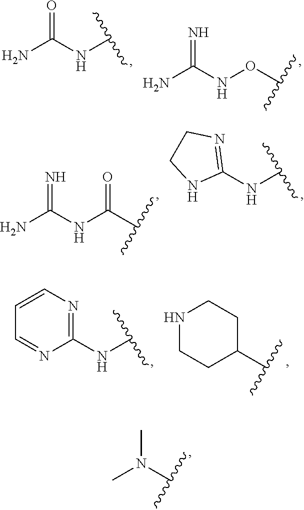

or a protonated form thereof; and (iii) at least two amino acids independently have a side chain comprising an aromatic or heteroaromatic group.

At least two amino acids can have no side chain or a side chain comprising

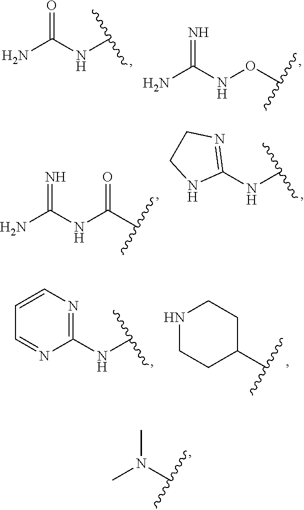

or a protonated form thereof. As used herein, when no side chain is present, the amino acid has two hydrogen atoms on the carbon atom(s) (e.g., —CH₂—) linking the amine and carboxylic acid.

The amino acid having no side chain can be glycine or β-alanine.

The cCPP can comprise from 6 to 20 amino acid residues which form the cCPP, wherein: (i) at least one amino acid can be glycine, β-alanine, or 4-aminobutyric acid residues; (ii) at least one amino acid can have a side chain comprising an aryl or heteroaryl group; and (iii) at least one amino acid has a side chain comprising a guanidine group,

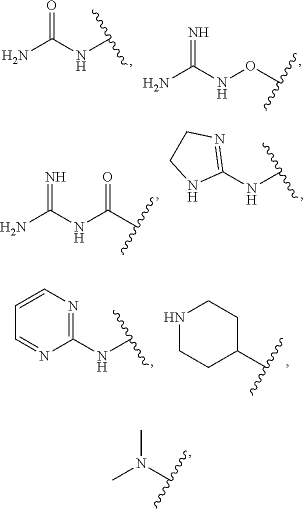

or a protonated form thereof.

The cCPP can comprise from 6 to 20 amino acid residues which form the cCPP, wherein: (i) at least two amino acid can independently be glycine, β-alanine, or 4-aminobutyric acid residues; (ii) at least one amino acid can have a side chain comprising an aryl or heteroaryl group; and (iii) at least one amino acid has a side chain comprising a guanidine group,

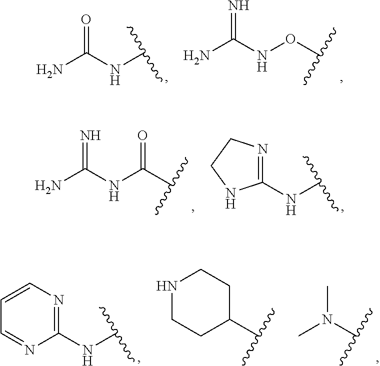

or a protonated form thereof.

The cCPP can comprise from 6 to 20 amino acid residues which form the cCPP, wherein: (i) at least three amino acids can independently be glycine, β-alanine, or 4-aminobutyric acid residues; (ii) at least one amino acid can have a side chain comprising an aromatic or heteroaromatic group; and (iii) at least one amino acid can have a side chain comprising a guanidine group,

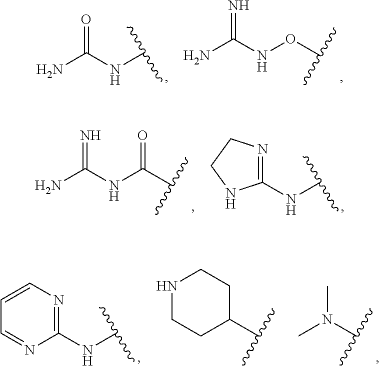

or a protonated form thereof.

### Glycine and Related Amino Acid Residues

The cCPP can comprise (i) 1, 2, 3, 4, 5, or 6 glycine, β-alanine, 4-aminobutyric acid residues, or combinations thereof. The cCPP can comprise (i) 2 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, Q-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise (i) 1, 2, 3, 4, 5, or 6 glycine residues. The cCPP can comprise (i) 2 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues. The cCPP can comprise (i) 2 or 3 glycine residues. The cCPP can comprise (i) 1 or 2 glycine residues.

The cCPP can comprise (i) 3, 4, 5, or 6 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise at least three glycine residues. The cCPP can comprise (i) 3, 4, 5, or 6 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues

In embodiments, none of the glycine, β-alanine, or 4-aminobutyric acid residues in the cCPP are contiguous. Two or three glycine, β-alanine, 4- or aminobutyric acid residues can be contiguous. Two glycine, β-alanine, or 4-aminobutyric acid residues can be contiguous.

In embodiments, none of the glycine residues in the cCPP are contiguous. Each glycine residues in the cCPP can be separated by an amino acid residue that cannot be glycine. Two or three glycine residues can be contiguous. Two glycine residues can be contiguous

Amino Acid Side Chains with an Aromatic or Heteroaromatic Group

The cCPP can comprise (ii) 2, 3, 4, 5 or 6 amino acid residues independently having a side chain comprising an aromatic or heteroaromatic group. The cCPP can comprise (ii) 2 amino acid residues independently having a side chain comprising an aromatic or heteroaromatic group. The cCPP can comprise (ii) 3 amino acid residues independently having a side chain comprising an aromatic or heteroaromatic group. The cCPP can comprise (ii) 4 amino acid residues independently having a side chain comprising an aromatic or heteroaromatic group. The cCPP can comprise (ii) 5 amino acid residues independently having a side chain comprising an aromatic or heteroaromatic group. The cCPP can comprise (ii) 6 amino acid residues independently having a side chain comprising an aromatic or heteroaromatic group. The cCPP can comprise (ii) 2, 3, or 4 amino acid residues independently having a side chain comprising an aromatic or heteroaromatic group. The cCPP can comprise (ii) 2 or 3 amino acid residues independently having a side chain comprising an aromatic or heteroaromatic group.

The cCPP can comprise (ii) 2, 3, 4, 5 or 6 amino acid residues independently having a side chain comprising an aromatic group. The cCPP can comprise (ii) 2 amino acid residues independently having a side chain comprising an aromatic group. The cCPP can comprise (ii) 3 amino acid residues independently having a side chain comprising an aromatic group. The cCPP can comprise (ii) 4 amino acid residues independently having a side chain comprising an aromatic group. The cCPP can comprise (ii) 5 amino acid residues independently having a side chain comprising an aromatic group. The cCPP can comprise (ii) 6 amino acid residues independently having a side chain comprising an aromatic group. The cCPP can comprise (ii) 2, 3, or 4 amino acid residues independently having a side chain comprising an aromatic group. The cCPP can comprise (ii) 2 or 3 amino acid residues independently having a side chain comprising an aromatic group.

The aromatic group can be a 6- to 14-membered aryl. Aryl can be phenyl, naphthyl or anthracenyl, each of which is optionally substituted. Aryl can be phenyl or naphthyl, each of which is optionally substituted. The heteroaromatic group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aromatic or heteroaromatic group can each independently be bis(homonaphthylalanine), homonaphthylalanine, naphthylalanine, phenylglycine, bis(homophenylalanine), homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 341,1′-biphenyl-4-yl)-alanine, 3-(3-benzothienyl)-alanine or tyrosine, each of which is optionally substituted with one or more substituents. The amino acid having a side chain comprising an aromatic or heteroaromatic group can each independently be selected from:

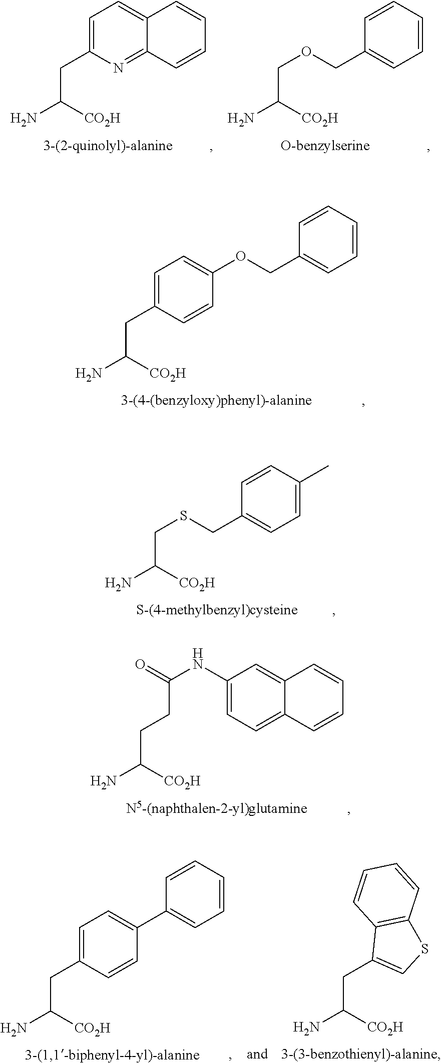

wherein the H on the N-terminus and/or the H on the C-terminus are replaced by a peptide bond.

The amino acid residue having a side chain comprising an aromatic or heteroaromatic group can each be independently a residue of phenylalanine, naphthylalanine, phenylglycine, homophenylalanine, homonaphthylalanine, bis(homophenylalanine), bis-(homonaphthylalanine), tryptophan, or tyrosine, each of which is optionally substituted with one or more substituents. The amino acid residue having a side chain comprising an aromatic group can each independently be a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine. The amino acid residue having a side chain comprising an aromatic group can each independently be a residue of phenylalanine, naphthylalanine, phenylglycine, homophenylalanine, or homonaphthylalanine, each of which is optionally substituted with one or more substituents. The amino acid residue having a side chain comprising an aromatic group can each be independently a residue of phenylalanine, naphthylalanine, homophenylalanine, homonaphthylalanine, bis(homonaphthylalanine), or bis(homonaphthylalanine), each of which is optionally substituted with one or more substituents. The amino acid residue having a side chain comprising an aromatic group can each be independently a residue of phenylalanine or naphthylalanine, each of which is optionally substituted with one or more substituents. At least one amino acid residue having a side chain comprising an aromatic group can be a residue of phenylalanine. At least two amino acid residues having a side chain comprising an aromatic group can be residues of phenylalanine. Each amino acid residue having a side chain comprising an aromatic group can be a residue of phenylalanine.

In embodiments, none of the amino acids having the side chain comprising the aromatic or heteroaromatic group are contiguous. Two amino acids having the side chain comprising the aromatic or heteroaromatic group can be contiguous. Two contiguous amino acids can have opposite stereochemistry. The two contiguous amino acids can have the same stereochemistry. Three amino acids having the side chain comprising the aromatic or heteroaromatic group can be contiguous. Three contiguous amino acids can have the same stereochemistry. Three contiguous amino acids can have alternating stereochemistry.

The amino acid residues comprising aromatic or heteroaromatic groups can be L-amino acids. The amino acid residues comprising aromatic or heteroaromatic groups can be D-amino acids. The amino acid residues comprising aromatic or heteroaromatic groups can be a mixture of D- and L-amino acids.

The optional substituent can be any atom or group which does not significantly reduce (e.g., by more than 50%) the cytosolic delivery efficiency of the cCPP, e.g., compared to an otherwise identical sequence which does not have the substituent. The optional substituent can be a hydrophobic substituent or a hydrophilic substituent. The optional substituent can be a hydrophobic substituent. The substituent can increase the solvent-accessible surface area (as defined herein) of the hydrophobic amino acid. The substituent can be halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

While not wishing to be bound by theory, it is believed that amino acids having an aromatic or heteroaromatic group having higher hydrophobicity values (i.e., amino acids having side chains comprising aromatic or heteroaromatic groups) can improve cytosolic delivery efficiency of a cCPP relative to amino acids having a lower hydrophobicity value. Each hydrophobic amino acid can independently have a hydrophobicity value greater than that of glycine. Each hydrophobic amino acid can independently be a hydrophobic amino acid having a hydrophobicity value greater than that of alanine. Each hydrophobic amino acid can independently have a hydrophobicity value greater or equal to phenylalanine. Hydrophobicity may be measured using hydrophobicity scales known in the art. Table 2 lists hydrophobicity values for various amino acids as reported by Eisenberg and Weiss (Proc. Natl. Acad. Sci. U.S.A 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A 1981; 78(6):3824-3828), and Janin (Nature. 1979; 277(5696):491-492), the entirety of each of which is herein incorporated by reference. Hydrophobicity can be measured using the hydrophobicity scale reported in Engleman, et al.

TABLE 2
Amino Acid Hydrophobicity

| Amino | | Eisenberg | Engleman | Kyrie and | Hoop and | |
|---|---|---|---|---|---|---|
| Acid | Group | and Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gln | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

The size of the aromatic or heteroaromatic groups may be selected to improve cytosolic delivery efficiency of the cCPP. While not wishing to be bound by theory, it is believed that a larger aromatic or heteroaromatic group on the side chain of amino acid may improve cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. The size of the hydrophobic amino acid can be measured in terms of the molecular weight of the hydrophobic amino acid, and the larger hydrophobic amino acid has a side chain with a molecular weight of at least about 90 g/mol, or at least about 130 g/mol, or at least about 141 g/mol. The size of the amino acid can be measured in terms of the SASA of the hydrophobic side chain. The hydrophobic amino acid can have a side chain with a SASA of greater than or equal to alanine, or greater than or equal to glycine. Larger hydrophobic amino acids can have a side chain with a SASA greater than alanine, or greater than glycine. The hydrophobic amino acid can have an aromatic or heteroaromatic group with a SASA greater than or equal to about piperidine-2-carboxylic acid, greater than or equal to about tryptophan, greater than or equal to about phenylalanine, or greater than or equal to about naphthylalanine. A first hydrophobic amino acid (AA_{H1}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². A second hydrophobic amino acid (AA_{H2}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². The side chains of AA_{H1} and AA_{H2} can have a combined SASA of at least about 350 Å², at least about 360 Å², at least about 370 Å², at least about 380 Å², at least about 390 Å², at least about 400 Å², at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². AA_{H2} can be a hydrophobic amino acid residue with a side chain having a SASA that is less than or equal to the SASA of the hydrophobic side chain of AA_{H1}. By way of example, and not by limitation, a cCPP having a Nal-Arg motif may exhibit improved cytosolic delivery efficiency compared to an otherwise identical cCPP having a Phe-Arg motif; a cCPP having a Phe-Nal-Arg motif may exhibit improved cytosolic delivery efficiency compared to an otherwise identical cCPP having a Nal-Phe-Arg motif; and a phe-Nal-Arg motif may exhibit improved cytosolic delivery efficiency compared to an otherwise identical cCPP having a nal-Phe-Arg motif.

As used herein, “hydrophobic surface area” or “SASA” refers to the surface area (reported as square Ångstroms; Å²) of an amino acid side chain that is accessible to a solvent. SASA can be calculated using the ‘rolling ball’ algorithm developed by Shrake & Rupley (J Mol Biol. 79 (2): 351-71), which is herein incorporated by reference in its entirety for all purposes. This algorithm uses a “sphere” of solvent of a particular radius to probe the surface of the molecule. A typical value of the sphere is 1.4 Å, which approximates to the radius of a water molecule.

SASA values for certain side chains are shown below in Table 3. The SASA values described herein are based on the theoretical values listed in Table 3 below, as reported by Tien, et al. (PLOS ONE 8(11): e80635. https://doi.org/10.1371/journal.pone.0080635), which is herein incorporated by reference in its entirety for all purposes.

TABLE 3
Amino Acid SASA Values

| | | | Miller et al. | Rose et al. |
|---|---|---|---|---|
| Residue | Theoretical | Empirical | (1987) | (1985) |
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

### Amino Acid Residues Having a Side Chain Comprising a Guanidine Group, Guanidine Replacement Group, or Protonated Form Thereof

As used herein, guanidine refers to the structure:

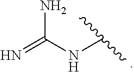 [SMILES: *NC(=N)N]

As used herein, a protonated form of guanidine refers to the structure:

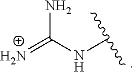 [SMILES: *NC(N)=[NH2+]]

Guanidine replacement groups refer to functional groups on the side chain of amino acids that will be positively charged at or above physiological pH1 or those that can recapitulate the hydrogen bond donating and accepting activity of guanidinium groups.

The guanidine replacement groups facilitate cell penetration and delivery of therapeutic agents while reducing toxicity associated with guanidine groups or protonated forms thereof. The cCPP can comprise at least one amino acid having a side chain comprising a guanidine or guanidinium replacement group. The cCPP can comprise at least two amino acids having a side chain comprising a guanidine or guanidinium replacement group. The cCPP can comprise at least three amino acids having a side chain comprising a guanidine or guanidinium replacement group

The guanidine or guanidinium group can be an isostere of guanidine or guanidinium. The guanidine or guanidinium replacement group can be less basic than guanidine.

As used herein, a guanidine replacement group refers to

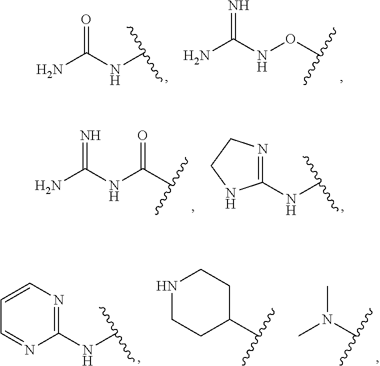

or a protonated form thereof.

The disclosure relates to a cCPP comprising from 4 to 20 amino acids residues, wherein: (i) at least one amino acid has a side chain comprising a guanidine group, or a protonated form thereof; (ii) at least one amino acid residue has no side chain or a side chain comprising

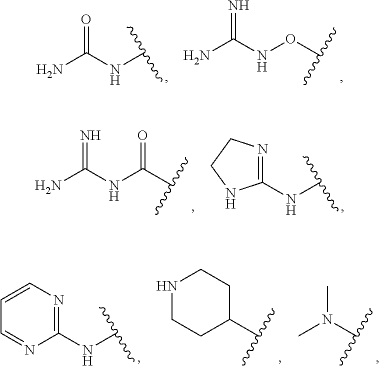

or a protonated form thereof; and (iii) at least two amino acids residues independently have a side chain comprising an aromatic or heteroaromatic group.

At least two amino acids residues can have no side chain or a side chain comprising

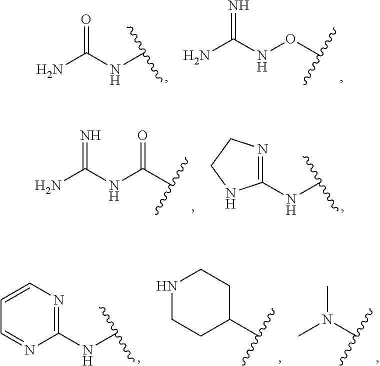

or a protonated form thereof. As used herein, when no side chain is present, the amino acid residue have two hydrogen atoms on the carbon atom(s) (e.g., —CH₂—) linking the amine and carboxylic acid.

The cCPP can comprise at least one amino acid having a side chain comprising one of the following moieties:

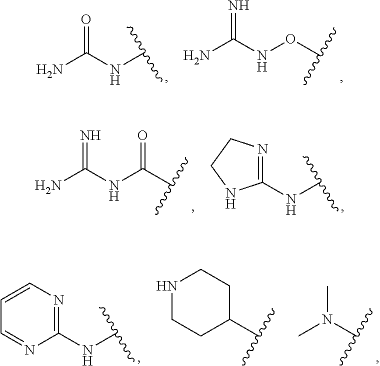

or a protonated form thereof.

The cCPP can comprise at least two amino acids each independently having one of the following moieties:

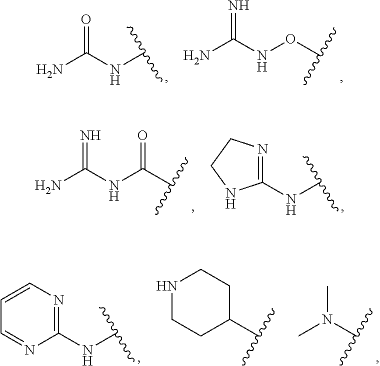

or a protonated form thereof. At least two amino acids can have a side chain comprising the same moiety selected from:

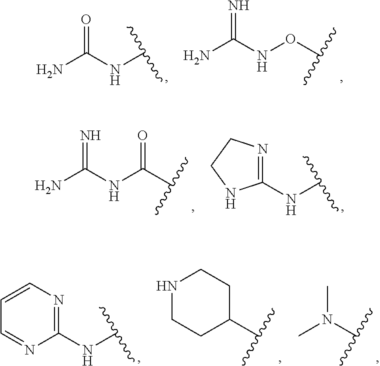

or a protonated form thereof. At least one amino acid can have a side chain comprising

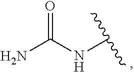 [SMILES: *NC(N)=O]

or a protonated form thereof. At least two amino acids can have a side chain comprising

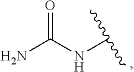 [SMILES: *NC(N)=O]

or a protonated form thereof. One, two, three, or four amino acids can have a side chain comprising

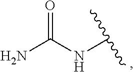 [SMILES: *NC(N)=O]

or a protonated form thereof. One amino acid can have a side chain comprising

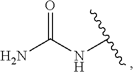 [SMILES: *NC(N)=O]

or a protonated form thereof. Two amino acids can have a side chain comprising

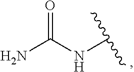 [SMILES: *NC(N)=O]

or a protonated form thereof.

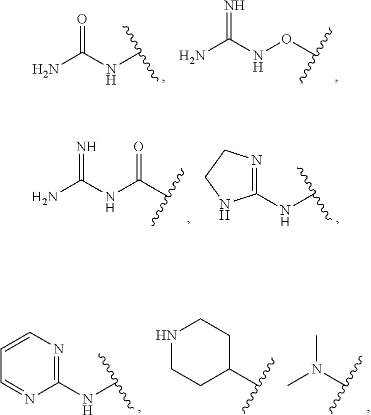

or a protonated form thereof, can be attached to the terminus of the amino acid side chain.

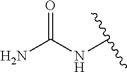 [SMILES: *NC(N)=O]

can be attached to the terminus of the amino acid side chain.

The cCPP can comprise (iii) 2, 3, 4, 5 or 6 amino acid residues independently having a side chain comprising a guanidine group, guanidine replacement group, or a protonated form thereof. The cCPP can comprise (iii) 2 amino acid residues independently having a side chain comprising a guanidine group, guanidine replacement group, or a protonated form thereof. The cCPP can comprise (iii) 3 amino acid residues independently having a side chain comprising a guanidine group, guanidine replacement group, or a protonated form thereof. The cCPP can comprise (iii) 4 amino acid residues independently having a side chain comprising a guanidine group, guanidine replacement group, or a protonated form thereof. The cCPP can comprise (iii) 5 amino acid residues independently having a side chain comprising a guanidine group, guanidine replacement group, or a protonated form thereof. The cCPP can comprise (iii) 6 amino acid residues independently having a side chain comprising a guanidine group, guanidine replacement group, or a protonated form thereof. The cCPP can comprise (iii) 2, 3, 4, or 5 amino acid residues independently having a side chain comprising a guanidine group, guanidine replacement group, or a protonated form thereof. The cCPP can comprise (iii) 2, 3, or 4 amino acid residues independently having a side chain comprising a guanidine group, guanidine replacement group, or a protonated form thereof. The cCPP can comprise (iii) 2 or 3 amino acid residues independently having a side chain comprising a guanidine group, guanidine replacement group, or a protonated form thereof. The cCPP can comprise (iii) at least one amino acid residue having a side chain comprising a guanidine group or protonated form thereof. The cCPP can comprise (iii) two amino acid residues having a side chain comprising a guanidine group or protonated form thereof. The cCPP can comprise (iii) three amino acid residues having a side chain comprising a guanidine group or protonated form thereof.

The amino acid residues can independently have the side chain comprising the guanidine group, guanidine replacement group, or the protonated form thereof that are not contiguous. Two amino acid residues can independently have the side chain comprising the guanidine group, guanidine replacement group, or the protonated form thereof can be contiguous. Three amino acid residues can independently have the side chain comprising the guanidine group, guanidine replacement group, or the protonated form thereof can be contiguous. Four amino acid residues can independently have the side chain comprising the guanidine group, guanidine replacement group, or the protonated form thereof can be contiguous. The contiguous amino acid residues can have the same stereochemistry. The contiguous amino acids can have alternating stereochemistry.

The amino acid residues independently having the side chain comprising the guanidine group, guanidine replacement group, or the protonated form thereof, can be L-amino acids. The amino acid residues independently having the side chain comprising the guanidine group, guanidine replacement group, or the protonated form thereof, can be D-amino acids. The amino acid residues independently having the side chain comprising the guanidine group, guanidine replacement group, or the protonated form thereof, can be a mixture of L- or D-amino acids.

Each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. Each amino acid residue having the side chain comprising the guanidine group, or the protonated form thereof, can independently be a residue of arginine or a protonated form thereof.

Each amino acid having the side chain comprising a guanidine replacement group, or protonated form thereof, can independently be

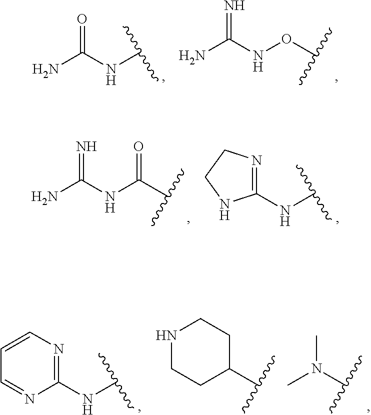

or a protonated form thereof.

Without being bound by theory, it is hypothesized that guanidine replacement groups have reduced basicity, relative to arginine and in some cases are uncharged at physiological pH (e.g., a —N(H)C(O)), and are capable of maintaining the bidentate hydrogen bonding interactions with phospholipids on the plasma membrane that is believed to facilitate effective membrane association and subsequent internalization. The removal of positive charge is also believed to reduce toxicity of the cCPP.

Those skilled in the art will appreciate that the N- and/or C-termini of the above non-natural aromatic hydrophobic amino acids, upon incorporation into the peptides disclosed herein, form amide bonds.

The cCPP can comprise a first amino acid having a side chain comprising an aromatic or heteroaromatic group and a second amino acid having a side chain comprising an aromatic or heteroaromatic group, wherein an N-terminus of a first glycine forms a peptide bond with the first amino acid having the side chain comprising the aromatic or heteroaromatic group, and a C-terminus of the first glycine forms a peptide bond with the second amino acid having the side chain comprising the aromatic or heteroaromatic group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may refer to an amino acid located at the N-terminus of the peptide sequence.

The cCPP can comprise an N-terminus of a second glycine forms a peptide bond with an amino acid having a side chain comprising an aromatic or heteroaromatic group, and a C-terminus of the second glycine forms a peptide bond with an amino acid having a side chain comprising a guanidine group, or a protonated form thereof.

The cCPP can comprise a first amino acid having a side chain comprising a guanidine group, or a protonated form thereof, and a second amino acid having a side chain comprising a guanidine group, or a protonated form thereof, wherein an N-terminus of a third glycine forms a peptide bond with a first amino acid having a side chain comprising a guanidine group, or a protonated form thereof, and a C-terminus of the third glycine forms a peptide bond with a second amino acid having a side chain comprising a guanidine group, or a protonated form thereof.

The cCPP can comprise a residue of asparagine, aspartic acid, glutamine, glutamine acid, or homoglutamine. The cCPP can comprise a residue of asparagine. The cCPP can comprise a residue of glutamine.

The cCPP can comprise a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

While not wishing to be bound by theory, it is believed that the chirality of the amino acids in the cCPPs may impact cytosolic uptake efficiency. The cCPP can comprise at least one D amino acid. The cCPP can comprise one to fifteen D amino acids. The cCPP can comprise one to ten D amino acids. The cCPP can comprise 1, 2, 3, or 4 D amino acids. The cCPP can comprise 2, 3, 4, 5, 6, 7, or 8 contiguous amino acids having alternating D and L chirality. The cCPP can comprise three contiguous amino acids having the same chirality. The cCPP can comprise two contiguous amino acids having the same chirality. At least two of the amino acids can have the opposite chirality. The at least two amino acids having the opposite chirality can be adjacent to each other. At least three amino acids can have alternating stereochemistry relative to each other. The at least three amino acids having the alternating chirality relative to each other can be adjacent to each other. At least four amino acids have alternating stereochemistry relative to each other. The at least four amino acids having the alternating chirality relative to each other can be adjacent to each other. At least two of the amino acids can have the same chirality. At least two amino acids having the same chirality can be adjacent to each other. At least two amino acids have the same chirality and at least two amino acids have the opposite chirality. The at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. The amino acid residues that form the cCPP can all be L-amino acids. The amino acid residues that form the cCPP can all be D-amino acids.

At least two of the amino acids can have a different chirality. At least two amino acids having a different chirality can be adjacent to each other. At least three amino acids can have different chirality relative to an adjacent amino acid. At least four amino acids can have different chirality relative to an adjacent amino acid. At least two amino acids have the same chirality and at least two amino acids have a different chirality. One or more amino acid residues that form the cCPP can be achiral. The cCPP can comprise a motif of 3, 4, or 5 amino acids, wherein two amino acids having the same chirality can be separated by an achiral amino acid. The cCPPs can comprise the following sequences: D-X-D; D-X-D-X; D-X-D-X-D; L-X-L; L-X-L-X; or L-X-L-X-L, wherein X is an achiral amino acid. The achiral amino acid can be glycine.

An amino acid having a side chain comprising:

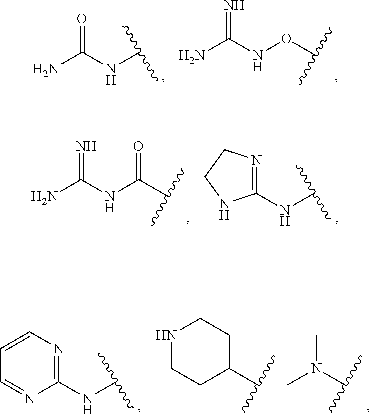

or a protonated form thereof, can be adjacent to an amino acid having a side chain comprising an aromatic or heteroaromatic group. An amino acid having a side chain comprising

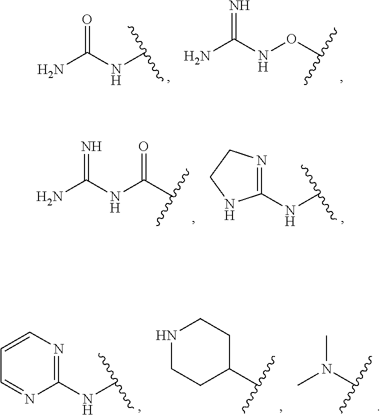

or a protonated form thereof, can be adjacent to at least one amino acid having a side chain comprising a guanidine or protonated form thereof. An amino acid having a side chain comprising a guanidine or protonated form thereof can be adjacent to an amino acid having a side chain comprising an aromatic or heteroaromatic group. Two amino acids having a side chain comprising:

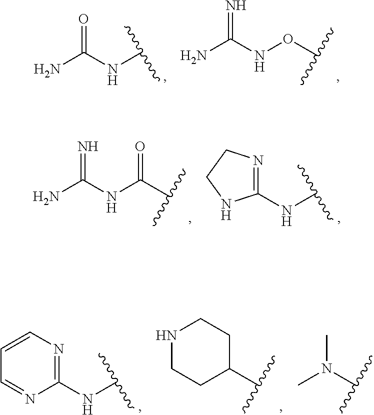

or protonated forms there, can be adjacent to each other. Two amino acids having a side chain comprising a guanidine or protonated form thereof are adjacent to each other. The cCPPs can comprise at least two contiguous amino acids having a side chain can comprise an aromatic or heteroaromatic group and at least two non-adjacent amino acids having a side chain comprising:

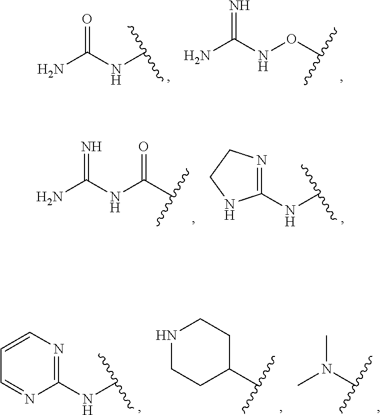

or a protonated form thereof. The cCPPs can comprise at least two contiguous amino acids having a side chain comprising an aromatic or heteroaromatic group and at least two non-adjacent amino acids having a side chain comprising

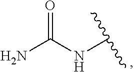 [SMILES: *NC(N)=O]

or a protonated form thereof. The adjacent amino acids can have the same chirality. The adjacent amino acids can have the opposite chirality. Other combinations of amino acids can have any arrangement of D and L amino acids, e.g., any of the sequences described in the preceding paragraph.

At least two amino acids having a side chain comprising:

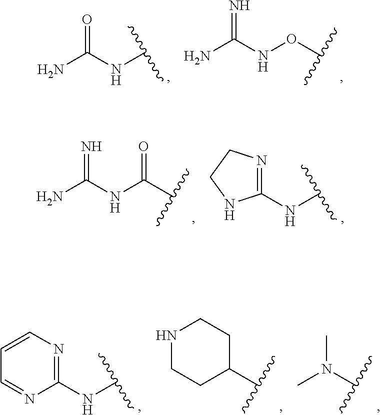

or a protonated form thereof, are alternating with at least two amino acids having a side chain comprising a guanidine group or protonated form thereof.

The cCPP can comprise the structure of Formula (A):

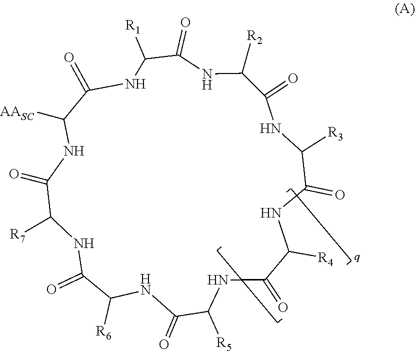

or a protonated form thereof,
wherein:
R₁, R₂, and R₃ are each independently H or an aromatic or heteroaromatic side chain of an amino acid;
   at least one of R₁, R₂, and R₃ is an aromatic or heteroaromatic side chain of an amino acid; R₄, R₅, R₆, R₇ are independently H or an amino acid side chain;
   at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine;
   AA_{SC} is an amino acid side chain; and
   q is 1, 2, 3 or 4;
   wherein the cyclic peptide of Formula (A) is not FfΦRrRrQ (SEQ ID NO: 151).

The cCPP can comprise the structure of Formula (I):

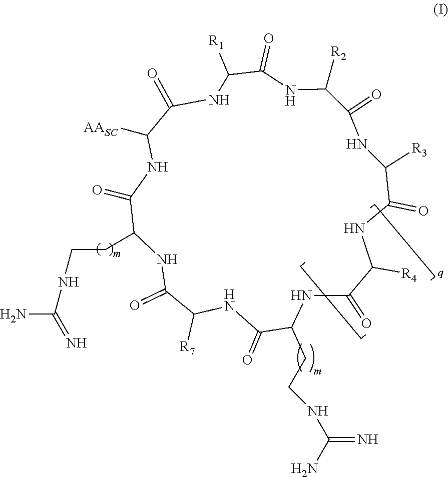

or a protonated form thereof,
wherein:
R₁, R₂, and R₃ can each independently be H or an amino acid residue having a side chain comprising an aromatic group;
   at least one of R₁, R₂, and R₃ is an aromatic or heteroaromatic side chain of an amino acid;
   R₄ and R₇ are independently H or an amino acid side chain;
   AA_{SC} is an amino acid side chain;
   q is 1, 2, 3 or 4; and
   each m is independently an integer 0, 1, 2, or 3.

R₁, R₂, and R₃ can each independently be H, -alkylene-aryl, or -alkylene-heteroaryl. R₁, R₂, and R₃ can each independently be H, —C₁₋₃alkylene-aryl, or —C₁₋₃alkylene-heteroaryl. R₁, R₂, and R₃ can each independently be H or -alkylene-aryl. R₁, R₂, and R₃ can each independently be H or —C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, —C₁₋₃alkylene-Ph or —C₁₋₃alkylene-Naphthyl. R₁, R₂, and R₃ can each independently be H, —CH₂Ph, or —CH₂Naphthyl. R₁, R₂, and R₃ can each independently be H or —CH₂Ph.

R₁, R₂, and R₃ can each independently be the side chain of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

R₁ can be the side chain of tyrosine. R₁ can be the side chain of phenylalanine. Rⱼ can be the side chain of 1-naphthylalanine. R₁ can be the side chain of 2-naphthylalanine. R₁ can be the side chain of tryptophan. R₁, can be the side chain of 3-benzothienylalanine. R₁, can be the side chain of 4-phenylphenylalanine. R₁ can be the side chain of 3,4-difluorophenylalanine. R₁ can be the side chain of 4-trifluoromethylphenylalanine. R₁ can be the side chain of 2,3,4,5,6-pentafluorophenylalanine. R₁ can be the side chain of homophenylalanine. R₁ can be the side chain of 0-homophenylalanine. R₁ can be the side chain of 4-tert-butyl-phenylalanine. R₁ can be the side chain of 4-pyridinylalanine. R₁ can be the side chain of 3-pyridinylalanine. R₁ can be the side chain of 4-methylphenylalanine. R₁ can be the side chain of 4-fluorophenylalanine. R₁ can be the side chain of 4-chlorophenylalanine. R₁ can be the side chain of 3-(9-anthryl)-alanine.

R₂ can be the side chain of tyrosine. R₂ can be the side chain of phenylalanine. R₂ can be the side chain of 1-naphthylalanine. Rⱼ can be the side chain of 2-naphthylalanine. R₂ can be the side chain of tryptophan. R₂ can be the side chain of 3-benzothienylalanine. R₂ can be the side chain of 4-phenylphenylalanine. R₂ can be the side chain of 3,4-difluorophenylalanine. R₂ can be the side chain of 4-trifluoromethylphenylalanine. R₂ can be the side chain of 2,3,4,5,6-pentafluorophenylalanine. R₂ can be the side chain of homophenylalanine. R₂ can be the side chain of β-homophenylalanine. R₂ can be the side chain of 4-tert-butyl-phenylalanine. R₂ can be the side chain of 4-pyridinylalanine. R₂ can be the side chain of 3-pyridinylalanine. R₂ can be the side chain of 4-methylphenylalanine. R₂ can be the side chain of 4-fluorophenylalanine. R₂ can be the side chain of 4-chlorophenylalanine. R₂ can be the side chain of 3-(9-anthryl)-alanine.

R₃ can be the side chain of tyrosine. R₃ can be the side chain of phenylalanine. R; can be the side chain of 1-naphthylalanine. R₃ can be the side chain of 2-naphthylalanine. R₃ can be the side chain of tryptophan. R₃ can be the side chain of 3-benzothienylalanine. R₃ can be the side chain of 4-phenylphenylalanine. R₃ can be the side chain of 3,4-difluorophenylalanine. R₃ can be the side chain of 4-trifluoromethylphenylalanine. R₃ can be the side chain of 2,3,4,5,6-pentafluorophenylalanine. R₃ can be the side chain of homophenylalanine. R₃ can be the side chain of β-homophenylalanine. R₃ can be the side chain of 4-tert-butyl-phenylalanine. R₃ can be the side chain of 4-pyridinylalanine. R₃ can be the side chain of 3-pyridinylalanine. R₃ can be the side chain of 4-methylphenylalanine. R₃ can be the side chain of 4-fluorophenylalanine. R₃ can be the side chain of 4-chlorophenylalanine. R₃ can be the side chain of 3-(9-anthryl)-alanine.

R₄ can be H, -alkylene-aryl, -alkylene-heteroaryl. R₄ can be H, —C₁₋₃alkylene-aryl, or —C₁₋₃alkylene-heteroaryl. R₄ can be H or -alkylene-aryl. R₄ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₄ can be H, —C₁₋₃alkylene-Ph or —C₁₋₃alkylene-Naphthyl. R₄ can be H or the side chain of an amino acid in Table 1 or Table 3. R₄ can be H or an amino acid residue having a side chain comprising an aromatic group. R₄ can be H, —CH₂Ph, or —CH₂Naphthyl. R₄ can be H or —CH₂Ph.

R₅ can be H, -alkylene-aryl, -alkylene-heteroaryl. R₅ can be H, —C₁₋₃alkylene-aryl, or —C₁₋₃alkylene-heteroaryl. R₅ can be H or -alkylene-aryl. R₅ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₅ can be H, —C₁₋₃alkylene-Ph or —C₁₋₃alkylene-Naphthyl. R₅ can be H or the side chain of an amino acid in Table 1 or Table 3. R₄ can be H or an amino acid residue having a side chain comprising an aromatic group. R₅ can be H, —CH₂Ph, or —CH₂Naphthyl. R₄ can be H or —CH₂Ph.

R₆ can be H, -alkylene-aryl, -alkylene-heteroaryl. R₆ can be H, —C₁₋₃alkylene-aryl, or —C₁₋₄alkylene-heteroaryl. R₅ can be H or -alkylene-aryl. R₅ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₆ can be H, —C₁₋₃alkylene-Ph or —C₁₋₃alkylene-Naphthyl. R₆ can be H or the side chain of an amino acid in Table 1 or Table 3. R₅ can be H or an amino acid residue having a side chain comprising an aromatic group. R₆ can be H, —CH₂Ph, or -CH₂Naphthyl. R₆ can be H or —CH₂Ph.

R₇ can be H, -alkylene-aryl, -alkylene-heteroaryl. R₇ can be H, —C₁₋₃alkylene-aryl, or —C₁₋₃alkylene-heteroaryl. R₇ can be H or -alkylene-aryl. R₇ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₇ can be H, —C₁₋₃alkylene-Ph or —C₁₋₃alkylene-Naphthyl. R₇ can be H or the side chain of an amino acid in Table 1 or Table 3. R₇ can be H or an amino acid residue having a side chain comprising an aromatic group. R₇ can be H, —CH₂Ph, or -CH₂Naphthyl. R₇ can be H or —CH₂Ph.

One, two or three of R₁, R₂, R₃, R₄, R₅, R₆, and R₇ can be —CH₂Ph. One of R₁, R₂, R₃, R₄, R₅, R₆, and R₇ can be —CH₂Ph. Two of R₁, R₂, R₃, R₄, R₅, R₆, and R₇ can be —CH₂Ph. Three of R₁, R₂, R₃, R₄, R₅, R₆, and R₇ can be —CH₂Ph. At least one of R₁, R₂, R₃, R₄, R₅, R₆, and R₇ can be —CH₂Ph. No more than four of R₁, R₂, R₃, R₄, R₅, R₆, and R₇ can be —CH₂Ph.

One, two or three of R₁, R₂, R₃, and R₄ are —CH₂Ph. One of R₁, R₂, R₁, and R₄ is —CH₂Ph. Two of R₁, R₂, R₃, and R₄ are —CH₂Ph. Three of R₁, R₂, R₃, and R₄ are —CH₂Ph. At least one of R₁, R₂, R₃, and R₄ is —CH₂Ph.

One, two or three of R₁, R₂, R₃, R₄, R₅, R₆, and R₇ can be H. One of R₁, R₂, R₃, R₄, R₅, R₆, and R₇ can be H. Two of R₁, R₂, R₁, R₄, R₅, R₆, and R₇ are H. Three of R₁, R₂, R₃, R₅, R₆, and R₇ can be H. At least one of R₁, R₂, R₃, R₄, R₅, R₆, and R₇ can be H. No more than three of R₁, R₂, R₃, R₄, R₅, R₆, and R₇ can be —CH₂Ph.

One, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

At least one of R₄, R₅, R₆, and R₇ can be side chain of 3-guanidino-2-aminopropionic acid. At least one of R₄, R₅, R₆, and R₇ can be side chain of 4-guanidino-2-aminobutanoic acid. At least one of R₄, R₅, R₆, and R₇ can be side chain of arginine. At least one of R₄, R₁, R₆, and R₇ can be side chain of homoarginine. At least one of R₄, R₅, R₆, and R₇ can be side chain of N-methylarginine. At least one of R₄, R₃, R₆, and R₇ can be side chain of N,N-dimethylarginine. At least one of R₄, R %, R₆, and R₇ can be side chain of 2,3-diaminopropionic acid. At least one of R₄, R %, R₆, and R₇ can be side chain of 2,4-diaminobutanoic acid, lysine. At least one of R₄, R₅, R₆, and R₇ can be side chain of N-methyllysine. At least one of R₄, R₅, R₆, and R₇ can be side chain of N,N-dimethyllysine. At least one of R₄, R₅, R₆, and R₇ can be side chain of N-ethyllysine. At least one of R₄, R₅, R₆, and R₇ can be side chain of N,N,N-trimethyllysine, 4-guanidinophenylalanine. At least one of R₄, R₅, R₆, and R₇ can be side chain of citrulline. At least one of R₄, R₅, R₆, and R₇ can be side chain of N,N-dimethyllysine, β-homoarginine. At least one of R₄, R₅, R₆, and R₇ can be side chain of 3-(1-piperidinyl)alanine.

At least two of R₄, R₅, R₆, and R₇ can be side chain of 3-guanidino-2-aminopropionic acid. At least two of R₄, R₅, R₆, and R₇ can be side chain of 4-guanidino-2-aminobutanoic acid. At least two of R₄, R₅, R₆, and R₇ can be side chain of arginine. At least two of R₄, R₅, R₆, and R₇ can be side chain of homoarginine. At least two of R₄, R₅, R₆, and R₇ can be side chain of N-methylarginine. At least two of R₄, R₅, R₆, and R₇ can be side chain of N,N-dimethylarginine. At least two of R₄, R₅, R₆, and R₇ can be side chain of 2,3-diaminopropionic acid. At least two of R₄, R₅, R₆, and R₇ can be side chain of 2,4-diaminobutanoic acid, lysine. At least two of R₄, R₅, R₆, and R₇ can be side chain of N-methyllysine. At least two of R₄, R₅, R₆, and R₇ can be side chain of N,N-dimethyllysine. At least two of R₄, R₅, R₆, and R₇ can be side chain of N-ethyllysine. At least two of R₄, R₁, R₆, and R₇ can be side chain of N,N,N-trimethyllysine, 4-guanidinophenylalanine. At least two of R₄, R₅, R₆, and R₇ can be side chain of citrulline. At least two of R₄, R₅, R₆, and R₇ can be side chain of N,N-dimethyllysine, β-homoarginine. At least two of R₄, R₅, R₆, and R₇ can be side chain of 3-(1-piperidinyl)alanine.

At least three of R₄, R₅, R₆, and R₇ can beside chain of 3-guanidino-2-aminopropionic acid. At least three of R₄, R₅, R₆, and R₇ can be side chain of 4-guanidino-2-aminobutanoic acid. At least three of R₄, R₅, R₆, and R₇ can be side chain of arginine. At least three of R₄, R₅, R₆, and R₇ can be side chain of homoarginine. At least three of R₄, R₅, R₆, and R₇ can be side chain of N-methylarginine. At least three of R₄, R₅, R₅, and R₇ can be side chain of N,N-dimethylarginine. At least three of R₄, R₅, R₆, and R₇ can be side chain of 2,3-diaminopropionic acid. At least three of R₄, R₅, R₆, and R₇ can be side chain of 2,4-diaminobutanoic acid, lysine. At least three of R₄, R₅, R₆, and R₇ can be side chain of N-methyllysine. At least three of R₄, R₅, R₆, and R₇ can be side chain of N,N-dimethyllysine. At least three of R₄, R₅, R₆, and R₇ can be side chain of N-ethyllysine. At least three of R₄, R₁, R₆, and R₇ can be side chain of N,N,N-trimethyllysine, 4-guanidinophenylalanine. At least three of R₄, R₅, R₆, and R₇ can be side chain of citrulline. At least three of R₄, R₅, R₆, and R₇ can be side chain of N,N-dimethyllysine, β-homoarginine. At least three of R₄, R₅, R₆, and R₇ can be side chain of 3-(1-piperidinyl)alanine.

AA_{SC} can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to the glutamine residue.

q can be 1, 2, or 3. q can 1 or 2. q can be 1. q can be 2. q can be 3. q can be 4.

m can be 1-3. m can be 1 or 2. m can be 0. m can be 1. m can be 2. m can be 3.

The cCPP of Formula (A) can comprise the structure of Formula (I)

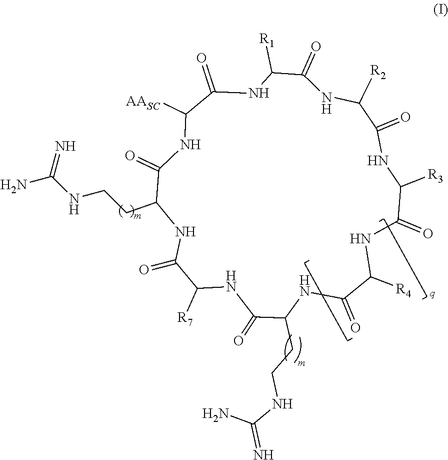

or protonated form thereof, wherein AA_{SC}, R₁, R₂, R₃, R₄, R₇, m and q are as defined herein

The cCPP of Formula (A) can comprise the structure of Formula (I-a) or Formula (I-b):

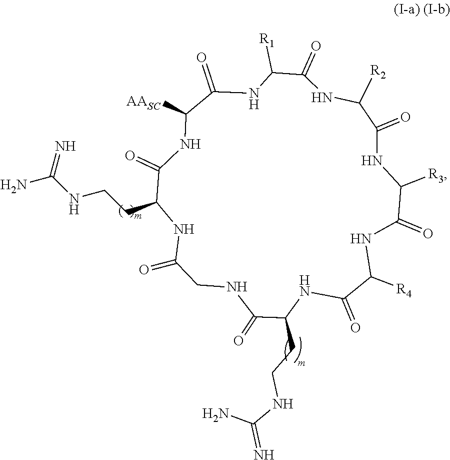

or protonated form thereof, wherein AA_{SC}, R₁, R₂, R₃, R₄, and m are

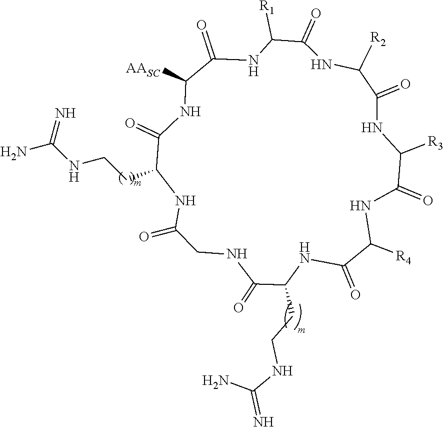

e as defined herein.

The cCPP of Formula (A) can comprise the structure of Formula (I-1), (I-2), (I-3) or (I-4):

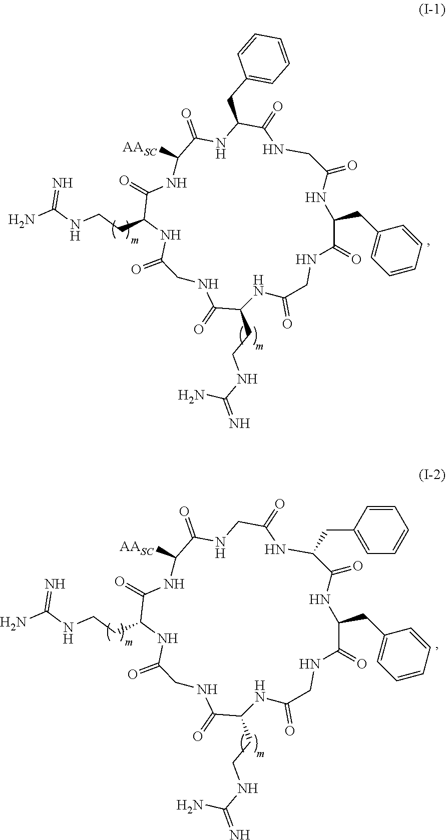

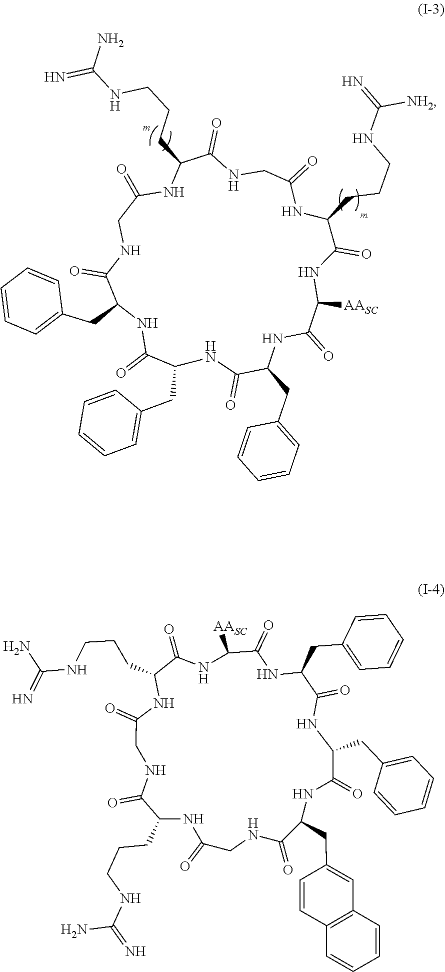

or protonated form thereof, wherein AA_{SC} and m are as defined herein.

The cCPP of Formula A can corn rise the structure of Formula I-5 or I-6:

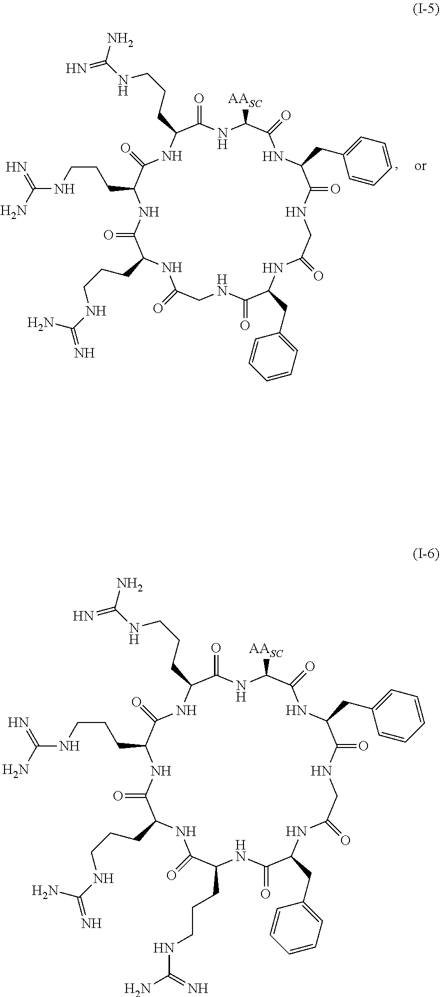

or protonated form thereof, wherein AA_{SC} is as defined herein.

The cCPP of Formula (A) can comprise the structure of Formula (I-1)

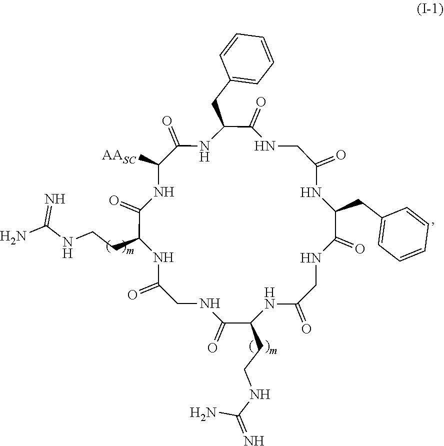

or a protonated form thereof, wherein AA_{SC} and m are as defined herein.

The cCPP of Formula (A) can comprise the structure of Formula (I-2):

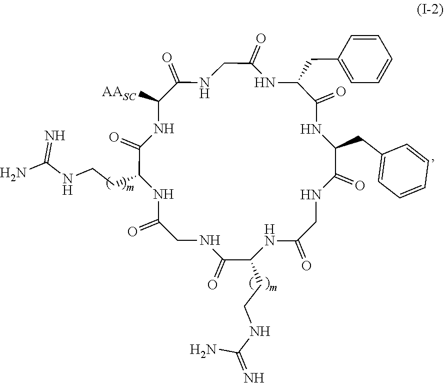

or a protonated form thereof, wherein AA_{SC} and m are as defined herein.

The cCPP of Formula (A) can comprise the structure of Formula (I-3):

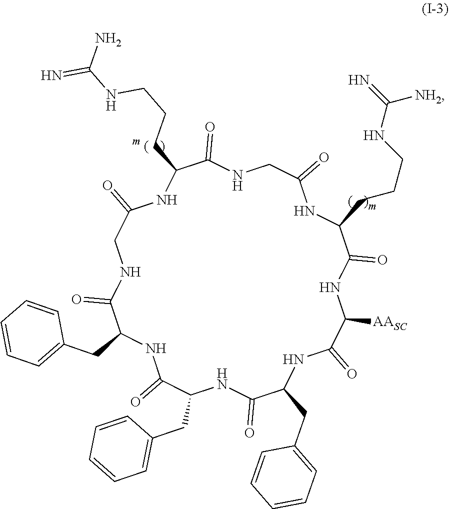

or a protonated form thereof, wherein AA_{SC} and m are as defined herein.

The cCPP of Formula (A) can comprise the structure of Formula (I4):

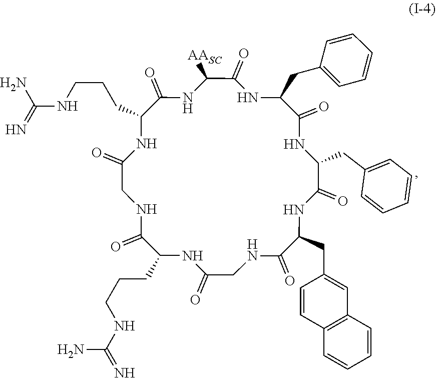

or a protonated form thereof, wherein AA_{SC} and m are as defined herein.

The cCPP of Formula (A) can comprise the structure of Formula (I-5):

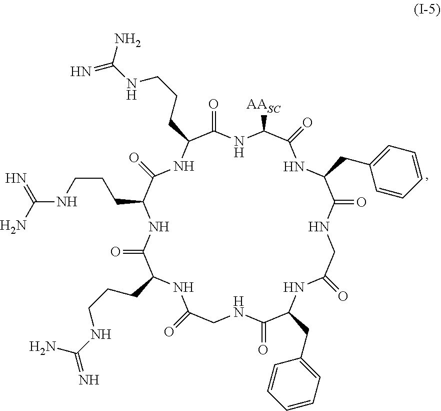

or a protonated form thereof, wherein AA_{SC} and m are as defined herein.

The cCPP of Formula (A) can comprise the structure of Formula (I-6):

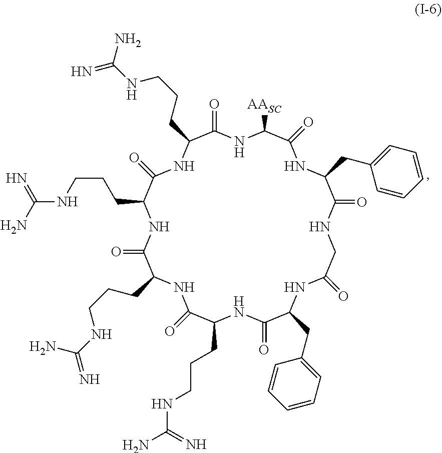

or a protonated form thereof, wherein AA_{SC} and m are as defined herein.

The cCPP can comprise one of the following sequences: FGFGRGR (SEQ ID NO: 126); GfFGrGr (SEQ ID NO: 230), FfΦGRGR (SEQ ID NO: 231); FfFGRGR (SEQ ID NO: 232); or FfΦGrGr (SEQ ID NO: 231). The cCPP can have one of the following sequences: FGFCD; GfFGrGrQ (SEQ ID NO: 233), FfΦGRGRQ (SEQ ID NO: 234); FfFGRGRQ; (SEQ ID NO: 235) or FfΦGrGrQ (SEQ ID NO: 234).

The disclosure also relates to a cCPP having the structure of Formula (II):

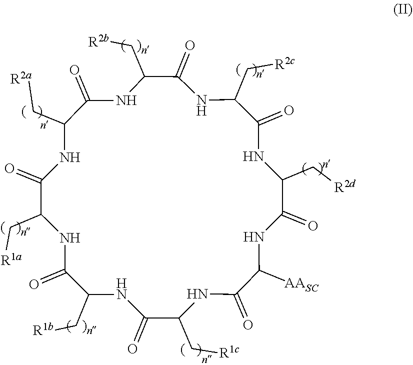

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is

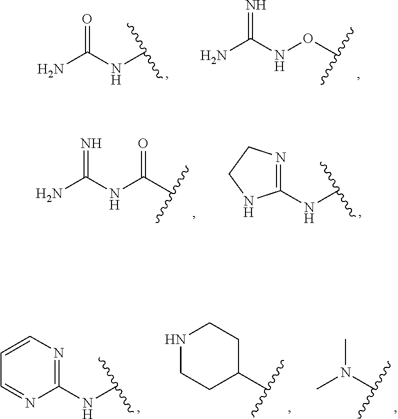

or a protonated form thereof;
at least one of R^{2a}, R^{2b}, R^{2c} and R^{2d} is guanidine or a protonated form thereof;
each n″ is independently an integer 0, 1, 2, 3, 4, or 5;
each n′ is independently an integer from 0, 1, 2, or 3; and
if n′ is 0 then R^{2a}, R^{2b}, R^{2c} or R^{2d} is absent.

At least two of R^{2a}, R^{2b}, R^{2c} and R^{2d} can be

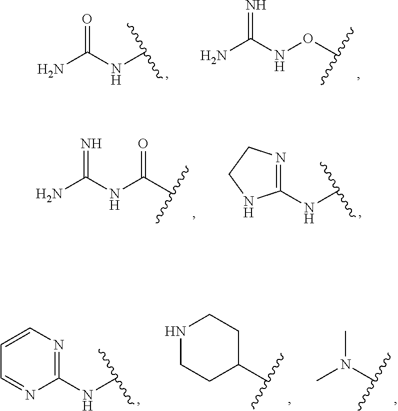

or a protonated form thereof. Two or three of R^{2a}, R^{2b}, R^{2c} and R^{2d} can be

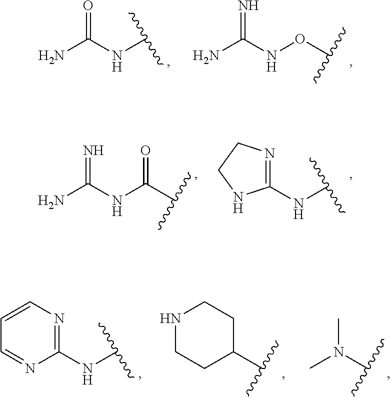

or a protonated form thereof. One of R^{2a}, R^{2b}, R^{2c} and R^{2d} can be

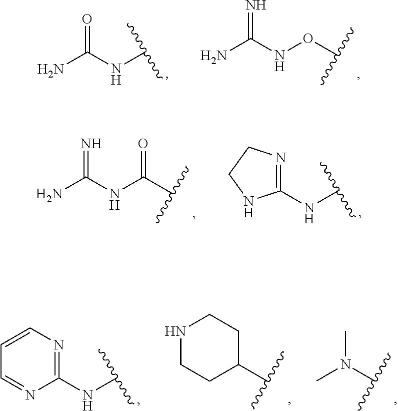

a protonated form thereof. At least one of R^{2a}, R^{2b}, R^{2c} and R^{2d} can be

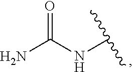 [SMILES: *NC(N)=O]

or a protonated form thereof, and the remaining of R^{2a}, R^{2b}, R^{2c} and R^{2d} can be guanidine or a protonated form thereof. At least two of R^{2a}, R^{2b}, R^{2c} and R^{2d} can be

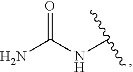 [SMILES: *NC(N)=O]

or a protonated form thereof, and the remaining of R^{2a}, R^{2b}, R^{2c} and R^{2d} can be guanidine, or a protonated form thereof.

All of R^{2a}, R^{2b}, R^{2c} and R^{2d} can be

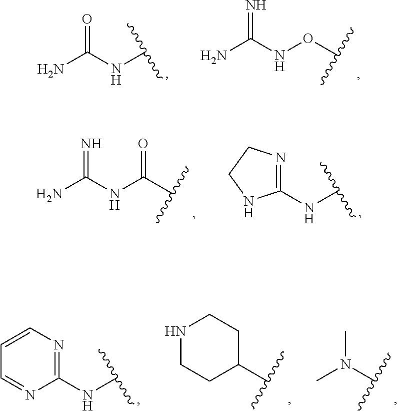

or a protonated form thereof. At least of R^{2a}, R^{2b}, R^{2c} and R^{2d} can be

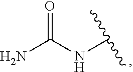 [SMILES: *NC(N)=O]

or a protonated form thereof, and the remaining of R^{2a}, R^{2b}, R^{2c} and R^{2d} can be guaninide or a protonated form thereof. At least two R^{2a}, R^{2b}, R^{2c} and R^{2d} groups can be

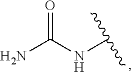 [SMILES: *NC(N)=O]

or a protonated form thereof, and the remaining of R^{2a}, R^{2b}, R^{2c} and R^{2d} are guanidine, or a protonated form thereof.

Each of R^{2a}, R^{2b}, R^{2c} and R^{2d} can independently be 2,3-diaminopropionic acid, 2,4-diaminobutyric acid, the side chains of ornithine, lysine, methyllysine, dimethyllysine, trimethyllysine, homo-lysine, serine, homo-serine, threonine, allo-threonine, histidine, 1-methylhistidine, 2-aminobutanedioic acid, aspartic acid, glutamic acid, or homo-glutamic acid.

AA_{SC} can be

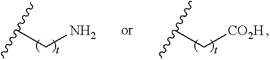

wherein t can be an integer from 0 to 5. AA_{SC} can be

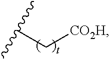

wherein t can be an integer from 0 to 5. t can be 1 to 5. t is 2 or 3. t can be 2. t can be.

R^{1a}, R^{1b}, and R^{1c} can each independently be 6- to 14-membered aryl. R^{1a}, R^{1b}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1b}, and R^{1c} can each be independently selected pyridyl, quinolyl, or isoquinolyl.

Each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

Each n″ can independently be an integer from 1 to 3. Each n″ can independently be 2 or 3. Each n″ can be 2. Each n″ can be 3. At least one n″ can be 0. At least one n″ can be 1. At least one n″ can be 2. At least one n″ can be 3.

Each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′ is 3.

The cCPP of Formula (II) can have the structure of Formula (II-1):

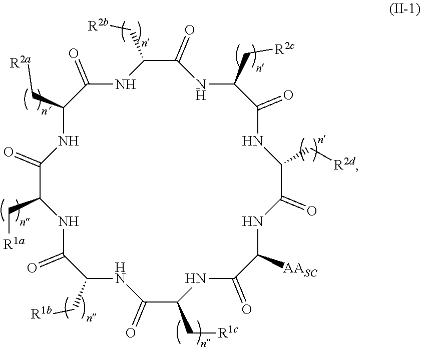

wherein R^{1a}, R^{1b}, R^{1c}, R^{2c}, R^{2b}, R^{2c}, R^{2d}, AA_{SC}, n′ and n″ are as defined herein.

The cCPP of Formula (II) can have the structure of Formula (IIa):

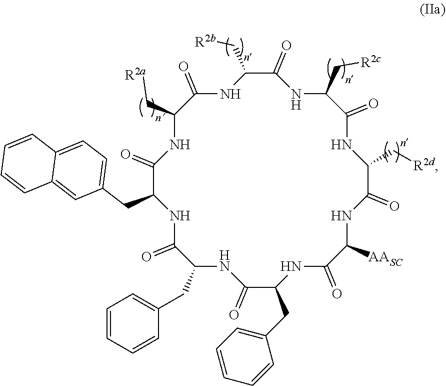

wherein R^{1a}, R^{1b}, R^{1c}, R^{2a}, R^{2b}, R^{2c}, R^{2d}, AA_{SC} and n′ are as defined herein.

The cCPP of formula (II) can have the structure of Formula (IIb):

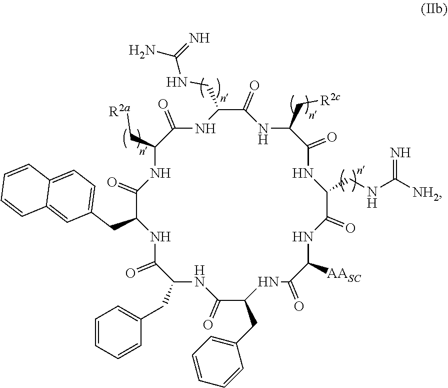

wherein R^{2a}, R^{2b}, AA_{SC}, and n′ are as defined herein.

The cCPP can have the structure of Formula (IIb):

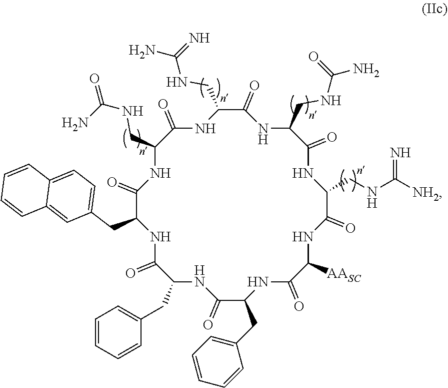

or a protonated form thereof,
wherein:
AA_{SC} and n′ are as defined herein.

The cCPP of Formula (IIa) has one of the following structures:

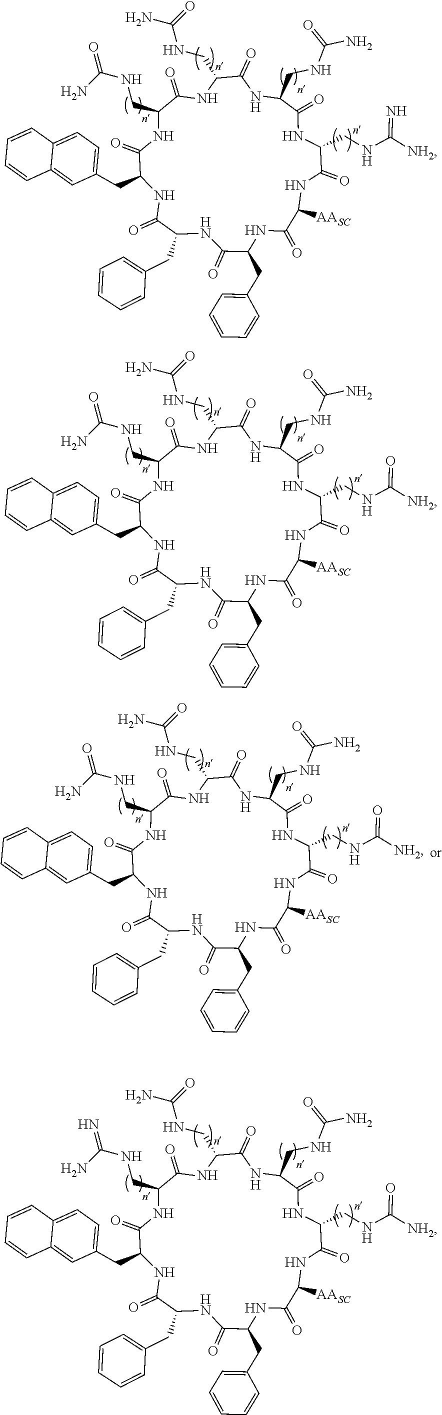

wherein AA_{SC} and n are as defined herein.

The cCPP of Formula Ha has one of the following structures:

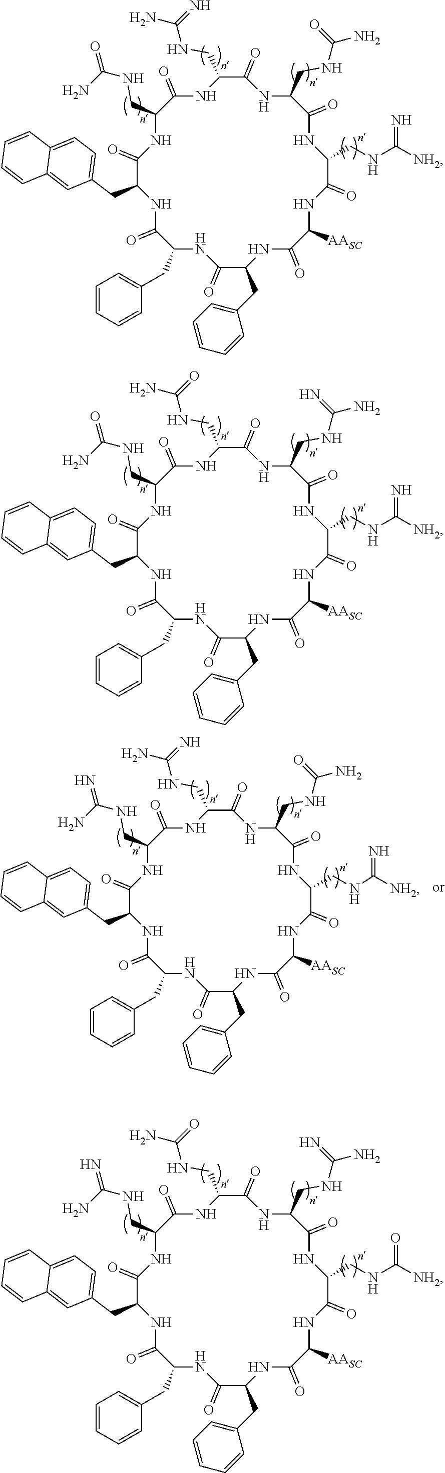

wherein AA_{SC} and n are as defined herein

The cCPP of Formula (IIa) has one of the following structures:

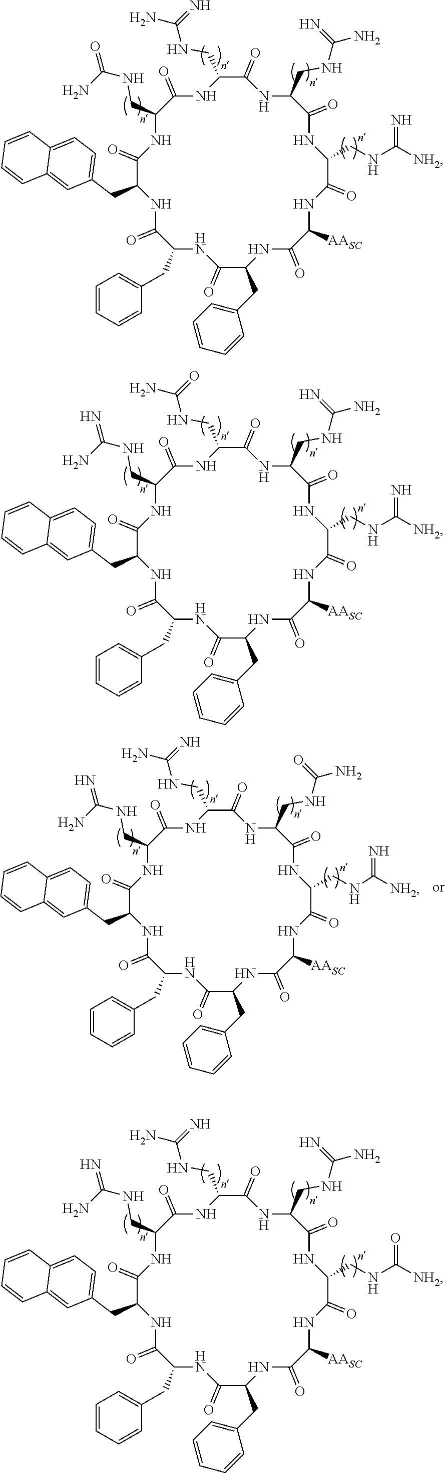

wherein AA_{SC} and n are as defined herein.

The cCPP of Formula (II) can have the structure:

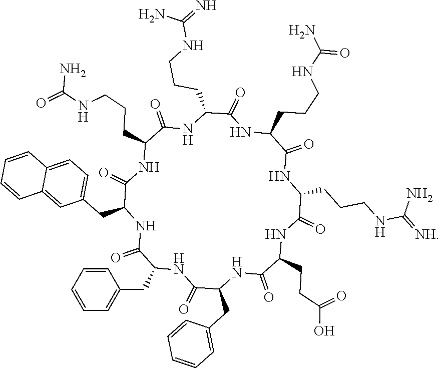 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP of Formula (II) can have the structure:

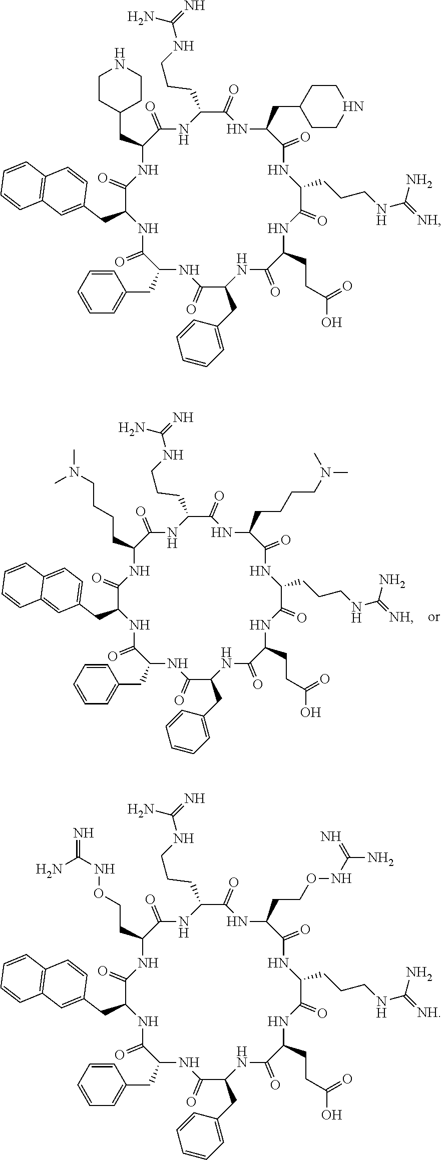

The cCPP can have the structure of Formula (III):

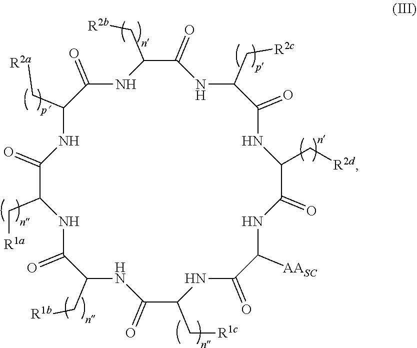

wherein:
AA_{SC} is an amino acid side chain;
R^{1a}, R^{1b}, and R^{1c} are each independently a 6- to 14-membered aryl or a 6- to 14-membered heteroaryl;
R^{2a} and R^{2c} are each independently H,

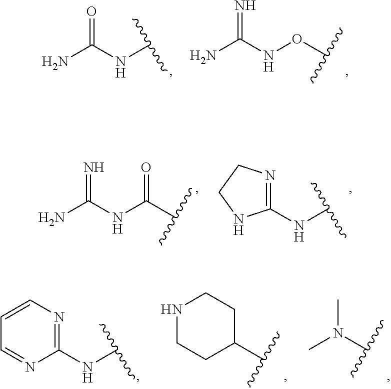

or a protonated form thereof;
R^{2b} and R^{2d} are each independently guanidine or a protonated form thereof;
each n″ is independently an integer from 1 to 3;
each n′ is independently an integer from 1 to 5; and
each p′ is independently an integer from 0 to 5.

The cCPP of Formula (III) can have the structure of Formula (III-1):

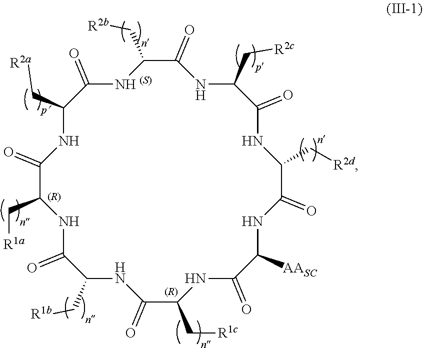

wherein:
AA_{SC}, R^{2a}, R^{2b}, R^{2c}, R^{2d}, n′, n″, and p′ are as defined herein.

The cCPP of Formula (III) can have the structure of Formula (IIIa):

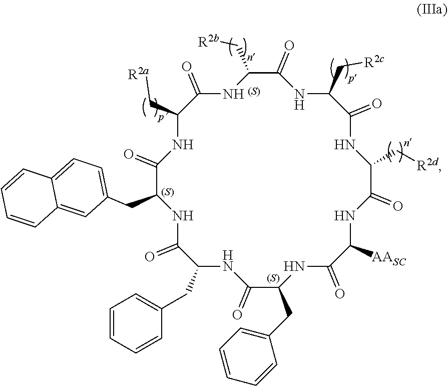

wherein:
AA_{SC}, R^{2a}, R^{2c}, R^{2b}, R^{2d} n′, n″, and p′ are as defined herein.

In Formulas (III), (III-1), and (IIIa), R^{a} and R^{c} can be H. R^{a} and R^{c} can be H and R^{b} and R^{d} can each independently be guanidine or protonated form thereof. R^{a} can be H. R^{b} can be H. p′ can be 0. R^{a} and R^{c} can be H and each p′ can be 0.

In Formulas (III), (III-1), and (IIa), R₄ and R^{c} can be H, R^{b} and R^{d} can each independently be guanidine or protonated form thereof, n″ can be 2 or 3, and each p′ can be 0.

p′ can 0. p′ can 1. p′ can 2. p′ can 3. p′ can 4. p′ can be 5.

The cCPP can have the structure:

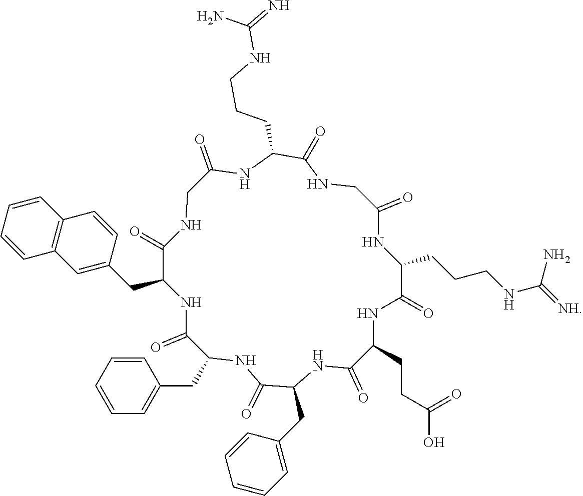 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]

The cCPP of Formula (A) can be selected from:

CPP Sequence

(FfΦRrRrQ)
(SEQ ID NO: 151)
(Ff(Cit-r-Cit-rQ)
(SEQ ID NO: 236)
(FfΦGrGrQ)
(SEQ ID NO: 234)
FfFGRGRQ
(SEQ ID NO: 235)
(FGFGRGRQ)
(SEQ ID NO: 128)
(GfFGrGrQ)
(SEQ ID NO: 233)
(FGFGRRRQ)
(SEQ ID NO: 130)
or
(FGFRRRRQ)
(SEQ ID NO: 129)

The cCPP of Formula (A) can be selected from:

CPP Sequence

FΦRRRRQ
(SEQ ID NO: 240)
fΦRrRrQ
(SEQ ID NO: 131)
FfΦRrRrQ
(SEQ ID NO: 151)
FfΦCit-r-Cit-rQ
(SEQ ID NO: 236)
FfΦGrGrQ
(SEQ ID NO: 234)
FfΦRGRGQ
(SEQ ID NO: 241)
FIFGRGRQ
(SEQ ID NO: 235)
FGFGRGRQ
(SEQ ID NO: 128)
GfFGrGrQ
(SEQ ID NO: 233)
FGFGRRRQ
(SEQ ID NO: 130)
or
FGFRRRRQ
(SEQ ID NO: 129)

AA_{SC} can be conjugated to a linker.

### Linker

The cCPP of the disclosure can be conjugated to a linker. The linker can link a cargo to the cCPP. The linker can be attached to the side chain of an amino acid of the cCPP, and the cargo can be attached at a suitable position on linker.

The linker can be any appropriate moiety which can conjugate a cCPP to one or more additional moieties, e.g., an exocyclic peptide (EP) and/or a cargo. Prior to conjugation to the cCPP and one or more additional moieties, the linker has two or more functional groups, each of which are independently capable of forming a covalent bond to the cCPP and one or more additional moieties. If the cargo is an oligonucleotide, the linker can be covalently bound to the 5′ end of the cargo or the 3′ end of the cargo. The linker can be covalently bound to the 5′ end of the cargo. The linker can be covalently bound to the 3′ end of the cargo. If the cargo is a peptide, the linker can be covalently bound to the N-terminus or the C-terminus of the cargo. The linker can be covalently bound to the backbone of the oligonucleotide or peptide cargo. The linker can be any appropriate moiety which conjugates a cCPP described herein to a cargo such as an oligonucleotide, peptide or small molecule.

The linker can comprise hydrocarbon linker.

The linker can comprise a cleavage site. The cleavage site can be a disulfide, or caspase-cleavage site (e.g, Val-Cit-PABC).

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more —(R¹-J-R²)z″- subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R³ is independently selected from H, alkyl, alkenyl, alkynyl, carbocyclyl, and heterocyclyl, each of which is optionally substituted, and z″ is an integer from 1 to 50; (viii) —(R¹-J)z″- or -(J-R¹)z″-, wherein each of R¹, at each instance, is independently alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ is an integer from 1 to 50; or (ix) the linker can comprise one or more of (i) through (x).

The linker can comprise one or more D or L amino acids and/or —(R¹-J-R²)z″-, wherein each of R¹ and R², at each instance, are independently alkylene, each J is independently C, NR³, —NR³C(O)—, S, and O, wherein R⁴ is independently selected from H and alkyl, and z″ is an integer from 1 to 50; or combinations thereof.

The linker can comprise a —(OCH₂CH₂)_{z′}— (e.g., as a spacer), wherein z′ is an integer from 1 to 23, e.g., 2, 3, 4, 5, 6, 7, 8, 9, 10, 11, 12, 13, 14, 15, 16, 17, 18, 19, 20, 21, 22, or 23. “—(OCH₂CH₂) z′ can also be referred to as polyethylene glycol (PEG).

The linker can comprise one or more amino acids. The linker can comprise a peptide. The linker can comprise a —(OCH₂CH₂)_{z′}—, wherein z′ is an integer from 1 to 23, and a peptide. The peptide can comprise from 2 to 10 amino acids. The linker can further comprise a functional group (FG) capable of reacting through click chemistry. FG can be an azide or alkyne, and a triazole is formed when the cargo is conjugated to the linker.

The linker can comprises (i) a β alanine residue and lysine residue; (ii) -(J-R¹)z″; or (iii) a combination thereof. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O.

The linker can comprise (i) residues of β-alanine, glycine, lysine, 4-aminobutyric acid, 5-aminopentanoic acid, 6-aminohexanoic acid or combinations thereof; and (ii) —(R¹-J)z″- or -(J-R¹)z″. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O. The linker can comprise glycine, beta-alanine, 4-aminobutyric acid, 5-aminopentanoic acid, 6-aminohexanoic acid, or a combination thereof.

The linker can be a trivalent linker. The linker can have the structure:

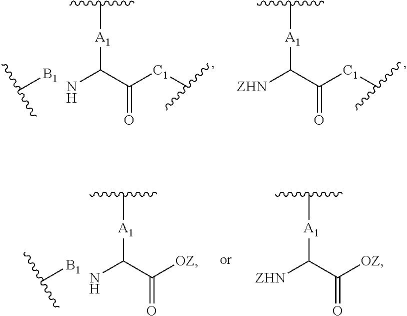

wherein A₁, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂—(CH₂O)ₙ—S—S—(CH₂O)ₙ—COOH], or caspase-cleavage site (Val-Cit-PABC).

The hydrocarbon can be a residue of glycine or beta-alanine.

The linker can be bivalent and link the cCPP to a cargo. The linker can be bivalent and link the cCPP to an exocyclic peptide (EP).

The linker can be trivalent and link the cCPP to a cargo and to an EP.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)—, or a combination thereof.

The linker can have the structure:

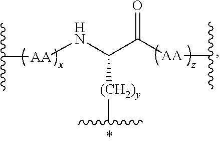

wherein: each AA is independently an amino acid residue; * is the point of attachment to the AA_{SC}, and AA_{SC} is side chain of an amino acid residue of the cCPP; x is an integer from 1-10; y is an integer from 1-5; and z is an integer from 1-10. x can be an integer from 1-5. x can be an integer from 1-3. x can be 1. y can be an integer from 2-4. y can be 4. z can be an integer from 1-5. z can be an integer from 1-3. z can be 1. Each AA can independently be selected from glycine, β-alanine, 4-aminobutyric acid, 5-aminopentanoic acid, and 6-aminohexanoic acid.

The cCPP can be attached to the cargo through a linker (“L”). The linker can be conjugated to the cargo through a bonding group (“M”).

The linker can have the structure:

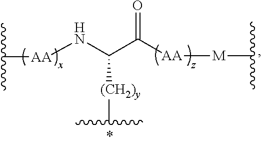

wherein: x is an integer from 1-10; y is an integer from 1-5; z is an integer from 1-10; each AA is independently an amino acid residue; * is the point of attachment to the AA_{SC}, and AA_{SC} is side chain of an amino acid residue of the cCPP; and M is a bonding group defined herein.

The linker can have the structure:

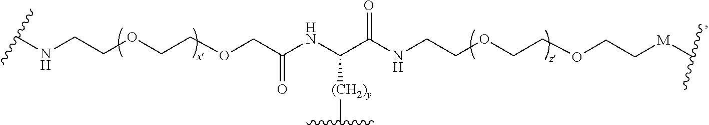

wherein: x′ is an integer from 1-23; y is an integer from 1-5; z′ is an integer from 1-23; * is the point of attachment to the AA_{SC}, and AA_{SC} is a side chain of an amino acid residue of the cCPP; and M is a bonding group defined herein.

The linker can have the structure:

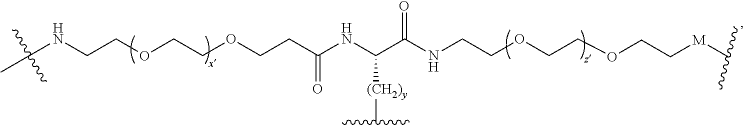

wherein: x′ is an integer from 1-23; y is an integer from 1-5; z′ is an integer from 1-23; * is the point of attachment to the AA_{SC}, and AA_{SC} is a side chain of an amino acid residue of the cCPP; and M is a bonding group defined herein.

The linker can have the structure:

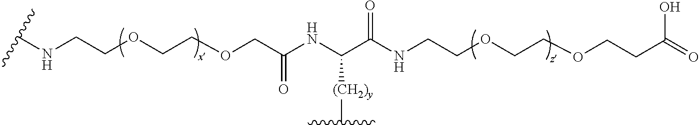

wherein: x′ is an integer from 1-23; y is an integer from 1-5; and z′ is an integer from 1-23; * is the point of attachment to the AA_{SC}, and AA_{SC} is a side chain of an amino acid residue of the cCPP.

x can be an integer from 1-10, e.g., 1, 2, 3, 4, 5, 6, 7, 8, 9, or 10, inclusive of all ranges and subranges therebetween.

x′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. x′ can be an integer from 5-15. x′ can be an integer from 9-13. x′ can be an integer from 1-5. x′ can be 1.

y can be an integer from 1-5, e.g., 1, 2, 3, 4, or 5, inclusive of all ranges and subranges therebetween. y can be an integer from 2-5. y can be an integer from 3-5. y can be 3 or 4. y can be 4 or 5. y can be 3. y can be 4. y can be 5.

z can be an integer from 1-10, e.g., 1, 2, 3, 4, 5, 6, 7, 8, 9, or 10, inclusive of all ranges and subranges therebetween.

z′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. z′ can be an integer from 5-15. z′ can be an integer from 9-13. z′ can be 11.

As discussed above, the linker or M (wherein M is part of the linker) can be covalently bound to cargo at any suitable location on the cargo. The linker or M (wherein M is part of the linker) can be covalently bound to the 3′ end of oligonucleotide cargo or the 5′ end of an oligonucleotide cargo. The linker or M (wherein M is part of the linker) can be covalently bound to the N-terminus or the C-terminus of a peptide cargo. The linker or M (wherein M is part of the linker) can be covalently bound to the backbone of an oligonucleotide or a peptide cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can be bound to the side chain of aspartic acid, glutamic acid, glutamine, asparagine, or lysine, or a modified side chain of glutamine or asparagine (e.g., a reduced side chain having an amino group), on a peptide cargo. The linker can be bound to the side chain of lysine on the peptide cargo.

The linker can have a structure:

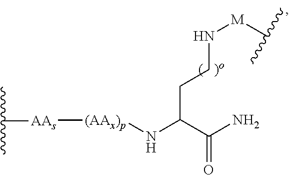

wherein
M is a group that conjugates L to a cargo, for example, an oligonucleotide;
AAₛ is a side chain or terminus of an amino acid on the cCPP;
each AAₓ is independently an amino acid residue;
o is an integer from 0 to 10; and
p is an integer from 0 to 5.

The linker can have a structure:

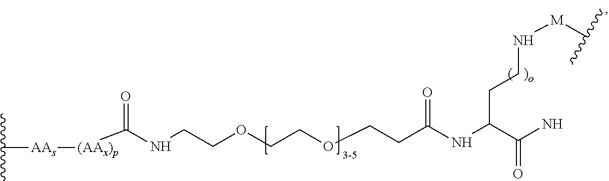

wherein
M is a group that conjugates L to a cargo, for example, an oligonucleotide;
AAₛ is a side chain or terminus of an amino acid on the cCPP;
each AAₓ is independently an amino acid residue;
o is an integer from 0 to 10; and
p is an integer from 0 to 5.

M can comprise an alkylene, alkenylene, alkynylene, carbocyclyl, or heterocyclyl, each of which is optionally substituted. M can be selected from:

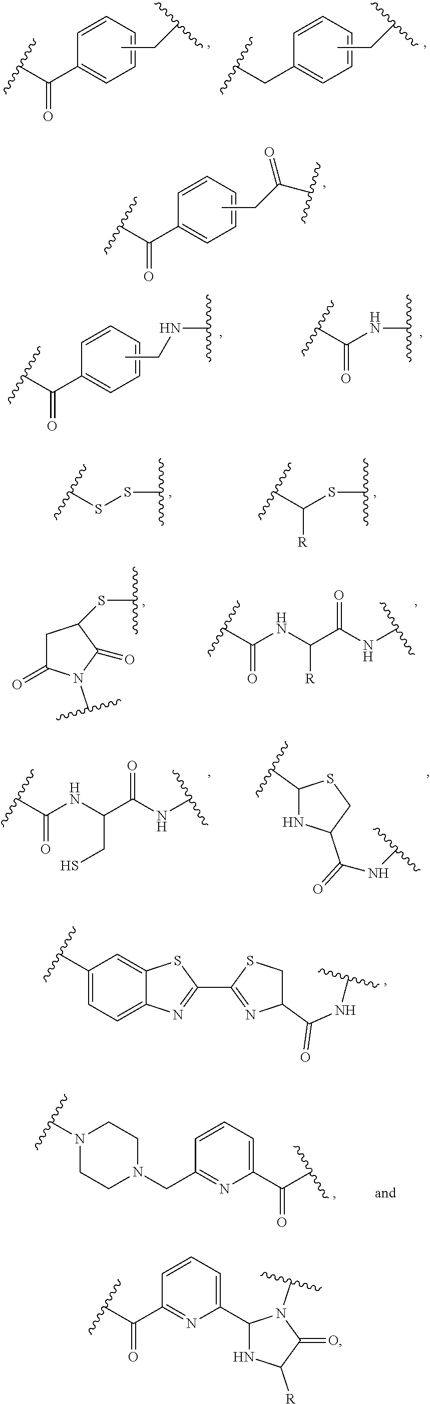

wherein R is alkyl, alkenyl, alkynyl, carbocyclyl, or heterocyclyl.

M can be selected from:

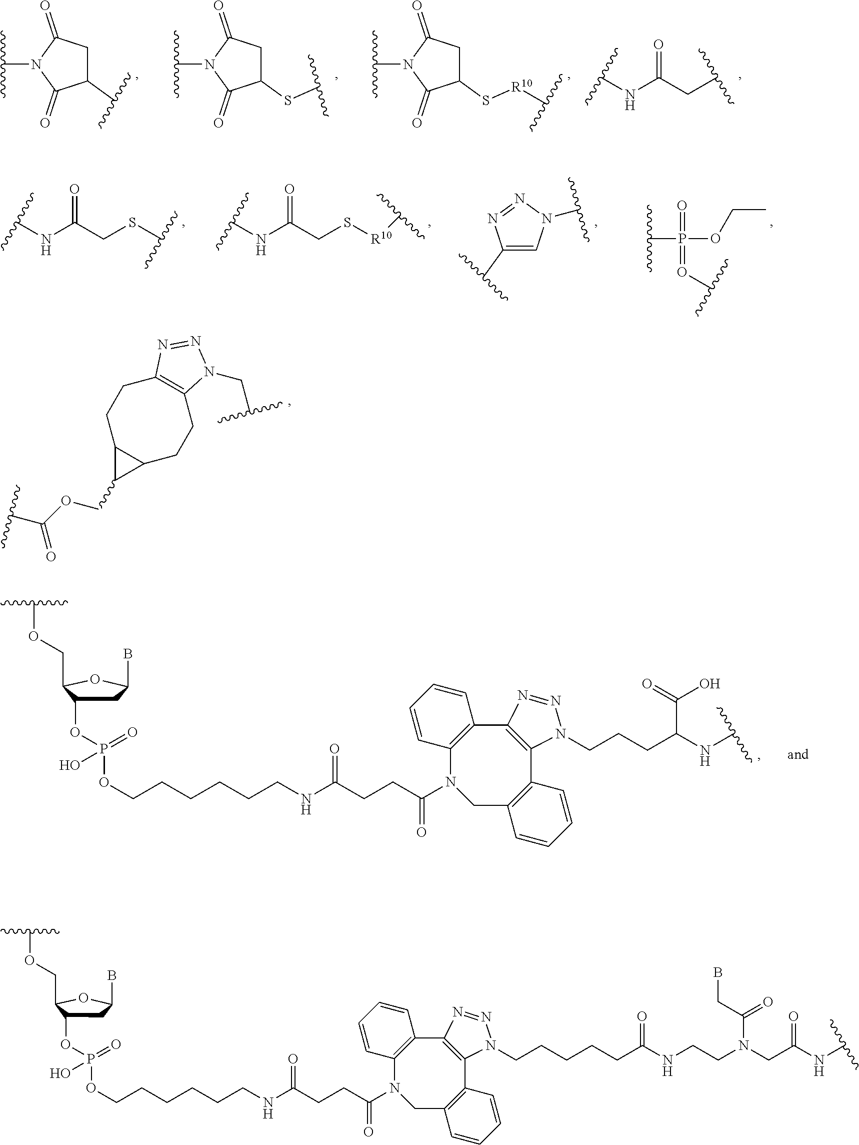

wherein: R¹⁰ is alkylene, cycloalkyl, or

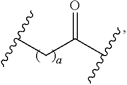

wherein a is 0 to 10.

M can be

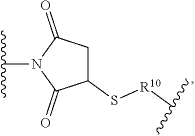

### R¹⁰ can be

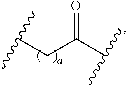

### and a is 0 to 10. M can be

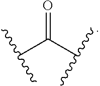 [SMILES: *C(*)=O]

M can be a heterobifunctional crosslinker, e.g.,

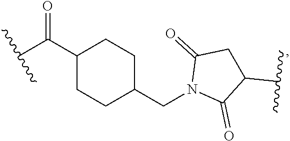 [SMILES: *C(=O)C1CCC(CN2C(=O)CC(*)C2=O)CC1]

which is disclosed in Williams et al. Curr. Protoc Nucleic Acid Chem. 2010, 42, 4.41.1-4.41.20, incorporated herein by reference its entirety.

M can be —C(O)—.

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be an AA_{SC} as defined herein.

Each AAₓ is independently a natural or non-natural amino acid. One or more AAₓ can be a natural amino acid. One or more AAₓ can be a non-natural amino acid. One or more AAₓ can be a β-amino acid. The β-amino acid can be β-alanine.

o can be an integer from 0 to 10, e.g., 0, 1, 2, 3, 4, 5, 6, 7, 8, 9, and 10. o can be 0, 1, 2, or 3. o can be 0. o can be 1. o can be 2. o can be 3.

p can be 0 to 5, e.g., 0, 1, 2, 3, 4, or 5. p can be 0. p can be 1. p can be 2. p can be 3. p can be 4. p can be 5.

The linker can have the structure:

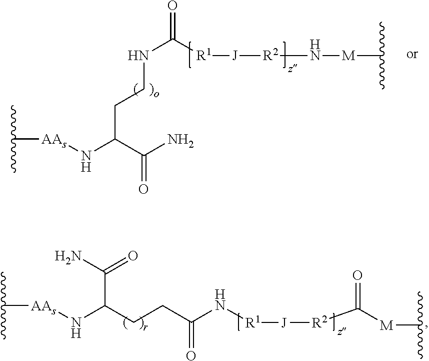

wherein M, AAₛ, each —(R¹-J-R²)z″-, o and z″ are defined herein; r can be 0 or 1.

r can be 0. r can be 1.

The linker can have the structure:

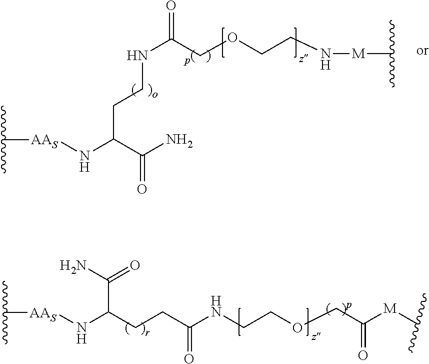

wherein each of M, AAₛ, o, p, q, r and z″ can be as defined herein.

z″ can bean integer from 1 to 50, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, inclusive of all ranges and values therebetween. z″ can be an integer from 5-20. z″ can be an integer from 10-15.

The linker can have the structure:

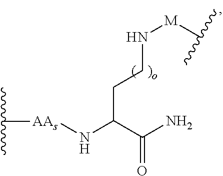

wherein:
M, AAₛ and o are as defined herein.

Other non-limiting examples of suitable linkers include:

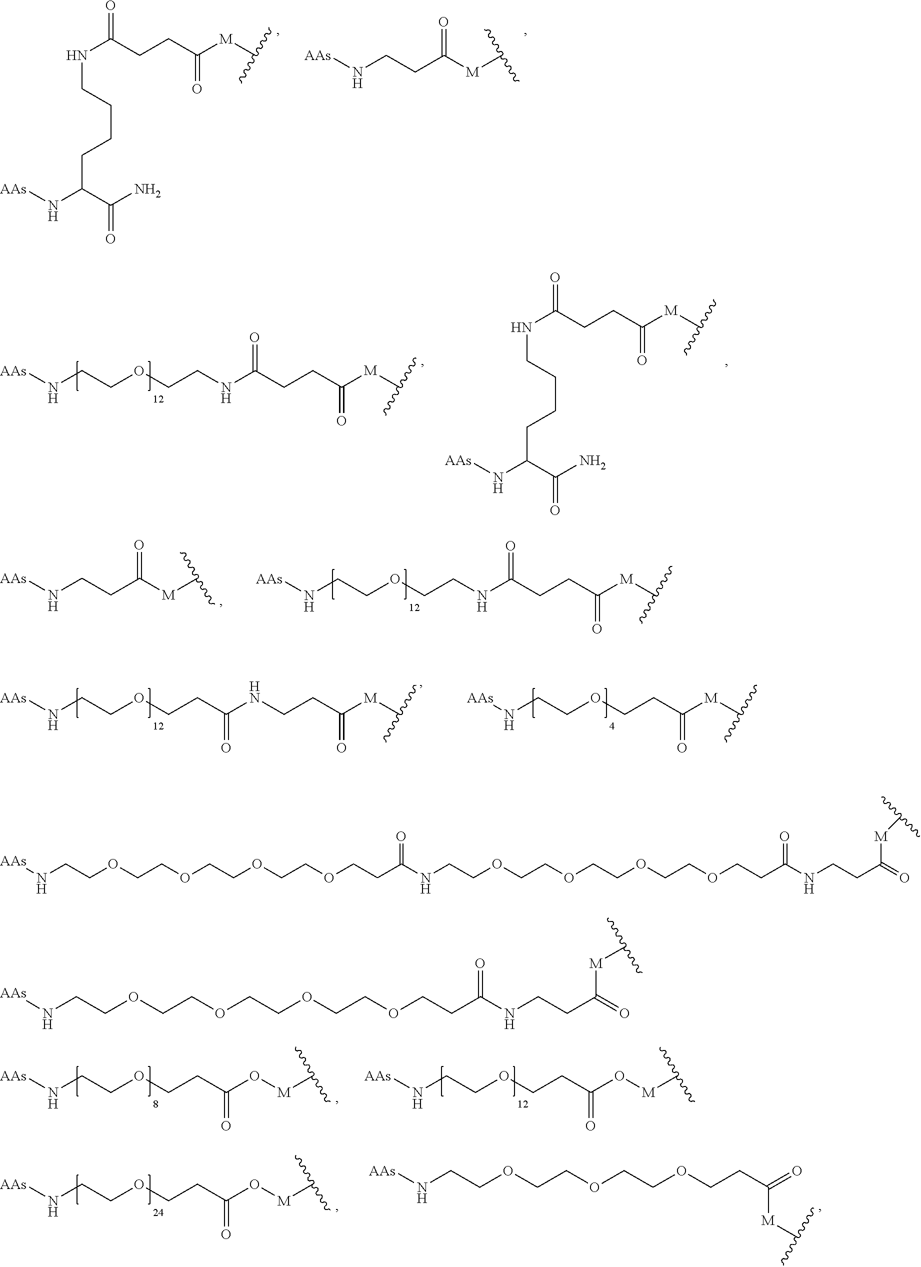

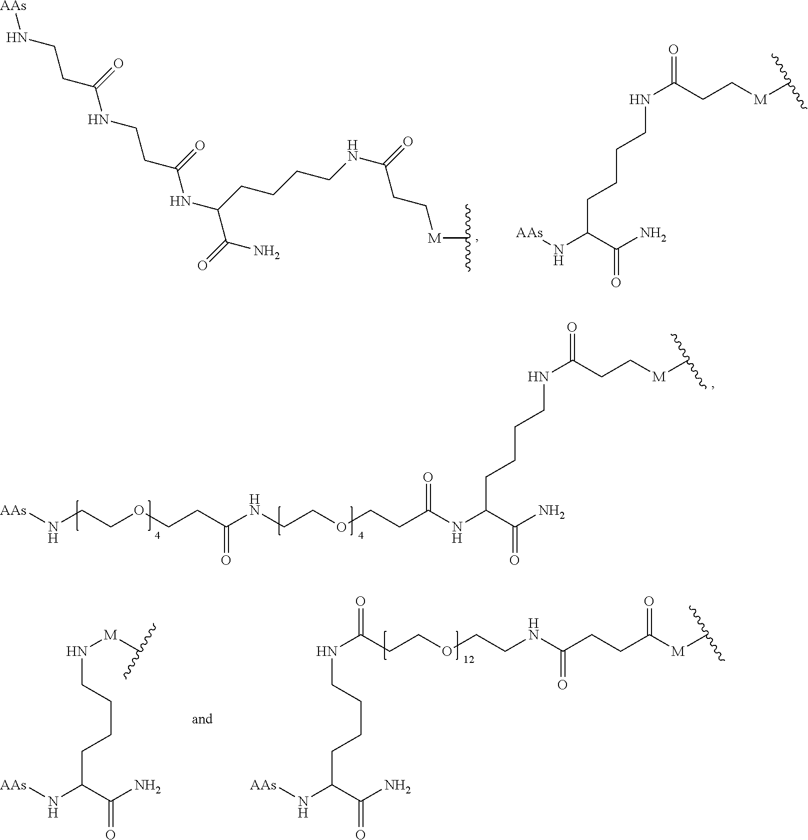

wherein M and AAₛ are as defined herein.

Provided herein is a compound comprising a cCPP and an AC that is complementary to a target in a pre-mRNA sequence further comprising L, wherein the linker is conjugated to the AC through a bonding group (M), wherein M is

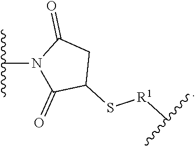

Provided herein is a compound comprising acCPP and a cargo that comprises an antisense compound (AC), for example, an antisense oligonucleotide, that is complementary to a target in a pre-mRNA sequence, wherein the compound further comprises L, wherein the linker is conjugated to the AC through a bonding group (M), wherein M is selected from:

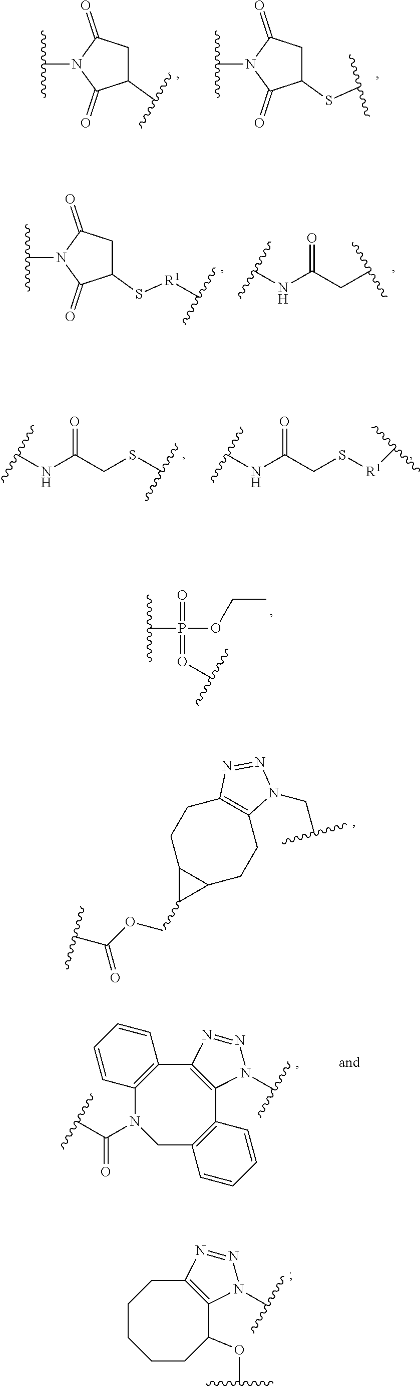

wherein: R¹ is alkylene, cycloalkyl, or

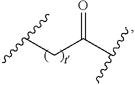

wherein t′ is 0 to 10 wherein each R is independently an alkyl, alkenyl, alkynyl, carbocyclyl, or heterocyclyl, wherein R¹ is

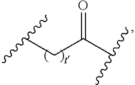

and t′ is 2.

The linker can have the structure:

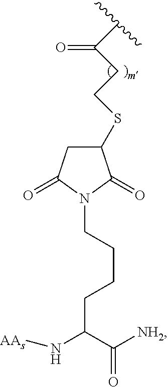

wherein AAₛ is as defined herein, and m′ is 0-10.

The linker can be of the formula:

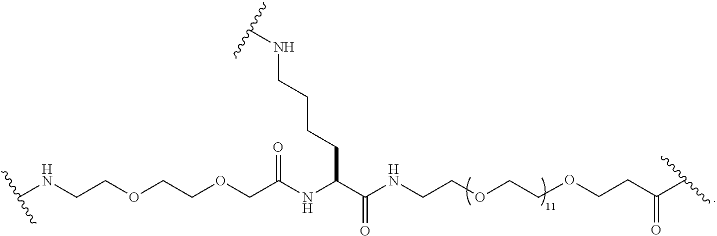

The linker can be of the formula:

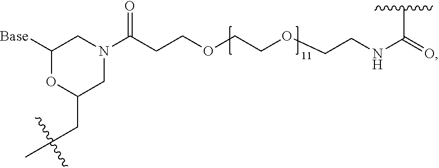

wherein “base” corresponds to a nucleobase at the 3′ end of a cargo phosphorodiamidate morpholino oligomer.

The linker can be of the formula:

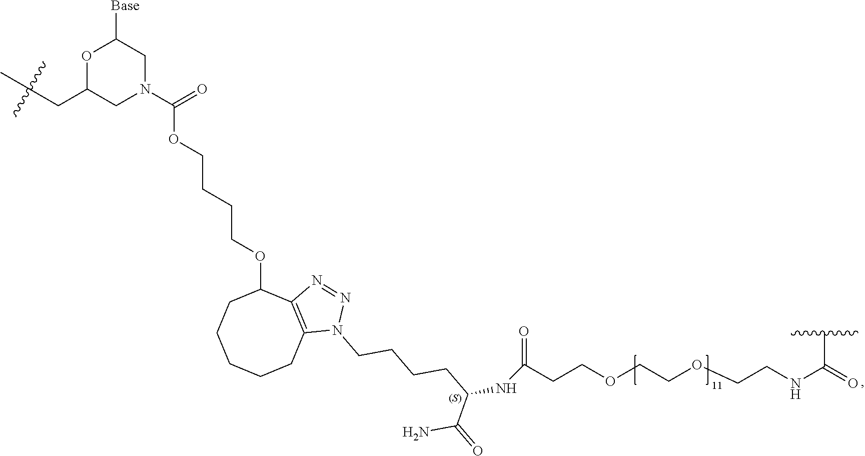

wherein “base” corresponds to a nucleobase at the 3′ end of a cargo phosphorodiamidate morpholino oligomer.

The linker can be of the formula:

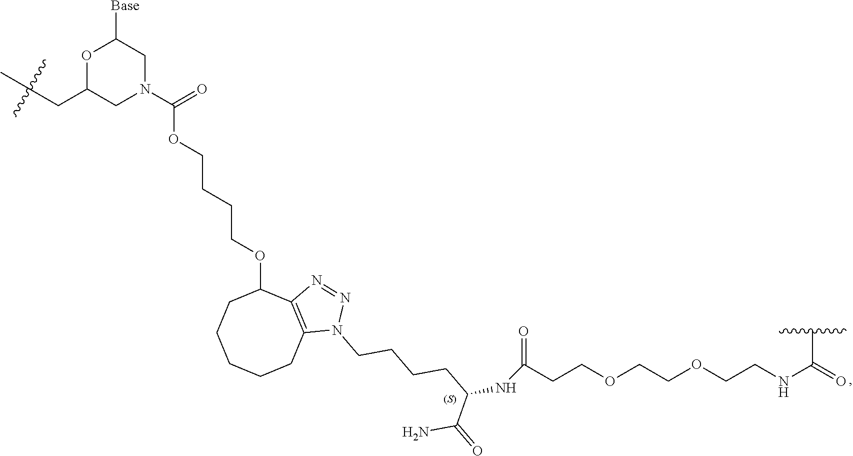

wherein “base” corresponds to a nucleobase at the 3′ end of a cargo phosphorodiamidate morpholino oligomer.

The linker can be of the formula:

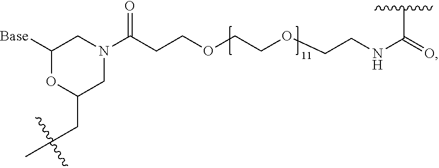

wherein “base” corresponds to a nucleobase at the 3′ end of a cargo phosphorodiamidate morpholino oligomer.

The linker can be of the formula:

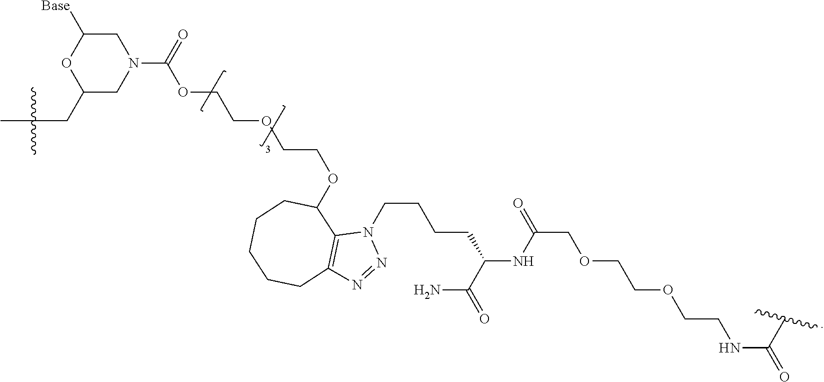

The linker can be covalently bound to a cargo at any suitable location on the cargo. The linker is covalently bound to the 3′ end of cargo or the 5′ end of an oligonucleotide cargo The linker can be covalently bound to the backbone of a cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

cCPP-Linker Conjugates

The cCPP can be conjugated to a linker defined herein. The linker can be conjugated to an AA_{SC} of the cCPP as defined herein.

The linker can comprise a —(OCH₂CH₂)_{z′}— subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂)_{z′} is also referred to as PEG. The cCPP-linker conjugate can have a structure selected from Table 4:

(SEQ ID NO: 242)
cyclo(FfΦ-4gp-r-4gp-rQ)-PEG4-K-NH₂
(SEQ ID NO: 236)
cyclo(FfΦ-Cit-r-Cit-rQ)-PEG4-K-NH₂
(SEQ ID NO: 243)
cyclo(FfΦ-Pia-r-Pia-rQ)-PEG4-K-NH₂
(SEQ ID NO: 244)
cyclo(FfΦ-Dml-r-Dml-rQ)-PEG4-K-NH₂
(SEQ ID NO: 236)
cyclo(FfΦ-Cit-r-Cit-rQ)-PEG12-OH
(SEQ ID NO: 245)
cyclo(fΦR-Cit-R-Cit-Q)-PEG12-OH

The linker can comprise a —(OCH₂CH₂)_{z′}— subunit, wherein z′ is an integer from 1 to 23, and a peptide subunit. The peptide subunit can comprise from 2 to 10 amino acids. The cCPP-linker conjugate can have a structure selected from Table 5:

(SEQ ID NOs: 246 and 247)
Ac-PKKKRKV-K(cyclo[FfQ-R-r-Cit-rQ])-
PEG₁₂-K(N3)-NH₂
(SEQ ID NOs: 246 and 248)
Ac-PKKKRKV-K(cyclo[FfΦ-Cit-r-R-rQ])-
PEG₁₂-K(N₃)-NH₂
(SEQ ID NOs: 246 and 249)
Ac-PKKKRKV-K(cyclo(FfΦR-cit-R-cit-Q))-
PEG₁₂-K(N3)-NH₂
(SEQ ID NOs: 103 and 236)
Ac-PKKKRKV-PEG2-K(cyclo[FfΦ-Cit-r-
Cit-rQ])-B-k(N₃)-NH₂
(SEQ ID NOs: 103 and 236)
Ac-PKKKRKV-
PEG2-K(cyclo[FfΦ-Cit-r-Cit-rQ])-
PEG2-k(N₃)-NH₂
(SEQ ID NOs: 103 and 236)
Ac-PKKKRKV-PEG2-K(cyclo[FfΦ-Cit-r-
Cit-rQ])-PEG4-k(N₃)-NH₂
(SEQ ID NOs: 246 and 236)
Ac-PKKKRKV-K(cyclo[FfΦ-Cit-r-Cit-rQ])-
PEG12-k(N₃)-NH₂
(SEQ ID NOs: 103 and 236)
Ac-pkkkrkv-
PEG2-K(cyclo[FfΦ-Cit-r-Cit-rQ])-
PEG12-k(N₃)-NH₂
(SEQ ID NO: 236)
Ac-rrv-
PEG2-K(cyclo[FfΦ-Cit-r-Cit-rQ])-
PEG12-OH
(SEQ ID NOs: 103 and 236)
Ac-PKKKRKV-
PEG2-K(cyclo[FfΦ-Cit-r-Cit-r-Q])-
PEG12-k(N₃)-NH₂
(SEQ ID NOs: 110 and 236)
Ac-PKKK-Cit-KV-
PEG2-K(cyclo[FfΦ-Cit-r-Cit-r-Q])-
PEG12-k(N₃)-NH₂
(SEQ ID NOs: 103 and 236)
Ac-PKKKRKV-
PEG2-K(cyclo[FfΦ-Cit-r-Cit-r-Q]-
PEG12-K(N₃)-NH₂

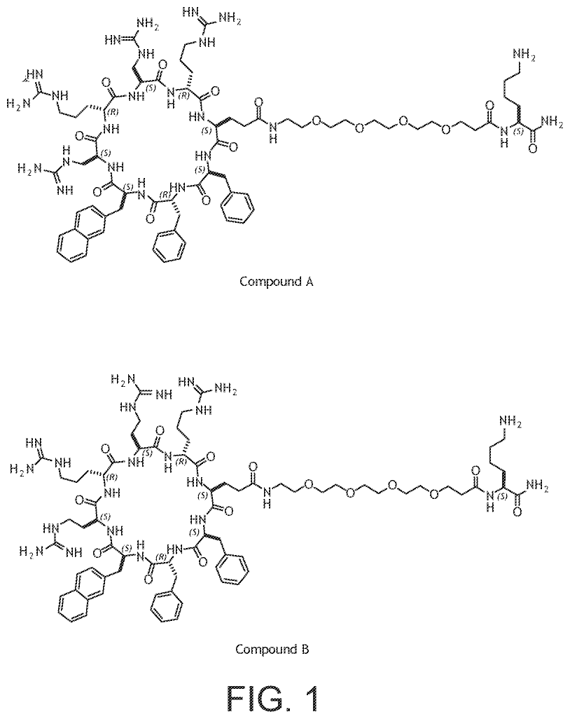

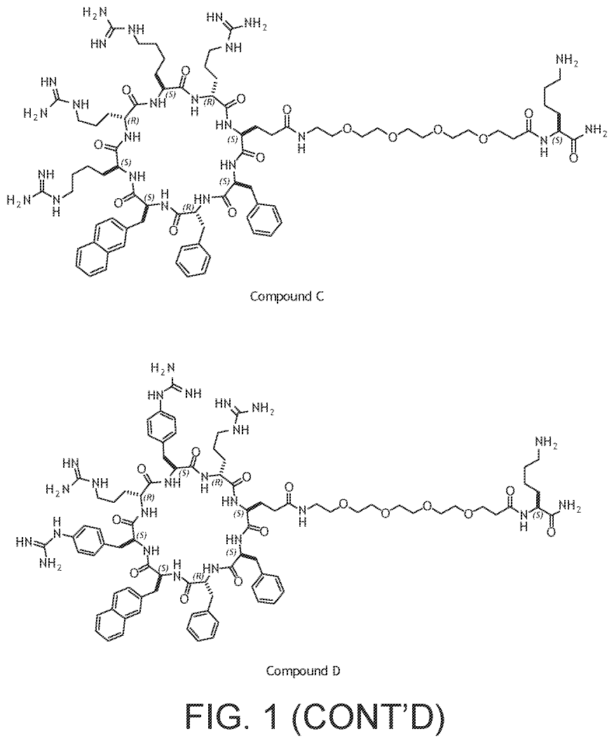

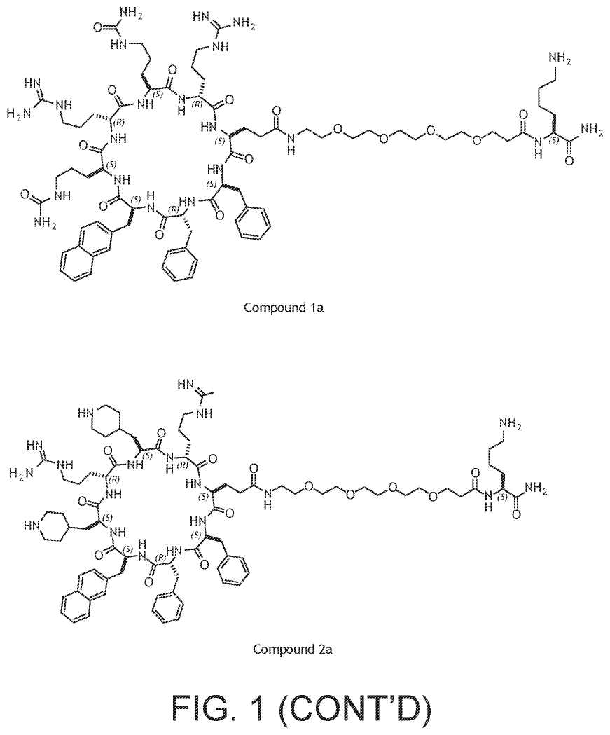

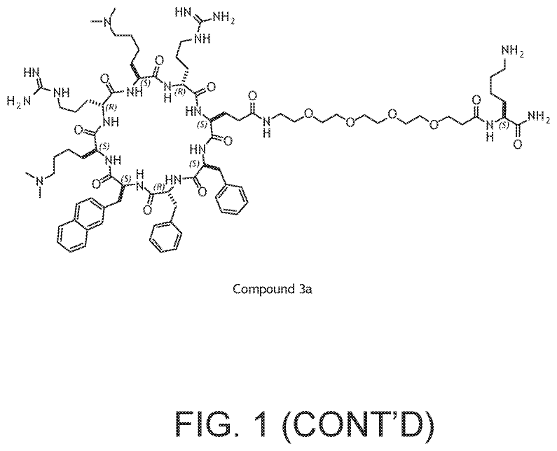

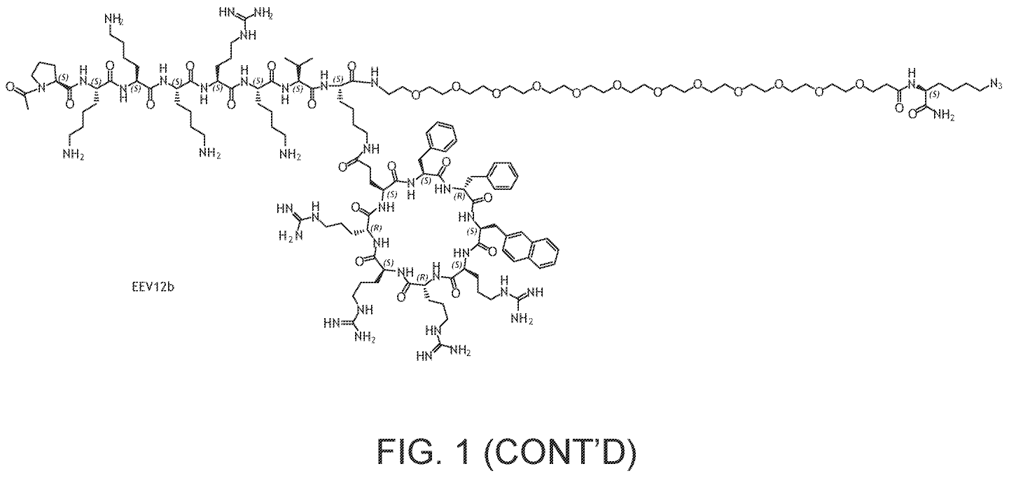

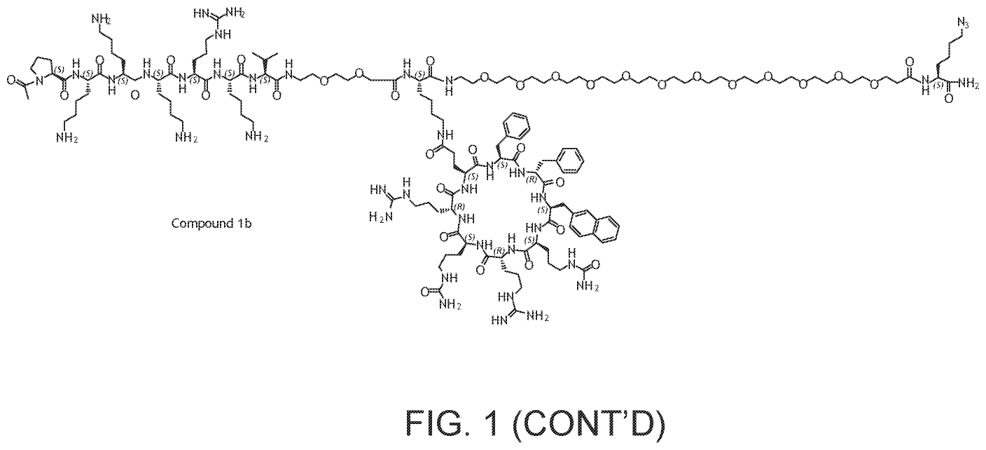

The cCPP-linker conjugate can have a structure shown in FIG. 1 (e.g., Compound 1a, Compound 1b, Compound 2a, or Compound 3a) or sequence listed in Table 4.

The cCPP-linker conjugate can have a sequence as listed in Table 5.

The cCPP-linker conjugate can be Ac-PKKKRKV-K(cyclo[FfΦGrGrQ])-PEG12-K(N₃)—NH₂.EEVs comprising a cyclic cell penetrating peptide (cCPP), linker and exocyclic peptide (EP) are provided. An EEV can comprise the structure of Formula (B):

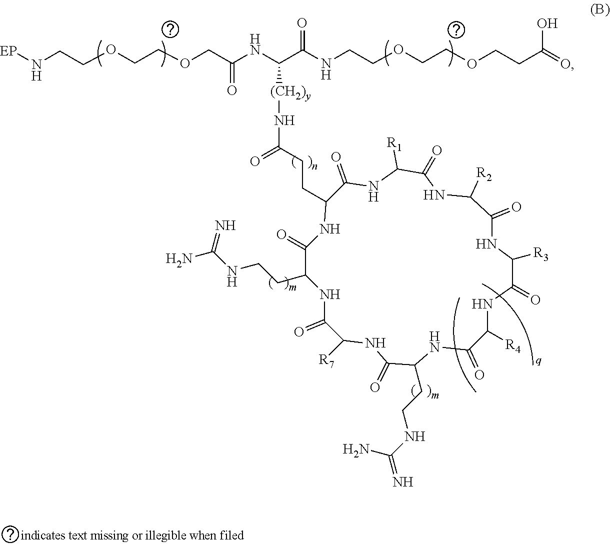

or a protonated form thereof, wherein:
R₁, R₂, and R₃ are each independently H or an aromatic or heteroaromatic side chain of an amino acid;
R₄ and R₇ are independently H or an amino acid side chain;
EP is an exocyclic peptide as defined herein;
each m is independently an integer from 0-3;
n is an integer from 0-2;
x′ is an integer from 1-20;
y is an integer from 1-5;
q is 1-4; and
z′ is an integer from 1-23.

R₁, R₂, R₁, R₄, R₇, EP, m, q, y, x′, z′ are as described herein.

n can be 0. n can be 1. n can be 2.

The EEV can comprise the structure of Formula (B-a) or (B-b):

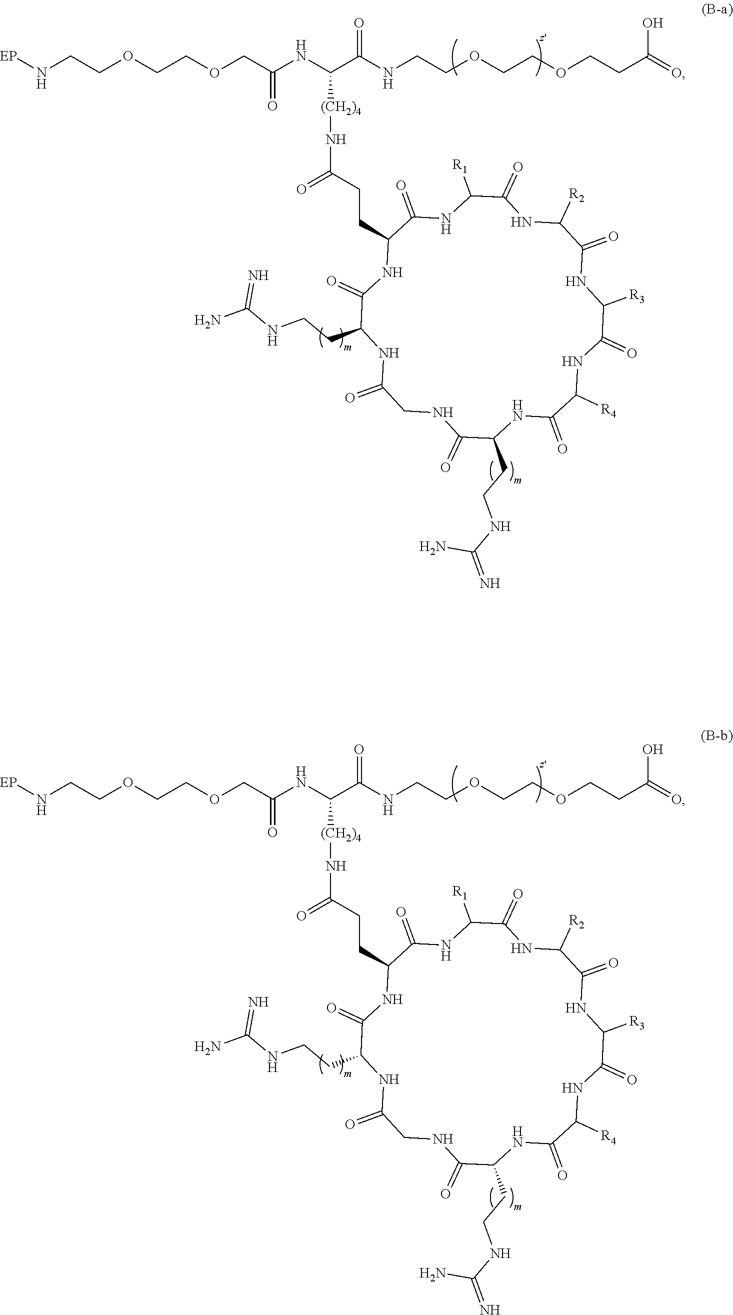

or a protonated form thereof, wherein EP, R¹, R², R³, R⁴, m and z′ are as defined above in Formula (B).

The EEV can comprises the structure of Formula (B-c):

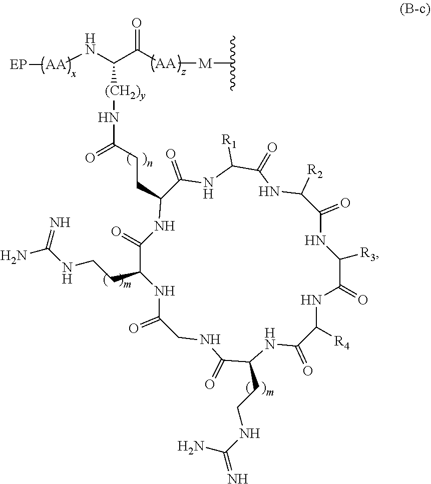

or a protonated form thereof, wherein EP, R¹, R², R³, R⁴, and m are as defined above in Formula (B); AA is an amino acid as defined herein; M is as defined herein; n is an integer from 0-2; x is an integer from 1-10; y is an integer from 1-5; and z is an integer from 1-10.

The EEV can have the structure of Formula (B-1), (B-2), (B-3), or (B-4):

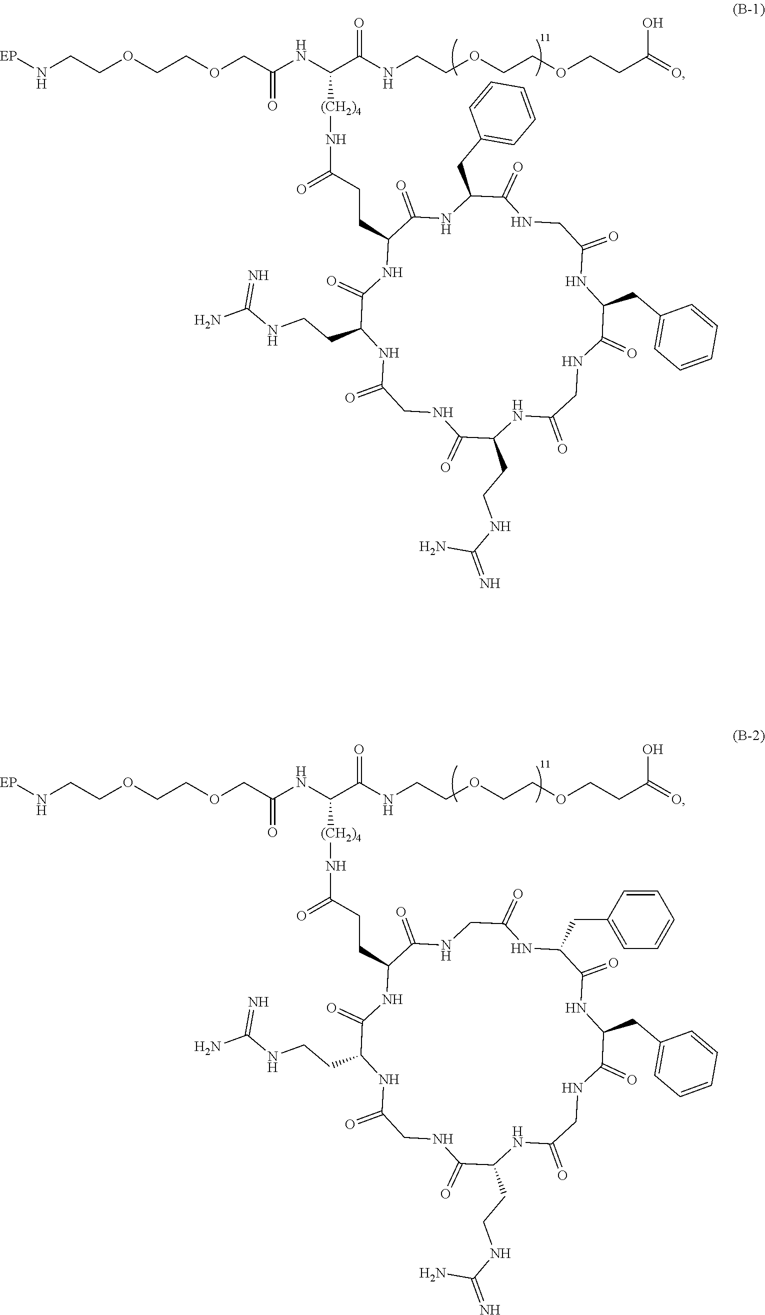

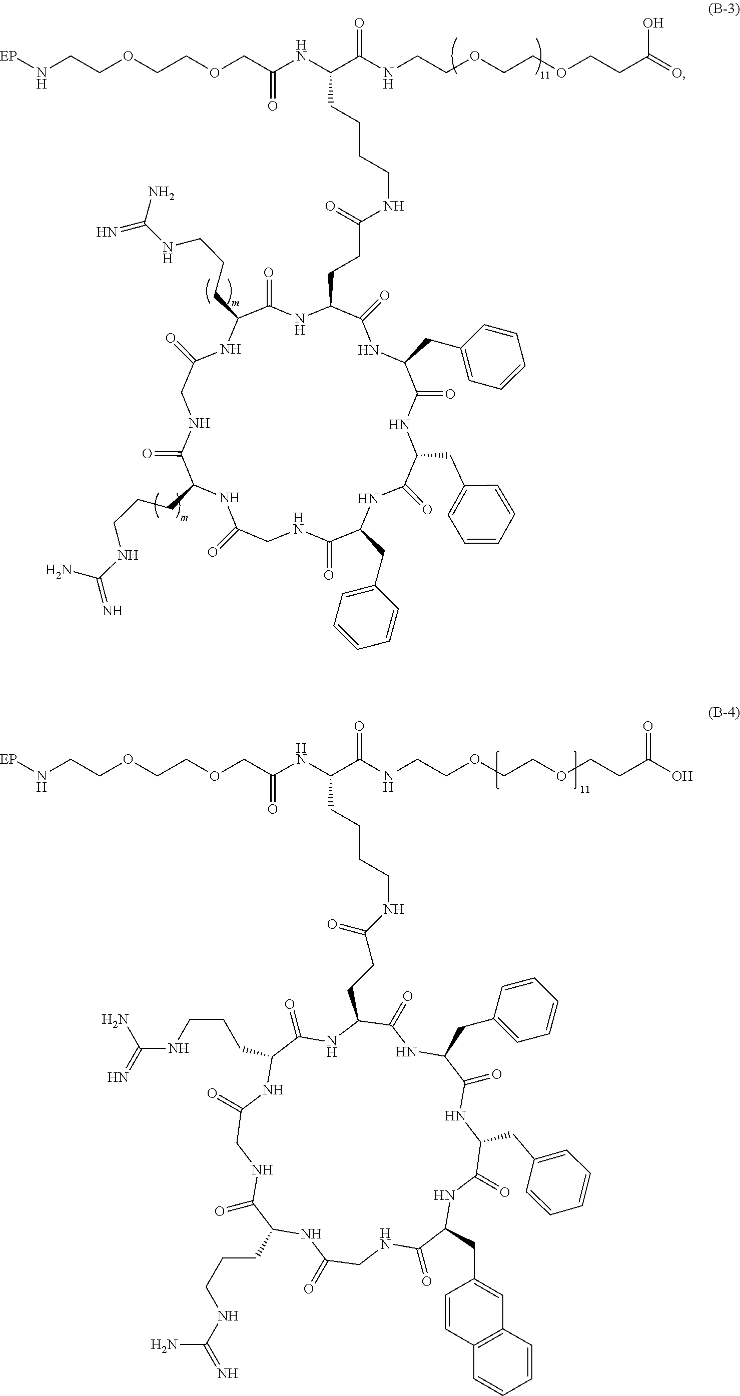

or a protonated form thereof, wherein EP is as defined above in Formula (B).

The EEV can comprise Formula (B) and can have the structure: Ac-PKKKRKVAEEA-K(cyclo[FGFGRGRQ])-PEG₁₂-OH (SEQ ID NOs: 250 and 128) or Ac-PK-KKR-KV-AEEA-K(cyclo[GfFGrGrQ])-PEG₁₂-OH (SEQ ID NOs: 250 and 233).

The EEV can comprise a cCPP of formula:

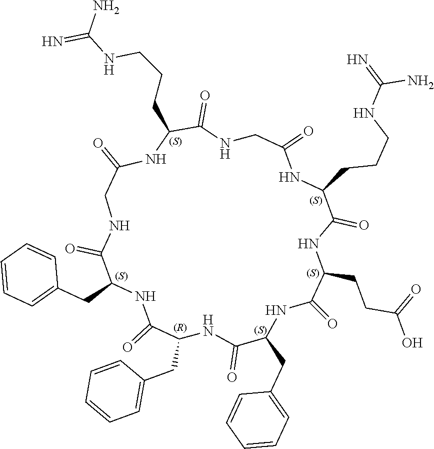

The EEV can comprise formula: Ac-PKKKRKV-miniPEG2-K(cyclo(FfFGRGRQ)-miniPEG2-K(N3) (SEQ ID NOs: 103 and 235).

The EEV can be:

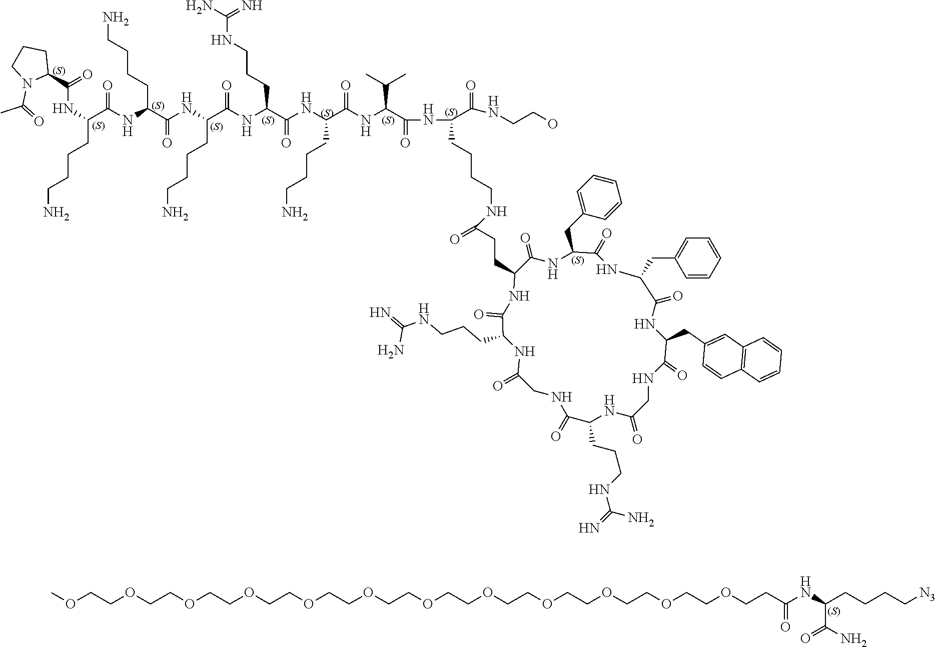

The EEV can be

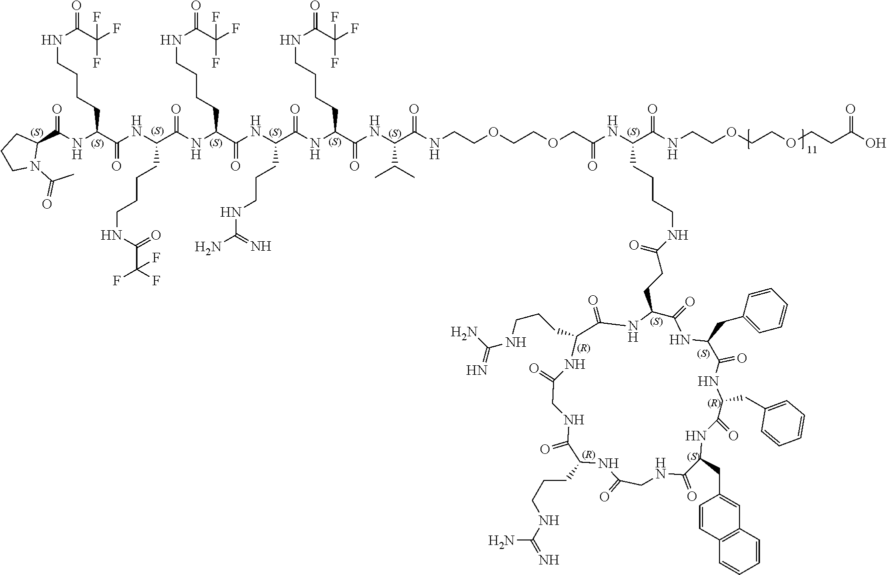

The EEV can be Ac-P-K(Tfa)-K(Tfa)-K(Tfa)-R-K(Tfa)-V-miniPEG-K(cyclo(Ff-Nal-GrGrQ)-PEG12-OH (SEQ ID NOs: 103 and 234).

The EEV can be

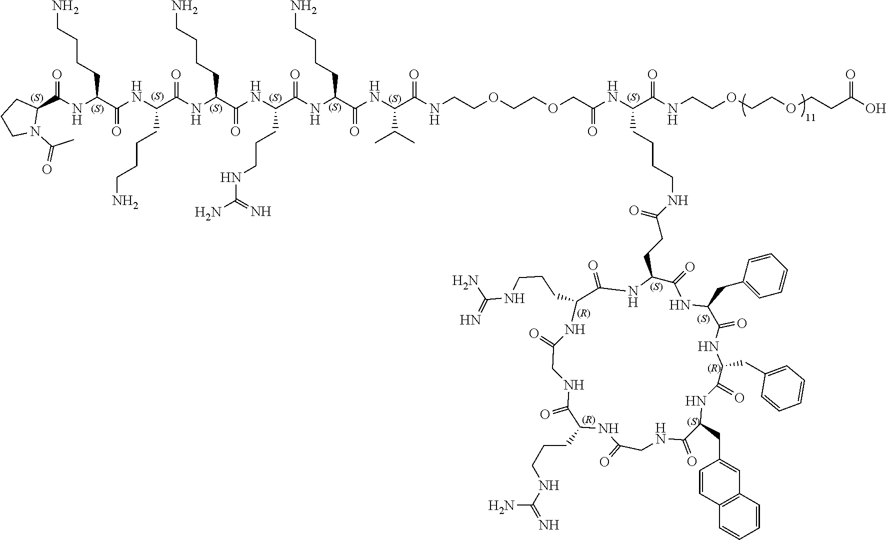

The EEV can be Ac-P-K-K-K-R-K-V-miniPEG-K(cyclo(Ff-Nal-GrGrQ)-PEG12-OH (SEQ ID NOs: 103 and 234).

The EEV can be

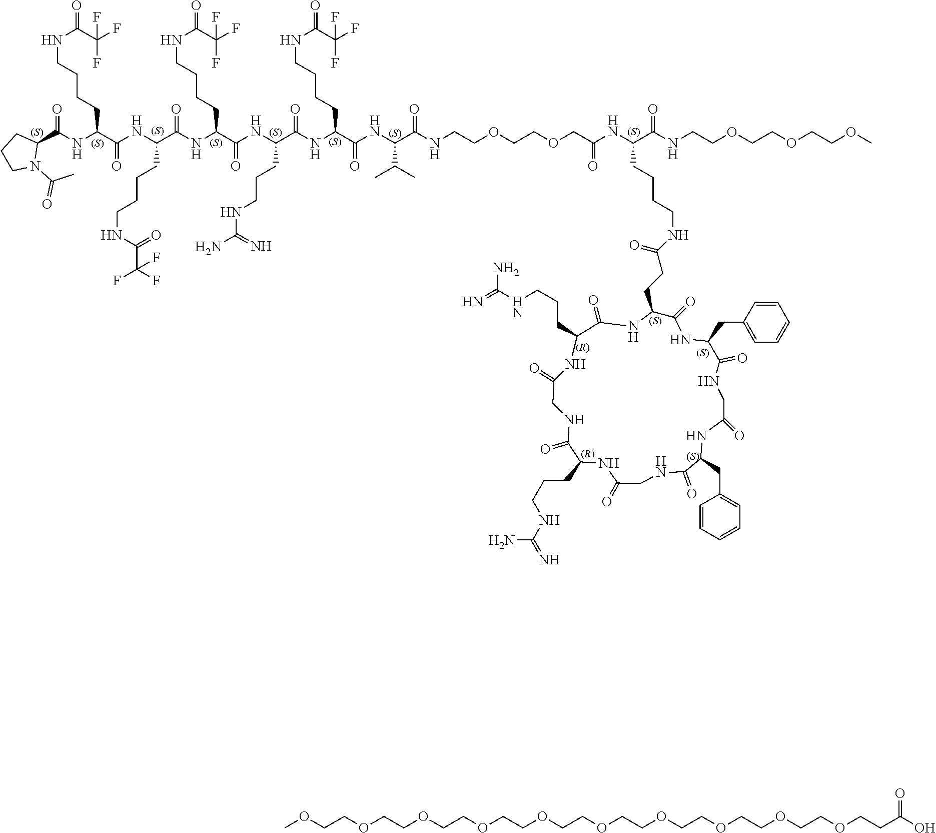

The EEV can be

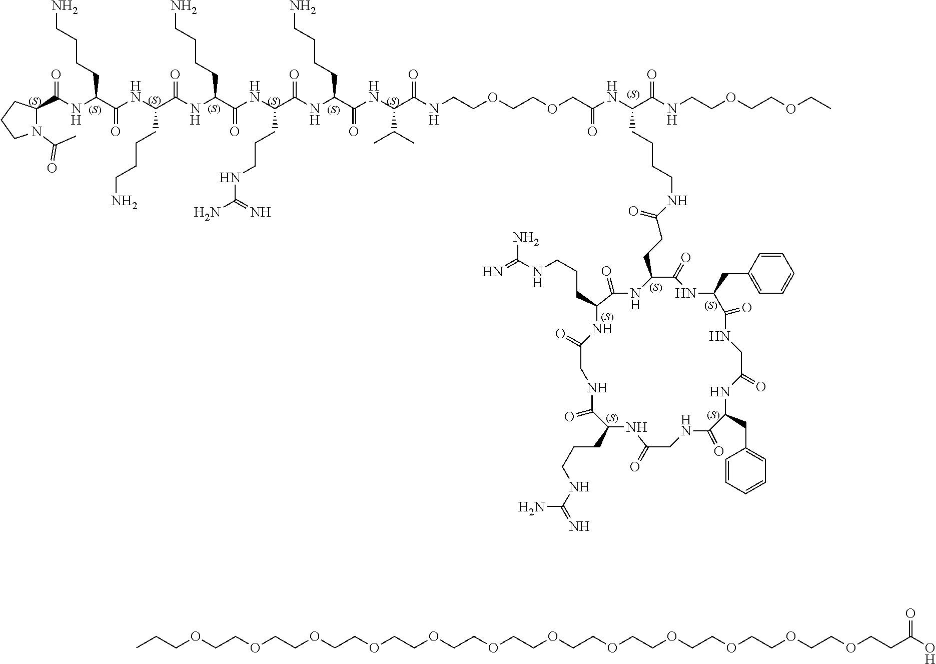

The EEV can be

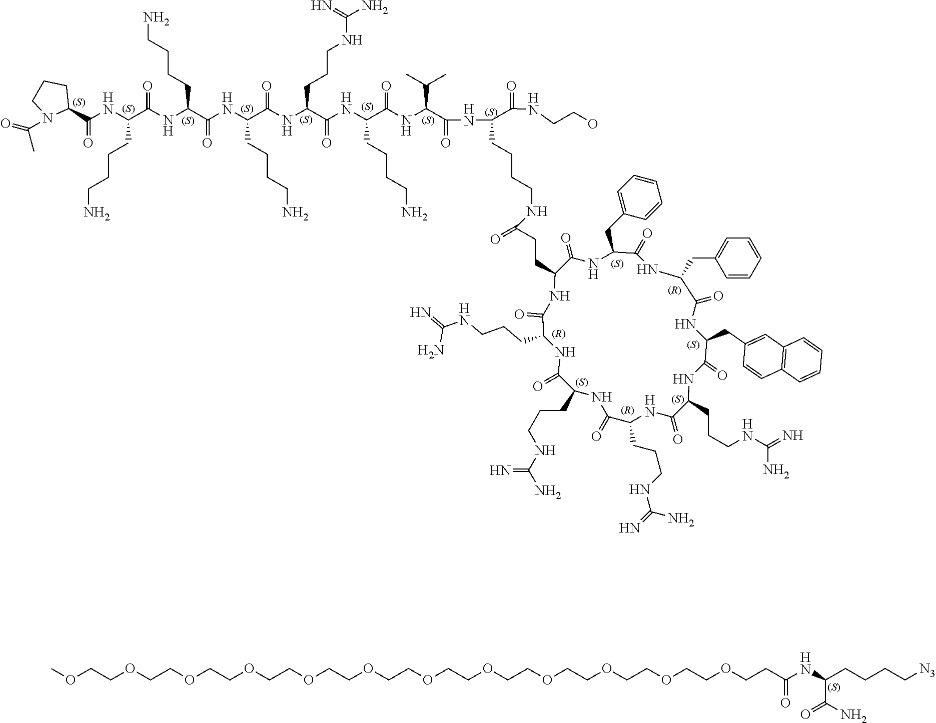

The EEV can be

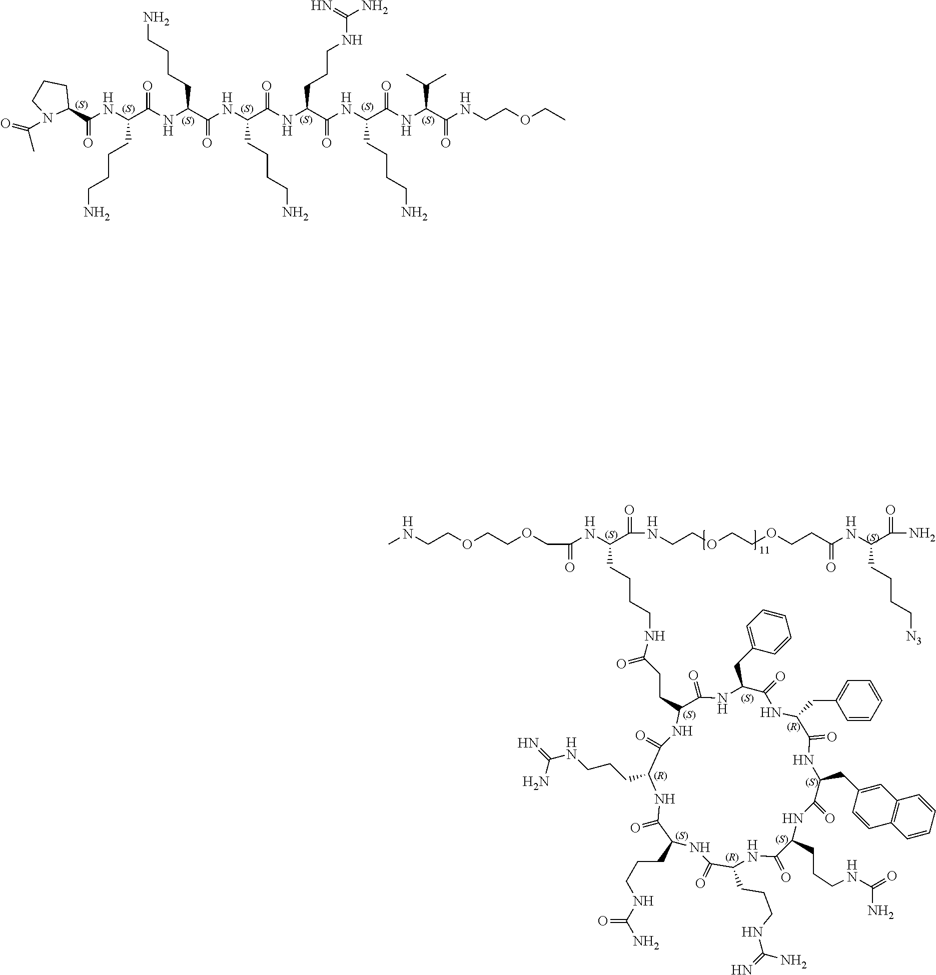

The EEV can be

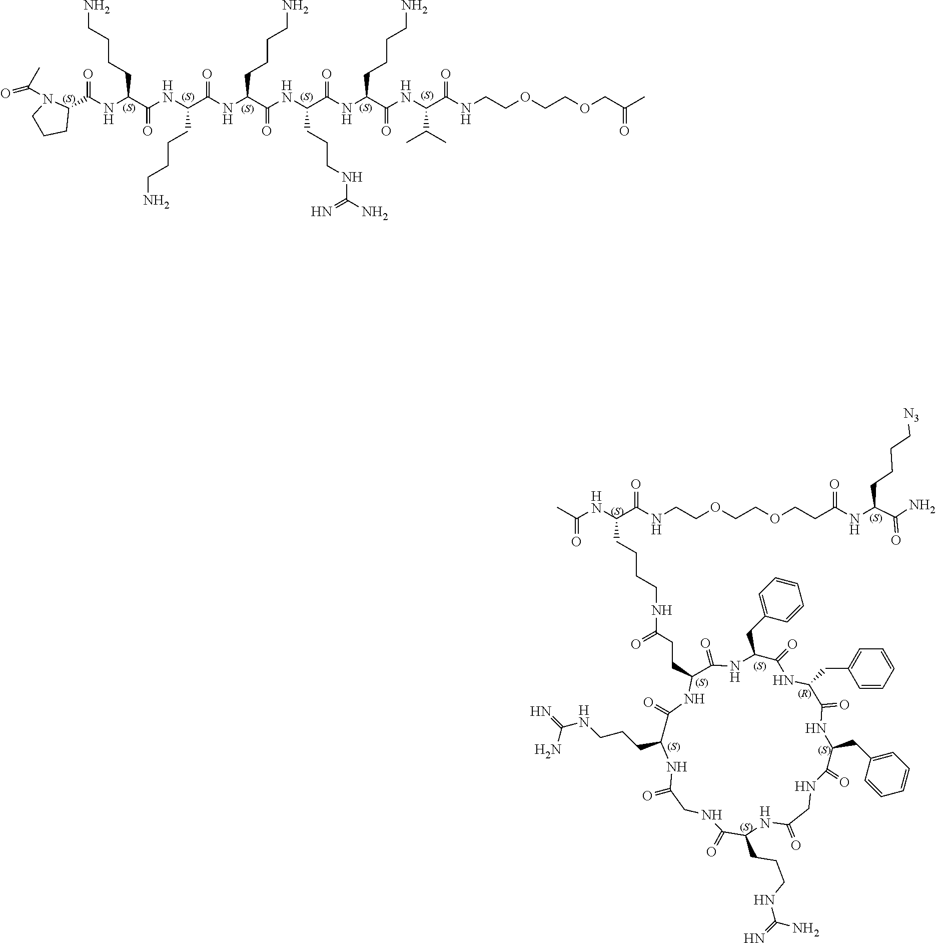

The EEV can be:

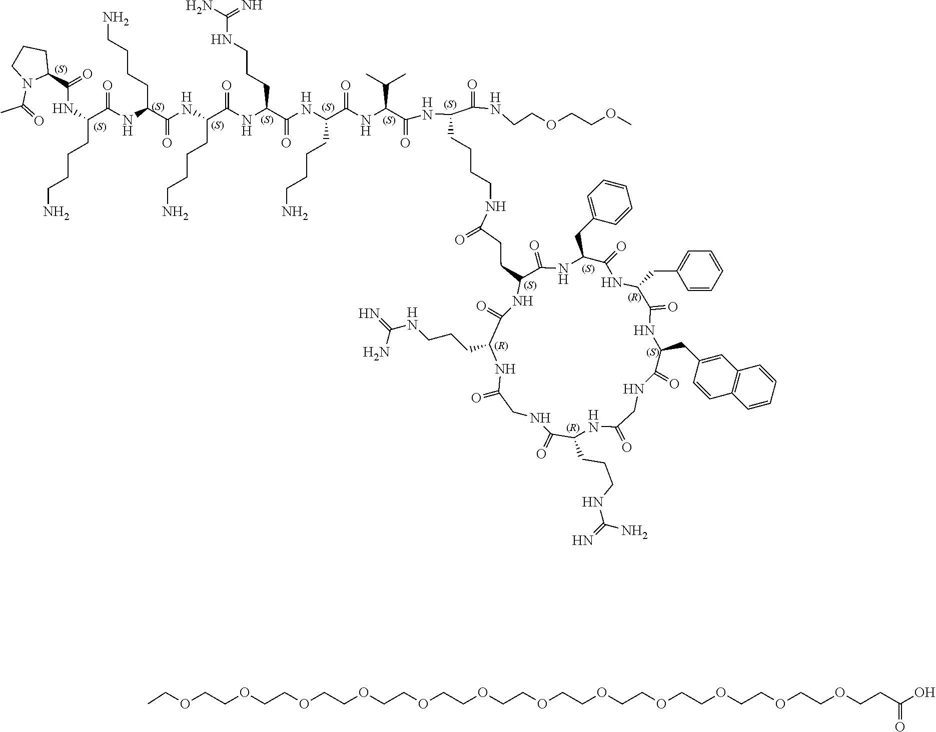

The EEV can be

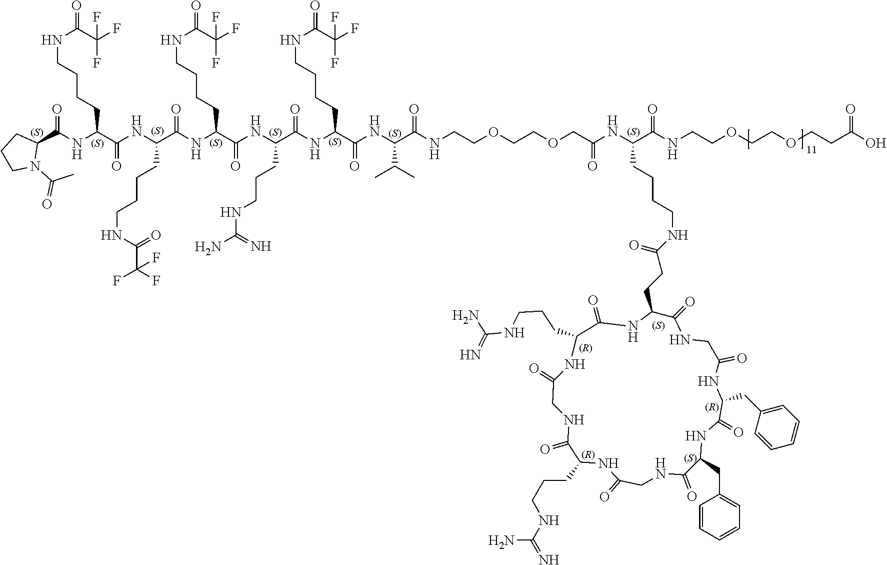

The EEV can be

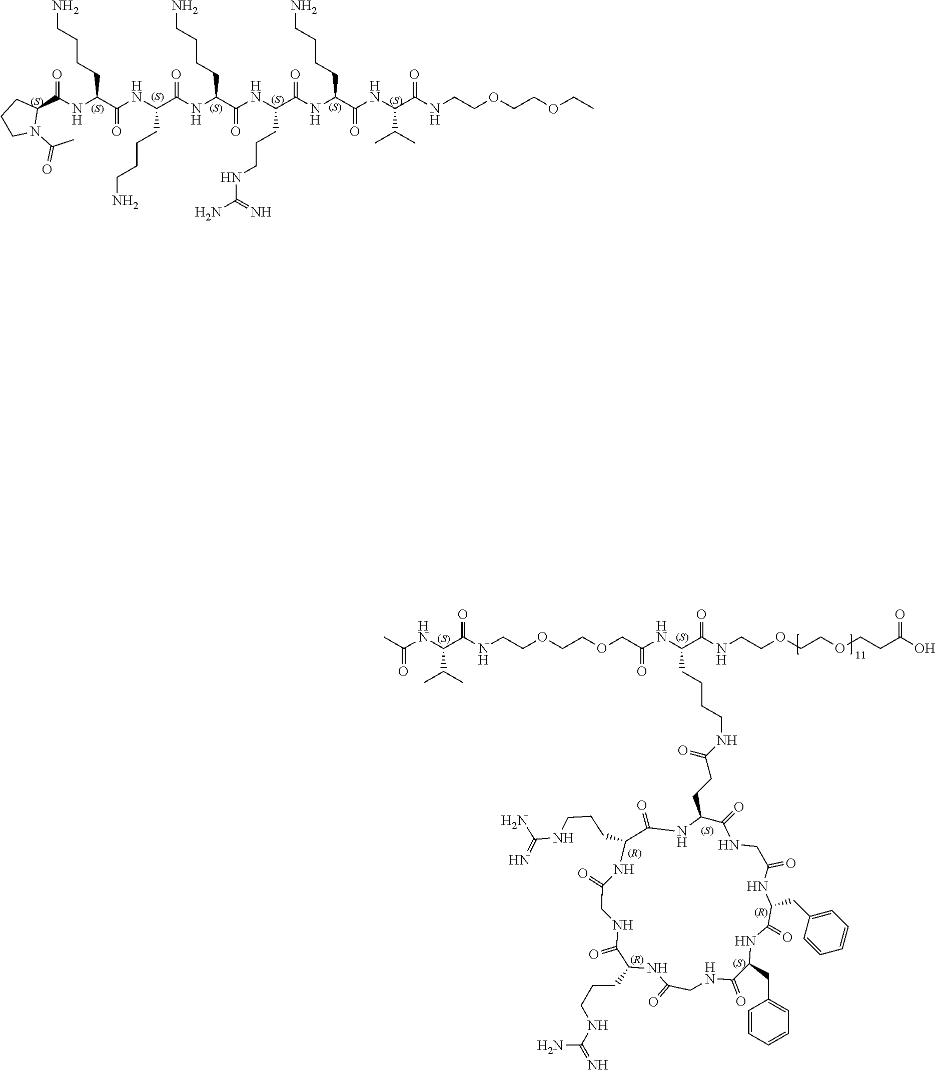

The EEV can be

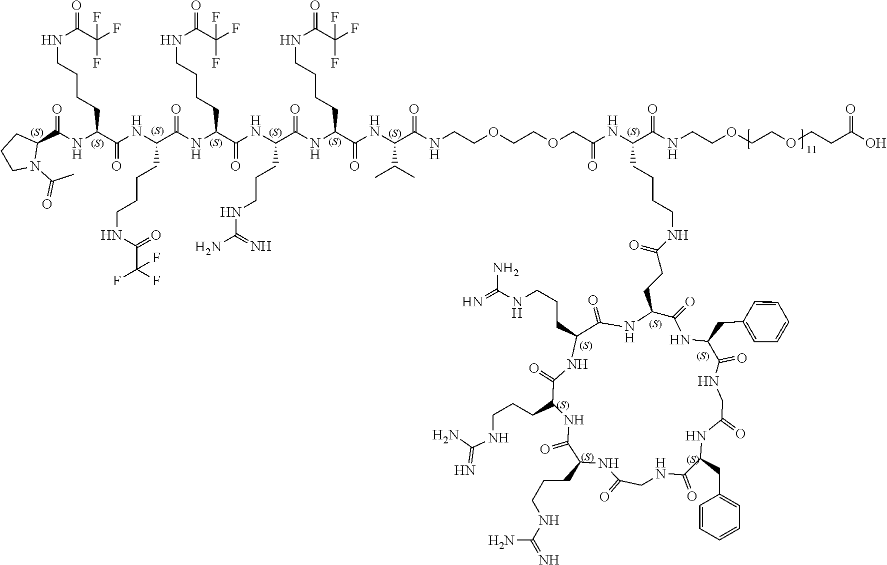

The EEV can be

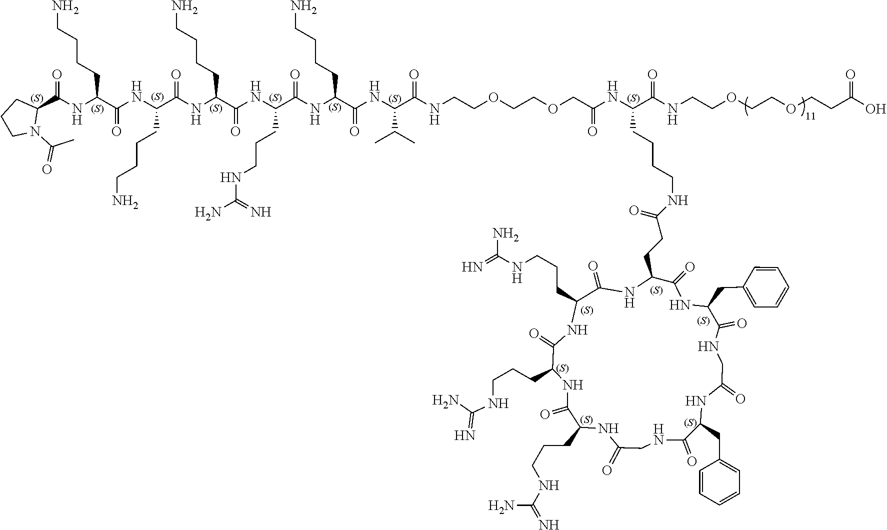

The EEV can be

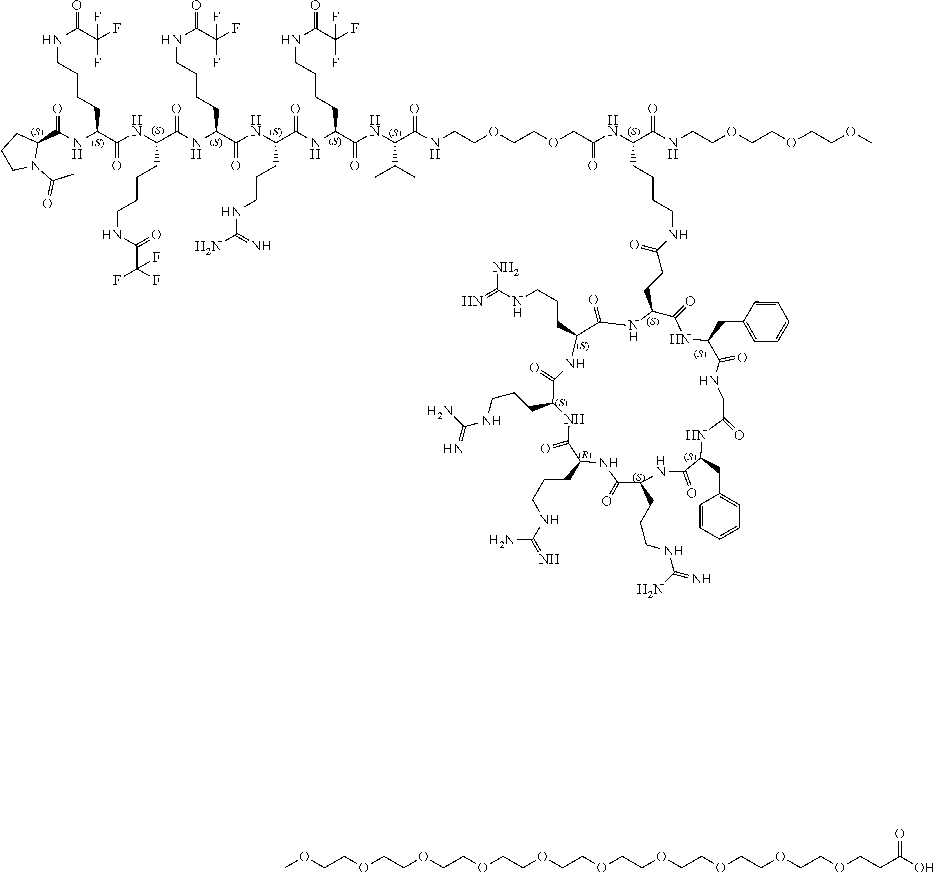

The EEV can be selected from

(SEQ ID NO: 236)
Ac-rr-miniPEG2-Dap[cyclo(FfΦ-Cit-r-Cit-rQ)]-PEG12-
OH
(SEQ ID NO: 236)
Ac-frr-PEG2-Dap(cyclo(FfΦ-Cit-r-Cit-rQ))-PEG12-OH
(SEQ ID NO: 236)
Ac-rfr-PEG2-Dap(cyclo(FfΦ-Cit-r-Cit-rQ))-PEG12-OH
(SEQ ID NO: 236)
Ac-rbfbr-PEG2-Dap(cyclo(FfΦ-Cit-r-Cit-rQ))-PEG12-
OH
(SEQ ID NO: 236)
Ac-rrr-PEG2-Dap(cyclo(FfΦ-Cit-r-Cit-rQ))-PEG12-OH
(SEQ ID NO: 236)
Ac-rbr-PEG2-Dap(cyclo(FfΦ-Cit-r-Cit-rQ))-PEG12-OH
(SEQ ID NO: 236)
Ac-rbrbr-PEG2-Dap(cyclo(FfΦ-Cit-r-Cit-rQ))-PEG12-
OH
(SEQ ID NO: 236)
Ac-hh-PEG2-Dap(cyclo(FfΦ-Cit-r-Cit-rQ))-PEG12-OH
(SEQ ID NO: 236)
Ac-hbh-PEG2-Dap(cyclo(FfΦ-Cit-r-Cit-rQ))-PEG12-OH
(SEQ ID NO: 236)
Ac-hbhbh-PEG2-Dap(cyclo(FfΦ-Cit-r-Cit-rQ))-PEG12-
OH
(SEQ ID NO: 236)
Ac-rbhbh-PEG2-Dap(cyclo(FfΦ-Cit-r-Cit-rQ))-PEG12-
OH
(SEQ ID NO: 236)
Ac-hbrbh-PEG2-Dap(cyclo(FfΦ-Cit-r-Cit-rQ))-PEG12-
OH
(SEQ ID NO: 236)
Ac-rr-Dap(cyclo(FfΦ-Cit-r-Cit-rQ))-b-OH
(SEQ ID NO: 236)
Ac-frr-Dap(cyclo(FfΦ-Cit-r-Cit-rQ))-b-OH
(SEQ ID NO: 236)
Ac-rfr-Dap(cyclo(FfΦ-Cit-r-Cit-rQ))-b-OH
(SEQ ID NO: 236)
Ac-rbfbr-Dap(cyclo(FfΦ-Cit-r-Cit-rQ))-b-OH
(SEQ ID NO: 236)
Ac-rrr-Dap(cyclo(FfΦ-Cit-r-Cit-rQ))-b-OH
(SEQ ID NO: 236)
Ac-rbr-Dap(cyclo(FfΦ-Cit-r-Cit-rQ))-b-OH
(SEQ ID NO: 236)
Ac-rbrbr-Dap(cyclo(FfΦ-Cit-r-Cit-rQ))-b-OH
(SEQ ID NO: 236)
Ac-hh-Dap(cyclo(FfΦ-Cit-r-Cit-rQ))-b-OH
(SEQ ID NO: 236)
Ac-hbh-Dap(cyclo(FfΦ-Cit-r-Cit-rQ))-b-OH
(SEQ ID NO: 236)
Ac-hbhbh-Dap(cyclo(FfΦ-Cit-r-Cit-rQ))-b-OH
(SEQ ID NO: 236)
Ac-rbhbh-Dap(cyclo(FfΦ-Cit-r-Cit-rQ))-b-OH
(SEQ ID NO: 236)
Ac-hbrbh-Dap(cyclo(FfΦ-Cit-r-Cit-rQ))-b-OH
(SEQ ID NOs: 64 and 234)
Ac-KKKK-miniPEG2-K(cyclo(Ff-Nal-GrGrQ))-miniPEG2-
K(N3)-NH2
(SEQ ID NOs: 70 and 234)
Ac-KGKK-miniPEG2-K(cyclo(Ff-Nal-GrGrQ))-miniPEG2-
K(N3)-NH2
(SEQ ID NOs: 71 and 234)
Ac-KKGK-miniPEG2-K(cyclo(Ff-Nal-GrGrQ))-miniPEG2-
K(N3)-NH2
(SEQ ID NO: 234)
Ac-KKK-miniPEG2-K(cyclo(Ff-Nal-GrGrQ))-miniPEG2-
K(N3)-NH2
(SEQ ID NO: 234)
Ac-KK-miniPEG2-K(cyclo(Ff-Nal-GrGrQ))-miniPEG2-
K(N3)-NH2
(SEQ ID NO: 234)
Ac-KGK-miniPEG2-K(cyclo(Ff-Nal-GrGrQ))-miniPEG2-
K(N3)-NH2
(SEQ ID NO: 234)
Ac-KBK-miniPEG2-K(cyclo(Ff-Nal-GrGrQ))-miniPEG2-
K(N3)-NH2
(SEQ ID NO: 234)
Ac-KBKBK-miniPEG2-K(cyclo(Ff-Nal-GrGrQ))-miniPEG2-
K(N3)-NH2
(SEQ ID NO: 234)
Ac-KR-miniPEG2-K(cyclo(Ff-Nal-GrGrQ))-miniPEG2-
K(N3)-NH2
(SEQ ID NO: 234)
Ac-KBR-miniPEG2-K(cyclo(Ff-Nal-GrGrQ))-miniPEG2-
K(N3)-NH2
(SEQ ID NOs: 103 and 234)
Ac-PKKKRKV-miniPEG2-K(cyclo(Ff-Nal-GrGrQ))-
miniPEG2-K(N3)-NH2
(SEQ ID NOs: 103 and 234)
Ac-PKKKRKV-miniPEG2-K(cyclo(Ff-Nal-GrGrQ))-
miniPEG2-K(N3)-NH2
(SEQ ID NOs: 104 and 234)
Ac-PGKKRKV-miniPEG2-K(cyclo(Ff-Nal-GrGrQ))-
miniPEG2-K(N3)-NH2
(SEQ ID NOs: 105 and 234)
Ac-PKGKRKV-miniPEG2-K(cyclo(Ff-Nal-GrGrQ))-
miniPEG2-K(N3)-NH2
(SEQ ID NOs: 106 and 234)
Ac-PKKGRKV-miniPEG2-K(cyclo(Ff-Nal-GrGrQ))-
miniPEG2-K(N3)-NH2
(SEQ ID NOs: 107 and 234)
Ac-PKKKGKV-miniPEG2-K(cyclo(Ff-Nal-GrGrQ))-
miniPEG2-K(N3)-NH2
(SEQ ID NOs: 108 and 234)
Ac-PKKKRGV-miniPEG2-K(cyclo(Ff-Nal-GrGrQ))-
miniPEG2-K(N3)-NH2
(SEQ ID NOs: 109 and 234)
Ac-PKKKRKG-miniPEG2-K(cyclo(Ff-Nal-GrGrQ))-
miniPEG2-K(N3)-NH2
(SEQ ID NOs: 76 and 234)
Ac-KKKRK-miniPEG2-K(cyclo(Ff-Nal-GrGrQ))-miniPEG2-
K(N3)-NH2
(SEQ ID NOs: 65 and 234)
Ac-KKRK-miniPEG2-K(cyclo(Ff-Nal-GrGrQ))-miniPEG2-
K(N3)-NH2
and
(SEQ ID NO: 234)
Ac-KRK-miniPEG2-K(cyclo(Ff-Nal-GrGrQ))-miniPEG2-
K(N3)-NH2.

The EEV can be selected from

(SEQ ID NOs: 103 and 234)
Ac-PKKKRKV-K(cyclo[FfΦGrGrQ1)-PEG₁₂-K(N₃)-NH₂
(SEQ ID NOs: 103 and 234)
Ac-PKKKRKV-miniPEG₂-K(cyclo[FfΦGrGrQ])-miniPEG₂-
K(N₃)-NH₂
(SEQ ID NOs: 103 and 128)
Ac-PKKKRKV-miniPEG₂-K(cyclo[FGFGRGRQ])-miniPEG₂-
K(N₃)-NH₂
(SEQ ID NO: 128)
Ac-KR-PEG₂-K(cyclo[FGFGRGRQ])-PEG₂-K(N₃)-NH₂
(SEQ ID NOs: 107 and 128)
Ac-PKKKGKV-PEG₂-K(cyclo[FGFGRGRQ])-PEG₂-K(N₃)-NH₂
(SEQ ID NOs: 109 and 128)
Ac-PKKKRKG-PEG₂-K(cyclo[FGFGRGRQ])-PEG₂-K(N₃)-NH₂
(SEQ ID NOs: 76 and 128)
Ac-KKKRK-PEG₂-K(cyclo[FGFGRGRQ])-PEG₂-K(N₃)-NH₂
(SEQ_ID NOs: 103 and 234)
Ac-PKKKRKV-miniPEG₂-K(cyclo[FFΦGRGRQ)-miniPEG₂-
K(N₃)-NH₂
(SEQ ID NOs: 103 and 234)
Ac-PKKKRKV-miniPEG₂-K(cyclo[βhFfΦGrGrQ])-miniPEG₂-
K(N₃)-NH₂
and
(SEQ ID NOs: 103 and 251)
Ac-PKKKRKV-miniPEG₂-K(cyclo[FfΦSrSrQ])-miniPEG₂-
K(N₃)-NH₂.

The EEV can be selected from

(SEQ ID NOs: 103 and 233)
Ac-PKKKRKV-miniPEG₂-K(cyclo(GfFGrGrQ])-PEG₁₂-OH
(SEQ ID NOs: 103 and 132)
Ac-PKKKRKV-miniPEG₂-K(cyclo[FGFKRKRQ])-PEG₁₂-OH
(SEQ ID NOs: 103 and 133)
Ac-PKKKRKV-miniPEG₂-K(cyclo[FGFRGRGQ])-PEG₁₂-OH
(SEQ ID NOs: 103 and 134)
Ac-PKKKRKV-miniPEG₂-K(cyclo[FGFGRGRGRQ])-PEG₁₂-OH
(SEQ ID NOs: 103 and 135)
Ac-PKKKRKV-miniPEG₂-K(cyclo[FGFGRrRQ])-PEG₁₂-OH
(SEQ ID NOs: 103 and 135)
Ac-PKKKRKV-miniPEG₂-K(cyclo[FGFGRRRQ])-PEG₁₂-OH
and
(SEQ ID NOs: 103 and 252)
Ac-PKKKRKV-miniPEG₂-K(cyclo[FGFRRRRQ])-PEG₁₂-OH.

The EEV can be selected from

(SEQ ID NOs: 109 and 128)
Ac-KKKRKG-miniPEG₂-K(cyclo[FGFGRGRQ])-PEG₁₂-OH
(SEQ ID NOs: 76 and 128)
Ac-KKKRK-miniPEG₂-K(cyclo[FGFGRGRQ])-PEG₁₂-OH
(SEQ ID NOs: 79 and 128)
Ac-KKRKK-PEG₄-K(cyclo[FGFGRGRQ])-PEG₁₂-OH
(SEQ ID NOs: 78 and 128)
Ac-KRKKK-PEG₄-K(cyclo[FGFGRGRQ])-PEG₁₂-OH
(SEQ ID NOs: 80 and 128)
Ac-KKKKR-PEG₄-K(cyclo[FGFGRGRQ])-PEG₁₂-OH
(SEQ ID NOs: 77 and 128)
Ac-RKKKK-PEG₄-K(cyclo[FGFGRGRQ])-PEG₁₂-OH
and
(SEQ ID NOs: 76 and 128)
Ac-KKKRK-PEG₄-K(cyclo[FGFGRGRQ])-PEG₁₂-OH.

The EEV can be selected from

(SEQ ID NOs: 103 and 128)
Ac-PKKKRKV-PEG₂-K(cyclo[FGFGRGRQ])-PEG₂-K(N₃)-NH₂
(SEQ ID NOs: 103 and 128)
Ac-PKKKRKV-PEG₂-K(cyclo[FGFGRGRQ])-PEG₁₂-OH
(SEQ ID NOs: 103 and 233)
Ac-PKKKRKV-PEG₂-K(cyclo[GfFGrGrQ])-PEG₂-K(N₃)-NH₂
and
(SEQ ID NOs: 103 and 233)
Ac- PKKKRKV-PEG₂-K(cyclo[GfFGrGrQ])-PEG₁₂-OH.

The cargo can be a protein and the EEV can be selected from:

(SEQ ID NOs: 103 and 234)
Ac-PKKKRKV-PEG₂-K(cyclo[Ff-Nal-GrGrQ])-PEG₁₂-OH
(SEQ ID NOs: 103 and 236)
Ac-PKKKRKV-PEG₂-K(cyclo[Ff-Nal-Cit-r-Cit-rQ])-
PEG₁₂-OH
(SEQ ID NOs: 103 and 235)
Ac-PKKKRKV-PEG₂-K(cyclo[FfF-GRGRQ])-PEG₁₂-OH
(SEQ ID NOs: 103 and 128)
Ac-PKKKRKV-PEG₂-K(cyclo[FGFGRGRQ])-PEG₁₂-OH
(SEQ ID NOs: 103 and 233)
Ac-PKKKRKV-PEG₂-K(cyclo[GfFGrGrQ])-PEG₁₂-OH
(SEQ ID NOs: 103 and 130)
Ac-PKKKRKV-PEG₂-K(cyclo[FGFGRRRQ])-PEG₁₂-OH
(SEQ ID NOs: 103 and 136)
Ac-PKKKRKV-PEG₂-K(cyclo[FGFRRRRQ])-PEG₁₂-OH
(SEQ ID NO: 234)
Ac-rr-PEG₂-K(cyclo[Ff-Nal-GrGrQ])-PEG₁₂-OH
(SEQ ID NO: 236)
Ac-rr-PEG₂-K(cyclo[Ff-Nal-Cit-r-Cit-rQ])-PEG₁₂-OH
(SEQ ID NO: 235)
Ac-rr-PEG₂-K(cyclo[FfF-GRGRQ])-PEG₁₂-OH
(SEQ ID NO: 128)
Ac-rr-PEG₂-K(cyclo[FGFGRGRQ])-PEG₁₂-OH
(SEQ ID NO: 233)
Ac-rr-PEG₂-K(cyclo[GfFGrGrQ])-PEG₁₂-OH
(SEQ ID NO: 130)
Ac-rr-PEG₂-K(cyclo[FGFGRRRQ])-PEG₁₂-OH
(SEQ ID NO: 129)
Ac-rr-PEG₂-K(cyclo[FGFRRRRQ])-PEG₁₂-OH
(SEQ ID NO: 234)
Ac-rrr-PEG₂-K(cyclo[Ff-Nal-GrGrQ])-PEG₁₂-OH
(SEQ ID NO: 236)
Ac-rrr-PEG₂-K(cyclo[Ff-Nal-Cit-r-Cit-rQ])-PEG₁₂-OH
(SEQ ID NO: 235)
Ac-rrr-PEG₂-K(cyclo[FfF-GRGRQ])-PEG₁₂-OH
(SEQ ID NO: 128)
Ac-rrr-PEG₂-K(cyclo[FGFGRGRQ])-PEG₁₂-OH
(SEQ ID NO: 233)
Ac-rrr-PEG₂-K(cyclo[GfFGrGrQ])-PEG₁₂-OH
(SEQ ID NO: 130)
Ac-rrr-PEG₂-K(cyclo[FGFGRRRQ])-PEG₁₂-OH
(SEQ ID NO: 129)
Ac-rrr-PEG₂-K(cyclo[FGFRRRRQ])-PEG₁₂-OH
(SEQ ID NO: 234)
Ac-rhr-PEG₂-K(cyclo[Ff-Nal-GrGrQ])-PEG₁₂-OH
(SEQ ID NO: 236)
Ac-rhr-PEG₂-K(cyclo[Ff-Nal-Cit-r-Cit-rQ])-PEG₁₂-OH
(SEQ ID NO: 235)
Ac-rhr-PEG₂-K(cyclo[FfF-GRGRQ])-PEG₁₂-OH
(SEQ ID NO: 128)
Ac-rhr-PEG₂-K(cyclo[FGFGRGRQ])-PEG₁₂-OH
(SEQ ID NO: 233)
Ac-rhr-PEG₂-K(cyclo[GfFGrGrQ])-PEG₁₂-OH
(SEQ ID NO: 130)
Ac-rhr-PEG₂-K(cyclo[FGFGRRRQ])-PEG₁₂-OH
Ac-rhr-PEG₂-K(cyclo[FGFRRRRQ])-PEG₁₂-OH
(SEQ ID NO: 234)
Ac-rbr-PEG₂-K(cyclo[Ff-Nal-GrGrQ])-PEG₁₂-OH
(SEQ ID NO: 236)
Ac-rbr-PEG₂-K(cyclo[Ff-Nal-Cit-r-Cit-rQ])-PEG₁₂-OH
(SEQ ID NO: 235)
Ac-rbr-PEG₂-K(cyclo[FfF-GRGRQ])-PEG₁₂-OH
(SEQ ID NO: 128)
Ac-rbr-PEG₂-K(cyclo[FGFGRGRQ])-PEG₁₂-OH
(SEQ ID NO: 233)
Ac-rbr-PEG₂-K(cyclo[GfFGrGrQ])-PEG₁₂-OH
(SEQ ID NO: 130)
Ac-rbr-PEG₂-K(cyclo[FGFGRRRQ])-PEG₁₂-OH
(SEQ ID NO: 129)
Ac-rbr-PEG₂-K(cyclo[FGFRRRRQ])-PEG₁₂-OH
(SEQ ID NO: 234)
Ac-rbrbr-PEG₂-K(cyclo[Ff-Nal-GrGrQ])-PEG1-OH
(SEQ ID NO: 236)
Ac-rbrbr-PEG₂-K(cyclo[Ff-Nal-Cit-r-Cit-rQ])-PEG₁₂-
OH
(SEQ ID NO: 235)
Ac-rbrbr-PEG₂-K(cyclo[FfF-GRGRQ])-PEG₁₂-OH
(SEQ ID NO: 128)
Ac-rbrbr-PEG₂-K(cyclo[FGFGRGRQ])-PEG₁₂-OH
(SEQ ID NO: 233)
Ac-rbrbr-PEG₂-K(cyclo[GfFGrGrQ])-PEG₁₂-OH
(SEQ ID NO: 130)
Ac-rbrbr-PEG₂-K(cyclo[FGFGRRRQ])-PEG₁₂-OH
(SEQ ID NO: 129)
Ac-rbrbr-PEG₂-K(cyclo[FGFRRRRQ])-PEG₁₂-OH
(SEQ ID NO: 234)
Ac-rbhbr-PEG₂-K(cyclo[Ff-Nal-GrGrQ])-PEG₁₂-OH
(SEQ ID NO: 236)
Ac-rbhbr-PEG₂-K(cyclo[Ff-Nal-Cit-r-Cit-rQ])-PEG₁₂-
OH
(SEQ ID NO: 235)
Ac-rbhbr-PEG₂-K(cyclo[FfF-GRGRQ])-PEG₁₂-OH
(SEQ ID NO: 128)
Ac-rbhbr-PEG₂-K(cyclo[FGFGRGRQ])-PEG₁₂-OH
(SEQ ID NO: 233)
Ac-rbhbr-PEG₂-K(cyclo[GfFGrGrQ])-PEG₁₂-OH
(SEQ ID NO: 130)
Ac-rbhbr-PEG₂-K(cyclo[FGFGRRRQ])-PEG₁₂-OH
(SEQ ID NO: 129)
Ac-rbhbr-PEG₂-K(cyclo[FGFRRRRQ])-PEG₁₂-OH
(SEQ ID NO: 234)
Ac-hbrbh-PEG₂-K(cyclo[Ff-Nal-GrGrQ])-PEG₁₂-OH
(SEQ ID NO: 236)
Ac-hbrbh-PEG₂-K(cyclo[Ff-Nal-Cit-r-Cit-rQ])-PEG₁₂-
OH
(SEQ ID NO: 235)
Ac-hbrbh-PEG₂-K(cyclo[FfF-GRGRQ])-PEG₁₂-OH
(SEQ ID NO: 128)
Ac-hbrbh-PEG₂-K(cyclo[FGFGRGRQ])-PEG₁₂-OH
(SEQ ID NO: 233)
Ac-hbrbh-PEG₂-K(cyclo[GfFGrGrQ])-PEG₁₂-OH
(SEQ ID NO: 130)
Ac-hbrbh-PEG₂-K(cyclo[FGFGRRRQ])-PEG₁₂-OH
and
(SEQ ID NO: 129)
Ac-hbrbh-PEG₂-K(cyclo[FGFRRRRQ])-PEG₁₂-OH

wherein b is beta-alanine, and the exocyclic sequence can be D or L stereochemistry.

### Cargo

The cell penetrating peptide (CPP), such as a cyclic cell penetrating peptide (e.g., cCPP), can be conjugated to a cargo. The cargo can be a therapeutic moiety. The cargo can be conjugated to a terminal carbonyl group of a linker. At least one atom of the cyclic peptide can be replaced by a cargo or at least one lone pair can form a bond to a cargo. The cargo can be conjugated to the cCPP by a linker. The cargo can be conjugated to an AA_{SC} by a linker. At least one atom of the cCPP can be replaced by a therapeutic moiety or at least one lone pair of the cCPP forms a bond to a therapeutic moiety. A hydroxyl group on an amino acid side chain of the cCPP can be replaced by a bond to the cargo. A hydroxyl group on a glutamine side chain of the cCPP can be replaced by a bond to the cargo. The cargo can be conjugated to the cCPP by a linker. The cargo can be conjugated to an AA_{SC} by a linker.

The cargo can comprise one or more detectable moieties, one or more therapeutic moieties, one or more targeting moieties, or any combination thereof. The cargo can be a peptide, oligonucleotide, or small molecule. The cargo can be a peptide sequence or a non-peptidyl therapeutic agent. The cargo can be an antibody or an antigen binding fragment thereof, including, but not limited to an scFv or nanobody.

The cargo can comprise one or more additional amino acids (e.g., K, UK, TRV); a linker (e.g., bifunctional linker LC-SMCC); coenzyme A; phosphocoumaryl amino propionic acid (pCAP); 8-amino-3,6-dioxaoctanoic acid (miniPEG); L-2,3-diaminopropionic acid (Dap or J); L-β-naphthylalanine; L-pipecolic acid (Pip); sarcosine; trimesic acid; 7-amino-4-methylcourmarin (Amc); fluorescein isothiocyanate (FITC); L-2-naphthylalanine; norleucine; 2-aminobutyric acid; Rhodamine B (Rho); Dexamethasone (DEX); or combinations thereof.

The cargo can comprise any of those listed in Table 6, or derivatives or combinations thereof.

TABLE 6
Example cargo moieties

| SEQ ID NO | Abbreviation | Sequence* |
|---|---|---|
| 1 | R₅ | RRRRR |
| 2 | A₅ | AAAAA |
| 3 | F₄ | FFFF |
| 4 | PCP | DE(pCAP)LI |
| 5 | A₇ | AAAAAAA |
| 6 | | RARAR |
| 7 | | DADAD |
| 8 | | DΩUD |
| 9 | | UTRV |
| 10 | | D-pThr-Pip-Nal |

*pCAP, phosphocoumaryl amino propionic acid; Ω, norleucine; U, 2-aminobutyric acid; D-pThr is D-phosphothreonine, Pip is L-piperidine-2-carboxylate.

### Detectable Moiety

The compound can include a detectable moiety. The detectible moiety can be attached to a cell penetrating peptide (CPP) at the amino group, the carboxylate group, or the side chain of any of the amino acids of the CPP (e.g., at the amino group, the carboxylate group, or the side chain of any amino acid in the cCPP). The detectable moiety can be attached to a cyclic cell penetrating peptide (cCPP) at the side chain of any amino acid in the cCPP. The cargo can include a detectable moiety. The cargo can include a therapeutic agent and a detectable moiety. The detectable moiety can include any detectable label. Examples of suitable detectable labels include, but are not limited to, a UV-Vis label, a near-infrared label, a luminescent group, a phosphorescent group, a magnetic spin resonance label, a photosensitizer, a photocleavable moiety, a chelating center, a heavy atom, a radioactive isotope, an isotope detectable spin resonance label, a paramagnetic moiety, a chromophore, or any combination thereof. The label can be detectable without the addition of further reagents.

The detectable moiety can be a biocompatible detectable moiety, such that the compounds can be suitable for use in a variety of biological applications. “Biocompatible” and “biologically compatible”, as used herein, generally refer to compounds that are, along with any metabolites or degradation products thereof, generally non-toxic to cells and tissues, and which do not cause any significant adverse effects to cells and tissues when cells and tissues are incubated (e.g., cultured) in their presence.

The detectable moiety can contain a luminophore such as a fluorescent label or near-infrared label. Examples of suitable luminophores include, but are not limited to, metal porphyrins; benzoporphyrins; azabenzoporphyrine; napthoporphyrin; phthalocyanine; polycyclic aromatic hydrocarbons such as diimine, pyrenes; azo dyes; xanthene dyes; boron dipyoromethene, aza-boron dipyoromethene, cyanine dyes, metal-ligand complex such as bipyridine, bipyridyls, phenanthroline, coumarin, and acetylacetonates of ruthenium and iridium; acridine, oxazine derivatives such as benzophenoxazine; aza-annulene, squaraine; 8-hydroxyquinoline, polymethines, luminescent producing nanoparticle, such as quantum dots, nanocrystals; carbostyril; terbium complex; inorganic phosphor; ionophore such as crown ethers affiliated or derivatized dyes; or combinations thereof. Specific examples of suitable luminophores include, but are not limited to, Pd (II) octaethylporphyrin; Pt (II)-octaethylporphyrin; Pd (II) tetraphenylporphyrin; Pt (II) tetraphenylporphyrin; Pd (II) meso-tetraphenylporphyrin tetrabenzoporphine; Pt (II) meso-tetraphenyl metrylbenzoporphyrin; Pd (11) octaethylporphyrin ketone; Pt (II) octaethylporphyrin ketone; Pd (II) meso-tetra(pentafluorophenyl)porphyrin; Pt (II) meso-tetra (pentafluorophenyl) porphyrin; Ru (11) tris(4,7-diphenyl-1,10-phenanthroline) (Ru (dpp)₃); Ru (II) tris(1,10-phenanthroline) (Ru(phen)₃), tris(2,2′-bipyridine)ruthenium (II) chloride hexahydrate (Ru(bpy)₃); erythrosine B; fluorescein; fluorescein isothiocyanate (FITC); eosin; iridium (III) ((N-methyl-benzimidazol-2-yl)-7-(diethylamino)-coumarin)); 92enzothiazole) ((benzothiazol-2-yl)-7-(diethylamino)-coumarin))-2-(acetylacetonate); Lumogen dyes; Macroflex fluorescent red; Macrolex fluorescent yellow; Texas Red; rhodamine B; rhodamine 6G; sulfur rhodamine; m-cresol; thymol blue; xylenol blue; cresol red; chlorophenol blue; bromocresol green; bromcresol red; bromothymol blue; Cy2; a Cy3; a Cy5; a Cy5.5; Cy7; 4-nitirophenol; alizarin; phenolphthalein; o-cresolphthalein; chlorophenol red; calmagite; bromo-xylenol; phenol red; neutral red; nitrazine; 3,4,5,6-tetrabromphenolphtalein; congo red; fluor‘sc’in; eosin; 2′,7′-dichlorofluorescein; 5(6)-carboxy-fluorecsein; carboxynaphthofluorescein; 8-hydroxypyrene-1,3,6-trisulfonic acid; semi-naphthorhodafluor; semi-naphthofluorescein; tris (4,7-diphenyl-1,10-phenanthroline) ruthenium (II) dichloride; (4,7-diphenyl-1,10-phenanthroline) ruthenium (II) tetraphenylboron; platinum (II) octaethylporphyin; dialkylcarbocyanine; dioctadecylcycloxacarbocyanine; fluorenylmethyloxycarbonyl chloride; 7-amino-4-methylcourmarin (Amc); green fluorescent protein (GFP); and derivatives or combinations thereof.

The detectable moiety can include Rhodamine B (Rho), fluorescein isothiocyanate (FITC), 7-amino-4-methylcourmarin (Amc), green fluorescent protein (GFP), or derivatives or combinations thereof.

The detectible moiety can be attached to a cell penetrating peptide (CPP) at the amino group, the carboxylate group, or the side chain of any of the amino acids of the cell penetrating peptide (e.g., at the amino group, the carboxylate group, or the side chain of any amino acid in the cCPP).

### Therapeutic Moiety

The disclosed compounds can comprise a therapeutic moiety. The cargo can comprise a therapeutic moiety. The detectable moiety can be linked to a therapeutic moiety or a detectable moiety can also serve as the therapeutic moiety. Therapeutic moiety refers to a group that when administered to a subject will reduce one or more symptoms of a disease or disorder. The therapeutic moiety can comprise a peptide, protein (e.g., enzyme, antibody or fragment thereof), small molecule, or oligonucleotide.

The therapeutic moiety can comprise a wide variety of drugs, including antagonists, for example enzyme inhibitors, and agonists, for example a transcription factor which results in an increase in the expression of a desirable gene product (although as will be appreciated by those in the art, antagonistic transcription factors can also be used), are all included. In addition, therapeutic moiety includes those agents capable of direct toxicity and/or capable of inducing toxicity towards healthy and/or unhealthy cells in the body. Also, the therapeutic moiety can be capable of inducing and/or priming the immune system against potential pathogens.

The therapeutic moiety can, for example, comprise an anticancer agent, antiviral agent, antimicrobial agent, anti-inflammatory agent, immunosuppressive agent, anesthetics, or any combination thereof.

The therapeutic moiety can comprise an anticancer agent. Example anticancer agents include 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C₂₂₅, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, lbritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The therapeutic moiety can also comprise a biopharmaceutical such as, for example, an antibody.

The therapeutic moiety can comprise an antiviral agent, such as ganciclovir, azidothymidine (AZT), lamivudine (3TC), etc.

The therapeutic moiety can comprise an antibacterial agent, such as acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; Lomefloxacin hydrochloride; lomefloxacin mesylate; loracarbef; mafenide; meciocycline; meciocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin; oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillin G benzathine; penicillin G potassium; penicillin G procaine; penicillin G sodium; penicillin V; penicillin V benzathine; penicillin V hydrabamine; penicillin V potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin B sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; or zorbamycin.

The therapeutic moiety can comprise an anti-inflammatory agent.

The therapeutic moiety can comprise dexamethasone (Dex).

The therapeutic moiety can comprise a therapeutic protein. For example, some people have defects in certain enzymes (e.g., lysosomal storage disease). Such enzymes/proteins can be delivered to human cells by linking the enzyme/protein to a cyclic cell penetrating peptide (cCPP) disclosed herein. The disclosed cCPP have been tested with proteins (e.g., GFP, PTP1B, actin, calmodulin, troponin C) and shown to work.

The therapeutic moiety can be an anti-infective agent. The term “anti-infective agent” refers to agents that are capable of killing, inhibiting, or otherwise slowing the growth of an infectious agent. The term “infectious agent” refers to pathogenic microorganisms, such as bacteria, viruses, fungi, and intracellular or extracellular parasites. The anti-infective agent can be used to treat an infectious disease, as infectious diseases are caused by infectious agents.

The infectious agent can be a Gram-negative bacteria. The Gram-negative bacteria can be of a genus selected from Escherichia, Proteus, Salmonella, Klebsiella, Providencia, Enterobacter, Burkholderia, Pseudomonas, Acinetobacter, Aeromonas, Haemophilus, Yersinia, Neisseria, Erwinia, Rhodopseudomonas and Burkholderia. The infectious agent can be a Gram-positive bacteria. The Gram-positive bacteria can be of a genus selected from Lactobacillus, Azorhizobium, Streptococcus, Pediococcus, Photobacterium, Bacillus, Enterococcus, Staphylococcus, Clostridium, Butyrivibrio, Sphingomonas, Rhodococcus and Streptomyces. The infectious agent can be an acid-fast bacteria of the Mycobacterium genus, such as Mycobacterium tuberculosis, Mycobacterium bovis. Mycobacterium avium and Mycobacterium leprae. The infectious agent can be of the genus Nocardia. The infectious agent can be selected from any one of the following species Nocardia asteroides, Nocardia brasiliensis and Nocardia caviae.

The infectious agent can be a fungus. The fungus can be from the genus Mucor. The fungus can be from the genus Crytococcus. The fungus can be from the genus Candida. The fungus can be selected from any one of Mucor racemosus, Candida albicans, Crytococcus neoformans, or Aspergillus fumingatus.

The infectious agent can be a protozoa. The protozoa can be of the genus Plasmodium (e.g., P. falciparum, P. vivax, P. ovale, or P. malariae). The protozoa causes malaria.

Illustrative organisms include Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia.

The infectious agent can be a parasite. The parasite can be cryptosporidium. The parasite can be an endoparasite. The endoparasite can be heartworm, tapeworm, or flatworm. The parasite can be an epiparasite. The parasite causes a disease selected from acanthamoebiasis, babesiosis, balantidiasis, blastocystosis, coccidiosis, amoebiasis, giardiasis, isosporiasis, cystosporiasis, leishmaniasis, primary amoebic meningoencephalitis, malaria, rhinosporidiosis, toxoplasmosis, trichomoniasis, trypanomiasis, Chagas disease, or scabies.

The infectious agent can be a virus. Non-limiting examples of viruses include sudden acute respiratory coronavirus 2 (SARS-CoV-2), sudden acute respiratory coronavirus (SARS-CoV), Middle East Respiratory virus (MERS), influenza, Hepatitis C virus, Dengue virus, West Nile virus, Ebola virus, Hepatitis B, Human immunodeficiency virus (HIV), herpes simplex, Herpes zoster, and Lassa virus.

The anti-infective agent can be an antiviral agent. Non-limiting examples of antiviral agents include nucleoside or nucleotide reverse transcriptase inhibitors, such as zidovudine (AZT), didanosine (ddl), zalcitabine (ddC), stavudine (d4T), lamivudine (3TC), emtricitabine, abacavir succinate, elvucitabine, adefovir dipivoxil, lobucavir (BMS-180194) lodenosine (FddA) and tenofovir including tenofovir disoproxil and tenofovir disoproxil fumarate salt, non-nucleoside reverse transcriptase inhibitors, such as nevirapine, delaviradine, efavirenz, etravirine and rilpivirine, protease inhibitros, such as ritonavir, tipranavir, saquinavir, nelfinavir, indinavir, amprenavir, fosamprenavir, atazanavir, lopinavir, darunavir (TMC-114), lasinavir and brecanavir (VX-385), cellular entry inhibitors, such as CCR5 antagonists (e.g., maraviroc, vicriviroc, INCB9471 and TAK-652) and CXCR4 antagonists (AMD-11070), fusion inhibitors, such as enfuvirtide, integrase inhibitors, such as raltegravir, BMS-707035, and elvitegravir, Tat inhibitors, such as didehydro-cortistatin A (dCA), maturation inhibitors, such as berivimat, immunomodulating agents, such as levamisole, and other antiviral agents, such as hydroxyurea, ribavirin, interleukin 2 (IL-2), interleukin 12 (IL-12), pensafuside, peramivir, zanamivir, oseltamivir phosphate, baloxavir marboxil,

The anti-infective agent can be an antibiotic. Non-limiting examples of antibiotics include aminoglycosides, such as amikacin, gentamicin, kanamycin, neomycin, netilmicin, streptomycin and tobramycin; cabecephems, such as loracarbef; carbapenems, such as ertapenem, imipenem/cilastatin and meropenem; cephalosporins, such as cefadroxil, cefazolin, cephalexin, cefaclor, cefamandole, cephalexin, cefoxitin, cefprozil, cefuroxime, cefixime, cefdinir, cefditoren, cefoperazone, cefotaxime, cefpodoxime, ceftazidime, ceftibuten, ceftizoxime, ceftriaxone and cefepime; macrolides, such as azithromycin, clarithromycin, dirithromycin, erythromycin and troleandomycin; monobactam; penicillins, such as amoxicillin, ampicillin, carbenicillin, cloxacillin, dicloxacillin, nafcillin, oxacillin, penicillin G, penicillin V, piperacillin and ticarcillin; polypeptides, such as bacitracin, colistin and polymyxin B; quinolones, such as ciprofloxacin, enoxacin, gatifloxacin, levofloxacin, lomefloxacin, moxifloxacin, norfloxacin, ofloxacin and trovafloxacin; sulfonamides, such as mafenide, sulfacetamide, sulfamethizole, sulfasalazine, sulfisoxazole and trimethoprim-sulfamethoxazole; tetracyclines, such as demeclocycline, doxycycline, minocycline, oxytetracycline and tetracycline; and vancomycin. The anti-infective agent can be a steroidal anti-inflammatory agent. Non-limiting examples of steroidal anti-inflammatory agents include fluocinolone, triamcinolone, triamcinoline acetonide, betamethasone, betamethasone diproprionate, diflucortolone, fluticasone, cortisone, hydrocortisone, mometasone, methylprednisolone, beclomethasone diproprionate, clobetasol, prednisone, prednisolone, meythylprednisolone, betamethasone, budesonide, and dexamethasone. The anti-infective agent can be a non-steroidal anti-inflammatory agent. Non-limiting examples of non-steroidal anti-inflammatory agents include celocoxib, nimesulide, rofecoxib, meclofenamic acid, meclofenamate sodium, flunixin, fluprofen, flurbiprofen, sulindac, meloxicam, piroxicam, etodolac, fenoprofen, fenbuprofen, ketoprofen, suprofen, diclofenac, bromfenac sodium, phenylbutazone, thalidomide and indomethacin.

The anti-infective agent can be an anti-fungal. Non-limiting examples of anti-fungals include amphotericin B, caspofungin, fluconazole, flucytosine, itraconazole, ketoconazole, amrolfine, butenafine, naftifine, terbinafine, elubiol, econazole, econaxole, itraconazole, isoconazole, imidazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, flutrimazole, nystatin, pimaricin, natamycin, tolnaftate, mafenide, dapsone, actofunicone, griseofulvin, potassium iodide, Gentian Violet, ciclopirox, ciclopirox olamine, haloprogin, undecylenate, silver sulfadiazine, undecylenic acid, undecylenic alkanolamide, and Carbol-Fuchsin.

The therapeutic moiety can be an analgesic or pain-relieving agent. Non-limiting examples of analgesics or pain-relieving agents include aspirin, acetaminophen, ibuprofen, naproxen, procaine, lidocaine, tetracaine, dibucaine, benzocaine, p-buthylaminobenzoic acid 2-(diethylamino) ethyl ester HCI, mepivacaine, piperocaine, and dyclonine

The therapeutic moiety can bean antibody or an antigen-binding fragment. Antibodies and antigen-binding fragments can be derived from any suitable source, including human, mouse, camelid (e.g., camel, alpaca, llama), rat, ungulates, or non-human primates (e.g., monkey, rhesus macaque).

It should furthermore be understood that the cargos including anti-infective agents and other therapeutic moieties described herein include possible salts thereof, of which pharmaceutically acceptable salts are of course especially relevant for the therapeutic applications. Salts include acid addition salts and basic salts. Examples of acid addition salts are hydrochloride salts, fumarate, oxalate, etc. Examples of basic salts are salts where the (remaining) counter ion can be selected from alkali metals, such as sodium and potassium, alkaline earth metals, such as calcium salts, potassium salts, and ammonium ions (⁺N(R′)₄, where the R's independently designate optionally substituted C₁₋₆-alkyl, optionally substituted C₂₋₆-alkenyl, optionally substituted aryl, or optionally substituted heteroaryl).

The therapeutic moiety can be an oligonucleotide. The oligonucleotide can be an antisense compound (AC). The oligonucleotide can include, for example, but is not limited to, antisense oligonucleotides, small interfering RNA (siRNA), microRNA (miRNA), ribozymes, immune stimulating nucleic acids, antagomir, antimir, microRNA mimic, supermir, Ul adaptors, CRISPR machinery and aptamers. The term “antisense oligonucleotide” or simply “antisense” is meant to include oligonucleotides that are complementary to a targeted polynucleotide sequence. Non-limiting examples of antisense oligonucleotides for treating Duchenne muscular dystrophy may be found in US Pub. No. 2019/0365918, US Pub. No. US2020/0040336, U.S. Pat. Nos. 9,499,818, and 9,447,417 each of which is incorporated by reference in its entirety for all purposes.

The therapeutic moiety can be used to treat any one of the following diseases: neuromuscular disorders, Pompe disease, β-thalassemia, dystrophin Kobe, Duchenne muscular dystrophy, Becker muscular dystrophy, diabetes, Alzheimer's disease, cancer, cystic fibrosis, Merosin-deficient congenital muscular dystrophy type 1A (MDC1A), proximal spinal muscular atrophy (SMA), Huntington's disease, Huntington disease-like 2 (HDL2), myotonic dystrophy, spinocerebellar ataxia, spinal and bulbar muscular atrophy (SBMA), dentatorubral-pallidoluysian atrophy (DRPLA), amyotrophic lateral sclerosis, frontotemporal dementia, Fragile X syndrome, fragile X mental retardation 1 (FMR1), fragile X mental retardation 2 (FMR2), Fragile XE mental retardation (FRAXE), Friedreich's ataxia (FRDA), fragile X-associated tremor/ataxia syndrome (FXTAS), myoclonic epilepsy, oculopharyngeal muscular dystrophy (OPMD), syndromic or non-syndromic X-linked mental retardation, myotonic dystrophy, myotonic dystrophy type 1, myotonic dystrophy type 2, epilepsy, Dravet syndrome, or Alzheimer's disease. The therapeutic moiety can be used to treat a cancer selected from glioma, acute myeloid leukemia, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, or melanoma. The therapeutic moiety can be used to treat an ocular disease. Non-limiting examples of ocular diseases include refractive errors, macular degeneration, cataracts, diabetic retinopathy, glaucoma, amblyopia, or strabismus.

The therapeutic moiety can comprise a targeting moiety. The targeting moiety can comprise, for example, a sequence of amino acids that can target one or more enzyme domains. The targeting moiety can comprise an inhibitor against an enzyme that can play a role in a disease, such as cancer, cystic fibrosis, diabetes, obesity, or combinations thereof. The targeting moiety targets one or more of the following genes: FMR1, AFF2, FAN, DMPK, SCA8, PPP2R2B, ATN1, DRPLA, HTT, AR, ATXN1, ATXN2, ATXN3, CACNA1A, ATXV7, TBP, ATP7B, HTT, SCN1A, BRCA1, LAMA2, CD33, VEGF, ABCA4, CEP290, RHO, UISH2A, OPA1, CNGB3, PRPF31, GYS1, or RPGR. The therapeutic moiety can be an antisense compound (AC) described in U.S. Publication No. 2019/0365918, which is incorporated by reference herein in its entirety. For example, the targeting moiety can comprise any of the sequences listed in Table 7.

TABLE 7
Example targeting moieties

| SEQ ID NO | Abbreviation * | Sequence |
|---|---|---|
| 11 | PΘGΛYR | Pro-Pip-Gly-F₂Pmp-Tyr-Arg |
| 12 | SΘIΛΛR | Ser-Pip-Ile-F₂Pmp-F₂Pmp-Arg |
| 13 | IHIΛIR | Ile-His-Ile-F₂Pmp-Ile-Arg |
| 14 | AaIΛΘR | Ala-(D-Ala)-Ile-F₂Pmp-Pip-Arg |
| 15 | ΣSΘΛvR | Fpa-Ser-Pip-F₂Pmp-(D-Val)-Arg |
| 16 | ΘnPΛAR | Pip-(D-Asn)-Pro-F₂Pmp-Ala-Arg |
| 17 | TΨAΛGR | Tyr-Phg-Ala-F₂Pmp-Gly-Arg |
| 18 | AHIΛaR | Ala-His-Ile- F₂Pmp-(D-Ala)-Arg |
| 19 | GnGΛpR | Gly-(D-Asn)-Gly-F₂Pmp-(D-Pro)-Arg |
| 20 | fQθΛIR | (D-Phe)-Gln-Pip-F₂Pmp-Ile-Arg |
| 21 | SPGΛHR | Ser-Pro-Gly-F₂Pmp-His- Arg |
| 22 | θYIΛHR | Pip-Tyr-Ile-F₂Pmp-His-Arg |
| 23 | SyPΛHR | Ser-(D-Val)-Pro-F₂Pmp-His-Arg |
| 24 | AIPΛnR | Ala-Ile-Pro-F₂Pmp-(D-Asn)- Arg |
| 25 | ΣSIΛQF | Fpa-Ser-Ile-F₂Pmp-Gln- Arg |
| 26 | AaΨΛfR | Ala-(D-Ala)-Phg-F₂Pmp-(D-Phe)-Arg |
| 27 | ntΨΛWR | (D-Asn)-(D-Thr)-Phg-F₂Pmp-Phg-Arg |
| 28 | IPΨΛΩR | Ile-Pro-Phg-F₂Pmp-Nle-Arg |
| 29 | QΘΣΛΘR | Gln-Pip-Fpa-F₂Pmp-Pip-Arg |
| 30 | nAΣΛGR | (D-Asn)-Ala-Fpa-F₂Pmp-Gly-Arg |
| 31 | ntYΛAR | (D-Asn)-(D-Thr)-Tyr-F₂Pmp-Ala-Arg |
| 32 | eAΨΛVR | (D-Glu)-Ala-Phg-F₂Pmp-(D-Val)-Arg |
| 33 | IvΨΛAR | Ile-(D-Val)-Phg-F₂Pmp-Ala-Arg |
| 34 | YtΨΛAR | Tyr-(D-Thr)-Phg-F₂Pmp-Ala-Arg |
| 35 | nΘΨΛIR | (D-Asn)-Pip-Phg-F₂Pmp-Ile-Arg |
| 36 | ΘnWΛHR | Pip-(D-Asn)-Trp-F₂Pmp-His-Arg |
| 37 | YΘvΛIR | Tyr-Pip-(D-Val)-F₂Pmp-Ile- Arg |
| 38 | nSAΛGR | (D-Asn)-Ser-(D-Ala)-F₂Pmp-Gly-Arg |
| 39 | tnvΛaR | (D-Thr)-(D-Asn)-(D-Val)-F₂Pmp-(D-Ala)-Arg |
| 40 | ntvΛtR | (D-Asn)-(D-Thr)-(D-Val)-F₂Pmp-(D-Thr)-Arg |
| 41 | SItΛYR | Ser-Ile-(D-Thr)-F₂Pmp-Tyr-Arg |
| 42 | nΣnΛlR | (D-Asn)-Fpa-(D-Asn)-F₂Pmp-(D-Leu)-Arg |
| 43 | YnnΛΩR | Tyr-(D-Asn)-(D-Asn)-F₂Pmp-Nle-Arg |
| 44 | nYnΛGR | (D-Asn)-Tyr-(D-Asn)-F₂Pmp-Gly-Arg |
| 45 | AWnΛAR | Ala-Trp-(D-Asn)-F₂Pmp-Ala-Arg |
| 46 | VtHΛYR | (D-Val)-(D-Thr)-His-F₂Pmp-Tyr-Arg |
| 47 | PΨHΛΘR | Pro-Phg-His-F₂Pmp-Pip-Arg |
| 48 | nΨHΛGR | (D-Asn)-Phg-His-F₂Pmp-Gly-Arg |
| 49 | PAHΛGR | Pro-Ala-His-F₂Pmp-Gly-Arg |
| 50 | AYHΛIR | Ala-Tyr-His-F₂Pmp-Ile-Arg |
| 51 | nΘeΛYR | (D-Asn)-Pip-(D-Glu)-F₂Pmp-Tyr-Arg |
| 52 | vSSΛtR | (D-Val)-Ser-Ser-F₂Pmp-(D-Thr)-Arg |
| 53 | aΞt′ ϑ Φ′YNK | ((D-Ala)-Sar-(D-pThr)-Pp-Nal-Tyr-Gln)-Lys |
| 54 | Tm(aΞt′ϑΦ′RA)Dap | Tm((D-Ala)-Sar-(D-pThr)-Pp-Nal-Arg-Ala)-Dap |
| 55 | Tm(aΞt′ϑΦ′RAa)Dap | Tm((D-Ala)-Sar-(D-pThr)-Pp-Nal-Arg-Ala-(D- |
| | | Ala))-Dap |
| 56 | Tm(aΞtϑΦ′RAa)Dap | Tm((D-Ala)-Sar-(D-Thr)-Pp-Nal-Arg-Ala-(D- |
| | | Ala))-Dap |
| 57 | Tm(aΞtaΦ′RAa)Dap | Tm((D-Ala)-Sar-(D-Thr)-(D-Ala)-Nal-Arg-Ala-(D- |
| | | Ala))-Dap |

*Fpa, Σ: L-4-fluorophenylalanine; Pip, Θ: L-homoproline; Nle, Ω: L-norleucine; Phg, Ψ L-phenylglycine; F₂Pmp, Λ: L-4-(phosphonodifluoromethyl)phenylalanine; Dap, L-2,3-diaminopropionic acid; Nal, Φ′: L-B-naphthylalanine; Pp, ϑ: L-pipecolic acid; Sar, Ξ: sarcosine; Tm, trimesic acid.

The targeting moiety and cell penetrating peptide can overlap. That is, the residues that form the cell penetrating peptide can also be part of a sequence that forms a targeting moiety, and vice versa.

The therapeutic moiety can be attached to the cell penetrating peptide at the amino group, the carboxylate group, or the side chain of any of the amino acids of the cell penetrating peptide (e.g., at the amino group, the carboxylate group, or the side chain or any of amino acid of the cCPP). The therapeutic moiety can be attached to a detectable moiety.

The therapeutic moiety can comprise a targeting moiety that can act as an inhibitor against Ras (e.g., K-Ras), PTP1B, Pin1, Grb2 SH2, CAL PDZ, and the like, or combinations thereof.

Ras is a protein that in humans is encoded by the RAS gene. The normal Ras protein performs an essential function in normal tissue signaling, and the mutation of a Ras gene is implicated in the development of many cancers. Ras can act as a molecular on/off switch, once it is turned on Ras recruits and activates proteins necessary for the propagation of growth factor and other receptors' signal. Mutated forms of Ras have been implicated in various cancers, including lung cancer, colon cancer, pancreatic cancer, and various leukemias.

Protein-tyrosine phosphatase 1B (PTP1B) is a prototypical member of the PTP superfamily and plays numerous roles during eukaryotic cell signaling. PTP1B is a negative regulator of the insulin signaling pathway, and is considered a promising potential therapeutic target, in particular for the treatment of type II diabetes. PIP1B has also been implicated in the development of breast cancer.

Pin1 is an enzyme that binds to a subset of proteins and plays a role as a post phosphorylation control in regulating protein function. Pin1 activity can regulate the outcome of proline-directed kinase signaling and consequently can regulate cell proliferation and cell survival. Deregulation of Pin1 can play a role in various diseases. The up-regulation of Pin1 may be implicated in certain cancers, and the down-regulation of Pin1 may be implicated in Alzheimer's disease. Inhibitors of Pin1 can have therapeutic implications for cancer and immune disorders.

Grb2 is an adaptor protein involved in signal transduction and cell communication. The Grb2 protein contains one SH2 domain, which can bind tyrosine phosphorylated sequences. Grb2 is widely expressed and is essential for multiple cellular functions. Inhibition of Grb2 function can impair developmental processes and can block transformation and proliferation of various cell types.

It was recently reported that the activity of cystic fibrosis membrane conductance regulator (CFTR), a chloride ion channel protein mutated in cystic fibrosis (CF) patients, is negatively regulated by CFTR-associated ligand (CAL) through its PDZ domain (CAL-PDZ) (Wolde, M et al. J. Biol. Chem. 2007, 282, 8099). Inhibition of the CFTR/CAL-PDZ interaction was shown to improve the activity of ΔPhe508-CFTR, the most common form of CFTR mutation (Cheng, S H et al. Cell 1990, 63, 827; Kerem, B S et al. Science 1989, 245, 1073), by reducing its proteasome-mediated degradation (Cushing, P R et al. Angew. Chem. Int. Ed. 2010, 49, 9907). Thus, disclosed herein is a method for treating a subject having cystic fibrosis by administering an effective amount of a compound or composition disclosed herein. The compound or composition administered to the subject can comprise a therapeutic moiety that can comprise a targeting moiety that can act as an inhibitor against CAL PDZ. Also, the compositions or compositions disclosed herein can be administered with a molecule that corrects the CFTR function.

The therapeutic moiety can be attached to the cyclic peptide at an amino group or carboxylate group, or a side chain of any of the amino acids of the cyclic peptide (e.g., at an amino group or the carboxylate group on the side chain of an amino acid of the cyclic peptide). In some examples, the therapeutic moiety can be attached to a detectable moiety.

Also disclosed herein are compositions comprising the compounds described herein.

Also disclosed herein are pharmaceutically-acceptable salts and prodrugs of the disclosed compounds. Pharmaceutically-acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The therapeutic moiety can include a therapeutic polypeptide, an oligonucleotide or a small molecule. The therapeutic polypeptide can include a peptide inhibitor. The therapeutic polypeptide can include a binding reagent that specifically binds to a target of interest. The binding reagent can include an antibody or antigen-binding fragment thereof that specifically binds to a target of interest. The antigen-binding fragments can include a Fab fragment, a F(ab′) fragment, a F(ab′)₂ fragment, a Fv fragment, a minibody, a diabody, a nanobody, a single domain antibody (dAb), a single-chain variable fragment (scFv), or a multispecific antibody.

The oligonucleotide can include an antisense compound (AC). The AC can include a nucleotide sequence complementary to a target nucleotide sequence encoding a protein target of interest.

The therapeutic moiety (TM) can be conjugated to a chemically reactive side chain of an amino acid of the cCPP. Any amino acid side chain on the cCPP which is capable of forming a covalent bond, or which may be so modified, can be used to link the TM to the cCPP. The amino acid on the cCPP can be a natural or non-natural amino acid. The chemically reactive side chain can include an amine group, a carboxylic acid, an amide, a hydroxyl group, a sulfhydryl group, a guanidinyl group, a phenolic group, a thioether group, an imidazolyl group, or an indolyl group. The amino acid of the cCPP to which the TM is conjugated can include lysine, arginine, aspartic acid, glutamic acid, asparagine, glutamine, serine, threonine, tyrosine, cysteine, arginine, tyrosine, methionine, histidine, tryptophan or analogs thereof. The amino acid on the cCPP used to conjugate the TM can be ornithine, 2,3-diaminopropionic acid, or analogs thereof. The amino acid can be lysine, or an analog thereof. The amino acid can be glutamic acid, or an analog thereof. The amino acid can be aspartic acid, or an analog thereof. The side chain can be substituted with a bond to the TM or a linker.

The TM can include a therapeutic polypeptide and the cCPP can be conjugated to a chemically reactive side chain of an amino acid of the therapeutic polypeptide. Any amino acid side chain on the TM which is capable of forming a covalent bond, or which may be so modified, can be used to link the cCPP to the TM. The amino acid on the TM can be a natural or non-natural amino acid. The chemically reactive side chain can include an amine group, a carboxylic acid, an amide, a hydroxyl group, a sulfhydryl group, a guanidinyl group, a phenolic group, a thioether group, an imidazolyl group, or an indolyl group. The amino acid of the TM to which the cCPP is conjugated can include lysine, arginine, aspartic acid, glutamic acid, asparagine, glutamine, serine, threonine, tyrosine, cysteine, arginine, tyrosine, methionine, histidine, tryptophan or analogs thereof. The amino acid on the TM used to conjugate the cCPP can be ornithine, 2,3-diaminopropionic acid, or analogs thereof. The amino acid can be lysine, or an analog thereof. The amino acid can be glutamic acid, or an analog thereof. The amino acid can be aspartic acid, or an analog thereof. The side chain of the TM ca be substituted with a bond to the cCPP or a linker.

The TM can be an antisense compound (AC) that includes an oligonucleotide where the 5′ or 3′ end of the oligonucleotide is conjugated to a chemically reactive side chain of an amino acid of the cCPP. The AC can be chemically conjugated to the cCPP through a moiety on the 5′ or 3′ end of the AC. The chemically reactive side chain of the cCPP can include an amine group, a carboxylic acid, an amide, a hydroxyl group, a sulfhydryl group, a guanidinyl group, a phenolic group, a thioether group, an imidazolyl group, or an indolyl group. The amino acid of the cCPP to which the AC is conjugated can include lysine, arginine, aspartic acid, glutamic acid, asparagine, glutamine, serine, threonine, tyrosine, cysteine, arginine, tyrosine, methionine, histidine or tryptophan. The amino acid of the cCPP to which the AC is conjugated can include lysine or cysteine.

### Oligonucleotides

The compounds can include a cyclic cell penetrating peptide (cCPP) conjugated to an antisense compound (AC) as the therapeutic moiety. The AC can include an antisense oligonucleotide, siRNA, microRNA, antagomir, aptamer, ribozyme, immunostimulatory oligonucleotide, decoy oligonucleotide, supermir, miRNA mimic, miRNA inhibitor, or combinations thereof.

### Antisense Oligonucleotides

The therapeutic moiety can include an antisense oligonucleotide. The term “antisense oligonucleotide” or simply “antisense” refers to oligonucleotides that are complementary to a targeted polynucleotide sequence. Antisense oligonucleotides can include single strands of DNA or RNA that are complementary to a chosen sequence, e.g. a target gene mRNA.

The antisense oligonucleotides may modulate one or more aspects of protein transcription, translation, and expression and functions via hybridization of the antisense oligonucleotide with a target nucleic acid. Hybridization of the antisense oligonucleotide to its target sequence can suppress expression of the target protein. Hybridization of the antisense oligonucleotide to its target sequence can suppress expression of one or more target protein isoforms. Hybridization of the antisense oligonucleotide to its target sequence can upregulate expression of the target protein. Hybridization of the antisense oligonucleotide to its target sequence can downregulate expression of the target protein.

The antisense compound can inhibit gene expression by binding to a complementary mRNA. Binding to the target mRNA can lead to inhibition of gene expression either by preventing translation of complementary mRNA strands by binding to it or by leading to degradation of the target mRNA. Antisense DNA can be used to target a specific, complementary (coding or non-coding) RNA. If binding takes places this DNA/RNA hybrid can be degraded by the enzyme RNase H. The antisense oligonucleotide can include from about 10 to about 50 nucleotides, about to about 30 nucleotides, or about 20 to about 25 nucleotides. The term also encompasses antisense oligonucleotides that may not be fully complementary to the desired target gene. Thus, compounds disclosed herein can be utilized in instances where non-target specific-activities are found with antisense, or where an antisense sequence containing one or more mismatches with the target sequence is desired.

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established.

Methods of producing antisense oligonucleotides are known in the art and can be readily adapted to produce an antisense oligonucleotide that targets any polynucleotide sequence of interest. Selection of antisense oligonucleotide sequences specific for a given target sequence is based upon analysis of the chosen target sequence and determination of secondary structure, Tm, binding energy, and relative stability. Antisense oligonucleotides may be selected based upon their relative inability to form dimers, hairpins, or other secondary structures that would reduce or prohibit specific binding to the target mRNA in a host cell. Target regions of the mRNA include those regions at or near the AUG translation initiation codon and those sequences that are substantially complementary' to 5′ regions of the mRNA. These secondary structure analyses and target site selection considerations can be performed, for example, using v. 4 of the OLIGO primer analysis software (Molecular Biology Insights) and/or the BLASTN 2.0.5 algorithm software (Altschul et ai, Nucleic Acids Res. 1997, 25(17):3389-402).

### RNA Interference Nucleic Acids

The therapeutic moiety can be a RNA interference (RNAi) molecule or a small interfering RNA molecule. RNA interference methods using RNAi or siRNA molecules may be used to disrupt the expression of a gene or polynucleotide of interest.

Small interfering RNAs (siRNAs) are RNA duplexes normally from about 16 to about 30 nucleotides long that can associate with a cytoplasmic multi-protein complex known as RNAi-induced silencing complex (RISC). RISC loaded with siRNA mediates the degradation of homologous mRNA transcripts, therefore siRNA can be designed to knock down protein expression with high specificity. Unlike other antisense technologies, siRNA function through a natural mechanism evolved to control gene expression through non-coding RNA. A variety of RNAi reagents, including siRNAs targeting clinically relevant targets, are currently under pharmaceutical development, as described, e.g., in de Fougerolles, A. et al, Nature Reviews 6:443-453 (2007).

While the first described RNAi molecules were RNA:RNA hybrids that include both an RNA sense and an RNA antisense strand, it has now been demonstrated that DNA sense:RNA antisense hybrids, RNA sense:DNA antisense hybrids, and DNA:DNA hybrids are capable of mediating RNAi (Lamberton, J. S. and Christian, A. T., (2003) Molecular Biotechnology 24:111-119). RNAi molecules can be used that include any of these different types of double-stranded molecules. In addition, it is understood that RNAi molecules may be used and introduced to cells in a variety of forms. RNAi molecules can encompass any and all molecules capable of inducing an RNAi response in cells, including, but not limited to, double-stranded oligonucleotides that include two separate strands, i.e. a sense strand and an antisense strand, e.g., small interfering RNA (siRNA); double-stranded oligonucleotide that includes two separate strands that are linked together by non-nucleotidyl linker; oligonucleotides that include a hairpin loop of complementary sequences, which forms a double-stranded region, e.g., shRNAi molecules, and expression vectors that express one or more polynucleotides capable of forming a double-stranded polynucleotide alone or in combination with another polynucleotide.

A “single strand siRNA compound” as used herein, is an siRNA compound which is made up of a single molecule. It may include a duplexed region, formed by intra-strand pairing, e.g., it may be, or include, a hairpin or pan-handle structure. Single strand siRNA compounds may be antisense with regard to the target molecule.

A single strand siRNA compound may be sufficiently long that it can enter the RISC and participate in RISC mediated cleavage of a target mRNA. A single strand siRNA compound is at least about 14, at least about 15, at least about 20, at least about 25, at least about 30, at least about 35, at least about 40, or up to about 50 nucleotides in length. The single strand siRNA is less than about 200, about 100, or about 60 nucleotides in length.

Hairpin siRNA compounds may have a duplex region equal to or at least about 17, about 18, about 19, about 20, about 21, about 22, about 23, about 24, or about 25 nucleotide pairs. The duplex region may be equal to or less than about 200, about 100, or about 50 nucleotide pairs in length. Ranges for the duplex region are from about 15 to about 30, from about 17 to about 23, from about 19 to about 23, and from about 19 to about 21 nucleotides pairs in length. The hairpin may have a single strand overhang or terminal unpaired region. The overhangs may be from about 2 to about 3 nucleotides in length. The overhang can be at the sense side of the hairpin or on the antisense side of the hairpin.

A “double stranded siRNA compound” as used herein, is an siRNA compound which includes more than one, and in some cases two, strands in which interchain hybridization can form a region of duplex structure.

The antisense strand of a double stranded siRNA compound may be equal to or at least about 14, about 15, about 16 about 17, about 18, about 19, about 20, about 25, about 30, about 40, or about 60 nucleotides in length. It may be equal to or less than about 200, about 100, or about 50 nucleotides in length. Ranges may be from about 17 to about 25, from about 19 to about 23, and from about 19 to about 21 nucleotides in length. As used herein, term “antisense strand” means the strand of an siRNA compound that is sufficiently complementary to a target molecule, e.g. a target RNA.

The sense strand of a double stranded siRNA compound may be equal to or at least about 14, about 15, about 16, about 17, about 18, about 19, about 20, about 25, about 30, about 40, or about 60 nucleotides in length. It may be equal to or less than about 200, about 100, or about 50, nucleotides in length. Ranges may be from about 17 to about 25, from about 19 to about 23, and from about 19 to about 21 nucleotides in length.

The double strand portion of a double stranded siRNA compound may be equal to or at least about 14, about 15, about 16, about 17, about 18, about 19, about 20, about 21, about 22, about 23, about 24, about 25, about 30, about 40, or about 60 nucleotide pairs in length, It may be equal to or less than about 200, about 100, or about 50, nucleotides pairs in length. Ranges may be from about 15 to about 30, from about 17 to about 23, from about 19 to about 23, and from about 19 to about 21 nucleotides pairs in length.

The siRNA compound can be sufficiently large that it can be cleaved by an endogenous molecule, e.g., by Dicer, to produce smaller siRNA compounds, e.g., siRNAs agents.

The sense and antisense strands may be chosen such that the double-stranded siRNA compound includes a single strand or unpaired region at one or both ends of the molecule. Thus, a double-stranded siRNA compound may contain sense and antisense strands, paired to contain an overhang, e.g., one or two 5′ or 3′ overhangs, or a 3′ overhang of 1 to 3 nucleotides. The overhangs can be the result of one strand being longer than the other, or the result of two strands of the same length being staggered. Some embodiments will have at least one 3′ overhang. In embodiments, both ends of an siRNA molecule will have a 3′ overhang. The overhang can be 2 nucleotides.

The length for the duplexed region can be from about 15 to about 30, or about 18, about 19, about 20, about 21, about 22, or about 23 nucleotides in length, e.g., in the ssiRNA (siRNA with sticky overhangs) compound range discussed above. ssiRNA compounds can resemble in length and structure the natural Dicer processed products from long dsiRNAs. Embodiments in which the two strands of the ssiRNA compound are linked, e.g., covalently linked are also included. Hairpin, or other single strand structures which provide a double stranded region, and a 3′ overhang are included.

The siRNA compounds described herein, including double-stranded siRNA compounds and single-stranded siRNA compounds can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene.

As used herein, the phrase “mediates RNAi” refers to the ability to silence, in a sequence specific manner, a target RNA. While not wishing to be bound by theory, it is believed that silencing uses the RNAi machinery or process and a guide RNA, e.g., an ssiRNA compound of from about 21 to about 23 nucleotides.

An siRNA compound that is “sufficiently complementary” to a target RNA, e.g., a target mRNA, can silence production of protein encoded by the target mRNA. A siRNA compound that is “sufficiently complementary” to the RNA encoding a protein of interest, can silence production of the protein of interest encoded by the mRNA. The siRNA compound can be “exactly complementary” to a target RNA, e.g., the target RNA and the siRNA compound anneal, for example to form a hybrid made exclusively of Watson-Crick base pairs in the region of exact complementarity. A “sufficiently complementary” target RNA can include an internal region (e.g., of at least about 10 nucleotides) that is exactly complementary to a target RNA. In embodiments, the siRNA compound specifically discriminates a single-nucleotide difference. In this case, the siRNA compound only mediates RNAi if exact complementary is found in the region (e.g., within 7 nucleotides of) the single-nucleotide difference.

### MicroRNAs

The therapeutic moiety can be a microRNA molecule. MicroRNAs (miRNAs) are a highly conserved class of small RNA molecules that are transcribed from DNA in the genomes of plants and animals, but are not translated into protein. Processed miRNAs are single stranded 17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to ‘he 3’-untranslated region of specific mRNAs. RISC mediates down-regulation of gene expression through translational inhibition, transcript cleavage, or both. RISC is also implicated in transcriptional silencing in the nucleus of a wide range of eukaryotes.

### Antagomirs

The therapeutic moiety can be an antagomir. Antagomirs are RNA-like oligonucleotides that harbor various modifications for RNAse protection and pharmacologic properties, such as enhanced tissue and cellular uptake. They differ from normal RNA by, for example, comp'ete 2′-O-methylation of sugar, phosphorothioate backbone and, for example, a cholesterol-moiet' at 3′-end. Antagomirs may be used to efficiently silence endogenous miRNAs by forming duplexes that include the antagomir and endogenous miRNA, thereby preventing miRNA-induced gene silencing. An example of antagomir-mediated miRNA silencing is the silencing of miR-122, described in Krutzfeldt et al., Nature, 2005, 438: 685-689, which is expressly incorporated by reference herein in its entirety. Antagomir RNAs may be synthesized using standard solid phase oligonucleotide synthesis protocols. See U.S. patent application Ser. Nos. 11/502,158 and 11/657,341 (the disclosure of each of which are incorporated herein by reference).

An antagomir can include ligand-conjugated monomer subunits and monomers for oligonucleotide synthesis. Monomers are described in U.S. application Ser. No. 10/916,185, filed on Aug. 10, 2004. An antagomir can have a ZXY structure, such as is described in PCT Application No. PCT/US2004/07070 filed on Mar. 8, 2004. An antagomir can be complexed with an amphipathic moiety. Amphipathic moieties for use with oligonucleotide agents are described in PCT Application No. PCT/US2004/07070, filed on Mar. 8, 2004.

### Aptamers

The therapeutic moiety can be an aptamer. Aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules. See Eaton, Curr. Opin. Chem. Biol. 1: 10-16 (1997), Famulok, Curr. Opin. Struct. Biol. 9:324-9(1999), and Hermann and Patel, Science 287:820-5 (2000). Aptamers may be RNA or DNA based, and may include a riboswitch. A riboswitch is a part of an mRNA molecule that can directly bind a small target molecule, and whose binding of the target affects the gene's activity. Thus, an mRNA that contains a riboswitch is directly involved in regulating its own activity, depending on the presence or absence of its target molecule. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer may be prepared by any known method, including synthetic, recombinant, and purification methods, and may be used alone or in combination with other aptamers specific for the same target. Further, the term “aptamer” also includes “secondary aptamers” containing a consensus sequence derived from comparing two or more known aptamers to a given target. The aptamer can be an “intracellular aptamer”, or “intramer”, which specifically recognize intracellular targets. See Famulok et al., Chem Biol. 2001, Oct. 8(10):931-939; Yoon and Rossi, Adv Drug Deliv Rev. 2018, September, 134:22-35, each incorporated by reference herein.

### Ribozymes

The therapeutic moiety can be a ribozyme. Ribozymes are RNA molecules complexes having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). For example, a large number of ribozymes accelerate phosphoester transfer reactions with a high degree of specificity, often cleaving only one of several phosphoesters in an oligonucleotide substrate (Cech et al, Cell. 1981 December; 27(3 Pt 2):487-96; Michel and Westhof, J Mol Biol. 1990 Dec. 5; 216(3):585-610; Reinhold-Hurek and Shub, Nature. 1992 May 14; 357(6374): 173-6). This specificity has been attributed to the requirement that the substrate bind via specific base-pairing interactions to the internal guide sequence (“IGS”) of the ribozyme prior to chemical reaction.

At least six basic varieties of naturally-occurring enzymatic RNAs are known presently. Each can catalyze the hydrolysis of RNA phosphodiester bonds in trans (and thus can cleave other RNA molecules) under physiological conditions, In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis δ virus, group I intron or RNaseP RNA (in association with an RNA guide sequence) or Neurospora VS RNA motif, for example. Specific examples of hammerhead motifs are described by Rossi et al. Nucleic Acids Res. 1992 Sep. 11; 20(17):4559-65. Examples of hairpin motifs are described by Hampel et al. (Eur. Pat. Appl. Publ. No. EP 0360257), Hampel and Tritz, Biochemistry 1989 Jun. 13; 28(12):4929-33; Hampel et al, Nucleic Acids Res. 1990 Jan. 25; 18(2):299-304 and U.S. Pat. No. 5,631,359. An example of the hepatitis virus motif is described by Perrotta and Been, Biochemistry. 1992 Dec. 1; 31(47): 11843-52; an example of the RNaseP motif is described by Guerrier-Takada et al, Cell. 1983 December; 35(3 Pt 2):849-57; Neurospora VS RNA ribozyme motif is described by Collins (Saville and Collins, Cell. 1990 May 18; 61(4):685-96; Saville and Collins, Proc Natl Acad Sci USA. 1991 Oct. 1; 88(19):8826-30; Collins and Olive, Biochemistry. 1993 Mar. 23; 32(1 l):2795-9); and an example of the Group I intron is described in U.S. Pat. No. 4,987,071. Enzymatic nucleic acid molecules can have a specific substrate binding site which is complementary to one or more of the target gene DNA or RNA regions, and that they have nucleotide sequences within or surrounding that substrate binding site which impart an RNA cleaving activity to the molecule. Thus the ribozyme constructs need not be limited to specific motifs mentioned herein.

Methods of producing a ribozyme targeted to a polynucleotide sequence are known in the art. Ribozymes may be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein.

Ribozyme activity can be increased by altering the length of the ribozyme binding arms or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem H bases to shorten RNA synthesis times and reduce chemical requirements.

### Immunostimulatory Oligonucleotides

The therapeutic moiety can bean immunostimulatory oligonucleotide. Immunostimulatory oligonucleotides (ISS; single- or double-stranded) are capable of inducing an immune response when administered to a patient, which may be a mammal or other patient. ISS include, e.g., certain palindromes leading to hairpin secondary structures (see Yamamoto S., et al. (1992) J. Immunol. 148: 4072-4076), or CpG motifs, as well as other known ISS features (such as multi-G domains, see WO 96/11266).

The immune response may be an innate or an adaptive immune response. The immune system is divided into a more innate immune system, and acquired adaptive immune system of vertebrates, the latter of which is further divided into humoral cellular components. The immune response may be mucosal.

Immunostimulatory nucleic acids are considered to be non-sequence specific when it is not required that they specifically bind to and reduce the expression of a target polynucleotide in order to provoke an immune response. Thus, certain immunostimulatory nucleic acids may include a sequence corresponding to a region of a naturally occurring gene or mRNA, but they may still be considered non-sequence specific immunostimulatory nucleic acids.

The immunostimulatory nucleic acid or oligonucleotide can include at least one CpG dinucleotide. The oligonucleotide or CpG dinucleotide may be unmethylated or methylated. The immunostimulatory nucleic acid can include at least one CpG dinucleotide having a methylated cytosine. The nucleic acid can include a single CpG dinucleotide, wherein the cytosine in said CpG dinucleotide is methylated. The nucleic acid can include the sequence 5′ TAACGTTGAGGG′CAT 3′ (SEQ ID NO: 138). The nucleic acid can include at least two CpG dinucleotides, wherein at least one cytosine in the CpG dinucleotides is methylated. Each cytosine in the CpG dinucleotides present in the sequence can be methylated. The nucleic acid can include a plurality of CpG dinucleotides, wherein at least one of said CpG dinucleotides includes a methylated cytosine.

Additional specific nucleic acid sequences of oligonucleotides (ODNs) suitable for use in the compositions and methods are described in Raney et al, Journal of Pharmacology and Experimental Therapeutics, 298:1185-1192 (2001). ODNs used in the compositions and methods can have a phosphodiester (“PO”) backbone or a phosphorothioate (“PS”) backbone, and/or at least one methylated cytosine residue in a CpG motif.

### Decoy Oligonucleotides

The therapeutic moiety can be a decoy oligonucleotide. Because transcription factors recognize their relatively short binding sequences, even in the absence of surrounding genomic DNA, short oligonucleotides bearing the consensus binding sequence of a specific transcription factor can be used as tools for manipulating gene expression in living cells. This strategy involves the intracellular delivery of such “decoy oligonucleotides”, which are then recognized and bound by the target factor. Occupation of the transcription factor's DNA-binding site by the decoy renders the transcription factor incapable of subsequently binding to the promoter regions of target genes. Decoys can be used as therapeutic agents, either to inhibit the expression of genes that are activated by a transcription factor, or to upregulate genes that are suppressed by the binding of a transcription factor. Examples of the utilization of decoy oligonucleotides may be found in Mann et al., J. Clin. Invest, 2000, 106: 1071-1075, which is expressly incorporated by reference herein, in its entirety.

### Supermir

The therapeutic moiety can be a supermir. A supermir refers to a single stranded, double stranded or partially double stranded oligomer or polymer of ribonucleic acid (RNA) or deoxyribonucleic acid (DNA) or both or modifications thereof, which has a nucleotide sequence that is substantially identical to an miRNA and that is antisense with respect to its target, This term includes oligonucleotides composed of naturally-occurring nucleobases, sugars and covalent internucleoside (backbone) linkages and which contain at least one non-naturally-occurring portion which functions similarly. Such modified or substituted oligonucleotides have desirable properties such as, for example, enhanced cellular uptake, enhanced affinity for nucleic acid target and increased stability in the presence of nucleases. The supermir may not include a sense strand. The supermir may not self-hybridize to a significant extent. A supermir can have secondary structure, but it is substantially single-stranded under physiological conditions. A supermir that is substantially single-stranded is single-stranded to the extent that less than about 50% (e.g., less than about 40%, about 30%, about 20%, about 10%, or about 5%) of the supermir is duplexed with itself. The supermir can include a hairpin segment, e.g., sequence, for example, at ‘the 3’ end can self hybridize and form a duplex region, e.g., a duplex region of at least about 1, about 2, about 3, or about 4 or less than about 8, about 7, about 6, or about 5 nucleotides, or about 5 nucleotides. The duplexed region can be connected by a linker, e.g., a nucleotide linker, e.g., about 3, about 4, about 5, or about 6 dTs, e.g., modified dTs. The supermir can be duplexed with a shorter oligo, e.g., of about 5, about 6, about 7, about 8, about 9, or about 10 nucleotides in length, e.g., at one or both of the 3′ and 5′ end or at one end and in the non-terminal or middle of the supermir.

miRNA Mimics

The therapeutic moiety can be a miRNA mimic. miRNA mimics represent a class of molecules that can be used to imitate the gene silencing ability of one or more miRNAs. Thus, the term “microRNA mimic” refers to synthetic non-coding RNAs (i.e. the miRNA is not obtained by purification from a source of the endogenous miRNA) that are capable of entering the RNAi pathway and regulating gene expression. miRNA mimics can be designed as mature molecules (e.g. single stranded) or mimic precursors (e.g., pri- or pre-miRNAs). miRNA mimics can include nucleic acid (modified or modified nucleic acids) including oligonucleotides that include, without limitation, RNA, modified RNA, DNA, modified DNA, locked nucleic acids, or 2′-0,4′-C-ethylene-bridged nucleic acids (ENA), or any combination of the above (including DNA-RNA hybrids). In addition, miRNA mimics can include conjugates that can affect delivery, intracellular compartmentalization, stability, specificity, functionality, strand usage, and/or potency. In one design, miRNA mimics are double stranded molecules (e.g., with a duplex region of between about 16 and about 31 nucleotides in length) and contain one or more sequences that have identity with the mature strand of a given miRNA. Modifications can include 2′ modifications (including 2′-0 methyl modifications and 2′ F modifications) on one or both strands of the molecule and internucleotide modifications (e.g. phorphorthioate modifications) that enhance nucleic acid stability and/or specificity. In addition, miRNA mimics can include overhangs. The overhangs can include from about 1 to about 6 nucleotides on either' the 3′ or 5′ end of either strand and can be modified to enhance stability or functionality. The miRNA mimic can include a duplex region of from about 16 to about 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can include 2′ F modification of all of the Cs and Us, phosphorylation of the 5′ end of the oligonucleotide, and stabilized internucleotide linkages associated with a 2 nucleotide 3′ overhang.

miRNA Inhibitor

The therapeutic moiety can be a miRNA inhibitor. The terms “antimir” “microRNA inhibitor”, “miR inhibitor”, or “miRNA inhibitor” are synonymous and refer to oligonucleotides or modified oligonucleotides that interfere with the ability of specific miRNAs. In general, the inhibitors are nucleic acid or modified nucleic acids in nature including oligonucleotides that include RNA, modified RNA, DNA, modified DNA, locked nucleic acids (LNAs), or any combination of the above.

Modifications include 2′ modifications (including 2′-0 alkyl modifications and 2′ F modifications) and internucleotide modifications (e.g. phosphorothioate modifications) that can affect delivery, stability, specificity, intracellular compartmentalization, or potency. In addition, miRNA inhibitors can include conjugates that can affect delivery, intracellular compartmentalization, stability, and/or potency. Inhibitors can adopt a variety of configurations including single stranded, double stranded (RNA/RNA or RNA/DNA duplexes), and hairpin designs, in general, microRNA inhibitors include contain one or more sequences or portions of sequences that are complementary or partially complementary with the mature strand (or strands) of the miRNA to be targeted, in addition, the miRNA inhibitor may also include additional sequences located 5′ and 3′ to the sequence that is the reverse complement of the mature miRNA. The additional sequences may be the reverse complements of the sequences that are adjacent to the mature miRNA in the pri-miRNA from which the mature miRNA is derived, or the additional sequences may be arbitrary sequences (having a mixture of A, G, C, or U). One or both of the additional sequences can be arbitrary sequences capable of forming hairpins. The sequence that is the reverse complement of the miRNA may be flanked on the 5′ side and on the 3′ side by hairpin structures. Micro-RNA inhibitors, when double stranded, may include mismatches between nucleotides on opposite strands. Furthermore, micro-RNA inhibitors may be linked to conjugate moieties in order to facilitate uptake of the inhibitor into a cell. For example, a micro-RNA inhibitor may be linked to cholesteryl 5-(bis(4-methoxyphenyl)(phenyl)methoxy)-3 hydroxypentylcarbamate) which allows passive uptake of a micro-RNA inhibitor into a cell. Micro-RNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

### Antisense Compounds (ACs)

The therapeutic moiety includes an antisense compound (AC) that can alter one or more aspects of translation, or expression of a target gene. The principle behind antisense technology is that an antisense compound, which hybridizes to a target nucleic acid, modulates gene expression activities such as translation through one of a number of antisense mechanisms Antisense technology is an effective means for changing the expression of one or more specific gene products and can therefore prove to be useful in a number of therapeutic, diagnostic, and research applications.

The compounds described herein may contain one or more asymmetric centers and thus give rise to enantiomers, diastereomers, and other stereoisomeric configurations that may be defined, in terms of absolute stereochemistry, as (R) or (S), α or β, or as (D) or (L). Included in the antisense compounds provided herein are all such possible isomers, as well as their racemic and optically pure forms.

### Antisense Compound Hybridization Site

Antisense mechanisms rely on hybridization of the antisense compound to the target nucleic acid.

The AC can hybridize with a sequence from about 5 to about 50 nucleic acids in length, which can also be referred to as the length of the AC. The AC can be from about 5 to about 10, from about 10 to about 15, from about 15 to about 20, from about 20 to about 25, from about 25 to about 30, from about 30 to about 35, from about 35 to about 40, from about 40 to about 45, or from about 45 to about 50 nucleic acids in length. The AC can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 nucleic acids in length. The AC can be about 10 nucleic acids in length. The AC can be about 15 nucleic acids in length. The AC can be about 20 nucleic acids in length. The AC can be about 25 nucleic acids in length. The AC can be about 30 nucleic acids in length.

The AC may be less than about 100 percent complementary to a target nucleic acid sequence. As used herein, the term “percent complementary” refers to the number of nucleobases of an AC that have nucleobase complementarity with a corresponding nucleobase of an oligomeric compound or nucleic acid divided by the total length (number of nucleobases) of the AC. One skilled in the art recognizes that the inclusion of mismatches is possible without eliminating the activity of the antisense compound. The AC may contain up to about 20% nucleotides that disrupt base pairing of the AC to the target nucleic acid. The AC may contain no more than about 15%, no more than about 10%, no more than 5%, or no mismatches. The ACs can be at least about 80%, at least about 85%, at least about 90%, at least about 95%, at least about 96%, at least about 97%, at least about 98%, at least about 99% or about 100% complementary to a target nucleic acid. Percent complementarity of an oligonucleotide is calculated by dividing the number of complementary nucleobases by the total number of nucleobases of the oligonucleotide. Percent complementarity of a region of an oligonucleotide is calculated by dividing the number of complementary nucleobases in the region by the total number of nucleobases region.

Incorporation of nucleotide affinity modifications can allow for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or ΔTm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

### Antisense Mechanisms

The ACs according to the present disclosure may modulate one or more aspects of protein transcription, translation, and expression.

The AC can regulate transcription, translation, or protein expression through steric blocking. The following review article describes the mechanisms of steric blocking and applications thereof and is incorporated by reference herein in its entirety: Roberts et al. Nature Reviews Drug Discovery (2020) 19: 673-694.

The antisense mechanism functions via hybridization of an antisense compound with a target nucleic acid. The AC can hybridize to its target sequence and downregulate expression of the target protein. The AC can hybridize to its target sequence to downregulate expression of one or more target protein isomers. The AC can hybridize to its target sequence to upregulate expression of the target protein. The AC can hybridize to its target sequence to increase expression of one or more target protein isomers.

The efficacy of the ACs may be assessed by evaluating the antisense activity effected by their administration. As used herein, the term “antisense activity” refers to any detectable and/or measurable activity attributable to the hybridization of an antisense compound to its target nucleic acid. Such detection and or measuring may be direct or indirect. In embodiments, antisense activity is assessed by detecting and or measuring the amount of target protein. Antisense activity can be assessed by detecting and/or measuring the amount of target nucleic acids.

### Antisense Compound Design

Design of ACs according to the present disclosure will depend upon the sequence being targeted. Targeting an AC to a particular target nucleic acid molecule can be a multistep process. The process usually begins with the identification of a target nucleic acid whose expression is to be modulated. As used herein, the terms “target nucleic acid” and “nucleic acid encoding a target gene” encompass DNA encoding a selected target gene, RNA (including pre-mRNA and mRNA) transcribed from such DNA, and also cDNA derived from such RNA.

One of skill in the art will be able to design, synthesize, and screen antisense compounds of different nucleobase sequences to identify a sequence that results in antisense activity. For example, one may design an antisense compound that inhibits expression of a target protein. Methods for designing, synthesizing and screening antisense compounds for antisense activity against a preselected target nucleic acid can be found, for example in “Antisense Drug Technology, Principles, Strategies, and Applications” Edited by Stanley T. Crooke, CRC Press, Boca Raton, Florida, which is incorporated by reference in its entirety for any purpose.

Antisense compounds are provided that include from about 8 to about 30 linked nucleosides. The antisense compounds can include modified nucleosides, modified internucleoside linkages and/or conjugate groups.

The antisense compound can be a “tricyclo-DNA (tc-DNA)”, which refers to a class of constrained DNA analogs in which each nucleotide is modified by the introduction of a cyclopropane ring to restrict conformational flexibility of the backbone and to enhance the backbone geometry of the torsion angle γ. Homobasic adenine- and thymine-containing tc-DNAs form extraordinarily stable A-T base pairs with complementary RNAs.

### Nucleosides

The antisense compounds can include linked nucleosides. Some or all of the nucleosides can be modified nucleosides. One or more nucleosides can include a modified nucleobase. One or more nucleosides can include a modified sugar. Chemically modified nucleosides are routinely used for incorporation into antisense compounds to enhance one or more properties, such as nuclease resistance, pharmacokinetics or affinity for a target RNA. Non-limiting examples of nucleosides are provided in Khvorova et al. Nature Biotechnology (2017) 35: 238-248, which is incorporated by reference herein in its entirety.

In general, a nucleobase is any group that contains one or more atom or groups of atoms capable of hydrogen bonding to a base of another nucleic acid. In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The terms modified nucleobase and nucleobase mimetic can overlap but generally a modified nucleobase refers to a nucleobase that is fairly similar in structure to the parent nucleobase, such as for example a 7-deaza purine, a 5-methyl cytosine, or a G-clamp, whereas a nucleobase mimetic would include more complicated structures, such as for example a tricyclic phenoxazine nucleobase mimetic. Methods for preparation of the above noted modified nucleobases are well known to those skilled in the art.

ACs can include one or more nucleosides having a modified sugar moiety. The furanosyl sugar ring of a natural nucleoside can be modified in a number of ways including, but not limited to, addition of a substituent group, bridging of two non-geminal ring atoms to form a bicyclic nucleic acid (BNA) and substitution of an atom or group such as —S—, —N(R)— or —C(R1)(R2) for the ring oxygen at the 4′-position. Modified sugar moieties are well known and can be used to alter, typically increase, the affinity of the antisense compound for its target and/or increase nuclease resistance. A representative list of modified sugars includes but is not limited to non-bicyclic substituted sugars, especially non-bicyclic 2′-substituted sugars having a 2′-F, 2′-OCH3 or a 2′-O(CH2)2-OCH3 substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; and 6,600,032; and WO 2005/121371.

Nucleosides can include bicyclic modified sugars (BNA's), including LNA (4′-(CH₂)—O-2′ bridge), 2′-thio-LNA (4′-(CH2)-S-2′ bridge), 2′-amino-LNA (4′-(CH2)-NR-2′ bridge), ENA (4′-(CH2)2-O-2′ bridge), 4′-(CH2)3-2′ bridged BNA, 4′-(CH2CH(CH3))-2′ bridged BNA” cEt (4′-(CH(CH3)-O-2′ bridge), and cMOE BNAs (4′-(CH(CH2OCH3)-O-2′ bridge). Certain such BNA's have been prepared and disclosed in the patent literature as well as in scientific literature (See, e.g., Srivastava, et al. J. Am. Chem. Soc. 2007, ACS Advanced online publication, 10.1021/ja071106y, Albaek et al. J. Org. Chem., 2006, 71, 7731-7740, Fluiter, et al. Chembiochem 2005, 6, 1104-1109, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039, WO 2007/090071; Examples of issued US patents and published applications that disclose BNAs include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are “Locked Nucleic Acids” (LNAs) in which the 2′-hydroxyl group of the ribosyl sugar ring is linked to the 4′ carbon atom of the sugar ring thereby forming a 2′-C,4′-C-oxymethylene linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH2-) group bridging the 2′ oxygen atom and the 4′ carbon atom, for which the term LNA is used for the bicyclic moiety; in the case of an ethylene group in this position, the term ENA™ is used (Singh et al., Chem. Commun., 1998, 4, 455-456; ENA™: Morita et al., Bioorganic Medicinal Chemistry, 2003, 11, 2211-2226). LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A., 2000, 97, 5633-5638).

An isomer of LNA that has also been studied is alpha-L-LNA which has been shown to have superior stability against a 3′-exonuclease. The alpha-L-LNA's were incorporated into antisense gapmers and chimeras that showed potent antisense activity (Frieden et al., Nucleic Acids Research, 2003, 21, 6365-6372).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., WO 99/14226). Furthermore, synthesis of 2′-amino-LNA, a novel conformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

### Internucleoside Linkages

Described herein are internucleoside linking groups that link the nucleosides or otherwise modified monomer units together thereby forming an antisense compound. The two main classes of internucleoside linking groups are defined by the presence or absence of a phosphorus atom. Representative phosphorus containing internucleoside linkages include, but are not limited to, phosphodiesters, phosphotriesters, methylphosphonates, phosphoramidate, and phosphorothioates. Representative non-phosphorus containing internucleoside linking groups include, but are not limited to, methylenemethylimino (—CH2-N(CH3)-O-CH2-), thiodiester (—O—C(O)—S—), thionocarbamate (—O—C(O)(NH)—S—); siloxane (—O—Si(H)2-O—); and N,N′-dimethylhydrazine (—CH2-N(CH3)-N(CH3)-). Antisense compounds having non-phosphorus internucleoside linking groups are referred to as oligonucleosides. Modified internucleoside linkages, compared to natural phosphodiester linkages, can be used to alter, typically increase, nuclease resistance of the antisense compound. Internucleoside linkages having a chiral atom can be prepared racemic, chiral, or as a mixture. Representative chiral internucleoside linkages include, but are not limited to, alkylphosphonates and phosphorothioates. Methods of preparation of phosphorous-containing and non-phosphorous-containing linkages are well known to those skilled in the art.

A phosphate group can be linked to the 2′, 3′ or 5′ hydroxyl moiety of the sugar. In forming oligonucleotides, the phosphate groups covalently link adjacent nucleosides to one another to form a linear polymeric compound. Within oligonucleotides, the phosphate groups are commonly referred to as forming the internucleoside backbone of the oligonucleotide. The normal linkage or backbone of RNA and DNA is a 3′ to 5′ phosphodiester linkage.

### Conjugate Groups

Cargo can be modified by covalent attachment of one or more conjugate groups. In general, conjugate groups modify one or more properties of the cargo including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. The conjugate group can include polyethylene glycol (PEG). PEG can be conjugated to either the cargo or the cCPP. The cargo can include a peptide, oligonucleotide or small molecule.

Conjugate groups can include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996,277,923).

Linking groups or bifunctional linking moieties such as those known in the art are amenable to the compounds provided herein. Linking groups are useful for attachment of chemical functional groups, conjugate groups, reporter groups and other groups to selective sites in a parent compound such as for example an AC. In general a bifunctional linking moiety includes a hydrocarbyl moiety having two functional groups. One of the functional groups is selected to bind to a parent molecule or compound of interest and the other is selected to bind essentially any selected group such as chemical functional group or a conjugate group. Any of the linkers described here may be used. the linker can include a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. Bifunctional linking moieties can include amino, hydroxyl, carboxylic acid, thiol, unsaturations (e.g., double or triple bonds), and the like. Some nonlimiting examples of bifunctional linking moieties include 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other linking groups include, but are not limited to, substituted C1-C10 alkyl, substituted or unsubstituted C₂-C₁₀ alkenyl or substituted or unsubstituted C₂-C₁₀ alkynyl, wherein a nonlimiting list of substituent groups includes hydroxyl, amino, alkoxy, carboxy, benzyl, phenyl, nitro, thiol, thioalkoxy, halogen, alkyl, aryl, alkenyl and alkynyl.

The AC may be from about 5 to about 50 nucleotides in length. The AC may be from about to about 10 nucleotides in length. The AC may be from about 10 to about 15 nucleotides in length. The AC may be from about 15 to about 20 nucleotides in length. The AC may be from about 20 to about 25 nucleotides in length. The AC may be from about 25 to about 30 nucleotides in length. The AC may be from about 30 to about 35 nucleotides in length. The AC may be from about 35 to about 40 nucleotides in length. The AC may be from about 40 to about 45 nucleotides in length. The AC may be from about 45 to about 50 nucleotides in length.

### Clustered Regularly Interspaced Short Palindromic Repeats (CRISPR) Gene-Editing Machinery

The compounds can include one or more cCPP (or cCPP) conjugated to CRISPR gene-editing machinery. As used herein, “CRISPR gene-editing machinery” refers to protein, nucleic acids, or combinations thereof, which may be used to edit a genome. Non-limiting examples of gene-editing machinery include gRNAs, nucleases, nuclease inhibitors, and combinations and complexes thereof. The following patent documents describe CRISPR gene-editing machinery: U.S. Pat. Nos. 8,697,359, 8,771,945, 8,795,965, 8,865,406, 8,871,445, 8,889,356, 8,895,308, 8,906,616, 8,932,814, 8,945,839, 8,993,233, 8,999,641, U.S. patent application Ser. No. 14/704,551, and U.S. patent application Ser. No. 13/842,859. Each of the aforementioned patent documents is incorporated by reference herein in its entirety.

A linker can conjugate the cCPP to the CRISPR gene-editing machinery. Any linker described in this disclosure or that is known to a person of skill in the art may be utilized.

gRNA

The compound can include the cCPP conjugated to a gRNA. A gRNA targets a genomic loci in a prokaryotic or eukaryotic cell.

The gRNA can be a single-molecule guide RNA (sgRNA). A sgRNA includes a spacer sequence and a scaffold sequence. A spacer sequence is a short nucleic acid sequence used to target a nuclease (e.g., a Cas9 nuclease) to a specific nucleotide region of interest (e.g., a genomic DNA sequence to be cleaved). The spacer may be about 17-24 base pairs in length, such as about 20 base pairs in length. The spacer may be about 15, about 16, about 17, about 18, about 19, about 20, about 21, about 22, about 23, about 24, about 25, about 26, about 27, about 28, about 29, or about 30 base pairs in length. The spacer may be at least 15, at least 16, at least 17, at least 18, at least 19, at least 20, at least 21, at least 22, at least 23, at least 24, at least 25, at least 26, at least 27, at least 28, at least 29, or at least 30 base pairs in length. The spacer may be about 15, about 16, about 17, about 18, about 19, about 20, about 21, about 22, about 23, about 24, about 25, about 26, about 27, about 28, about 29, or about 30 base pairs in length. The spacer sequence can have between about 40% to about 80% GC content.

The spacer can target a site that immediately precedes a 5′ protospacer adjacent motif (PAM). The PAM sequence may be selected based on the desired nuclease. For example, the PAM sequence may be any one of the PAM sequences shown in the table below, wherein N refers to any nucleic acid, R refers to A or G, Y refers to C or T, W refers to A or T, and V refers to A or C or G.

TABLE 8

| PAM sequence | | |
|---|---|---|
| (5′ to 3′) | Nuclease | Isolated from |
| NGG | SpCas9 | Streptococcus pyogenes |
| NGRRT or | SaCas9 | Staphylococcus aureus |
| NGRRN | | |
| NNNNGATT | NmeCas9 | Neisseria meningitidis |
| NNNNRYAC | CjCas9 | Campylobacter jejuni |
| NNAGAAW | StCas9 | Streptococcus thermophiles |
| TTTV | LbCpf1 | Lachnospiraceae bacterium |
| TTTV | AsCpf1 | Acidaminococcus sp. |

A spacer may target a sequence of a mammalian gene, such as a human gene. The spacer may target a mutant gene. The spacer may target a coding sequence.

The scaffold sequence is the sequence within the sgRNA that is responsible for nuclease (e.g., Cas9) binding. The scaffold sequence does not include the spacer/targeting sequence. In embodiments, the scaffold may be about 1 to about 10, about 10 to about 20, about 20 to about 30, about 30 to about 40, about 40 to about 50, about 50 to about 60, about 60 to about 70, about 70 to about 80, about 80 to about 90, about 90 to about 100, about 100 to about 110, about 110 to about 120, or about 120 to about 130 nucleotides in length. The scaffold may be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, or about 125 nucleotides in length. The scaffold may be at least 10, at least 20, at least 30, at least 40, at least 50, at least 60, at least 70, at least 80, at least 90, at least 100, at least 110, at least 120, or at least 125 nucleotides in length.

The gRNA can be a dual-molecule guide RNA, e.g, crRNA and tracrRNA. The gRNA may further include a polyA tail.

A compound includes a cCPP conjugated to a nucleic acid that includes a gRNA. The nucleic acid can include about 1, about 2, about 3, about 4, about 5, about 6, about 7, about 8, about 9, about 10, about 11, about 12, about 13, about 14, about 15, about 16, about 17, about 18, about 19, or about 20 gRNAs. The gRNA can recognize the same target. The gRNA can recognize different targets. The nucleic acid that includes a gRNA includes a sequence encoding a promoter, wherein the promoter drives expression of the gRNA.

### Nuclease

The compounds can include a cyclic cell penetrating peptide (cCPP) conjugated to a nuclease. The nuclease can be a Type II, Type V-A, Type V-B, Type VC, Type V-U, or Type VI-B nuclease. The nuclease can be a transcription, activator-like effector nuclease (TALEN), a meganuclease, or a zinc-finger nuclease. The nuclease can be a Cas9, Cas12a (Cpf1), Cas12b, Cas12c, Tnp-B like, Cas13a (C2c2), Cas13b, or Cas14 nuclease. The nuclease can be a Cas9 nuclease or a Cpf1 nuclease.

The nuclease can be a modified form or variant of a Cas9, Cas12a (Cpf1), Cas12b, Cas12c, Tnp-B like, Cas13a (C₂c2), Cas13b, or Cas14 nuclease. The nuclease can be a modified form or variant of a TAL nuclease, a meganuclease, or a zinc-finger nuclease. A “modified” or “variant” nuclease is one that is, for example, truncated, fused to another protein (such as another nuclease), catalytically inactivated, etc. The nuclease may have at least about 800%, at least about 85%, at least about 90%, at least about 95%, at least about 98%, at least about 99%, or about 100% sequence identity to a naturally occurring Cas9, Cas12a (Cpf1), Cas12b, Cas12c, Tnp-B like, Cas13a (C₂c2), Cas13b, Cas14 nuclease, or a TALEN, meganuclease, or zinc-finger nuclease. The nuclease can be a Cas9 nuclease derived from S. pyogenes (SpCas9). The nuclease can have at least about 90%, at least about 95%, at least about 96%, at least about 97%, at least about 98%, or at least about 99% sequence identity to a Cas9 nuclease derived from S. pyogenes (SpCas9). The nuclease can be a Cas9 derived from S. aureus (SaCas9). The nuclease can have at least about 90%, at least about 95%, at least about 96%, at least about 97%, at least about 98%, or at least about 99% sequence identity to a Cas9 derived from S. aureus (SaCas9). Cpf1 can be a Cpf1 enzyme from Acidaminococcus (species BV3L6, UniProt Accession No. U2UMQ6). The nuclease can have at least about 90%, at least about 95%, at least about 96%, at least about 97%, at least about 98%, or at least about 99% sequence identity to a Cpf1 enzyme from Acidaminococcus (species BV3L6, UniProt Accession No. U2UMQ6).

Cpf1 can be a Cpf1 enzyme from Lachnospiraceae (species ND2006, UniProt Accession No. AOA182DWE3). The nuclease can have at least about 90%, at least about 95%, at least about 96%, at least about 97%, at least about 98%, or at least about 99% sequence identity to a Cpf1 enzyme from Lachnospiraceae. The sequence encoding the nuclease can be codon optimized for expression in mammalian cells. The sequence encoding the nuclease can be codon optimized for expression in human cells or mouse cells.

The compound can include a cCPP conjugated to a nuclease. The nuclease can be a soluble protein.

The compound can include a cCPP conjugated to a nucleic acid encoding a nuclease. The nucleic acid encoding a nuclease can include a sequence encoding a promoter, wherein the promoter drives expression of the nuclease.

gRNA and Nuclease Combinations

The compounds can include one or more cCPP conjugated to a gRNA and a nuclease. One or more cCPP can be conjugated to a nucleic acid encoding a gRNA and/or a nuclease. The nucleic acid encoding a nuclease and a gRNA can include a sequence encoding a promoter, wherein the promoter drives expression of the nuclease and the gRNA. The nucleic acid encoding a nuclease and a gRNA can include two promoters, wherein a first promoter controls expression of the nuclease and a second promoter controls expression of the gRNA. The nucleic acid encoding a gRNA and a nuclease can encode from about 1 to about 20 gRNAs, or from about 1, about 2, about 3, about 4, about 5, about 6, about 7, about 8, about 9, about 10, about 11, about 12, about 13, about 14, about 15, about 16, about 17, about 18, or about 19, and up to about 20 gRNAs. The gRNAs can recognize different targets. The gRNAs can recognize the same target.

The compounds can include a cyclic cell penetrating peptide (or cCPP) conjugated to a ribonucleoprotein (RNP) that includes a gRNA and a nuclease.

A composition that includes: (a) a cCPP conjugated to a gRNA and (b) a nuclease can be delivered to a cell. A composition that includes: (a) a cCPP conjugated to a nuclease and (b) an gRNA can be delivered to a cell.

A composition that includes: (a) a first cCPP conjugated to a gRNA and (b) a second cCPP conjugated to a nuclease can be delivered to a cell. The first cCPP and second cCPP can be the same. The first cCPP and second cCPP can be different.

### Genetic Element of Interest

The compounds can include a cyclic cell penetrating peptide (cCPP) conjugated to a genetic element of interest. A genetic element of interest can replace a genomic DNA sequence cleaved by a nuclease. Non-limiting examples of genetic elements of interest include genes, a single nucleotide polymorphism, promoter, or terminators.

### Nuclease Inhibitors

The compound can include a cyclic cell penetrating peptide (cCPP) conjugated to an inhibitor of a nuclease (e.g. a Cas9 inhibitor). A limitation of gene editing is potential off-target editing. The delivery of a nuclease inhibitor may limit off-target editing. The nuclease inhibitor can be a polypeptide, polynucleotide, or small molecule. Nuclease inhibitors are described in U.S. Publication No. 2020/087354, International Publication No. 2018/085288, U.S. Publication No. 2018/0382741, International Publication No. 2019/089761, International Publication No. 2020/068304, International Publication No. 2020/041384, and International Publication No. 2019/076651, each of which is incorporated by reference herein in its entirety.

### Therapeutic Polypeptides

### Antibodies

The therapeutic moiety can include an antibody or an antigen-binding fragment. Antibodies and antigen-binding fragments can be derived from any suitable source, including human, mouse, camelid (e.g., camel, alpaca, llama), rat, ungulates, or non-human primates (e.g., monkey, rhesus macaque).

The term “antibody” refers to an immunoglobulin (Ig) molecule capable of binding to a specific target, such as a carbohydrate, polynucleotide, lipid, or polypeptide, through at least one epitope recognition site located in the variable region of the Ig molecule. As used herein, the term encompasses intact polyclonal or monoclonal antibodies and antigen-binding fragments thereof. A native immunoglobulin molecule generally includes two heavy chain polypeptides and two light chain polypeptides. Each of the heavy chain polypeptides associate with a light chain polypeptide by virtue of interchain disulfide bonds between the heavy and light chain polypeptides to form two heterodimeric proteins or polypeptides (i.e., a protein comprised of two heterologous polypeptide chains). The two heterodimeric proteins then associate by virtue of additional interchain disulfide bonds between the heavy chain polypeptides to form an immunoglobulin protein or polypeptide.

The term “antigen-binding fragment” as used herein refers to a polypeptide fragment that contains at least one complementarity-determining region (CDR) of an immunoglobulin heavy and/or light chain that binds to at least one epitope of the antigen of interest. An antigen-binding fragment may comprise 1, 2, or 3 CDRs of a variable heavy chain (VH) sequence from an antibody that specifically binds to a target molecule. An antigen-binding fragment may comprise 1, 2, or 3 CDRs of a variable light chain (VL) sequence from an antibody that specifically binds to a target molecule. An antigen-binding fragment may comprise 1, 2, 3, 4, 5, or all 6 CDRs of a variable heavy chain (VH) and variable light chain (VL) sequence from antibodies that specifically bind to a target molecule. Antigen-binding fragments include proteins that comprise a portion of a full length antibody, generally the antigen binding or variable region thereof, such as Fab, F(ab′)2, Fab′, Fv fragments, minibodies, diabodies, single domain antibody (dAb), single-chain variable fragments (scFv), nanobodies, multispecific antibodies formed from antibody fragments, and any other modified configuration of the immunoglobulin molecule that can comprise an antigen-binding site or fragment of the required specificity.

The term “F(ab)” refers to two of the protein fragments resulting from proteolytic cleavage of IgG molecules by the enzyme papain. Each F(ab) can comprise a covalent heterodimer of the VH chain and VL chain and includes an intact antigen-binding site. Each F(ab) can be a monovalent antigen-binding fragment. The term “Fab′” refers to a fragment derived from F(ab′)2 and may contain a small portion of Fc. Each Fab′ fragment can be a monovalent antigen-binding fragment.

The term “F(ab′)2” refers to a protein fragment of IgG generated by proteolytic cleavage by the enzyme pepsin. Each F(ab′)2 fragment can comprise two F(ab′) fragments and can be therefore a bivalent antigen-binding fragment.

An “Fv fragment” refers to a non-covalent VH::VL heterodimer which includes an antigen-binding site that retains much of the antigen recognition and binding capabilities of the native antibody molecule, but lacks the CH1 and CL domains contained within a Fab. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

Bispecific Antibodies (BsAbs) are antibodies that can simultaneously bind two separate and unique antigens (or different epitopes of the same antigen). The therapeutic moiety can include a bispecific antibody that can simultaneously bind to two different targets of interest. The BsAbs may redirect cytotoxic immune effector cells for enhanced killing of tumor cells by antibody-dependent cell-mediated cytotoxicity (ADCC) and other cytotoxic mechanisms mediated by the effector cells.

Recombinant antibody engineering has allowed for the creation of recombinant bispecific antibody fragments comprising the variable heavy (VH) and light (VL) domains of the parental monoclonal antibodies (mabs). Non-limiting examples include scFv (single-chain variable fragment), BsDb (bispecific diabody), scBsDb (single-chain bispecific diabody), scBsTaFv (single-chain bispecific tandem variable domain), DNL-(Fab)3 (dock-and-lock trivalent Fab), sdAb (single-domain antibody), and BssdAb (bispecific single-domain antibody).

BsAbs with an Fc region are useful for carrying out Fc mediated effector functions such as ADCC and CDC. They have the half-life of normal IgG. On the other hand, BsAbs without the Fc region (bispecific fragments) rely solely on their antigen-binding capacity for carrying out therapeutic activity. Due to their smaller size, these fragments have better solid-tumor penetration rates. BsAb fragments do not require glycosylation, and they may be produced in bacterial cells. The size, valency, flexibility and half-life of BsAbs to suit the application.

Using recombinant DNA technology, bispecific IgG antibodies can be assembled from two different heavy and light chains expressed in the same cell line. Random assembly of the different chains results in the formation of nonfunctional molecules and undesirable HC homodimers. To address this problem, a second binding moiety (e.g., single chain variable fragment) may be fused to the N or C terminus of the H or L chain resulting in tetravalent BsAbs containing two binding sites for each antigen. Additional methods to address the LC-HC mispairing and HC homodimerization follow.

Knobs-into-holes BsAb IgG. H chain heterodimerization is forced by introducing different mutations into the two CH3 domains resulting in asymmetric antibodies. Specifically a “knob” mutation is made into one HC and a “hole” mutation is created in the other HC to promote heterodimerization.

Ig-scFv fusion. The direct addition of a new antigen-binding moiety to full length IgG results in fusion proteins with tetravalency. Examples include IgG C-terminal scFv fusion and IgG N-terminal scFv fusion.

Diabody-Fc fusion. This involves replacing the Fab fragment of an IgG with a bispecific diabody (derivative of the scFv).

Dual-Variable-Domain-IgG (DVD-IgG). VL and VH domains of IgG with one specificity were fused respectively to the N-terminal of VL and VH of an IgG of different specificity via a linker sequence to form a DVD-IgG.

The term “diabody” refers to a bispecific antibody in which VH and VL domains are expressed in a single polypeptide chain using a linker that is too short to allow for pairing between the two domains on the same chain, thereby forcing the domains to pair with complementary domains of another chain and creating two antigen-binding sites (see, e.g., Holliger et al., Proc. Natl. Acad. Sci. USA 90:6444-48 (1993) and Poljak et al., Structure 2:1121-23 (1994)). Diabodies may be designed to bind to two distinct antigens and are bi-specific antigen binding constructs.

The term “nanobody” or a “single domain antibody” refers to an antigen-binding fragment of a single monomeric variable antibody domain comprising one variable domain (VH) of a heavy-chain antibody. They possess several advantages over traditional monoclonal antibodies (mAbs), including smaller size (15 kD), stability in the reducing intracellular environment, and ease of production in bacterial systems (Schumacher et al., (2018) Nanobodies: Chemical Functionalization Strategies and Intracellular Applications. Angew. Chem. Int. Ed. 57, 2314; Siontorou, (2013) Nanobodies as novel agents for disease diagnosis and therapy. International Journal of Nanomedicine, 8, 4215-27). These features render nanobodies amendable to genetic and chemical modifications (Schumacher et al., (2018) Nanobodies: Chemical Functionalization Strategies and Intracellular Applications. Angew. Chem. Int. Ed. 57, 2314), facilitating their application as research tools and therapeutic agents (Bannas et al., (2017) Nanobodies and nanobody-based human heavy chain antibodies as antitumor therapeutics. Frontiers in Immunology, 8, 1603). Over the past decade, nanobodies have been used for protein immobilization (Rothbauer et al., (2008) A Versatile Nanotrap for Biochemical and Functional Studies with Fluorescent Fusion Proteins. Mol. Cell. Proteomics, 7, 282-289), imaging (Traenkle et al., (2015) Monitoring Interactions and Dynamics of Endogenous Beta-catenin With Intracellular Nanobodies in Living Cells. Mol. Cell. Proteomics, 14, 707-723), detection of protein-protein interactions (Herce et al., (2013) Visualization and targeted disruption of protein interactions in living cells. Nat. Commun, 4, 2660: Massa et al., (2014) Site-Specific Labeling of Cysteine-Tagged Camelid Single-Domain Antibody-Fragments for Use in Molecular Imaging. Bioconjugate Chem, 25, 979-988), and as macromolecular inhibitors (Truttmann et al., (2015) HypE-specific Nanobodies as Tools to Modulate HypE-mediated Target AMPylation. J. Biol. Chem. 290, 9087-9100).

The therapeutic moiety can bean antigen-binding fragment that binds to a target of interest. The antigen-binding fragment that binds to the target of interest may include 1, 2, or 3, CDRs of a variable heavy chain (VH) sequence from an antibody that specifically binds to the target of interest. The antigen-binding fragment that binds to the target of interest may include 1, 2, or 3 CDRs of a variable light chain (VL) sequence from an antibody that specifically binds to the target of interest. The antigen-binding fragment that binds to the target of interest may include 1, 2, 3, 4, 5, or all 6 CDRs of a variable heavy chain (VH) and/or a variable light chain (VL) sequence from an antibody that specifically binds to the target of interest. The antigen-binding fragment that binds to the target may be a portion of a full-length antibody, such as Fab, F(ab′)2, Fab′, Fv fragments, minibodies, diabodies, single domain antibody (dAb), single-chain variable fragments (scFv), nanobodies, multispecific antibodies formed from antibody fragments, or any other modified configuration of the immunoglobulin molecule that includes an antigen-binding site or fragment of the required specificity.

The therapeutic moiety can include a bispecific antibody. Bispecific Antibodies (BsAbs) are antibodies that can simultaneously bind two separate and unique antigens (or different epitopes of the same antigen).

The therapeutic moiety can include a “diabody”.

The therapeutic moiety can include a nanobody or a single domain antibody (which can also be referred to herein as sdAbs or VHH).

The therapeutic moiety can include a “minibody.” Minibodies (Mb) include a CH3 domain fused or linked to an antigen-binding fragment (e.g., a CH3 domain fused or linked to an scFv, a domain antibody, etc.). The term “Mb” can signify a CH3 single domain. A CH3 domain can signify a minibody. (S. Hu et al., Cancer Res., 56, 3055-3061, 1996). See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

The therapeutic moiety can include a “monobody”. The term “monobody” refers to a synthetic binding protein constructed using a fibronectin type III domain (FN3) as a molecular scaffold.

The therapeutic moiety can be an antibody mimetic. Antibody mimetics are compounds that, like antibodies, can specifically bind antigens, but that are not structurally related to antibodies. They are usually artificial peptides or proteins with a molar mass of about 3 to 20 kD (compared to the molar mass of antibodies at ˜150 kDa.). Examples of antibody mimetics include Affibody molecules (constructed on a scaffold of the domain of Protein A, See, Nygren (June 2008). FEBS J. 275 (11): 2668-76), Affilins (constructed on a scaffold of gamma-B crystalline or ubiquitin, See Ebersbach H et al. (September 2007). J. Mol. Biol. 372 (1): 172-85), Affimers (constructed on a Crystatin scaffold, See Johnson A et al., (Aug. 7, 2012). Anal. Chem. 84 (15): 6553-60), Affitins (constructed on a Sac7d from S. acidocaldarius scaffold, See Krehenbrink M et al., (November 2008). J. Mol. Biol. 383 (5): 1058-68), Alphabodies (constructed on a triple helix coiled coil scaffold, See Desmet, J et al., (5 Feb. 2014). Nature Communications. 5: 5237), Anticalins (constructs on scaffold of lipocalins, See Skerra A (June 2008). FEBS J. 275 (11): 2677-83), Avimers (constructed on scaffolds of various membrane receptors, See Silverman J et al. (December 2005). Nat. Biotechnol. 23 (12): 1556-61), DARPins (constructed on scaffolds of ankyrin repeat motifs, See Stumpp et al., (August 2008). Drug Discov. Today. 13 (15-16): 695-701), Fynomers (constructed on a scaffold of the SH3 domain of Fyn, See Grabulovski et al., (2007). J Biol Chem. 282 (5): 3196-3204), Kunitz domain peptides (constructed on scaffolds of the Kunitz domains of various protease inhibitors, See Nixon et al (March 2006). Curr Opin Drug Discov Dev. 9 (2): 261-8), and Monobodies (constructed on scaffolds of type III domain of fibronectin, See Koide et al (2007). Methods Mol. Biol. 352: 95-109).

The therapeutic moiety can include “designed ankryin repeats” or “DARPins”. DARPins are derived from natural ankyrin proteins comprised of at least three repeat motifs proteins, and usually comprise of four or five repeats.

The therapeutic moiety can include “dualvariable-domain-IgG” or “DVD-IgG”. DVD-IgGs are generated from two parental monoclonal antibodies by fusing VL and VH domains of IgG with one specificity to the N-terminal of VL and VH of an IgG of different specificity, respectively, via a linker sequence.

The therapeutic moiety can include a F(ab) fragment.

The therapeutic moiety can include a F(ab′)2 fragment.

The therapeutic moiety can include an Fv fragment.

The antigen-binding fragment can include a “single chain variable fragment” or “scFv”. An scFv refers to a fusion protein of the variable regions of the heavy (VH) and light chains (VL) of immunoglobulins, connected with a short linker peptide of ten to about 25 amino acids. Huston et al. (1988) Proc. Nat. Acad. Sci. USA 85(16):5879-5883. The linker can connect the N-terminus of the VH with the C-terminus of the VL, or vice versa. A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

The antigen binding construct can comprise two or more antigen-binding moieties. The antigen binding constructs can bind to two separate and unique antigens or to different epitopes of the same antigen. Knobs-into-holes BsAb IgG. H chain heterodimerization is forced by introducing different mutations into the two CH3 domains resulting in asymmetric antibodies. Specifically, a “knob” mutation is made into one HC and a “hole” mutation is created in the other HC to promote heterodimerization.

### Peptide Inhibitors

The therapeutic moiety can include a peptide. the peptide can act as an agonist, increasing activity of a target protein. The peptide can act as an antagonist, decreasing activity of a target protein. The peptide can be configured to inhibit protein-protein interaction (PPI). Protein-protein interactions (PPIs) are important in many biochemical processes, including transcription of nucleic acid and various post-translational modifications of translated proteins. PPIs can be experimentally determined by biophysical techniques such as X-ray crystallography, NMR spectroscopy, surface plasma resonance (SPR), bio-layer interferometry (BLI), isothermal titration calorimetry (ITC), radio-ligand binding, spectrophotometric assays and fluorescence spectroscopy. Peptides that inhibit protein-protein interaction can be referred to as peptide inhibitors.

The therapeutic moiety can include a peptide inhibitor. The peptide inhibitor can include from about 5 to about 100 amino acids, from about 5 to about 50 amino acids; from about 15 to about 30 amino acids; or from about 20 to about 40 amino acids. The peptide inhibitor can include one or more chemical modifications, for example, to reduce proteolytic degradation and/or to improve in vivo half-life. The peptide inhibitor can include one or more synthetic amino acids and/or a backbone modification. The peptide inhibitor can have an α-helical structure.

The peptide inhibitor can target the dimerization domain of a homodimeric or heterodimeric target protein of interest.

### Small Molecules

The therapeutic moiety can include a small molecule. The therapeutic moiety can include a small molecule kinase inhibitor. The therapeutic moiety can include a small molecule that inhibits a kinase that phosphorylates a target of interest. Inhibition of phosphorylation of target of interest can block nuclear translocation of the target of interest. The therapeutic moiety can include a small molecule inhibitor of MyD88.

### Compositions

Compositions are provided that include the compounds described herein.

Pharmaceutically acceptable salts and/or prodrugs of the disclosed compounds are provided. Pharmaceutically acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically acceptable base addition salts include sodium, potassium, calcium, ammonium, or magnesium salt. Examples of physiologically acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Mechanism of Oligonucleotide Therapeutics and Target Molecules

Many types of oligonucleotides are capable of modulating gene transcription, translation and/or protein function in cells. Non-limiting examples of such oligonucleotides include, e.g; small interfering RNA (siRNA), microRNA (miRNA), antisense oligonucleotides, ribozymes, plasmids, immune stimulating nucleic acids, antisense, antagomir, antimir, microRNA mimic, supermir, Ul adaptor, and aptamer. Additional examples include DNA-targeting, triplex-forming oligonucleotide, strand-invading oligonucleotide, and synthetic guide strand for CRISPR/Cas, These nucleic acids act via a variety of mechanisms. See Smith and Zain, Annu Rev Pharmacol Toxicol. 2019, 59:605-630, incorporated by reference herein.

Splice-switching antisense oligonucleotides are short, synthetic, antisense, modified nucleic acids that base-pair with a pre-mRNA and disrupt the normal splicing repertoire of the transcript by blocking the RNA-RNA base-pairing or protein-RNA binding interactions that occur between components of the splicing machinery and the pre-mRNA. Splicing of pre-mRNA is required for the proper expression of the vast majority of protein-coding genes, and thus, targeting the process offers a means to manipulate protein production from a gene. Splicing modulation is particularly valuable in cases of disease caused by mutations that lead to disruption of normal splicing or when interfering with the normal splicing process of a gene transcript may be therapeutic. Such antisense oligonucleotides offer an effective and specific way to target and alter splicing in a therapeutic manner. See Havens and Hastings, Nucleic Acids Res. 2016 Aug. 19; 44(14):6549-6563, incorporated by reference herein.

In the case of siRNA or miRNA, these nucleic acids can down-regulate intracellular levels of specific proteins through a process termed RNA interference (RNAi). Following introduction of siRNA or miRNA into the cell cytoplasm, these double-stranded RNA constructs can bind to a protein termed RISC. The sense strand of the siRNA or miRNA is displaced from the RISC complex providing a template within RISC that can recognize and bind mRNA with a complementary sequence to that of the bound siRNA or miRNA. Having bound the complementary mRNA the RISC complex cleaves the mRNA and releases the cleaved strands. RNAi can provide down-regulation of specific proteins by targeting specific destruction of the corresponding mRNA that encodes for protein synthesis.

The therapeutic applications of RNAi are extremely broad, since siRNA and miRNA constructs can be synthesized with any nucleotide sequence directed against a target protein. To date, siRNA constructs have shown the ability to specifically down-regulate target proteins in both in vitro and in vivo models, as well as in clinical studies.

Antisense oligonucleotides and ribozymes can also inhibit mRNA translation into protein. In the case of antisense constructs, these single stranded deoxynucleic acids have a complementary sequence to that of the target protein mRNA and can bind to the mRNA by Watson-Crick base pairing. This binding either prevents translation of the target mRNA and/or triggers RNase H degradation of the mRNA transcripts, Consequently, antisense oligonucleotides have tremendous potential for specificity of action (i.e., down-regulation of a specific disease-related protein). To date, these compounds have shown promise in several in vitro and in vivo models, including models of inflammatory disease, cancer, and HIV (reviewed in Agrawal, Trends in Biotech. 14:376-387 (1996)). Antisense can also affect cellular activity by hybridizing specifically with chromosomal DNA.

Immune-stimulating nucleic acids include deoxyribonucleic acids and ribonucleic acids. In the case of deoxyribonucleic acids, certain sequences or motifs have been shown to illicit immune stimulation in mammals. These sequences or motifs include the CpG motif, pyrimidine-rich sequences and palindromic sequences. It is believed that the CpG motif in deoxyribonucleic acids is specifically recognized by an endosomal receptor, tolllike receptor 9 (TLR-9), which then triggers both the innate and acquired immune stimulation pathway. Certain immune stimulating ribonucleic acid sequences have also been reported. It is believed that these RNA sequences trigger immune activation by binding to toll-like receptors 6 and 7 (TLR-6 and TLR-7). In addition, double-stranded RNA is also reported to be immune stimulating and is believed to activate via binding to TLR-3.

Non-limiting examples of mechanism and targets of antisense oligonucleotides (ASOs) to modulate gene transcription, translation and/or protein function are illustrated in Table 9A and 9B.

TABLE 9A
Mechanism of ASO Modulation and Target Molecules

| | Location | Subcellular | |
|---|---|---|---|
| Types | of target | Location | Mechanism |
| mRNA | Intracellular | cytoplasm | inhibition of |
| | | | translation |
| Pre-mRNA | Intracellular | nucleus | alternative splicing |
| micro-RNA | Intracellular | cytoplasm | miRNA inhibition |
| | | and nucleus | or activation |
| long non- | Intracellular | cytoplasm | inhibition of |
| coding RNA | | and nucleus | IncRNA function |
| Telomerase | Intracellular | cytoplasm | inhibition of |
| RNA component | | and nucleus | telomerase |
| Protein | Extra- and | cytoplasm | inhibition of |
| | intra-cellular | and nucleus | protein target |

TABLE 9B
Mechanism of ASO Modulation and Target Molecules

| | | | Examples |
|---|---|---|---|
| Mechanism | Target | Description | of drugs |
| Regulation of pre- | pre-mRNA | ASOs bind to pre-mRNA and alter | Nusinersen, |
| mRNA splicing | | the splicing by steric blocking, which | Eteplirsen |
| | | result in disruption of the recognition | |
| | | by splicing factors | |
| Regulation of RNA | pre-mRNA | ASOs containing DNA bases bind to | Mipomersen, |
| translation by | and mRNA | target RNA and induce the cleavage | Inotersen |
| recruiting RNase H | | of RNA by RNase H | |
| Regulation of RNA | mRNA | ASOs and duplex RNA can both | |
| translation by steric | | sterically block the translation | |
| blocking | | machinery to inhibit RNA translation | |
| | | or enhance RNA translation by | |
| | | blocking aberrant sites that reduce | |
| | | RNA translation | |
| Regulation of RNA | mRNA | siRNA and miRNA inhibit | Patisiran, |
| translation by RNAi | | translation by RNA interference and | Inclisiran, |
| | | induce the cleavage of target RNA | Fitusiran, |
| | | | Givosiran |
| Regulation of | protein | Aptamers bind with target proteins as | Pegaptanib |
| protein activity by | | antagonists | |
| binding with target | | | |
| proteins | | | |

Clustered regularly interspaced short palindromic repeats (CRISPR) and associated Cas proteins constitute the CRISPR-Cas system. CRISPR-Cas is a mechanism for gene-editing. The RNA-guided (e.g., gRNA) Cas9 endonuclease specifically targets and cleaves DNA in a sequence-dependent manner. The Cas9 endonuclease can be substituted with any nuclease of the disclosure. The gRNA targets a nuclease (e.g., a Cas9 nuclease) to a specific nucleotide region of interest (e.g., a genomic DNA sequence to be cleaved) and cleaves genomic DNA. Genomic DNA can then be replaced with a genetic element of interest.

Methods of Modulation of Tissue Distribution and/or Retention

Provided herein are compounds and methods for modulating tissue distribution and/or retention of a therapeutic agent in a subject. Compounds that modulate tissue distribution of a therapeutic agent can include a cyclic cell penetrating peptide (cCCP) and an exocyclic peptide (EP). Methods for modulating tissue distribution can comprise administering to the subject a compound that includes a cyclic cell penetrating peptide (cCPP) and an exocyclic peptide (EP). Modulation of tissue distribution or retention of a compound can be assessed by measurement of the amount, expression, function or activity of the compound in vivo in different tissues. The tissues can be different tissues of the same biological system, such as different types of muscle tissues or different tissues within the central nervous system. The tissue can be muscle tissue and there is modulation of distribution or retention of the compound in cardiac muscle tissue as compared to at least one other type of muscle tissue (e.g., skeletal muscle, including but not limited to diaphragm, tibialis anterior and triceps, or smooth muscle). The tissue can be CNS tissue and there is modulation of distribution or retention of the compound in at least one CNS tissue as compared to at least one other type of CNS tissue.

Any of the EPs described herein are suitable for inclusion in the compound used in the method. The EP can be PKKKRKV (SEQ ID NO: 103). The EP can be KK, KR, RR, KKK, KGK, KBK, KBR, KRK, KRR, RKK, RRR, KKKK (SEQ ID NO: 64), KKRK (SEQ ID NO: 65), KRKK (SEQ ID NO: 66), KRRK (SEQ ID NO: 67), RKKR (SEQ ID NO: 68), RRRR (SEQ ID NO: 69), KGKK (SEQ ID NO: 70), KKGK (SEQ ID NO: 71), KKKKK (SEQ ID NO: 75), KKKRK (SEQ ID NO: 76), KBKBK (SEQ ID NO: 81), KKKRKV (SEQ ID NO: 90), PKKKRKV (SEQ ID NO: 103), PGKKRKV (SEQ ID NO: 104), PKGKRKV (SEQ ID NO: 105), PKKGRKV (SEQ ID NO: 106), PKKKGKV (SEQ ID NO: 107), PKKKRGV (SEQ ID NO: 108) and PKKKRKG (SEQ ID NO: 109). The EP can be selected from KK, KR, RR, KKK, KGK, KBK, KBR, KRK, KRR, RKK, RRR, KKKK (SEQ ID NO: 64), KKRK (SEQ ID NO: 65), KRKK (SEQ ID NO: 66), KRRK (SEQ ID NO: 67), RKKR (SEQ ID NO: 68), RRRR (SEQ ID NO: 69), KGKK (SEQ ID NO: 70), KKGK (SEQ ID NO: 71), KKKKK (SEQ ID NO: 75), KKKRK (SEQ ID NO: 76), KBKBK (SEQ ID NO: 81), KKKRKV (SEQ ID NO: 90), PGKKRKV (SEQ ID NO: 104), PKGKRKV (SEQ ID NO: 105), PKKGRKV (SEQ ID NO: 106), PKKKGKV (SEQ ID NO: 107), PKKKRGV (SEQ ID NO: 108) and PKKKRKG (SEQ ID NO: 109).

The EP can comprise PKKKRKV (SEQ ID NO: 103), RR, RRR, RHR, RBR, RBRBR (SEQ ID NO: 99), RBHBR (SEQ ID NO: 100), or HBRBH (SEQ ID NO: 101), wherein B is beta-alanine. The amino acids in the EP can have D or L stereochemistry. The EP can be PKKKRKV (SEQ ID NO: 103), RR, RRR, RHR, RBR, RBRBR (SEQ ID NO: 99), RBHBR (SEQ ID NO: 100), or HBRBH (SEQ ID NO: 101), wherein B is beta-alanine. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV (SEQ ID NO: 103). The EP can consist of an NLS comprising the amino acid sequence PKKKRKV (SEQ ID NO: 13). The EP can comprise an NLS comprising an amino acid sequence selected from NLSKRPAAIKKAGQAKKKK (SEQ ID NO: 253), PAAKRVKLD (SEQ ID NO: 112), RQRRNELKRSF (SEQ ID NO: 113), RMRKFKNKGKDTAELRRRRVEVSVELR (SEQ ID NO: 114), KAKKDEQILKRRNV (SEQ ID NO: 115), VSRKRPRP (SEQ ID NO: 116), PPKKARED (SEQ ID NO: 117), PQPKKKPL (SEQ ID NO: 118), SALIKKKKKMAP (SEQ ID NO: 119), DRLRR (SEQ ID NO: 120), PKQKKRK (SEQ ID NO: 121), RKLKKKIKKL (SEQ ID NO: 122), REKKKFLKRR (SEQ ID NO: 123), KRKGDEVDGVDEVAKKKSKK (SEQ ID NO: 124) and RKCLQAGMNLEARKTKK (SEQ ID NO: 125). The EP can consist of an NLS comprising an amino acid sequence selected from NLSKRPAAIKKAGQAKKKK (SEQ ID NO: 253), PAAKRVKLD (SEQ ID NO: 112), RQRRNELKRSF (SEQ ID NO: 113), RMRKFKNKGKDTAELRRRRVEVSVELR (SEQ ID NO: 114), KAKKDEQILKRRNV (SEQ ID NO: 115), VSRKRPRP (SEQ ID NO: 116), PPKKARED (SEQ ID NO: 117), PQPKKKPL (SEQ ID NO: 118), SALIKKKKKMAP (SEQ ID NO: 119), DRLRR (SEQ ID NO: 120), PKQKKRK (SEQ ID NO: 121), RKLKKKIKKL (SEQ ID NO: 122), REKKKFLKRR (SEQ ID NO: 123), KRKGDEVDGVDEVAKKKSKK (SEQ ID NO: 124) and RKCLQAGMNLEARKTKK (SEQ ID NO: 125).

The amount, expression, function or activity of the compound may be increased at least 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450% or 500% in at least one tissue as compared to a second tissue.

The amount, expression, function or activity of the compound may be decreased at least 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450% or 500% in at least one tissue as compared to a second tissue.

Amount or expression of the compound can be assessed in different tissue types by methods known in the art, including but not limited to methodologies described in the Examples. Tissue can be prepared by standard methods. The amount or expression of the compound in different tissues can be measured by techniques well-established in the art, for example by LC-MS/MS, Western blot analysis or ELISA. The function or activity of the compound in different tissues can be measured by techniques established for assessing the relevant function or activity, such as use of RT-PCR to evaluate the activity of oligonucleotide-based therapeutic moieties. For example, for an antisense compound (AC) used as the therapeutic moiety (TM) to induces exon-skipping in a target mRNA of interest, RT-PCR can be used to quantify the level of exon-skipping in different tissues.

Tissue distribution and/or retention of the therapeutic agent in tissues of the central nervous system (CNS) can be modulated with a compound that includes a cyclic cell penetrating peptide (cCPP) and an exocyclic peptide (EP). The compound may be administered to the subject intrathecally and the compound may modulate tissue distribution and/or retention of the therapeutic agent in tissues of the central nervous system (CNS). Non-limiting examples of tissues of the CNS include cerebellum, cortex, hippocampus, olfactory bulb, spinal cord, dorsal root ganglion (DRG) and cerebrospinal fluid (CSF). The compound comprising a cCPP and an EP can be administered intrathecally and the level of expression, activity or function of the therapeutic agent may be higher in at least one CNS tissue as compared to another CNS tissue. The compound comprising a cCPP and an EP can be administered intrathecally and the level of expression, activity or function of the therapeutic agent may be lower in at least one CNS tissue as compared to another CNS tissue. The therapeutic agent can include a CD33-targeted therapeutic agent (e.g., a CD33-targeted antisense compound), wherein the compound is administered intrathecally. The compound comprising a cCPP and an EP can be administered intrathecally at a dosage of at least 1 mg/kg, 5 mg/kg, 10 mg/kg, 15 mg/kg, 20 mg/kg, 25 mg/kg or 50 mg/kg.

Method of modulating tissue distribution or retention of a therapeutic agent in the central nervous system (CNS) of a subject may comprise: administering intrathecally to the subject a compound comprising:
(a) a cyclic cell penetrating peptide (cCPP);
(b) a therapeutic moiety (TM) comprising the therapeutic agent; and
(c) an exocyclic peptide (EP) comprising at least one positively-charged amino acid residue, wherein the amount, expression, function or activity of the therapeutic agent is modulated at least 10% in at least one tissue of the CNS of the subject as compared to a second tissue of the CNS of the subject.

The amount, expression, function or activity of the therapeutic agent can be modulated at least 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450% or 500% in at least one tissue of the CNS of the subject as compared to a second tissue of the CNS of the subject.

Any of the therapeutic agents described herein for CNS-related diseases or disorders are suitable for inclusion in the compound used in the method. The therapeutic agent can include a CD33-targeted therapeutic agent, such as any of the CD33-targeted antisense compounds described herein.

Any of the CPPs described herein are suitable for inclusion in the compound used in the method. The CPP can be a cyclic CPP (cCPP).

The compound can be used to treat a subject with a central nervous system disease or disorder or a neuroinflammatory disease or disorder. In embodiments, the subject has Alzheimer's disease or Parkinson's disease.

Tissue distribution and/or retention of the therapeutic agent in different types of muscle tissues can be modulated. Non-limiting examples of muscle tissues include the diaphragm, cardiac (heart) muscle, tibialis anterior muscle, triceps muscle, other skeletal muscles and smooth muscle. A compound comprising a cCPP, EP and therapeutic agent can be administered and the level of expression, activity or function of the therapeutic agent can be higher in at least one muscle tissue as compared to another muscle tissue. A compound comprising a cCPP, EP and therapeutic agent can be administered and the level of expression, activity or function of the therapeutic agent can be lower in at least one muscle tissue as compared to another muscle tissue. The therapeutic agent can be a dystophin-targeted therapeutic agent (e.g., a DMD-targeted antisense compound). The compound can be administered at a dosage of at least 1 mg/kg, 5 mg/kg, 10 mg/kg, 15 mg/kg, 20 mg/kg, 25 mg/kg or 50 mg/kg.

A method of modulating tissue distribution or retention of a therapeutic agent in the muscular system of a subject comprises: administering to the subject a compound comprising:
(a) a cyclic cell penetrating peptide (cCPP);
(b) a therapeutic moiety (TM) comprising the therapeutic agent; and
(c) an exocyclic peptide (EP) comprising at least one positively-charged amino acid residue, wherein the amount, expression, function or activity of the therapeutic agent is modulated at least 10% in at least one tissue of the muscular system of the subject as compared to a second tissue of the muscular system of the subject.

The amount, expression, function or activity of the therapeutic agent can be modulated at least 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450% or 500% in at least one tissue of the muscular system of the subject as compared to a second tissue of the muscular system of the subject.

Any of the therapeutic agents described herein for muscular system-related diseases or disorders are suitable for inclusion in the compound used in the method. The therapeutic agent can be a DMD-targeted therapeutic agent, such as a DMD-targeted antisense compound.

Any of the CPPs described herein are suitable for inclusion in the compound used in the method. In embodiments, the CPP is a cyclic CPP (cCPP).

In embodiments, the subject has a neuromuscular disorder or a musculoskeletal disorder. In embodiments, the subject has Duchenne muscular dystrophy.

Diseases Associated with Aberrant Splicing and Exemplary Target Genes

The human genome comprises more than 40,000 genes, approximately half of which correspond to protein-coding genes. However, the number of human protein species is predicted to be orders of magnitude higher due to single amino acid polymorphisms, post translational modifications, and, importantly, alternative splicing. RNA splicing, generally taking place in the nucleus, is the process by which precursor messenger RNA (pre-mRNA) is transformed into mature messenger RNA (mRNA) by removing non-coding regions (introns) and joining together the remaining coding regions (exons). The resulting mRNA can then be exported from the nucleus and translated into protein. Alternative splicing, or differential splicing, is a regulated process during gene expression that results in a single gene coding for multiple proteins. In this process, particular exons of a gene may be included within or excluded from the final, processed mRNA produced from that gene. While alternative splicing is a normal phenomenon in eukaryotic organisms, and contributes to the biodiversity of proteins encoded by a genome, abnormal variations in splicing are heavily implicated in disease. A large proportion of human genetic disorders result from splicing variants; abnormal splicing variants contribute to the development of cancer; and splicing factor genes are frequently mutated in different types of cancer.

About 10% of ˜80,000 mutations reported in the human gene mutation database (HGMD) affect splice sites. In the HGMD, there are 3390 disease-causing mutations that occur at the +1 donor splice site. These mutations affect 2754 exons in 901 genes. The prevalence is even higher for neuromuscular disorders (NMDs) due to the unusually large size and multiexonic structure of genes encoding muscle structural proteins, further highlighting the importance of these mutations in NMDs.

Previously, the correction of point mutations, e.g. splice site mutations, has been attempted via the homology-directed repair (HDR) pathway, which is extremely inefficient in post-mitotic tissues such as skeletal muscles, hampering its therapeutic utility in NMD. In addition, standard gene therapy approaches to reintroduce corrected coding regions into the genome are impeded by the large size of genes encoding, e.g., muscular structural proteins. Furthermore, many existing therapies rely on inefficient introduction of the therapeutic compound into the disease cells, such that in vivo treatment is impractical and higher toxicities are experienced.

The target gene of the present disclosure may be any eukaryotic gene comprising one or more introns and one or more exons. The target gene can be a mammalian gene. The mammal can be a human, mouse, bovine, rat, pig, horse, chicken, sheep, or the like. The target gene can be a human gene.

The target gene can be a gene comprising mutations leading to aberrant splicing. The target gene can be a gene that comprises one or more mutations. The target gene can be a gene that comprises one or more mutations, such that transcription and translation of the target gene does not lead to a functional protein. The target gene can be a gene that comprises one or more mutations, such that transcription and translation of the target gene leads to a target protein that is less active or less functional than a wild type target protein.

The target gene can be a gene underlying a genetic disorder. The target gene may have abnormal gene expression in the central nervous system. The target gene can be a gene involved in the pathogenesis of a neuromuscular disorder (NMD). The target gene can be a gene involved in the pathogenesis of a musculoskeletal disorder (NMD). The neuromuscular disease can be Pompe disease, and the target gene can be GYS1.

Antisense compounds may be used to target genes comprising mutations that lead to aberrant splicing underlying a genetic disease in order to redirect splicing to give a desired splice product (Kole, Acta Biochimica Polonica, 1997, 44, 231-238).

CRISPR gene-editing machinery may be used to target aberrant genes for removal or to regulate gene transcription and translation.

The disease can include β-thalassemia (Dominski and Kole, Proc. Natl. Acad. Sci. USA, 1993, 90, 8673-8677; Sierakowska et al., Nucleosides & Nucleotides, 1997, 16, 1173-1182; Sierakowska et al., Proc. Natl. Acad. Sci. USA, 1996, 93, 12840-44; Lacerra et al., Proc. Natl. Acad. Sci. USA, 2000, 97, 9591-9596).

The disease can include dystrophin Kobe (Takeshima et al., J. Clin. Invest., 1995, 95, 515-520).

The disease can include Duchenne muscular dystrophy (Dunckley et al. Nucleosides & Nucleotides, 1997, 16, 1665-1668; Dunckley et al. Human Mol. Genetics, 1998, 5, 1083-90). The target gene can be the DMD gene, which codes for dystrophin. The protein consists of an N-terminal domain that binds to actin filaments, a central rod domain, and a C-terminal cysteine-rich domain that binds to the dystrophin-glycoprotein complex (Hoffman et al. 1987; Koenig et al. 1988; Yoshida and Ozawa 1990). Mutations in the DMD gene that interrupt the reading frame result in a complete loss of dystrophin function, which causes severe Duchenne muscular dystrophy (DMD) [MIM 310200]). The milder Becker muscular dystrophy (BMD [MIM 300376]), on the other hand, is the result of mutations in the same gene that are not frameshifting and result in an internally deleted but partially functional dystrophin that has retained its N- and C-terminal ends (Koenig et al. 1989; Di Blasi et al. 1996). Over two-thirds of patients with DMD and BMD have a deletion of >1 exon (den Dun-nen et al. 1989). Remarkably, patients have been described who exhibit very mild BMD and who lack up to 67% of the central rod domain (England et al. 1990; Winnard et al. 1993; Mirabella et al. 1998). This suggests that, despite large deletions, a partially functional dystrophin can be generated, provided that the deletions render the transcript in frame. These observations have led to the idea of using ACs to alter splicing so that the open reading frame is restored and the severe DMD phenotype is converted into a milder BMD phenotype. Several studies have shown therapeutic AC-induced single-exon skipping in cells derived from the mdx mouse model (Dunckley et al. 1998; Wilton et al. 1999; Mann et al. 2001, 2002; Lu et al. 2003) and various DMD patients (Takeshima et al. 2001; van Deutekom et al. 2001; Aartsma-Rus et al. 2002, 2003; De Angelis et al. 2002). The AC can be used to skip one or more exons selected from exons 2, 8, 11, 17, 19, 23, 29, 40, 41, 42, 43, 44, 45, 46, 48, 49, 50, 51, 52, 53, 55, and 59 of DMD. See Aartsma-Rus et al. 2002, incorporated by reference herein. The AC can be used to skip one or more exons selected from exons 8, 11, 43, 44, 45, 50, 51, 53, and 55 of DMD. Of all patients with DMD, ˜75% would benefit from the skipping of these exons. The skipping of exons flanking out-of-frame deletions or an in-frame exon containing a nonsense mutation can restore the reading frame and induce the synthesis of BMD-like dystrophins in treated cells. (van Deutekom et al. 2001; Aartsma-Rus et al. 2003). The AC hybridizing to its target sequence within a target DMD pre-mRNA can induce skipping of one or more exons. The AC can induce expression of a re-spliced target protein comprising an active fragment of dystrophin. Non-limiting examples of AC for exon 52 are described in US Pub. No. 2019/0365918, which is incorporated by reference in its entirety for all purposes. Compounds may comprise an EP, cCPP and a cargo that target the DMD gene.

Cyclic Cell Penetrating Peptides (cCPPs) Conjugated to a Cargo Moiety

The cyclic cell penetrating peptide (cCPP) can be conjugated to a cargo moiety.

The cargo moiety can be conjugated to cCPP through a linker. The cargo moiety can comprise therapeutic moiety. The therapeutic moiety can comprise an oligonucleotide, a peptide or a small molecule. The oligonucleotide can comprise an antisense oligonucleotide. The cargo moiety can be conjugated to the linker at the terminal carbonyl group to provide the following structure:

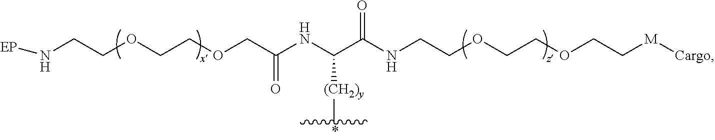

herein:
EP is an exocyclic peptide and M, AA_{SC}, Cargo, x′, y, and z′ are as defined above, * is the point of attachment to the AA_{SC}. x′ can be 1. y can be 4. z′ can be 11. —(OCH₂CH₂)_{x′}— and/or —(OCH₂CH₂)_{z′}— can be independently replaced with one or more amino acids, including, for example, glycine, beta-alanine, 4-aminobutyric acid, 5-aminopentanoic acid, 6-aminohexanoic acid, or combinations thereof.

An endosomal escape vehicle (EEV) can comprise a cyclic cell penetrating peptide (cCPP), an exocyclic peptide (EP) and linker, and can be conjugated to a cargo to form an EEV-conjugate comprising the structure of Formula (C):

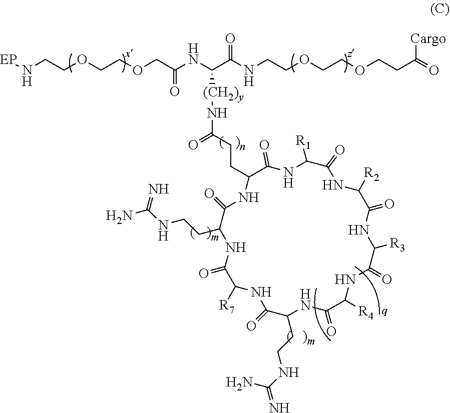

or a protonated for thereof,
wherein:
R₁, R₂, and R₃ can each independently be H or an amino acid residue having a side chain comprising an aromatic group;
   R₄ is H or an amino acid side chain;
   EP is an exocyclic peptide as defined herein;
   Cargo is a moiety as defined herein;
   each m is independently an integer from 0-3;
   n is an integer from 0-2;
   x′ is an integer from 2-20;
   y is an integer from 1-5;
   q is an integer from 1-4; and
   z′ is an integer from 2-20.

R₁, R₂, R₃, R₄, EP, cargo, m, n, x′, y, q, and z′ are as defined herein.

The EEV can be conjugated to a cargo and the EEV-conjugate can comprise the structure of Formula (C-a) or (C-b)

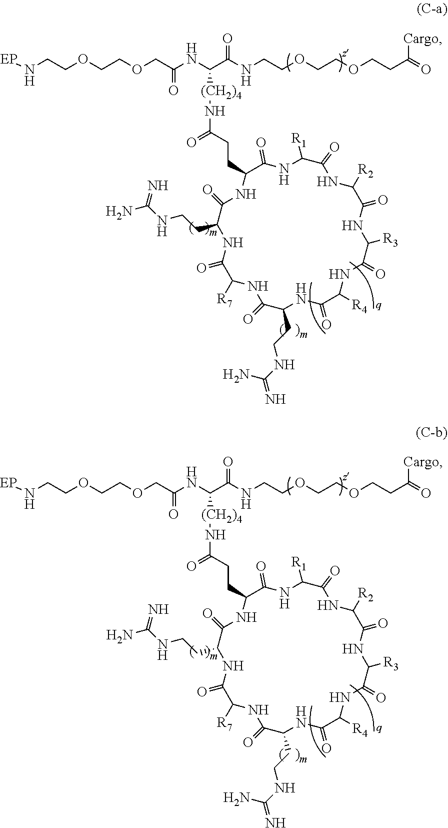

or a protonated form thereof, wherein EP, m and z are as defined above in Formula (C).

The EEV can be conjugated to a cargo and the EEV-conjugate can comprise the structure of Formula (C-c):

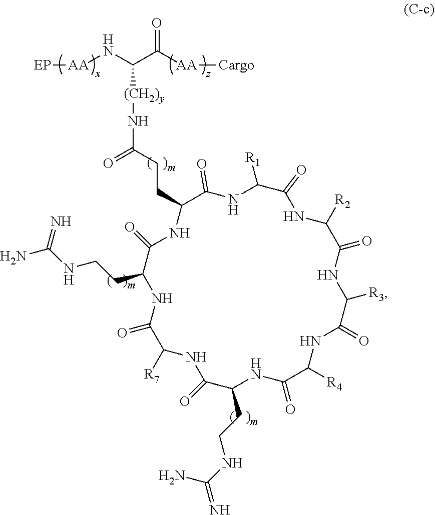

or a protonated form thereof, wherein EP, R¹, R², R³, R⁴, and m are as defined above in Formula (III); AA can be an amino acid as defined herein; n can be an integer from 0-2; x can be an integer from 1-10; y can be an integer from 1-5; and z can be an integer from 1-10.

The EEV can be conjugated to an oligonucleotide cargo and the EEV-oligonucleotide conjugate can comprises a structure of Formula (C-1), (C-2), (C-3), or (C-4):

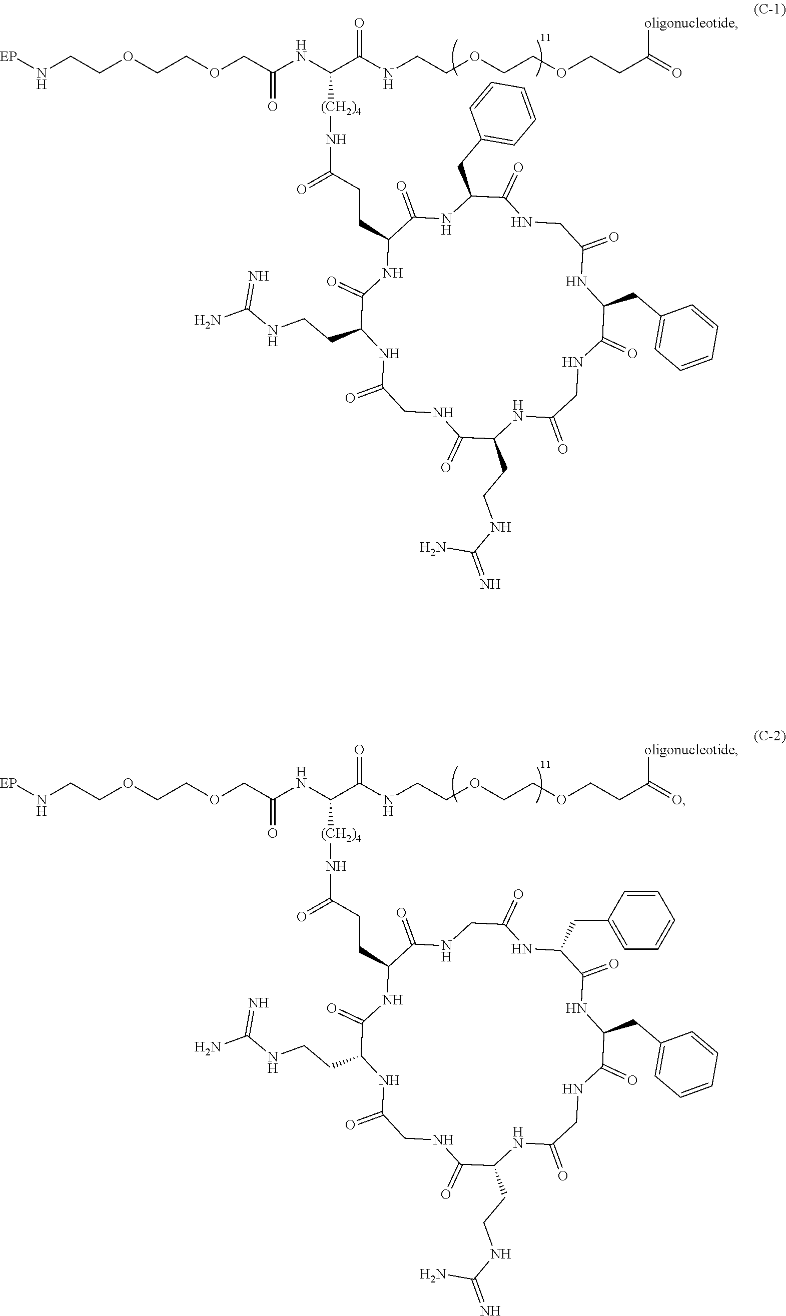

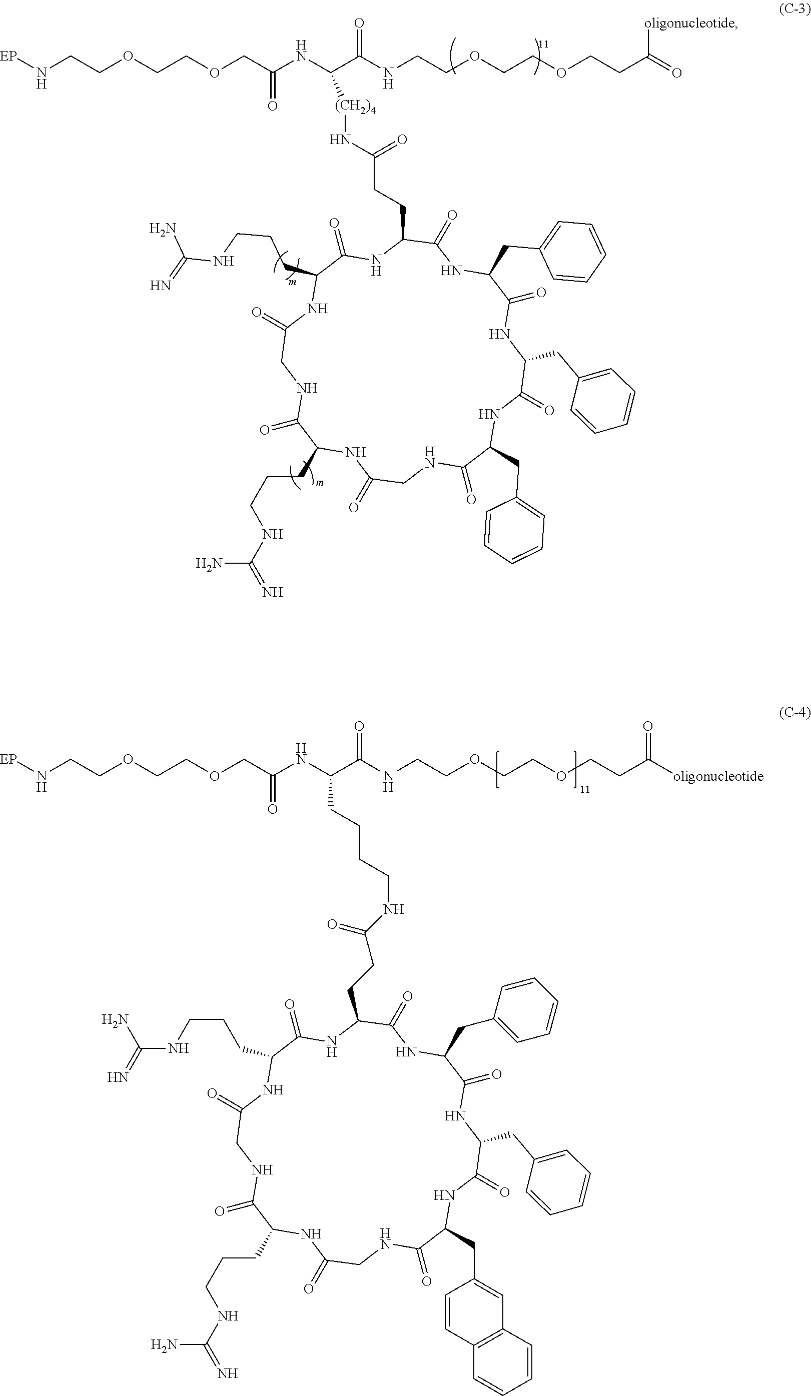

The EEV can be conjugated to an oligonucleotide cargo and the EEV-conjugate can comprise the structure:

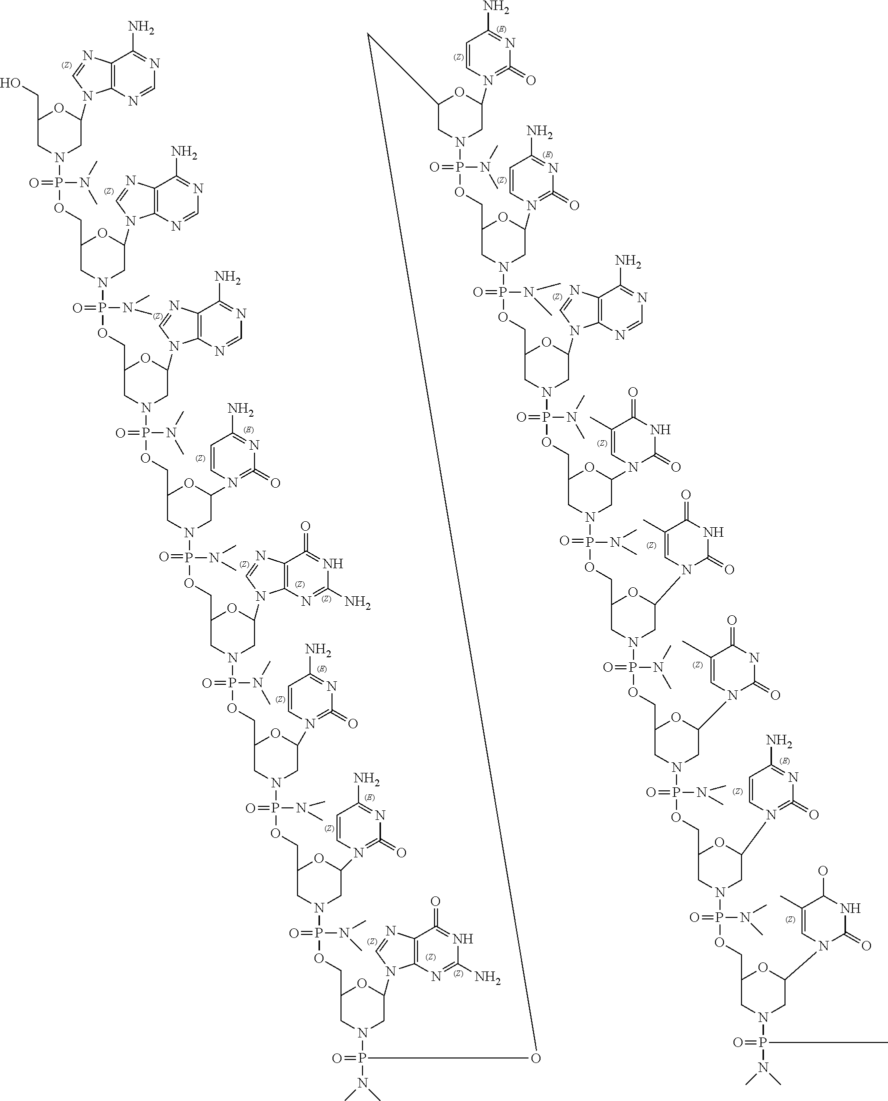

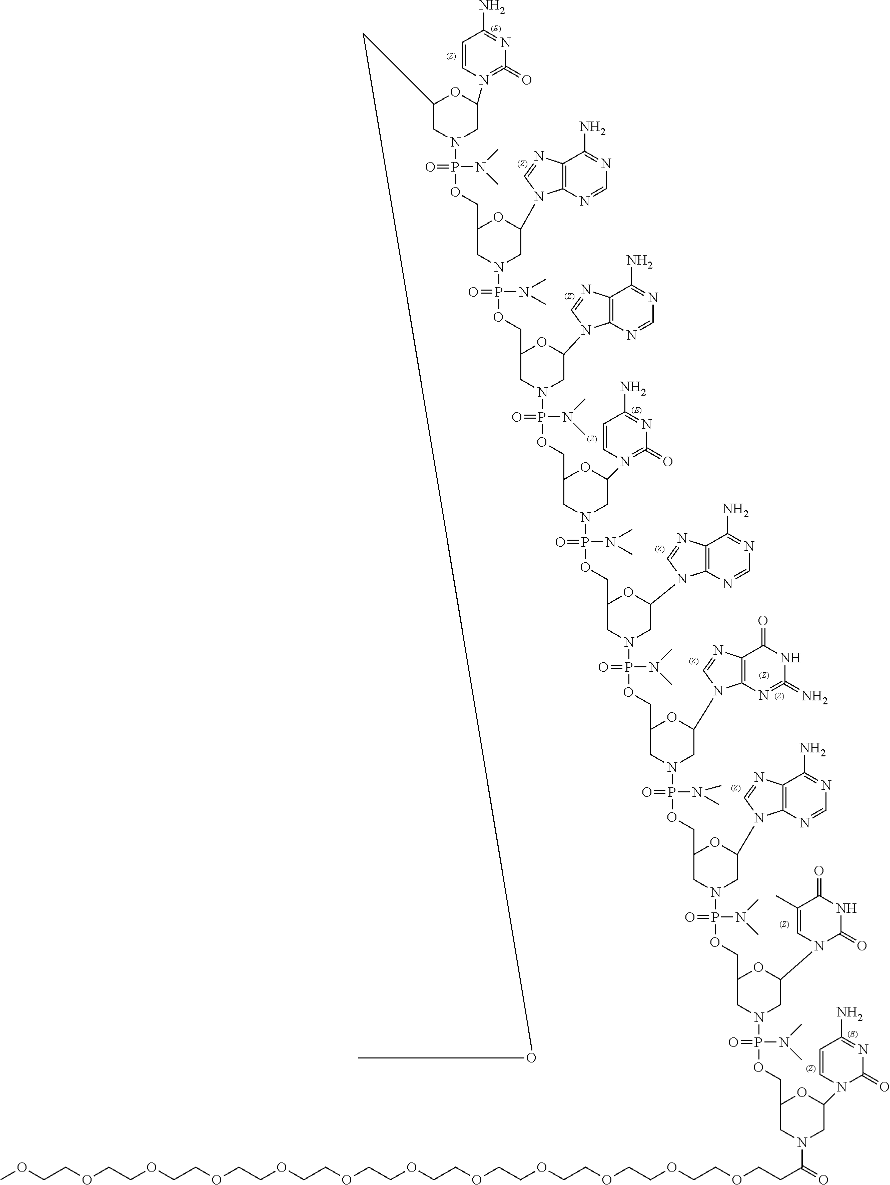

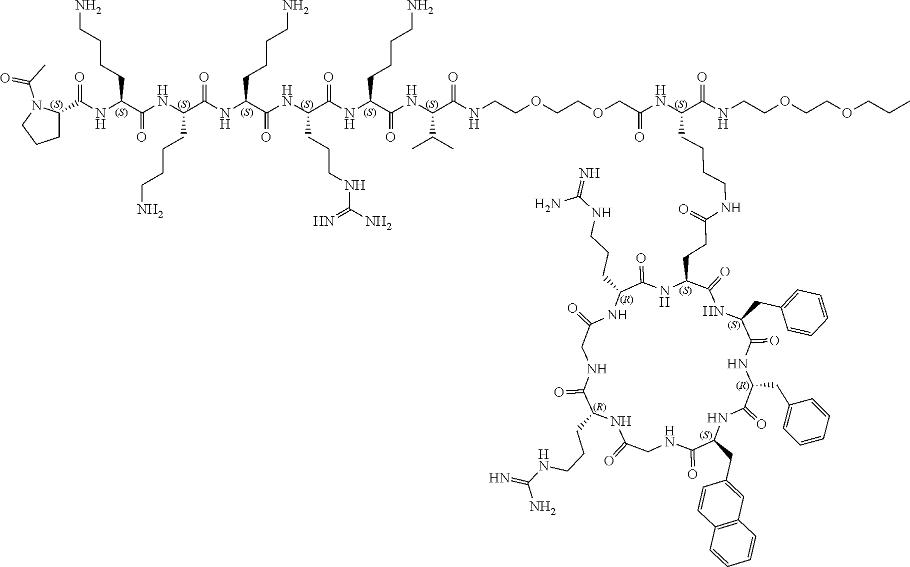

### Cytosolic Delivery Efficiency

Modifications to a cyclic cell penetrating peptide (cCPP) may improve cytosolic delivery efficiency. Improved cytosolic uptake efficiency can be measured by comparing the cytosolic delivery efficiency of a cCPP having a modified sequence to a control sequence. The control sequence does not include a particular replacement amino acid residue in the modified sequence (including, but not limited to arginine, phenylalanine, and/or glycine), but is otherwise identical.

As used herein cytosolic delivery efficiency refers to the ability of a cCPP to traverse a cell membrane and enter the cytosol of a cell. Cytosolic delivery efficiency of the cCPP is not necessarily dependent on a receptor or a cell type. Cytosolic delivery efficiency can refer to absolute cytosolic delivery efficiency or relative cytosolic delivery efficiency.

Absolute cytosolic delivery efficiency is the ratio of cytosolic concentration of a cCPP (or a cCPP-cargo conjugate) over the concentration of the cCPP (or the cCPP-cargo conjugate) in the growth medium. Relative cytosolic delivery efficiency refers to the concentration of a cCPP in the cytosol compared to the concentration of a control cCPP in the cytosol. Quantification can be achieved by fluorescently labeling the cCPP (e.g., with a FITC dye) and measuring the fluorescence intensity using techniques well-known in the art.

Relative cytosolic delivery efficiency is determined by comparing (i) the amount of a cCPP of the invention internalized by a cell type (e.g., HeLa cells) to (ii) the amount of a control cCPP internalized by the same cell type. To measure relative cytosolic delivery efficiency, the cell type may be incubated in the presence of a cCPP for a specified period of time (e.g., 30 minutes, 1 hour, 2 hours, etc.) after which the amount of the cCPP internalized by the cell is quantified using methods known in the art, e.g., fluorescence microscopy. Separately, the same concentration of the control cCPP is incubated in the presence of the cell type over the same period of time, and the amount of the control cCPP internalized by the cell is quantified.

Relative cytosolic delivery efficiency can be determined by measuring the IC₅₀ of a cCPP having a modified sequence for an intracellular target and comparing the IC₅₀ of the cCPP having the modified sequence to a control sequence (as described herein).

The relative cytosolic delivery efficiency of the cCPPs can be in the range of from about 50% to about 450% compared to cyclo(FfΦRrRrQ), e.g., about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 510%, about 520%, about 530%, about 540%, about 550%, about 560%, about 570%, about 580%, or about 590%, inclusive of all values and subranges therebetween. The relative cytosolic delivery efficiency of the cCPPs can be improved by greater than about 600% compared to a cyclic peptide comprising cyclo(FfΦRrRrQ).

The absolute cytosolic delivery efficacy of from about 40% to about 100%, e.g., about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, inclusive of all values and subranges therebetween.

The cCPPs of the present disclosure can improve the cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween.

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for aspartic acid and glutamic acid, benzyl and t-butyl and for cysteine, triphenylmethyl (trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxy-acetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl)phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

### Methods of Use

Also provided herein are methods of use of the compounds or compositions described herein. Also provided herein are methods for treating a disease or pathology in a subject in need thereof comprising administering to the subject an effective amount of any of the compounds or compositions described herein. The compounds of compositions can be used to treat any disease or condition that is amendable to treatment with the therapeutic moieties disclosed herein.

Also provided herein are methods of treating cancer in a subject. The methods include administering to a subject an effective amount of one or more of the compounds or compositions described herein, or a pharmaceutically acceptable salt thereof. The compounds and compositions described herein or pharmaceutically acceptable salts thereof are useful for treating cancer in humans, e.g., pediatric and geriatric populations, and in animals, e.g., veterinary applications. The disclosed methods can optionally include identifying a patient who is or can be in need of treatment of a cancer. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Further examples of cancers treatable by the compounds and compositions described herein include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

The methods of treatment or prevention of cancer described herein can further include treatment with one or more additional agents (e.g., an anti-cancer agent or ionizing radiation). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

For example, the compounds or compositions or pharmaceutically acceptable salts thereof as described herein can be combined into a pharmaceutical composition with an additional anti-cancer agent, such as 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Coil, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-1l, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The additional anti-cancer agent can also include biopharmaceuticals such as, for example, antibodies.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease.

Also described herein are methods of killing a tumor cell in a subject. The method includes contacting the tumor cell with an effective amount of a compound or composition as described herein, and optionally includes the step of irradiating the tumor cell with an effective amount of ionizing radiation. Additionally, methods of radiotherapy of tumors are provided herein. The methods include contacting the tumor cell with an effective amount of a compound or composition as described herein, and irradiating the tumor with an effective amount of ionizing radiation. As used herein, the term ionizing radiation refers to radiation comprising particles or photons that have sufficient energy or can produce sufficient energy via nuclear interactions to produce ionization. An example of ionizing radiation is x-radiation. An effective amount of ionizing radiation refers to a dose of ionizing radiation that produces an increase in cell damage or death when administered in combination with the compounds described herein. The ionizing radiation can be delivered according to methods as known in the art, including administering radiolabeled antibodies and radioisotopes.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection. Prophylactic administration can be used, for example, in the chemopreventative treatment of subjects presenting precancerous lesions, those diagnosed with early stage malignancies, and for subgroups with susceptibilities (e.g., family, racial, and/or occupational) to particular cancers. Therapeutic treatment involves administering to a subject a therapeutically effective amount of the compounds and compositions or pharmaceutically acceptable salts thereof as described herein after cancer is diagnosed.

In some examples of the methods of treating of treating cancer or a tumor in a subject, the compound or composition administered to the subject can comprise a therapeutic moiety that can comprise a targeting moiety that can act as an inhibitor against Ras (e.g., K-Ras), PTP1B, Pin1, Grb2 SH2, or combinations thereof.

The disclosed subject matter also concerns methods for treating a subject having a metabolic disorder or condition. An effective amount of one or more compounds or compositions disclosed herein can be administered to a subject having a metabolic disorder and who is in need of treatment thereof. In some examples, the metabolic disorder can comprise type II diabetes. In some examples of the methods of treating of treating the metabolic disorder in a subject, the compound or composition administered to the subject can comprise a therapeutic moiety that can comprise a targeting moiety that can act as an inhibitor against PTP1B. In one particular example of this method the subject is obese, and the method can comprise treating the subject for obesity by administering a composition as disclosed herein.

The disclosed subject matter also concerns methods for treating a subject having an immune disorder or condition. An effective amount of one or more compounds or compositions disclosed herein is administered to a subject having an immune disorder and who is in need of treatment thereof. In some examples of the methods of treating of treating the immune disorder in a subject, the compound or composition administered to the subject can comprise a therapeutic moiety that can comprise a targeting moiety that can act as an inhibitor against Pin1.

The disclosed subject matter also concerns methods for treating a subject having an inflammatory disorder or condition. An effective amount of one or more compounds or compositions disclosed herein can be administered to a subject having an inflammatory disorder and who is in need of treatment thereof.

The disclosed subject matter also concerns methods for treating a subject having cystic fibrosis. An effective amount of one or more compounds or compositions disclosed herein can be administered to a subject having cystic fibrosis and who is in need of treatment thereof. In some examples of the methods of treating the cystic fibrosis in a subject, the compound or composition administered to the subject can comprise a therapeutic moiety that can comprise a targeting moiety that can act as an inhibitor against CAL PDZ.

The compounds disclosed herein can be used for detecting or diagnosing a disease or condition in a subject. For example, a cCPP can comprise a targeting moiety and/or a detectable moiety that can interact with a target, e.g., a tumor.

### Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E.W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell can comprise attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The disclosed compositions are bioavailable and can be delivered orally. Oral compositions can be tablets, troches, pills, capsules, and the like, and can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein in one or more containers. The disclosed kits can optionally include pharmaceutically acceptable carriers and/or diluents. A kit can include one or more other components, adjuncts, or adjuvants as described herein. kit includes one or more anti-cancer agents, such as those agents described herein. A kit can include instructions or packaging materials that describe how to administer a compound or composition of the kit. Containers of the kit can be of any suitable material, e.g., glass, plastic, metal, etc., and of any suitable size, shape, or configuration. A compound and/or agent disclosed herein can be provided in the kit as a solid, such as a tablet, pill, or powder form. A compound and/or agent disclosed herein can be provided in the kit as a liquid or solution. A kit can comprise an ampoule or syringe containing a compound and/or agent disclosed herein in liquid or solution form.

### Certain Definitions

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, the term “cyclic cell penetrating peptide” or “cCPP” refers to a peptide that facilitates the delivery of a cargo, e.g., a therapeutic moiety, into a cell.

As used herein, the term “endosomal escape vehicle” (EEV) refers to a cCPP that is conjugated by a chemical linkage (i.e., a covalent bond or non-covalent interaction) to a linker and/or an exocyclic peptide (EP). The EEV can be an EEV of Formula (B).

As used herein, the term “EEV-conjugate” refers to an endosomal escape vehicle defined herein conjugated by a chemical linkage (i.e., a covalent bond or non-covalent interaction) to a cargo. The cargo can be a therapeutic moiety (e.g., an oligonucleotide, peptide or small molecule) that can be delivered into a cell by the EEV. The EEV-conjugate can be an EEV-conjugate of Formula (C).

As used herein, the term “exocyclic peptide” (EP) and “modulatory peptide” (MP) may be used interchangeably to refer to two or more amino acid residues linked by a peptide bond that can be conjugated to a cyclic cell penetrating peptide (cCPP) disclosed herein. The EP, when conjugated to a cyclic peptide disclosed herein, may alter the tissue distribution and/or retention of the compound. Typically, the EP comprises at least one positively charged amino acid residue, e.g., at least one lysine residue and/or at least one arginine residue. Non-limiting examples of EP are described herein. The EP can be a peptide that has been identified in the art as a “nuclear localization sequence” (NLS). Non-limiting examples of nuclear localization sequences include the nuclear localization sequence of the SV40 virus large T-antigen, the minimal functional unit of which is the seven amino acid sequence PKKKRKV (SEQ ID NO: 103), the nucleoplasmin bipartite NLS with the sequence NLSKRPAAIKKAGQAKKKK (SEQ ID NO: 253), the c-myc nuclear localization sequence having the amino acid sequence PAAKRVKLD (SEQ ID NO: 112) or RQRRNELKRSF (SEQ ID NO: 113), the sequence RMRKFKNKGKDTAELRRRRVEVSVELR (SEQ ID NO: 114), KAKKDEQILKRRNV (SEQ ID NO: 115) of the LBB domain from importin-alpha, the sequences VSRKRPRP (SEQ ID NO: 116) and PPKKARED (SEQ ID NO: 117) of the myoma T protein, the sequence PQPKKKPL (SEQ ID NO: 118) of human p53, the sequence SALIKKKKKMAP (SEQ ID NO: 119) of mouse c-abl IV, the sequences DRLRR (SEQ ID NO: 120) and PKQKKRK (SEQ ID NO: 121) of the influenza virus NS1, the sequence RKLKKKIKKL (SEQ ID NO: 122) of the Hepatitis virus delta antigen and the sequence REKKKFLKRR (SEQ ID NO: 123) of the mouse Mxl protein, the sequence KRKGDEVDGVDEVAKKKSKK (SEQ ID NO: 124) of the human poly(ADP-ribose) polymerase and the sequence RKCLQAGMNLEARKTKK (SEQ ID NO: 125) of the steroid hormone receptors (human) glucocorticoid. International Publication No. 2001/038547 describes additional examples of NLSs and is incorporated by reference herein in its entirety.

As used herein, “linker” or “L” refers to a moiety that covalently bonds one or more moieties (e.g., an exocyclic peptide (EP) and a cargo, e.g., an oligonucleotide, peptide or small molecule) to the cyclic cell penetrating peptide (cCPP). The linker can comprise a natural or non-natural amino acid or polypeptide. The linker can be a synthetic compound containing two or more appropriate functional groups suitable to bind the cCPP to a cargo moiety, to thereby form the compounds disclosed herein. The linker can comprise a polyethylene glycol (PEG) moiety. The linker can comprise one or more amino acids. The cCPP may be covalently bound to a cargo via a linker.

As used herein, the term “oligonucleotide” refers to an oligomeric compound comprising a plurality of linked nucleotides or nucleosides. One or more nucleotides of an oligonucleotide can be modified. An oligonucleotide can comprise ribonucleic acid (RNA) or deoxyribonucleic acid (DNA). Oligonucleotides can be composed of natural and/or modified nucleobases, sugars and covalent internucleoside linkages, and can further include non-nucleic acid conjugates.

The terms “peptide,” “protein,” and “polypeptide” are used interchangeably to refer to a natural or synthetic molecule comprising two or more amino acids linked by the carboxyl group of one amino acid to the alpha amino group of another. Two or more amino acid residues can be linked by the carboxyl group of one amino acid to the alpha amino group. Two or more amino acids of the polypeptide can be joined by a peptide bond. The polypeptide can include a peptide backbone modification in which two or more amino acids are covalently attached by a bond other than a peptide bond. The polypeptide can include one or more non-natural amino acids, amino acid analogs, or other synthetic molecules that are capable of integrating into a polypeptide. The term polypeptide includes naturally occurring and artificially occurring amino acids. The term polypeptide includes peptides, for example, that include from about 2 to about 100 amino acid residues as well as proteins, that include more than about 100 amino acid residues, or more than about 1000 amino acid residues, including, but not limited to therapeutic proteins such as antibodies, enzymes, receptors, soluble proteins and the like.

The term “therapeutic polypeptide” refers to a polypeptide that has therapeutic, prophylactic or other biological activity. The therapeutic polypeptide can be produced in any suitable manner. For example, the therapeutic polypeptide may isolated or purified from a naturally occurring environment, may be chemically synthesized, may be recombinantly produced, or a combination thereof.

The term “small molecule” refers to an organic compound with pharmacological activity and a molecular weight of less than about 2000 Daltons, or less than about 1000 Daltons, or less than about 500 Daltons. Small molecule therapeutics are typically manufactured by chemical synthesis.

As used herein, the term “contiguous” refers to two amino acids, which are connected by a covalent bond. For example, in the context of a representative cyclic cell penetrating peptide (cCPP) such as

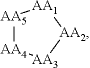

AA₁/AA₂, AA₂/AA₃, AA₃/AA₄, and AA₅/AA₁ exemplify pairs of contiguous amino acids.

A residue of a chemical species, as used herein, refers to a derivative of the chemical species that is present in a particular product. To form the product, at least one atom of the species is replaced by a bond to another moiety, such that the product contains a derivative, or residue, of the chemical species. For example, the cyclic cell penetrating peptides (cCPP) described herein have amino acids (e.g., arginine) incorporated therein through formation of one or more peptide bonds. The amino acids incorporated into the cCPP may be referred to residues, or simply as an amino acid. Thus, arginine or an arginine residue refers to

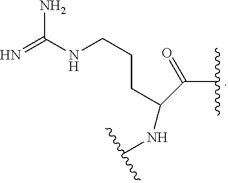 [SMILES: *NC(CCCNC(=N)N)C(*)=O]

The term “protonated form thereof” refers to a protonated form of an amino acid. For example, the guanidine group on the side chain of arginine may be protonated to form a guanidinium group. The structure of a protonated form of arginine is

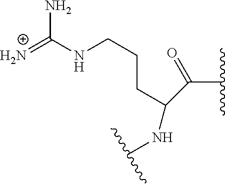 [SMILES: *NC(CCCNC(N)=[NH2+])C(*)=O]

As used herein, the term “chirality” refers to a molecule that has more than one stereoisomer that differs in the three-dimensional spatial arrangement of atoms, in which one stereoisomer is a non-superimposable mirror image of the other. Amino acids, except for glycine, have a chiral carbon atom adjacent to the carboxyl group. The term “enantiomer” refers to stereoisomers that are chiral. The chiral molecule can be an amino acid residue having a “D” and “L” enantiomer. Molecules without a chiral center, such as glycine, can be referred to as “achiral.”

As used herein, the term “hydrophobic” refers to a moiety that is not soluble in water or has minimal solubility in water. Generally, neutral moieties and/or non-polar moieties, or moieties that are predominately neutral and/or non-polar are hydrophobic. Hydrophobicity can be measured by one of the methods disclosed herein below.

As used herein “aromatic” refers to an unsaturated cyclic molecule having 4n+2 π electrons, wherein n is any integer. The term “non-aromatic” refers to any unsaturated cyclic molecule which does not fall within the definition of aromatic.

“Alkyl”, “alkyl chain” or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene”, “alkylene chain” or “alkylene group” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl”, “alkenyl chain” or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene”, “alkenylene chain” or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” or “alkoxy group” refers to the group —OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkylcarbamoyl” or “alkylcarbamoyl group” refers to the group —O—C(O)—NRₐR_{b}, where Rₐ and R_{b} are the same or different and are independently an alkyl, alkenyl, alkynyl, aryl, heteroaryl, as defined herein, or RₐR_{b} can be taken together to form a cycloalkyl group or heterocyclyl group, as defined herein. Unless stated otherwise specifically in the specification, an alkylcarbamoyl group can be optionally substituted.

“Alkylcarboxamidyl” or “alkylcarboxamidyl group” refers to the group —C(O)—NRₐR_{b}, where Rₐ and R_{b} are the same or different and are independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, an alkylcarboxamidyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heteroaryl” refers to a 5 to 20 membered ring system radical comprising hydrogen atoms, one to thirteen carbon atoms, one to six heteroatoms selected from nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH2SO₂R_{g}, —CH2SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

TABLE 10

| Abbreviation | IUPAC Name |
|---|---|
| HATU | 1-[Bis(dimethylamino)methylene]-1H-1,2,3-triazolo[4,5- |
| | b]pyridinium 3-oxide hexafluorophosphate |
| HOBt | 1-hydroxybenzotriazole |
| PyAOP | (7-Azabenzotriazol-1-yloxy)tripyrrolidinophosphonium |
| | hexafluorophosphate |
| PyOxim | [(E)-(1-cyano-2-ethoxy-2-oxoethylidene)amino]oxy- |
| | tripyrrolidin-1-ylphosphanium; hexafluorophosphate |
| Oxyma | Ethyl (2Z)-2-cyano-2-(hydroxyimino)acetate |
| HBTU | 3-[Bis(dimethylamino)methyliumyl]-3H-benzotriazol-1-oxide |
| | hexafluorophosphate |
| TBTU | 2-(1H-Benzotriazole-1-yl)-1,1,3,3-tetramethyluronium |
| | tetrafluoroborate |
| COMU | (1-Cyano-2-ethoxy-2-oxoethylidenaminooxy)dimethylamino- |
| | morpholino-carbenium hexafluorophosphate |
| DEPBT | Diethyl 4-oxo-1,2,3-benzotriazin-3(4H)-yl phosphate |

### Examples

### Example 1. Design and Synthesis of cCPPs Containing Arginine Derivatives and Conjugates Thereof

Materials and General Methods. Reagents for peptide synthesis and Rink amide resin (100-200 mesh, 0.54 mmol/g) were purchased from commercial suppliers. The purity of the peptides was assessed by analytical HPLC and the identity was confirmed by ESI mass spectrometric analyses.

cCPP design. Arginine has been implicated as a significant contributor to systemic organ toxicity for cell-penetrating peptides. Arginine residues, by virtue of the guanidinium functional group on the side-chain, are protonated and have a positive charge at physiological pH. This positive charge facilitates interaction with both the plasma and endolysosomal membranes which enables endocytosis and endosomal escape to deliver cargo modalities into the cytoplasm. Alternative residues that may be positively charged at physiological pH were incorporated into the cyclic scaffold and prepared as listed Table 11 and FIG. 1. The guanidinium functional group is also capable of forming bidentate hydrogen bonding interactions with phospholipids on the plasma membrane and it is understood that this is essential for effective membrane association and subsequent internalization. As an increasing number of positive charges leads to increasing systemic toxicity it was proposed that arginine replacements that are capable of forming bidentate hydrogen bonding interactions without a positive charge would retain activity while reducing toxicity as listed in Table 11 and FIG. 1.

TABLE 11
Cyclic Peptides Comprising Alternative Residues.

| Compound | Sequence |
|---|---|
| Compound A | cyclo(FfΦ-Agp-r-Agp-rQ)-PEG₄-K-NH₂ (control) (SEQ ID NO: 254) |
| Compound B | cyclo(FfΦ-Agb-r-Agb-rQ)-PEG₄-K-NH₂ (control) (SEQ ID NO: 254) |
| Compound C | cyclo(FfΦ-hR-r-hR-rQ)-PEG₄-K-NH₂ (control) (SEQ ID NO: 255) |
| Compound D | cyclo(FfΦ-4gp-r-4gp-rQ)-PEG₄-K-NH₂ (SEQ ID NO: 242) |
| Compound 1a | cyclo(FfΦ-Cit-r-Cit-rQ)-PEG₄-K-NH₂ (SEQ ID NO: 236) |
| Compound 2a | cyclo(FfΦ-Pia-r-Pia-rQ)-PEG₄-K-NH₂ (SEQ ID NO: 243) |
| Compound 3a | cyclo(FfΦ-Dml-r-Dml -rQ)-PEG₄-K-NH₂ (SEQ ID NO: 244) |
| Compound 1b | Ac-PKKKRKV-PEG₂-K(cyclo[FfΦ-Cit-r-Cit-r-Q]-PEG₁₂-K(N₃)-NH₂ |
| | (SEQ ID NOs: 246 and 236) |
| Compound 1c | cyclo(FfΦ-Cit-r-Cit-rQ)-PEG₁₂-OH (SEQ ID NO: 236) |
| Compound 7c | cyclo(fΦR-Cit-R-Cit-Q)-PEG₁₂-OH (SEQ ID NO: 249) |
| Compound 8d | Ac-PKKKRKV-K(cyclo[FfΦ-R-r-Cit-rQ])-PEG₁₂-K(N₃)-NH₂ (SEQ ID |
| | NOs: 246 and 247) |
| Compound 9d | Ac-PKKKRKV-K(cyclo[FfΦR-cit-R-cit-Q])-PEG₁₂-K(N₃)-NH₂ (SEQ ID |
| | NOs: 246 and 249) |
| Compound 10d | Ac-PKKKRKV-K(cyclo[FfΦ-Cit-r-R-rQ])-PEG₁₂-K(N₃)-NH₂ (SEQ ID |
| | NOs: 246 and 236) |
| Compound 1e | Ac-PKKKRKV-PEG₂-K(cyclo[FfΦ-Cit-r-Cit-rQ])-B-k(N₃)-NH₂ (SEQ ID |
| | NOs: 246 and 236) |
| Compound 1f | Ac-PKKKRKV-PEG₂-K(cyclo[FfΦ-Cit-r-Cit-rQ])-PEG₂-k(N₃)-NH₂ (SEQ |
| | ID NOs: 246 and 236) |
| Compound 1g | Ac-PKKKRKV-PEG₂-K(cyclo[FfΦ-Cit-r-Cit-rQ])-PEG₄-k(N₃)-NH₂ (SEQ |
| | ID NOs: 246 and 236) |
| Compound 1h | Ac-PKKKRKV-K(cyclo[FfΦ-Cit-r-Cit-rQ])-PEG₁₂-k(N₃)-NH₂ (SEQ ID |
| | NOs: 246 and 236) |
| Compound 1i | Ac-pkkkrkv-PEG₂-K(cyclo[FfΦ-Cit-r-Cit-rQ])-PEG₁₂-k(N₃)-NH₂ (SEQ |
| | ID NOs: 246 and 236) |
| Compound 1j | Ac-rrv-PEG₂-K(cyclo[FfΦ-Cit-r-Cit-rQ])-PEG₁₂-OH (SEQ ID NO: 236) |
| Compound 1k | Ac-PKKKRKV-PEG₂-K(cyclo[FfΦ-Cit-r-Cit-r-Q])-PEG₁₂-k(N₃)-NH₂ |
| | (SEQ ID NOs: 246 and 236) |
| Compound 1l | Ac-PKKK-Cit-KV-PEG₂-K(cyclo[FfΦ-Cit-r-Cit-r-Q])-PEG₁₂-k(N₃)-NH₂ |
| | SEQ ID NOs: 256 and 236) |
| Compound 1m | Ac-pkkkrkv-PEG₂-k(cyclo[FfΦ-Cit-r-Cit-rQ])-PEG₁₂-k(N₃)-NH₂ (SEQ ID |
| | NOs: 246 and 236) |

Φ = 3-(2-naphthyl)-L-alanine, Agp = L-2-amino-3-guanidinopropionic acid, Agb = L-2-4-guanidinobutanoic acid, hR = L-homoarginine, 4gp = 4-guanidino-L-phenylalanine, Cit = citrulline, Pia = 3-(4-piperidinyl)-L-alanine, Dml = N-dimethyl-L-lysine, B = beta-alanine. Lower-case letters denote D-amino acids.

Oligonucleotide design. An antisense compound (AC) was designed to skip exon 23 in the mouse dystrophin pre-mRNA, resulting in the formation of an internally-truncated form of dystrophin to assess the feasibility for utilizing compositions to address the disease state in the mdx mouse model of Duchenne Muscular Dystrophy (DMD)). The AC is a phosphorodiamidate morpholino oligomer (PMO) composed exclusively of phosphorodiamidate morpholino bases of which one sequence conjugated was 5′-GGCCAAACCTCGGCTTACCTGAAAT-3′ (SEQ ID NO: 139).

Cell penetrating peptide. A cell-penetrating peptide comprising the sequence Acetyl-Pro-Lys-Lys-Lys-Arg-Lys-Val-PEG₂-K(cyclo[Phe-D-Phe-2-Nal-Cit-D-Arg-Cit-D-Arg-γ-Glu)-PEG₁₂-K(N₃)—NH₂ (“Compound 1b”; (SEQ ID NOs: 246 and 236)) was formulated as a TFA salt.

Synthesis. The peptide was synthesized using standard Fmoc chemistry.
1. Add DMF to the vessel containing Rink amide MBHA Resin (0.3 mmol, 0.87 g, sub: 0.35 mmol/g) and swell for 2 hours.
2. Drain and then DMF wash 30 sec with 3 times.
3. Add 20% piperidine/DMF and mix for 30 min.
4. Drain and then DMF wash 30 sec with 5 times.
5. Add Fmoc-amino acid solution and mix for 30 seconds, then add coupling regents while N₂ bubbling for 30 min.
6. Repeat step 2 to 5 for the next amino acid coupling.
7. After coupling, the resin was washed with MeOH for 3 times and dried under reduced pressure.

TABLE 12

| | | |
|---|---|---|
| 1 | Fmoc-K(N₃)-OH (2.0 eg) | HATU (1.85 eq)/HOAt (2 eq)/DIEA (4.0 eq) |
| 2 | Fmoc-PEG₁₂-OH (2.0 eg) | HATU (1.85 eq)/HOAt (2 eq)/DIEA (4.0 eq) |
| 3 | Fmoc-K(Dde)-OH (2.0 eq) | HATU (1.85 eq)/HOAt (2 eq)/DIEA (4.0 eq) |
| 4 | Fmoc-miniPEG2-OH (2.0 eq) | HATU (1.85 eq)/HOAt (2 eq)/DIEA (4,0 eq) |
| 5 | Fmoc-Val-OH (3.0 eq) | HBTU (2.85 eq)/HOBt (3 eq)/DIEA (6.0 eq) |
| 6 | Fmoc-K(Boc)-OH (3.0 eq) | HBTU (2.85 eq)/HOBt (3 eq)/DIEA (6.0 eq) |
| 7 | Fmoc -Arg(Pbf)-OH (3.0 eq) | HBTU (2.85 eq)/HOBt (3 eq)/DIEA (6.0 eq) |
| 8 | Fmoc-K(Boc)-OH (3.0 eq) | HBTU (2.85 eq)/HOBt (3 eq)/DIEA (6.0 eq) |
| 9 | Fmoc-K(Boc)-OH (3.0 eq) | HBTU (2.85 eq)/HOBt (3 eq)/DIEA (6.0 eq) |
| 10 | Fmoc-K(Boc)-OH (3.0 eq) | HBTU (2.85 eq)/HOBt (3 eq)/DIEA (6.0 eq) |
| 11 | Fmoc-Pro-OH (3.0 eq) | HBTU (2.85 eq)/HOBt (3 eq)/DIEA (6.0 eq) |
| 12 | Ac₂O (6.0 eq) | DIEA (12.0 eq) |
| 13 | Fmoc-Glu-OAllyl (2.0 eg) | HATU (1.85 eq)/HOAt (2 eq)/DIEA (4.0 eq) |
| 14 | D-Fmoc-Arg(Pbf)-OH (3.0 eq) | HBTU (2.85 eq)/HOBt (3 eq)/DIEA (6.0 eq) |
| 15 | Fmoc-Cit-OH (2.0 eq) | HATU (1.85 eq)/HOAt (2 eq)/DIEA (4.0 eq) |
| 16 | D-Fmoc-Arg(Pbf)-OH (3.0 eq) | HBTU (2.85 eq)/HOBt (3 eq)/DIEA (6.0 eq) |
| 17 | Fmoc-Cit-OH (2.0 eq) | HATU (1.85 eq)/HOAt (2 eq)/DIEA (4.0 eq) |
| 18 | Fmoc-3-(2-Nal)-Ala-OH (2.0 eq) | HATU (1.85 eq)/HOAt (2 eq)/DIEA (4.0 eq) |
| 19 | D-Fmoc-Phe-OH (3.0 eg) | HBTU (2.85 eq)/HOBt (3 eq)/DIEA (6.0 eq) |
| 20 | Fmoc-Phe-OH (3.0 eq) | HBTU (2.85 eq)/HOBt (3 eq)/DIEA (6.0 eq) |
| 21 | Cyclization | PyAOP (1.85 eq)/HOAt (2 eq)/DIEA (4.0 eq) |

20% piperidine in DMF was used for Fmoc deprotection for 30 min. Dde was removed by 3% NH₂NH₂/DMF for 30 min twice. Allyl was removed by Pd(PPh₃)₄ and PhSiH₃. The coupling reaction was monitored by ninhydrin test, and the resin was washed with DMF for 5 times.

### Peptide Cleavage and Purification:

1. Add cleavage buffer (95% TFA/2.5% TIS/2.5% H₂O) to the flask containing the side chain protected peptide at room temperature and stir for 2.0 hours.
2. The peptide is precipitated with cold isopropyl ether and centrifuged (3 min at 3000 rpm).
3. Isopropyl ether washes two additional times.
4. Dry the crude peptide under vacuum 2 hours.
5. Purify the crude peptide by prep-HPLC (A: 0.075% TFA in H₂O, B: ACN) to give the final product (145.6 mg, 97.4% purity, 15.1% yield). Purity and identity was confirmed by analytical UPLC/MS.

Preparation of cCPP-PMO conjugates. Peptide-PMO was prepared as the 3′ conjugate via strain-promoted alkyne-azide cycloaddition. In brief, a solution of peptide-azide in nuclease-free water (1 mM) was added to the PMO-3′-cyclooctyne or cyclooctyne-5′-PMO solids. The mixture was vortexed to dissolve the peptide-PMO conjugate, centrifuged to settle the solution, and incubated at room temperature for 8-12 hours for completion as confirmed by LCMS (Q-TOF). For purifications, crude mixtures were diluted with DMSO, loaded onto a C18 reverse-phase column (150 mm*21.2 mm), flow rate of 20 mL/min and purified by an appropriate gradient using water with 0.05% TFA and acetonitrile as solvents. Desired fractions were pooled, pH of the solution was adjusted to 5-6 with 1N NaOH and the solution was lyophilized to afford a white powder. For in vitro and in vivo formulations, the conjugates were reconstituted in appropriate amount of sterile PBS or sterile saline to the desired concentration (2-10 mg/mL). All material was stored at −80° C. until use.

### Example 2. Determination of Cell Penetrating Activity Using the Chloroalkane Penetration Assay

Assay design. HEK293 cells were produced that stably express a HaloTag-ActA fusion protein (“HEK293-HaloTag”) which ensures fusion protein localization to the outer mitochondrial membrane with exposure to the cytosol. The HaloTag protein rapidly reacts covalently with short chloroalkane-containing compounds, occupying the active site and preventing further reactions. The chloroalkane penetration assay utilizes this reactivity in a pulse-chase assay where cells are first treated with cCPPs-chloroalkane of interest, followed by incubation with cell-permeable fluorescent dye tetramethylrhodamine-chloroalkane (“TMR-ct”). If cCPPs have gained access to the cytosol, they will react with HaloTag and prevent it from reacting with TMR-ct. The relative cell-penetrating efficiency of compounds can therefore be determined by their ability to reduce cellular TMR fluorescence after a washout period with this value expressed as IC₅₀.

Cell penetrating peptides. Cell penetrating peptides having the sequences indicated in Table 13 (below) were functionalized on the lysine residue with a chloroalkane tag of the sequence N¹-(2-(2-((5-chlorohexyl)oxy)ethoxy)ethyl-N⁴-(2-(2-(2-oxoethoxy)ethoxy)ethyl)succinamide (referred to as “chloroalkane” in Table 13), purified, and prepared as stock solutions in DMSO with concentration determined by A₂₈₀ or weight as applicable.

Determination of cell-penetrating efficiency. Compounds to be evaluated were prepared as stock solutions in DMSO and diluted in PBS before addition to HEK293-HaloTag ells as a serial dilution from 30 μM to 0.5 nM. Cells were incubated with the compounds at a given concentration for 24 h in the presence of FBS at 37° C. After incubation, cells were washed thoroughly with PBS and to the cells was added fresh, serum-free media containing 5 μM TMR-ct and incubated for 30 min. After incubation, the cells were washed and incubated in fresh media to wash-out any unreacted TMR-ct. Cells were then imaged and quantified for cellular fluorescence using high-content imaging. Values are normalized to vehicle-treated cells and an IC₅₀ calculated using a 4-parameter logarithmic fit in GraphPad PRISM v. 8.

Results. Data from the HaloTag experiments support that multiple arginine derivatives (see FIG. 1), including neutral residues such as citrulline, are equivalent or superior to arginine for enabling cell-penetration and cytosolic delivery of cCPPs in mammalian cells (Table 13).

TABLE 13
Cell-penetrating efficiency of cyclic peptides comprising arginine replacements.

| ID | Sequence | Halo Tag IC₅₀ (μM) |
|---|---|---|
| EEV12 | cyclo(FfΦRrRrQ)-PEG₄-K(chloroalkane)-NH₂ (SEQ ID | 0.792 |
| | NO: 151) | |
| Compound A | cyclo(FfΦ-Agp-r-Agp-rQ)-PEG₄-K(chloroalkane)-NH₂ | 0.626 |
| | (SEQ ID NO: 254) | |
| Compound B | cyclo(Ffd-Agb-r-Agb-rQ)-PEG₄-K(chloroalkane)-NH₂ | 0.695 |
| | (SEQ ID NO: 254) | |
| Compound C | cyclo(FfΦ-hR-r-hR-rQ)-PEG₄-K(chloroalkane)-NH₂ | 0.786 |
| | SEQ ID NO: 255) | |
| Compound D | cyclo(FfΦ-4gp-r-4gp-rQ)-PEG₄-K(chloroalkane)-NH₂ | 0.695 |
| | (SEQ ID NO: 242) | |
| Compound 1a | cyclo(FfΦ-Cit-r-Cit-rQ)-PEG₄-K(chloroalkane)-NH₂ | 0.849 |
| | (SEQ ID NO: 236) | |
| Compound 2a | cyclo(FfΦ-Pia-r-Pia-rQ)-PEG₄-K(chloroalkane)- | 0.881 |
| | NH₂(SEQ ID NO: 243) | |
| Compound 3a | cyclo(FfΦ-Dml-r-Dml-rQ)-PEG₄-K(chloroalkane)-NH₂ | 0.932 |
| | (SEQ ID NO: 244) | |

### Example 3. In Vitro Cytotoxicity and Membranolytic Activity of Arginine-Derivative cCPPs

Cell lines. Human fibroblasts (“WI38”), human primary renal proximal tubular epithelial cells (“RPTEC”), human umbilical vein endothelial cells (“HUVEC”), and a mixture of human peripheral blood mononuclear cells (“PBMC”) were utilized.

Cell viability. Compounds were synthesized as previously described and prepared as stock solutions in DMSO. Compounds were serially diluted in sterile saline to the desired concentration and added to plated WI38, RPTEC, HUVEC, or PBMCs in complete growth media containing 10% FBS and incubated for 24 h at 37° C. After 24 h, cell viability was assessed using CellTiter-Glo 2.0 (WI38) or CyQuant Green (RPTEC, HUVEC, PBMC) following the manufacturer's protocol. Values given for viability are given relative to vehicle-treated controls as 100/6 viable.

LDH release. The ability for cCPPs to disrupt the plasma membrane and cause LDH release was assessed. WI38, RPTEC, and HUVEC cells were maintained in complete growth media supplemented with 10% FBS and were treated with compounds serially diluted from DMSO stock solutions in PBS at the indicated concentrations for 1 h at 37° C., 5% CO₂. After 1 h, 50 μL cell culture media from each well was transferred into a clear 96-well plate and combined with 50 μL of the LDH reaction mixture and incubated for 30 min. at room temperature. After 30 min. the reaction was quenched with 50 μL of stop solution and absorbance was measured at 490 nm and background corrected using the absorbance at 680 nm. Values given are relative to cells lysed with 1% Triton-X100 representing 100% LDH activity.

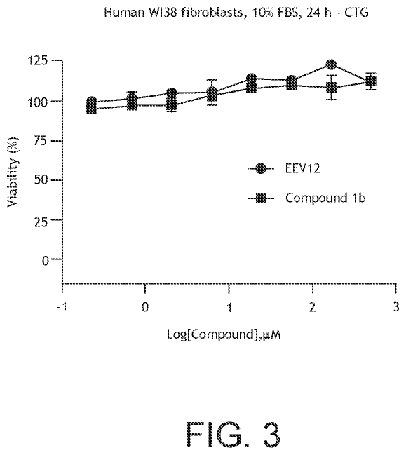

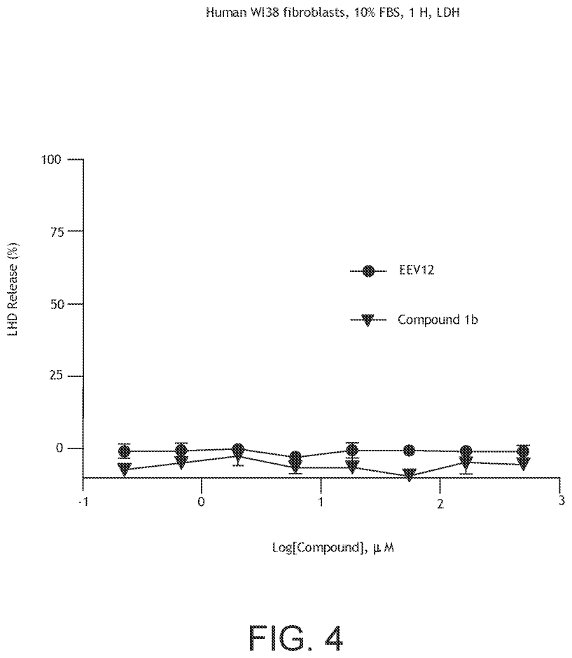

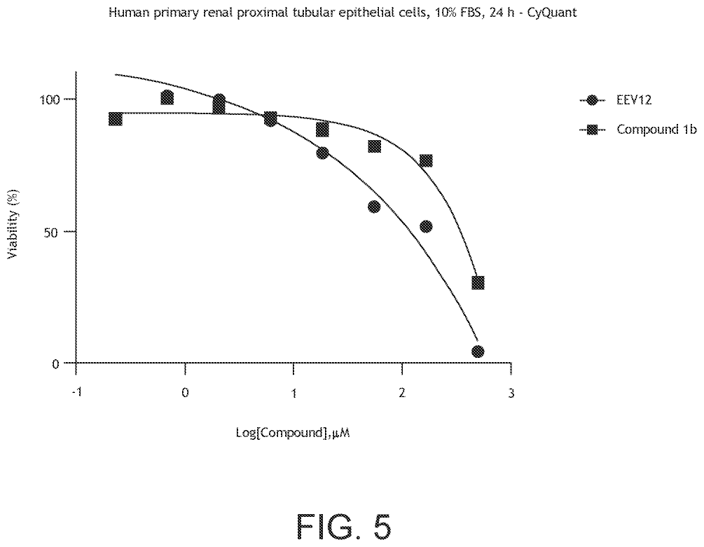

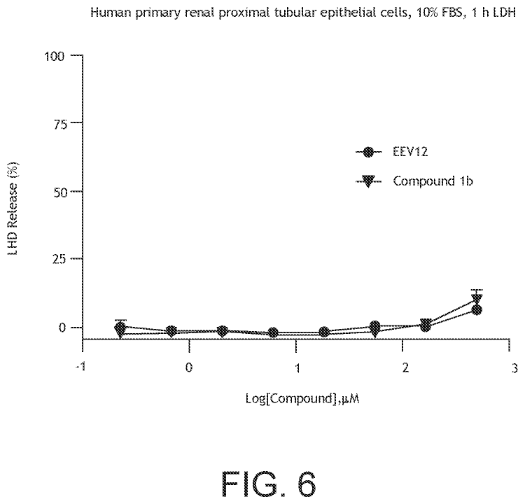

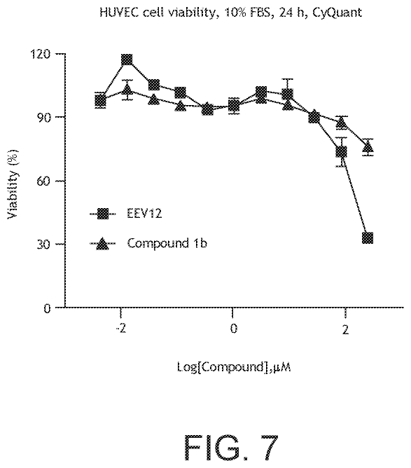

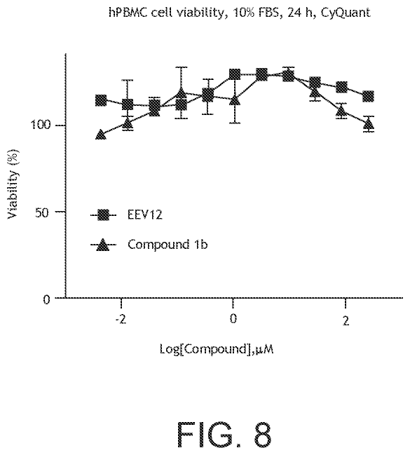

Results. Treatment with Compound 1b (Ac-PKKKRKV-PEG₂-K(cyclo[FfΦ-Cit-r-Cit-r-Q]-PEG₁₂-K(N₃)—NH₂; (SEQ ID NOs: 246 and 236)) resulted in non-significant losses in cell viability in WI38, HUVEC and hPBMCs, including no detectable LDH release indicating no measurable membrane damage upon compound treatment. Replacement of arginine residues with arginine analog citrulline reduced toxicity to RPTECs (Compound 1b vs. EEV12) even in the context of a molecule that bears more overall positive charge owing to the exocyclic residues. The results are shown in FIG. 3-8.

### Example 4. In Vivo Tolerability and Efficacy of cCPPs Containing Arginine Derivatives

Mice. Male C₅₇BL/6 mice were used for tolerability experiments. Efficacy studies used C₅₇BL/10ScSn-Dmdmdx/J (MDX) mice, which contain a C to T mutation resulting in a termination codon at position 2983 within exon 23 of the dystrophin gene (Dmd) on the X chromosome. Mice expressing this mutant allele produce a minimal dystrophin mRNA product and dystrophin protein and are thus a model of Duchenne's muscular dystrophy (“DMD”).

Study Design. cCPPs and cCPP-AC conjugates were prepared and characterized as described in Example 2 using sequences listed in Table 13 and with the structure indicated in FIG. 3. For tolerability studies in C₅₇BL/6 mice, the cCPP was used without conjugation. For efficacy studies in mdx mice, the cCPP-AC conjugate included a sequence from Table 13 and the AC which had the sequence 5′-GGCCAAACCTCGGCTTACCTGAAAT-3′ (SEQ ID NO: 139). The resulting conjugates comprised EEV12 and the AC (“EEV-MDX-PMO-1”) or Compound 1b (Ac-PKKKRKV-PEG₂-K(cyclo[FfΦ-Cit-r-Cit-r-Q]-PEG₁₂-K(N₃)—NH₂ (SEQ ID NO: 246 and 236) and AC (“EEV1-PMO-MDX-2”). For tolerability studies, compounds were formulated in sterile saline, pH 7.2 and administered to C57BL/6 mice via IV injection at doses of 5 mg/kg, 10 mg/kg, 20 mg/kg, and 40 mg/kg based on the body weight of the animal. Serum was collected 15 minutes post-injection and snap-frozen in liquid nitrogen and stored at −80° C. for further analysis. For efficacy studies, conjugates were formulated in sterile saline, pH 7.2, and administered to md& mice via IV injection at doses of 15 mg/kg, 30 mg/kg and 40 mg/kg based on the body weight of the animal. 7 days post-injection, animals were sacrificed and the indicated tissues were harvested, snap-frozen in liquid nitrogen and stored at −80° C. for future processing.

Quantification of histamine levels. Increases in serum histamine levels after compound administration can indicate systemic allergic response or which can preclude successful compound development as they manifest as deleterious clinical observations. Histamine in serum samples obtained 15 min. after IV compound administration in C57BL/6 mice were derivatized using phenylisothiocyanate (PITC) in a 0.1:1:10 mixture of PITC:ethanol:pyridine for 10 min. at room temperature to generate phenylthiocarbamyl (PTC) histamine. Samples were dried and reconstituted in 0.1% formic acid in acetonitrile before chromatographic separation and detection using ESI-MS using MRM transitions of 247.1-154.1 m/z. Quantification was performed using internal PTC-histamine controls and values were expressed as ng/mL serum histamine.

Detection of splicing correction by RT-PCR. The delivery of PMO can alter the splicing and result in a truncated dystrophin mRNA after exon 23 skipping. The detection of splicing correction process is measured by RT-PCR where extracted RNAs from tissues are first reverse-transcribed into cDNA and are further analyzed by nested PCR using two primer sets; forward primer 5′-CAGAATTCTGCCAATTGCTGAG-3′ (SEQ ID NO: 257) and reverse primer 5′-TTCTTCAGCTTGTGTCATCC-3′ (SEQ ID NO: 258) for the first round PCR (outer primer set) and forward primer 5′-CCCAGTCTACCACCCTATCAGAGC-3′ (SEQ ID NO: 259) and reverse primer 5′-CCTGCCTTTAAGGCTTCCTT-3′ (SEQ ID NO: 260) (inner primer set) for the second round PCR. The RT-PCR readout of tissues without splicing correction result in a 901 bp gene fragment and a new 689 bp gene fragment show up after splicing correction. The degree (percentage) of splicing correction detected by RT-PCR was calculated using the following equation: % correction=(intensity of 689 bp fragment band)/(intensity of 901 bp fragment band +intensity of 689 bp fragment band).

Detection of dystrophin expression by Western Blot. Lysis buffer (9% SDS, 4% glycerol, 5 mM Tris, and 5% beta-mercaptoethanol, along with HALT protease inhibitors) was added to minced mouse tissue from either mouse heart, transverse abdominis, quadriceps, or diaphragm. Metal beads were used in conjunction with a Qiagen Tissuelyser to mechanically homogenize the tissue. Lysate was cleared by centrifugation, and the supernatant was subjected either to SDS-PAGE using 3-8% Tris Acetate gels followed by transfer to nitrocellulose membranes and western blotting followed by fluorescent imaging using the LICOR system or to the Jess Simple Western system using the 66-440 kDa capillary matrices. Dystrophin was detected using the anti-dystrophin antibodies from Abcam (Ab52777 or Ab154168); alpha-actinin was detected using anti-alpha-actinin antibodies from R&D Systems (MAB8279) or Abcam (ab68167). Traditional western blot bands were quantified using LICOR software. Jess Simple Western peaks were fit and the area under the peaks was calculated using the Simple Western software. Each run included a standard curve using wildtype mouse lysate diluted with different amounts of mdx mouse lysate from the respective tissue. The dystrophin detected in each sample was normalized to alpha-actinin as a loading control, and a linear regression was performed for the standard curve, which was used to determine the amount of dystrophin in each sample as a percentage of wildtype dystrophin levels.

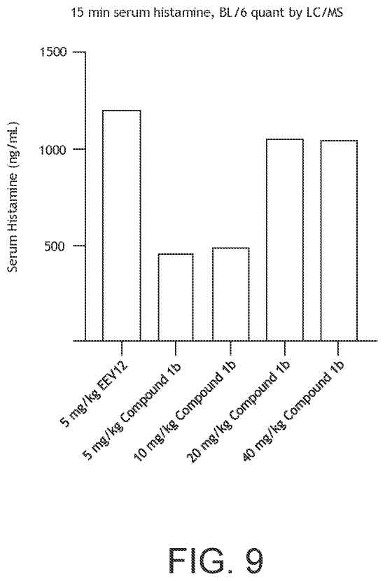

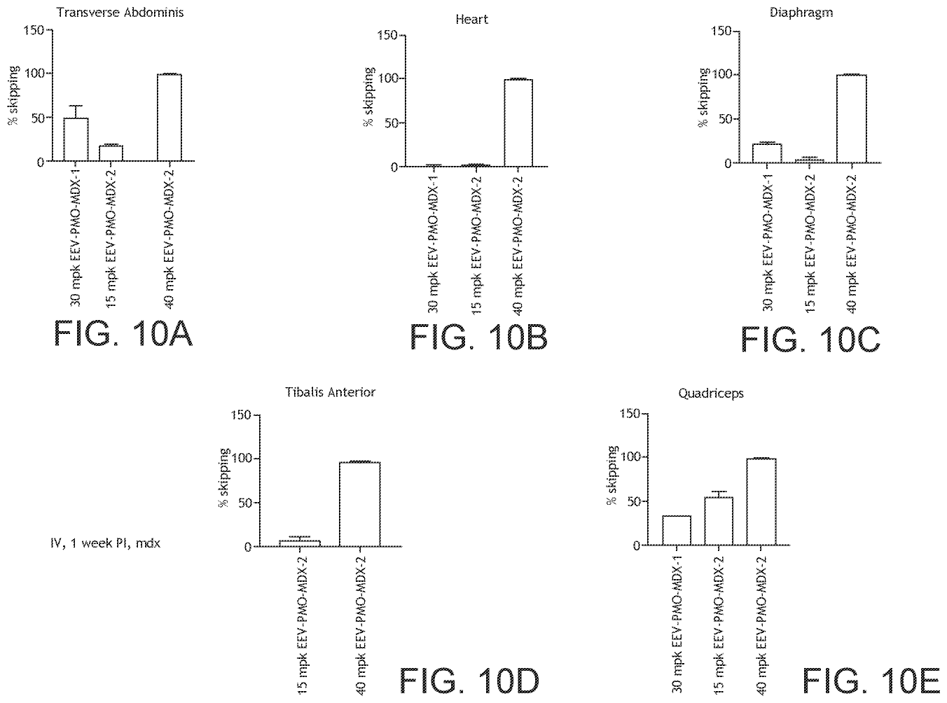

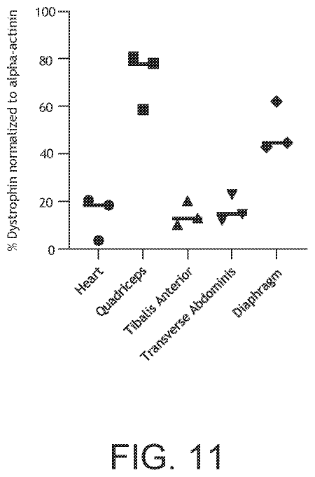

Results. Replacement of arginine residues with arginine analogs was capable of significantly reducing serum histamine levels after IV administration as demonstrated in FIG. 9. Histamine release after treatment with 5 mg/kg Compound 1b was ˜3-fold lower than 5 mg/kg EEV12, in spite of Compound 1b possessing 7 total positive charges, but 2 fewer arginine residues within the cCPP motif A dose of 20 mg/kg Compound 1b was necessary to see comparable histamine release to 5 mg/kg EEV12. Compound 1b possesses significantly enhanced tolerability owing to the replacement of arginine residues with non-positively charged citrulline residues. This tolerability enables higher dosing without adverse reactions. Incorporation of arginine residues also retained or enhanced in vivo efficacy as determined by RT-PCR in mdx mice. Treatment with 40 mg/kg EEV-MDX-PMO-2 (based on PMO concentration) resulted in significantly enhanced exon 23 skipping relative to 30 mg/kg EEV-MDX-PMO-1 in all tissues evaluated, including transverse abdominis, heart, diaphragm, tibalis anterior, and quadriceps as depicted in FIG. 10A-E. Exon skipping efficiency in mdx mice further translated to robust dystrophin product across transverse abdominis, heart, diaphragm, tibalis anterior, and quadriceps as determined by Western Blot and as depicted in FIG. 11. These findings demonstrate that arginine replacement with alternative residues that retain the unique hydrogen bonding capabilities of arginine, but without the positive charge, are able to render cCPPs cell-permeable and capable of successfully delivering cargo modalities to the cytosol and nucleus in vivo.

### Example 5: In Vivo Efficacy of cCPPs Containing Glycine

As above, efficacy studies used C57BL/10ScSn-Dmdmdx/J (MDX) mice, which contain a C to T mutation resulting in a termination codon at position 2983 within exon 23 of the dystrophin gene (Dmd) on the X chromosome. Mice expressing this mutant allele produce a minimal dystrophin mRNA product and dystrophin protein and are thus a model of Duchenne's muscular dystrophy (“DMD”).

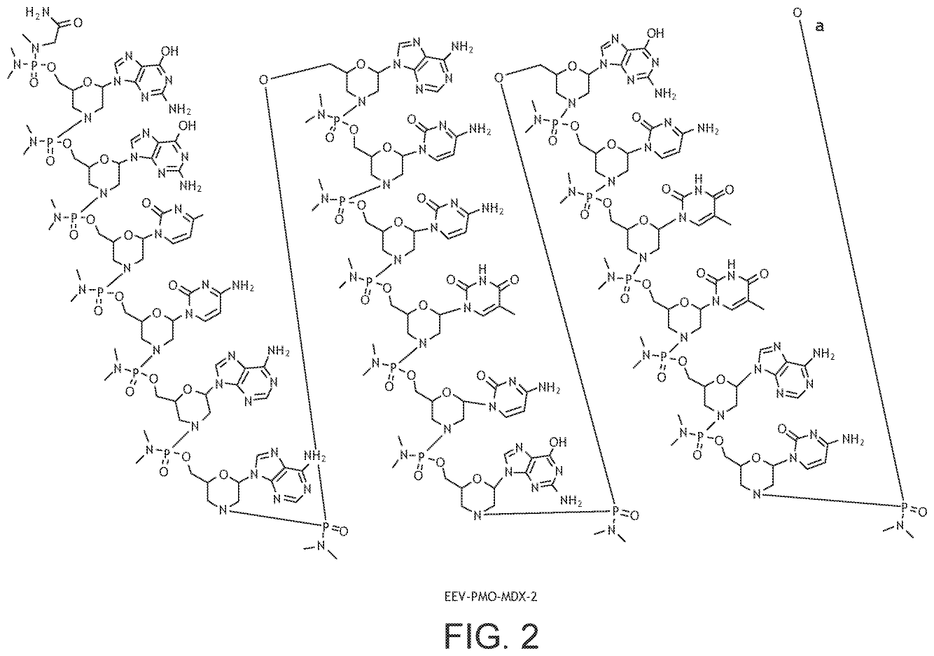

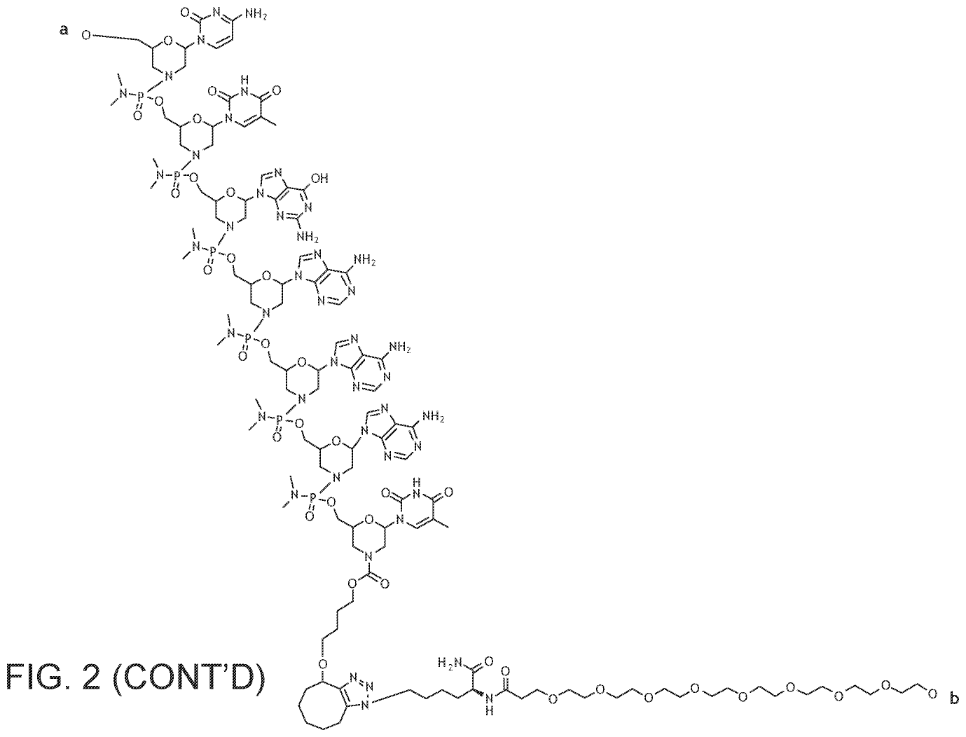

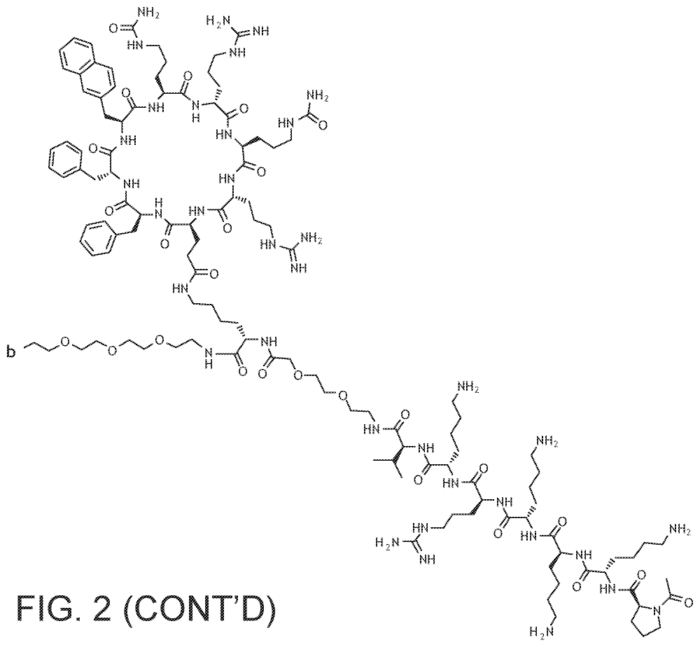

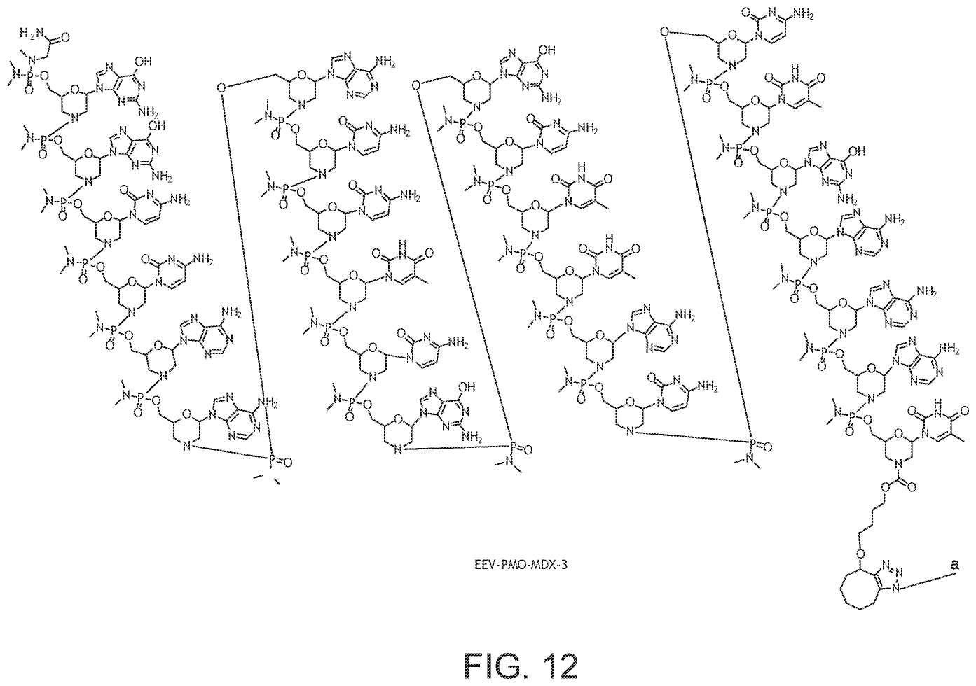

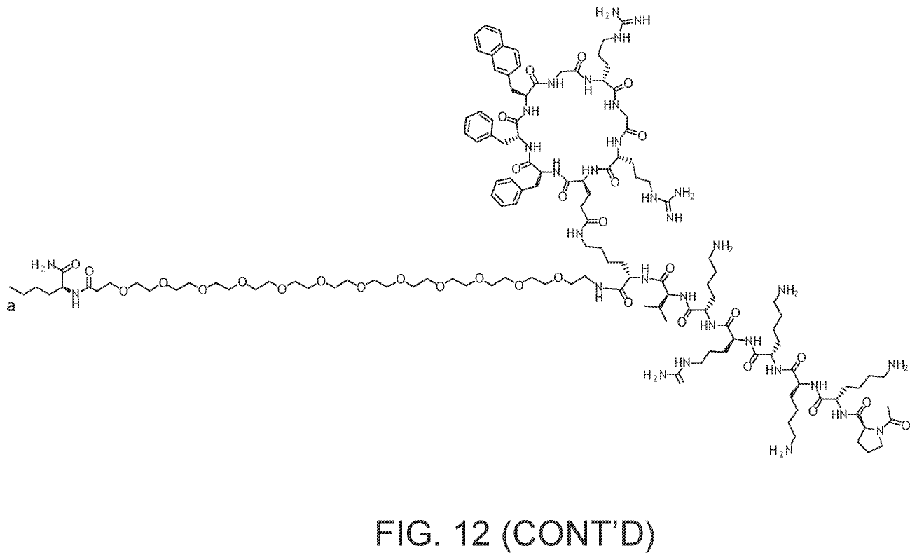

Study Design. For efficacy studies in mdc mice, cCPPs and cCPP-AC conjugates were prepared and characterized as described in Example 2 with the structures indicated in FIG. 2 and FIG. 12. The AC had the sequence 5′-GGCCAAACCTCGGCTTACCTGAAAT-3′ (SEQ ID NO: 139). Compound 4b had the sequence Ac-PKKKRKV-K(FfΦ-G-r-G-rQ)-PEG₁₂-K(N₃)—NH₂(SEQ ID NOs: 246 and 234). The resulting conjugate was EEV-MDX-PMO-3.

The conjugates were formulated in sterile saline, pH 7.2, and administered to md& mice via IV injection at a dose of 40 mg/kg based on the body weight of the animal. 3 days post-injection, animals were sacrificed and the indicated tissues were harvested, snap-frozen in liquid nitrogen and stored at −80° C. for future processing.

Detection of splicing correction by RT-PCR. The delivery of PMO can alter the splicing and result in a truncated dystrophin mRNA after exon 23 skipping. The detection of splicing correction process is measured by RT-PCR where extracted RNAs from tissues are first reverse-transcribed into cDNA and are further analyzed by nested PCR using two primer sets: forward primer 5′-CAGAATTCTGCCAATTGCTGAG-3′ (SEQ ID NO: 257) and reverse primer 5′-TTCTTCAGCTTGTGTCATCC-3′ (SEQ ID NO: 258) for the first round PCR (outer primer set) and forward primer 5′-CCCAGTCTACCACCCTATCAGAGC-3′ (SEQ ID NO: 259) and reverse primer 5′-CCTGCCTTTAAGGCTTCCTT-3′ (SEQ ID NO: 260 (inner primer set) for the second round PCR. The RT-PCR readout of tissues without splicing correction result in a 901 bp gene fragment and a new 689 bp gene fragment show up after splicing correction. The degree (percentage) of splicing correction detected by RT-PCR was calculated using the following equation: % correction=(intensity of 689 bp fragment band)/(intensity of 901 bp fragment band +intensity of 689 bp fragment band).

Detection of dystrophin expression by Western Blot. Lysis buffer (9% SDS, 4% glycerol, 5 mM Tris, and 5% beta-mercaptoethanol, along with HALT protease inhibitors) was added to minced mouse tissue from either mouse heart, transverse abdominis, quadriceps, or diaphragm. Metal beads were used in conjunction with a Qiagen Tissuelyser to mechanically homogenize the tissue. Lysate was cleared by centrifugation, and the supernatant was subjected either to SDS-PAGE using 3-8% Tris Acetate gels followed by transfer to nitrocellulose membranes and western blotting followed by fluorescent imaging using the LICOR system or to the Jess Simple Western system using the 66-440 kDa capillary matrices. Dystrophin was detected using the anti-dystrophin antibodies from Abcam (Ab52777 or Ab154168), HSP90 was detected using a HSP90 antibody from Cell Signaling Technology (4877). Traditional western blot bands were quantified using LICOR software. Jess Simple Western peaks were fit and the area under the peaks was calculated using the Simple Western software. Each run included a standard curve using wildtype mouse lysate diluted with different amounts of mdx mouse lysate from the respective tissue. The dystrophin detected in each sample was normalized to HSP90 as a loading control.

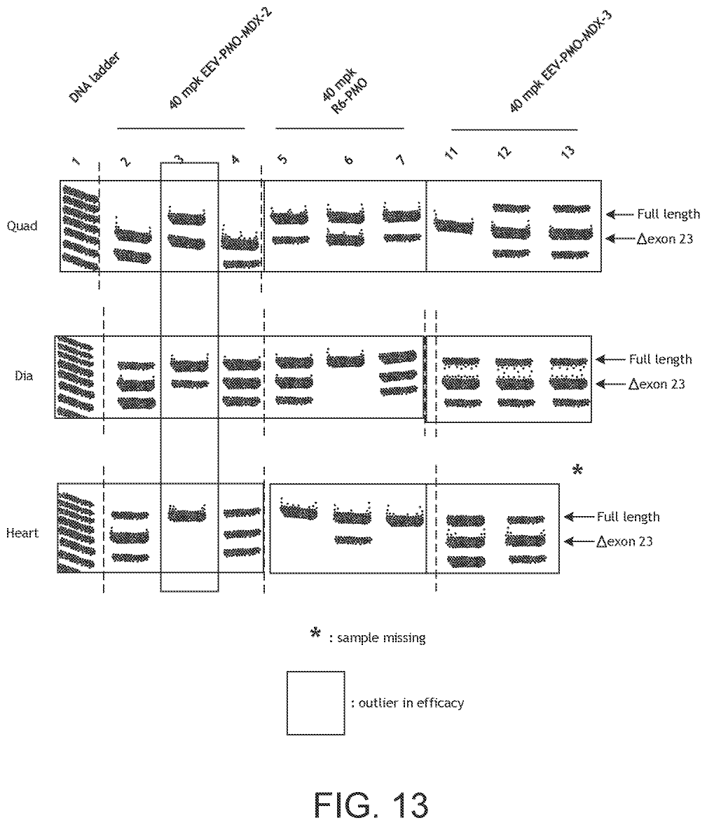

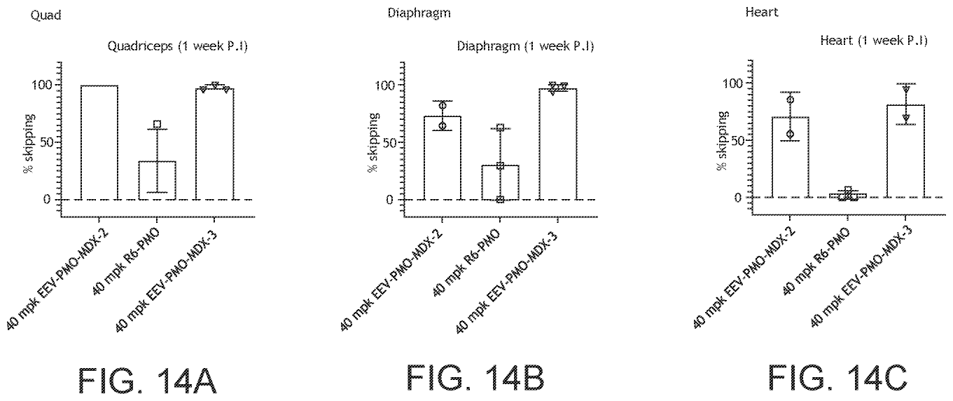

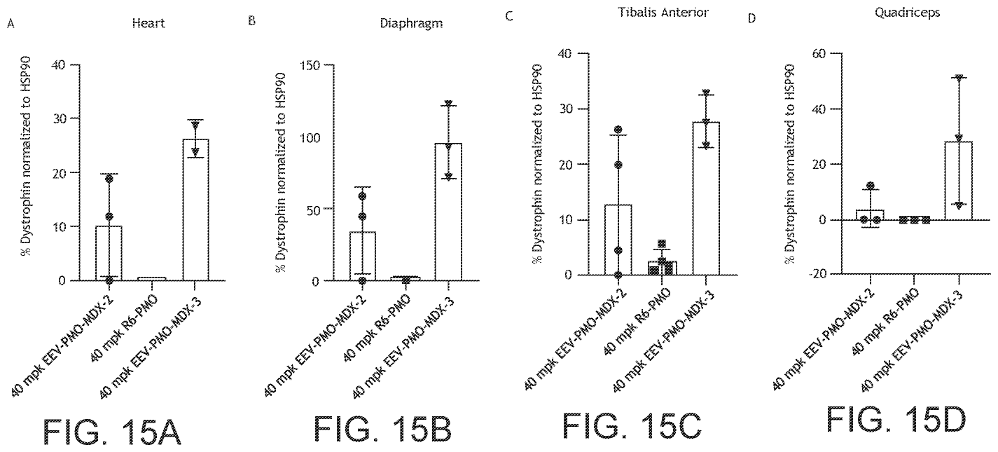

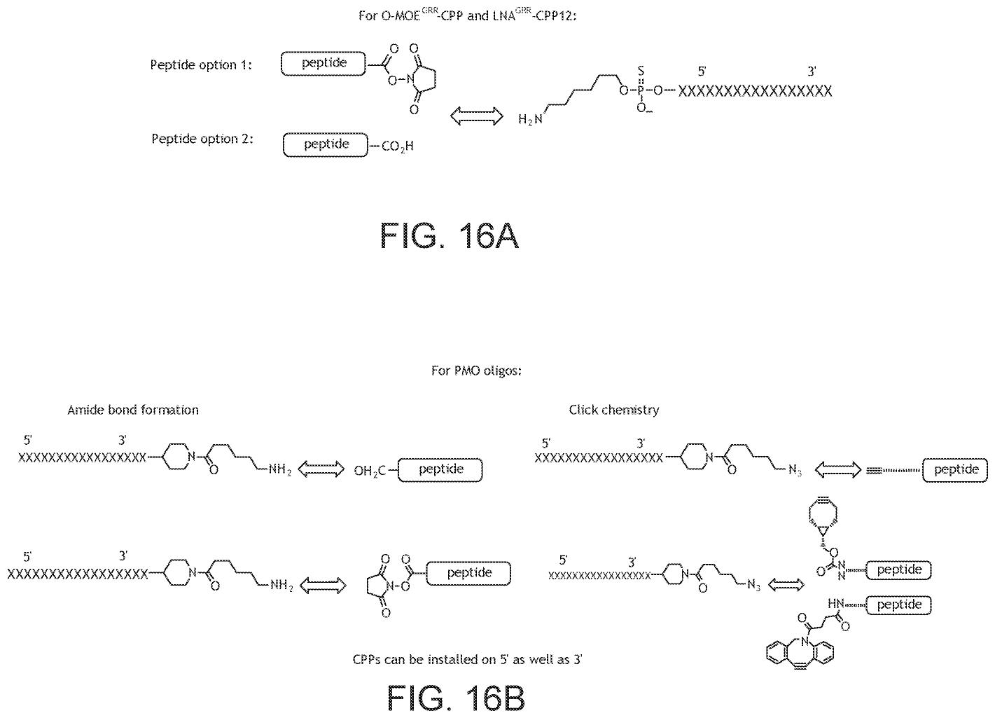

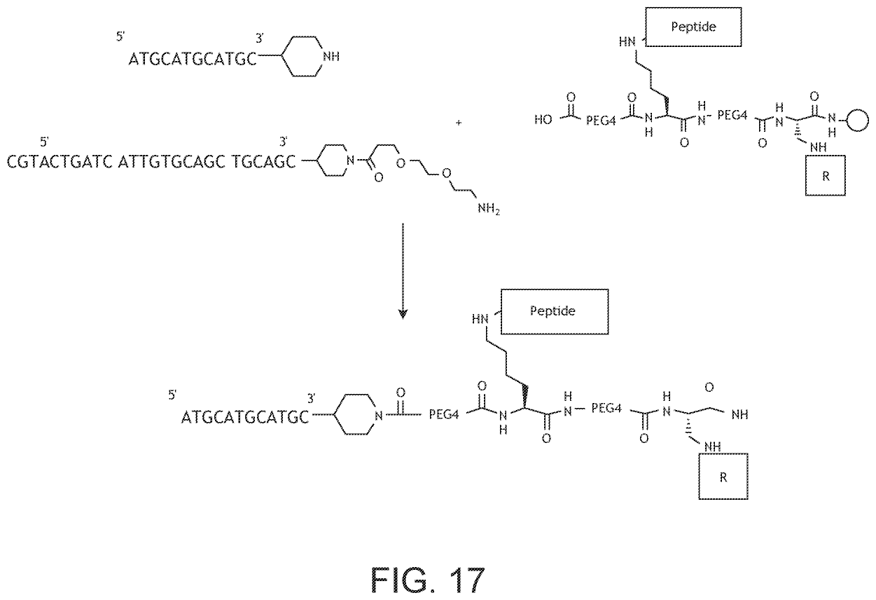

Results. Replacement of arginine residues with glycine retained or enhanced in vivo efficacy as determined by RT-PCR in mdx mice. PCR agarose gel images for exon 23 skipping in mdx mice 3 days after intravenous injection of 40 mpk of EEV-MDX-PMO-2 and 40 mpk of EEV-MDX-PMO-3 are shown in FIG. 13. Treatment with 40 mg/kg EEV-MDX-PMO-3 (based on PMO concentration) resulted in similar levels of efficacy as measured by exon 23 skipping compared to 40 mg/kg EEV-MDX-PMO-2 (based on PMO concentration) in all tissues evaluated, including quadriceps, diaphragm, and heart, as depicted in FIGS. 14A-C. Exon skipping efficiency in mdx mice further translated to robust dystrophin product across these tissues as determined by Western Blot and as depicted in FIG. 15A-D. These findings demonstrate that arginine replacement with glycine residues are able to render cCPPs cell-permeable and capable of successfully delivering cargo modalities to the cytosol and nucleus in vivo while enhancing tolerability.

### Example 6: Use of Cell-Penetrating Peptides Conjugated to Oligonucleotides for Splicing Correction of Exon 44 of DMD in hDMD and CD1 Mouse Models and in Non-Human Primates (NHP)

Purpose: This study employs hDMD and CD1 mouse models and a NHP model to study the effect of compounds comprising an antisense compound and a cell penetrating peptide. Each of the compounds contained the exocyclic sequence PKKKRKV (SEQ ID NO: 103).

Compounds Evaluated: The compounds evaluated in this study are shown in Table 14

TABLE 14
Compounds Evaluated in this Study

| | | Nucleic Acid Sequence of | |
|---|---|---|---|
| Compound | Peptide Sequence | AC (5′ - 3′) | Chemistry |
| EEV-PMO- | Ac-PKKKRKV-AEEA-Lys- | 5′- | PMO |
| DMD44-1 | (cyclo[FGFGRGRQ])-PEG₁₂-OH | AAACGCCGCCATTTCTC | |
| | (SEQ ID NO: 250) | AACAGATC-3′ (SEQ ID | |
| | | NO: 261) | |
| EEV-PMO- | Ac-PKKKRKV-AEEA- | 5′- | PMO |
| DMD44-2 | K(cyclo[GfFGrGrQ])-PEG₁₂-OH | AAACGCCGCCATTTCTC | |
| | (SEQ ID NO: 268) | AACAGATC-3′ (SEQ ID | |
| | | NO: 261) | |
| EEV-PMO- | Ac-PKKKRKV-AEEA- | 5′- | PMO |
| DMD44-3 | K(cyclo[FfΦGrGrQ])-PEG₁₂-OH | AAACGCCGCCATTTCTC | |
| | (SEQ ID NO: 275) | AACAGATC-3′ (SEQ ID | |
| | | NO: 261) | |

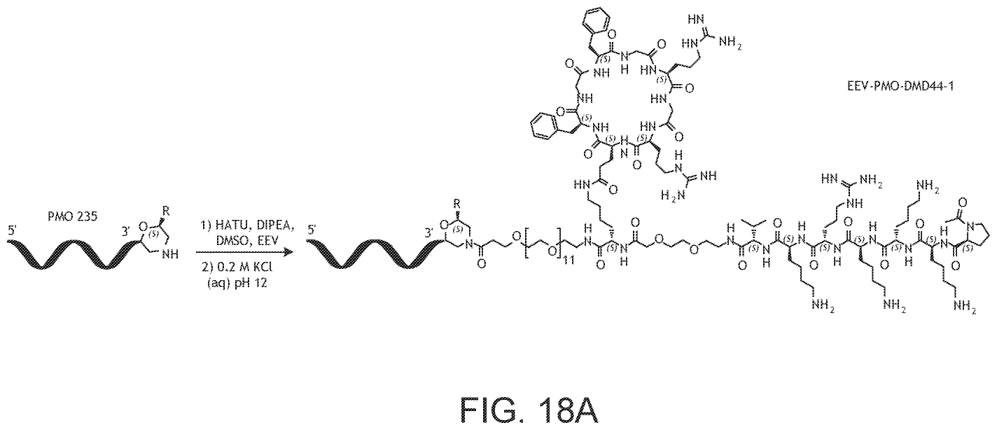

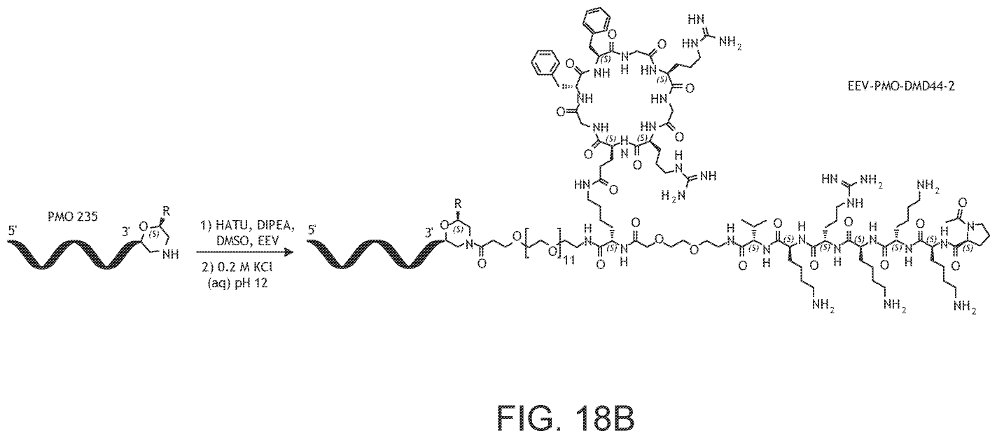

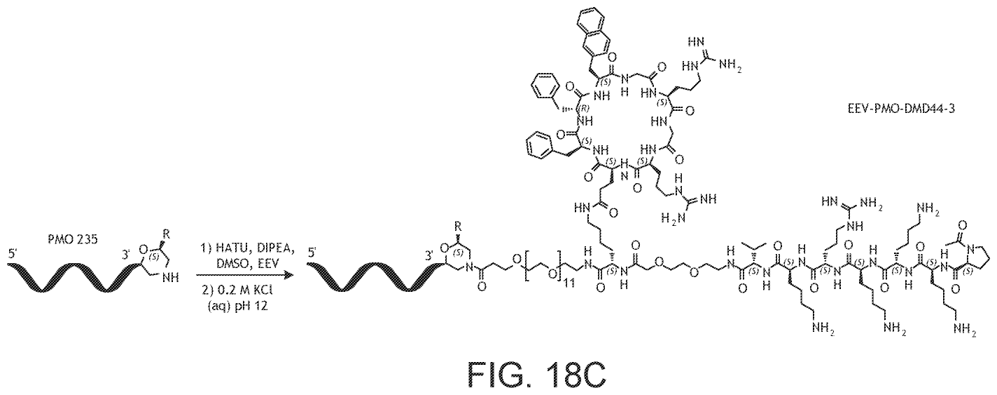

The structures of EEV-PMO-DMD44-1,2, and 3 are provided below. EEV-PMO-DMD44-1,2, and 3 are synthesized according to the scheme of FIG. 18A (EEV-PMO-DMD44-1), FIG. 18B (EEV-PMO-DMD44-2), and FIG. 18C (EEV-PMO-DMD44-3).

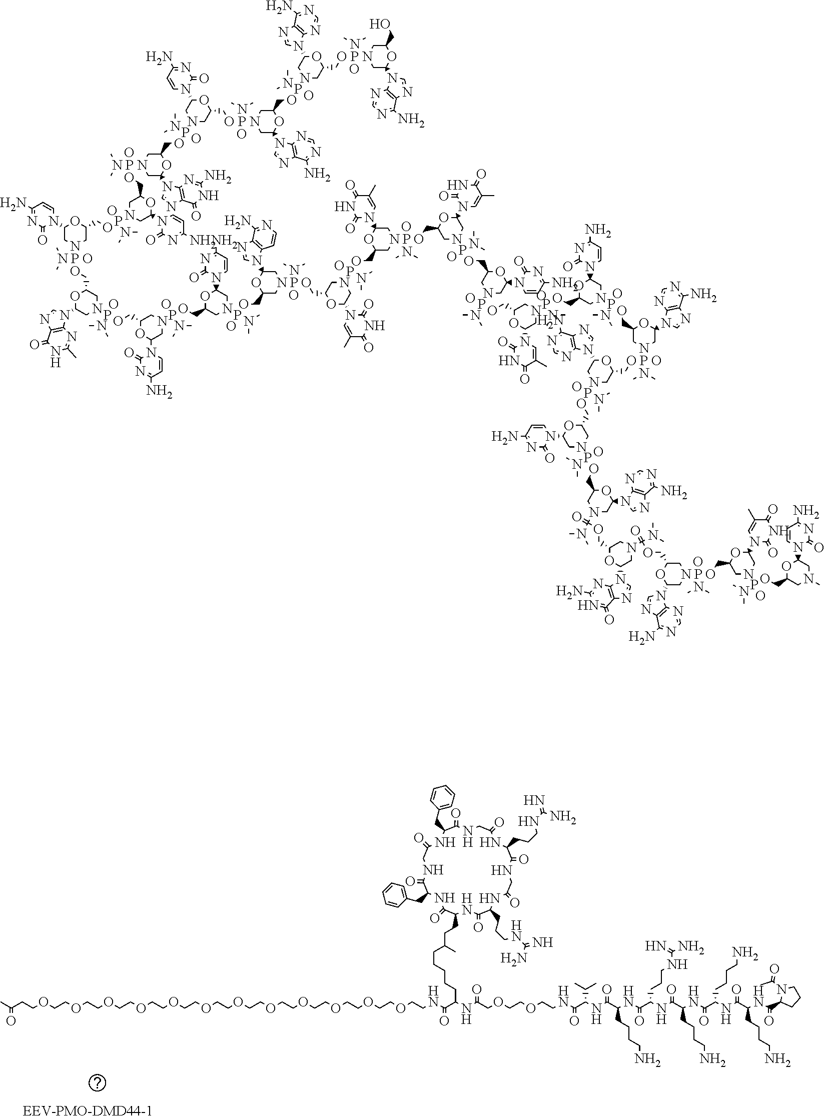

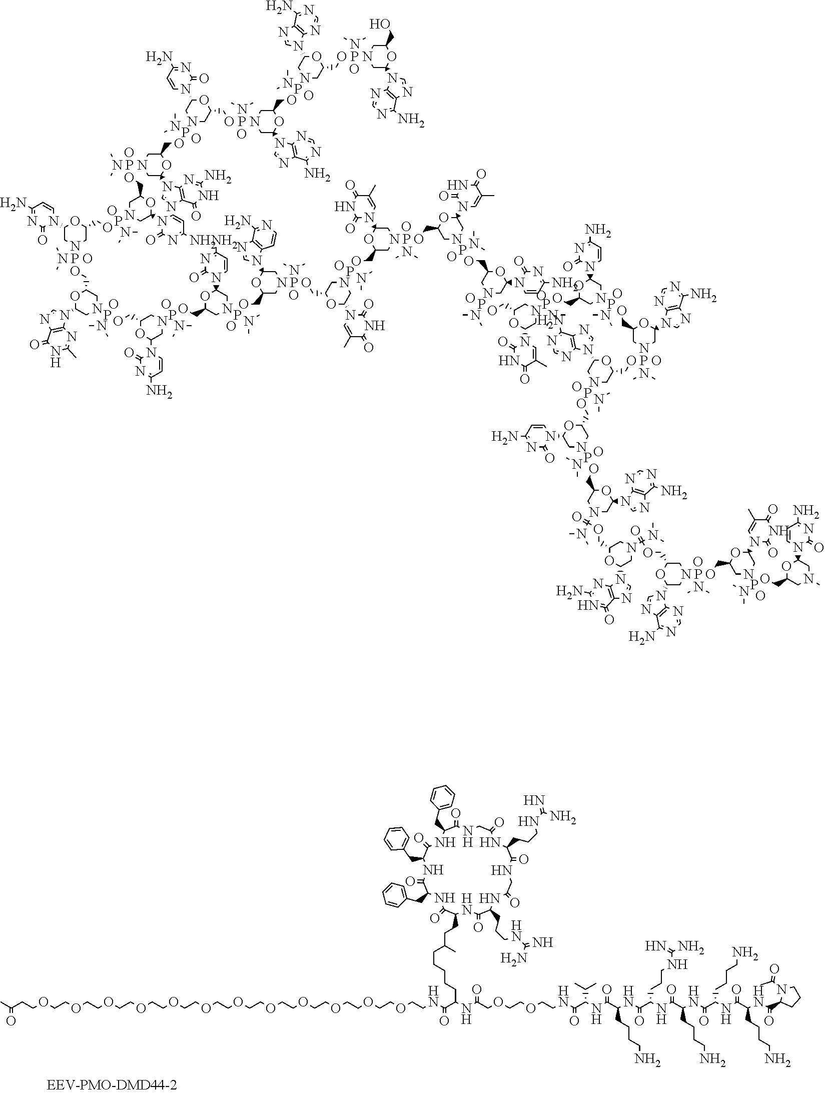

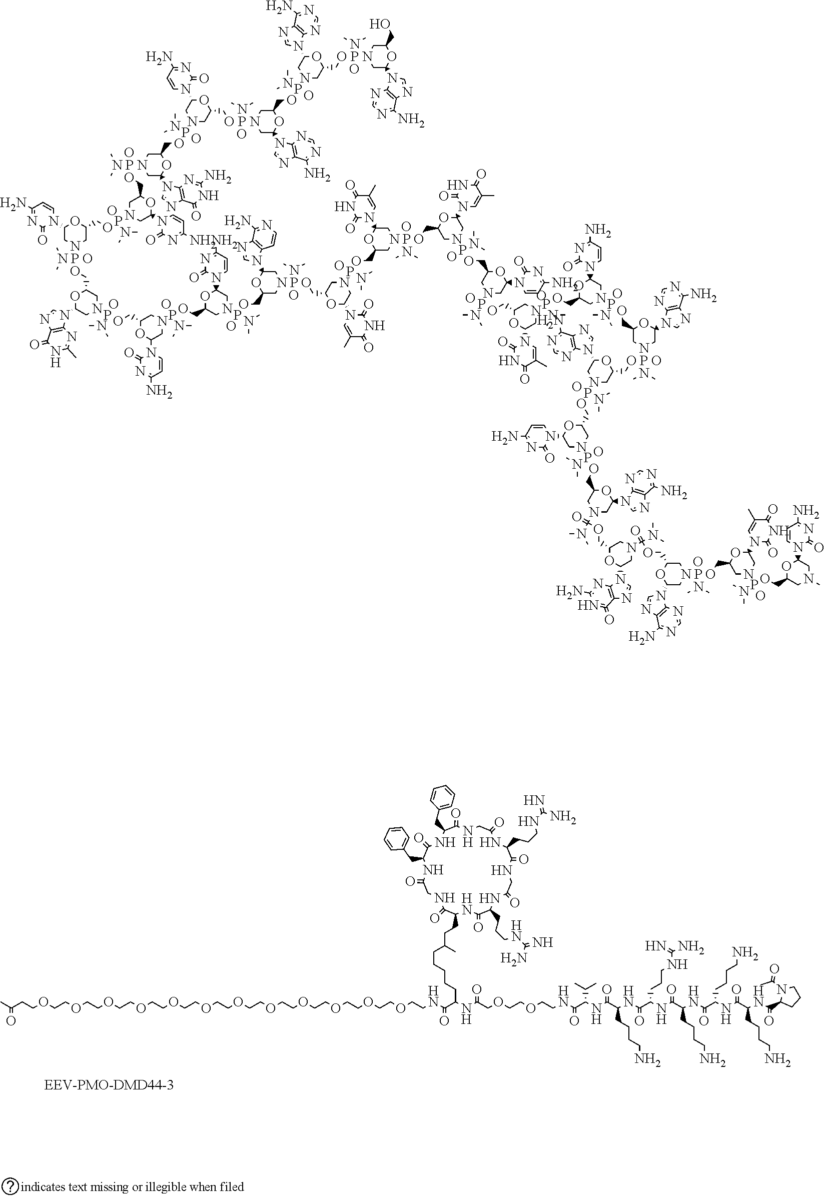

Compound Synthesis and Purification: The compounds were synthesized according to the following procedure. TFA-lysine protected cCPPs were reacted with the AC of Table 14 and subsequently deprotected to furnish a cCPP-AC conjugate. Briefly, the cCPP was pre-activated by reacting it with HATU (2.0 equiv) and DIPEA (2.0 equiv) in DMSO (10 mM, 1.8 mL). After 10 min at room temperature, the pre-activated solution was combined with a solution of AC in DMSO (10 mM, 1.8 mL) and mixed thoroughly. The reaction was incubated for 2 hours at room temperature. The reaction was monitored by LCMS (Q-TOF), using BEH C18 column (130 Å, 1.7 μm, 2.1 mm-50 mm), buffer A: water (0.1% FA), buffer B: acetonitrile (0.1% FA), flow rate: 0.4 mL/min, starting with 2% buffer B and ramping up to 98% over 3.4 min. Upon completion, in situ deprotection of TFA-protected lysines was initiated by dilution of the reaction mixture with 0.2 M KCl (aq) pH 12 (36 mL). The reaction was monitored by LCMS (Q-TOF), using the analysis method noted above. The crude mixture was loaded directly onto a C18 reverse-phase column (Oligo clarity column, 150 mm*21.2 mm). The crude product was then purified using a gradient of 5-20% over 60 min using water with 0.1% FA and acetonitrile as solvents and a flow rate of 20 mL/min. Fractions containing the desired product were pooled, and the pH of the solution was adjusted to 7 using 0.5 M NaOH. The solution was frozen and lyophilized, affording white powder. Formate salts were exchanged with chloride by reconstitution of the cCPP-AC conjugate in 1M NaCl in water and repeated washes through a 3-kD MW-cutoff amicon tube (centrifuged at 3500 rpm for 20-40 min). This process was performed three times with 1 M NaCl and three times with saline (0.9% NaCl, sterile, endotoxin-free). Conductivity of the last filtrate was assessed to confirm appropriate salt concentration. The solution was further diluted with saline to the desired formulation concentration and sterile filtered in a biosafety cabinet. The concentration of each formulation was remeasured post filtration.

EEV-PMO-DMD44-1 was obtained with 74% yield. The purity and identity of each formulation was assessed by liquid chromatography-mass spectrometry quadrupole time-of-flight mass spectrometry (QTOF-LCMS). EEV-PMO-DMD44-1 was determined to be 99% pure by RP-FA and 78% pure by CEX. The MW calod for C411H₆₆₁N₁₇₃O₁₃₀P₂₄, 10849.26. The MW identified by QTOF-LCMS was 10850.95. Formulations were further assayed for their endotoxin amount, residual free peptide, FA content and pH.

EEV-PMO-DMD44-2 was obtained with 70% yield. The purity and identity of each formulation was assessed by QTOF-LCMS. EEV-PMO-DMD44-2 was 99% pure by RP-FA and 78% pure by CEX. The MW calcd for C411H₆₆₁N₁₇₃O₁₃₀P₂₄, was 10849.26. The MW identified by QTOF-LCMS was 10850.88.

EEV-PMO-DMD44-3 was obtained with 68% yield. The purity and identity of each formulation was assessed by QTOF-LCMS. EEV-PMO-DMD44-3 was 86.3% pure by RP-FA (The impurity was unreacted AC). The MW calcd for C411H₆₆₁N₁₇₃O₁₃₀P₂₄, was 10989.45. The MW identified by QTOF-LCMS was 10990.07.

### Methods:

hDMD mouse model: hDMD mice were ordered from the Jackson Lab (STOCK Tg(DMD)₇₂Thoen/J; Stock No: 018900) and bred in-house. The hemizygous mice were further genotyped at Transnetyx. All groups were dosed 5 mL/kg per animal by intravenous (iv) injection and sacrificed after 5 days post injection. All animals were euthanized by CO₂ asphyxiation followed by terminal blood collection via cardiac puncture. Maximum obtainable volume of whole blood was collected into lithium heparin tubes and processed to plasma. A portion of plasma was analyzed for clinical chemistries by the Testing Facility (IDEXX) and the rest stored frozen at nominally −70° C. Tissues (Triceps, TA, diaphragm, heart, kidney, liver, Brain) were harvested and flash frozen in liquid nitrogen and stored at −80° C. for further evaluation of exon skipping and drug concentration measurements. Animals were age matched and assigned into eight (8) treatment groups according to Table 15. Group 1-1 (3 homo hDMD mice, 6 weeks old), 1-2 (3 homo hDMD mice, 6 weeks old), 1-3 (1 male, 1 female, hemi hDMD, 11 weeks old), 1-4 (1 male, 1 female, hemi DMD, 11 weeks old) received EEV-PMO-DMD44-1 at 10, 20, 40 and 80 milligrams per kilogram of body weight (mpk), respectively. Group 2-1 (3 homo hDMD mice, 6 weeks old), 2-2 (3 homo hDMD mice, 6 weeks), 2-3 (1 male, 1 female, hemi hDMD, 11 weeks), 2-4 (1 male, 1 female, hemi DMD, 11 weeks) received EEV-PMO-DMD44-2 at 10, 20, 40 and 80 mpk, respectively. All animals survived until their scheduled euthanasia time. Tissues were collected per protocol. The amount of AC and a cCPP-AC in various tissue samples was quantified by LC-MS. Exon skipping in different tissues were analyzed by RT-PCR and the quantification of exon 44 correction.

TABLE 15
Experimental design of hDMD Experiments

| | | | Dose | Dose | | Terminal |
|---|---|---|---|---|---|---|
| | Animal | Test | Level | Volume | Dosing | Time |
| Group | No. | Article | mg/kg | (5 mL/kg) | Regimen | Point |
| 1-1 | 3 | EEV-PMO- | 10 | 5 | IV | 5 days |
| | | DMD44-1 | | | | post |
| 1-2 | 3 | EEV-PMO- | 20 | | | injection |
| | | DMD44-1 | | | | |
| 1-3 | 3 | EEV-PMO- | 40 | | | |
| | | DMD44-1 | | | | |
| 1-4 | 3 | EEV-PMO- | 80 | | | |
| | | DMD44-1 | | | | |
| 2-1 | 2 | EEV-PMO- | 10 | | | |
| | | DMD44-2 | | | | |
| 2-2 | 2 | EEV-PMO- | 20 | | | |
| | | DMD44-2 | | | | |
| 2-3 | 2 | EEV-PMO- | 40 | | | |
| | | DMD44-2 | | | | |
| 2-4 | 2 | EEV-PMO- | 80 | | | |
| | | DMD44-2 | | | | |

CD1 mouse model: Tolerability of EEV-PMO-DMD44- and EEV-PMO-DMD44-2 were evaluated using CD1 male mice at 7 weeks age. They were ordered from the Charles River Lab and upon receiving, they were acclimated for 5 days prior to the injections. Animals were aged matched and assigned into nine (9) treatment groups according to Table 16 Group 1 (3 mice, saline); Group 2-1 (3 mice), 2-2 (2 mice), 2-3 (2 mice), 24 (2 mice), 2-5 (3 mice), 2-6 (3 mice) received EEV-PMO-DMD44-1 at 80, 100, 120, 160, 200 and 300 mpk, respectively. Group 3-1 (3 mice), 3-2 (2 mice), 3-3 (2 mice), 34 (2 mice), 3-5 (3 mice), 3-6 (3 mice) received EEV-PMO-DMD44-2 at 80, 100, 120, 160, 200 and 300 mpk, respectively.

TABLE 16
Experimental design of Tolerability Study in CD1 Mice

| | | | Dose | Dose | | Terminal |
|---|---|---|---|---|---|---|
| | Animal | Test | Level | Volume | Dosing | Time |
| Group | No. | Article | mg/kg | (5 mL/kg) | Regimen | Point |
| 1 | 3 | Saline | | 5 | IV | 7 days |
| 2-1 | 3 | EEV-PMO- | 80 | | | post |
| | | DMD44-1 | | | | injection |
| 2-2 | 2 | EEV-PMO- | 100 | | | |
| | | DMD44-1 | | | | |
| 2-3 | 2 | EEV-PMO- | 120 | | | |
| | | DMD44-1 | | | | |
| 2-4 | 2 | EEV-PMO- | 160 | | | |
| | | DMD44-1 | | | | |
| 2-5 | 3 | EEV-PMO- | 200 | | | |
| | | DMD44-1 | | | | |
| 2-6 | 3 | EEV-PMO- | 300 | | | |
| | | DMD44-1 | | | | |
| 3-1 | 2 | EEV-PMO- | 80 | | | |
| | | DMD44-2 | | | | |
| 3-2 | 2 | EEV-PMO- | 100 | | | |
| | | DMD44-2 | | | | |
| 3-3 | 2 | EEV-PMO- | 120 | | | |
| | | DMD44-2 | | | | |
| 3-4 | 2 | EEV-PMO- | 160 | | | |
| | | DMD44-2 | | | | |
| 3-5 | 3 | EEV-PMO- | 200 | | | |
| | | DMD44-2 | | | | |
| 3-6 | 3 | EEV-PMO- | 300 | | | |
| | | DMD44-2 | | | | |

NHP models: One female animal per compound (EEV-PMO-DMD44-1 and EEV-PMO-DMD44-2) was administered a 60-minute IV infusion with dosing volume of 10 mL/kg according to Table 17. Each testing article was formulated in saline at 4 mg/mL. Bloods and urine were taken at times indicated in Table 18 for further PK analysis. Biopsy at 2 days post injection was performed on biceps. Animals were sacrificed at day 7 post injection and skeletal muscles (quadriceps, diaphragm, biceps, deltoid, tibialis anterior (TiA), smooth muscles (esophagus, aorta, colon) and cardiac muscles (ventricle, atrium) were taken, pulverized, and stored at −80° C. for evaluation of exon skipping and biodistribution in tissues.

TABLE 17
Experimental design of NHP Study

| | Gender | | Dose | Dose | | Terminal |
|---|---|---|---|---|---|---|
| | of | Test | Level | Volume | Dosing | Time |
| Group | Animal | Article | mg/kg | (5 mL/kg) | Regimen | Point |
| 1 | Female | EEV-PMO- | 40 | 10 | IV | 7 days |
| | | DMD44-1 | | | infusion | post |
| 2 | Female | EEV-PMO- | 40 | | | injection |
| | | DMD44-1 | | | | |

TABLE 18
NHP Study Timepoints

| | | | Blood | | Sample | | Sampling time points |
|---|---|---|---|---|---|---|---|
| Group | matrix | Detect factor | volume | | volume | | post dose |
| 1-2 | Serum | Cytokine Panel | 0.5 | mL | 2 × 100 | μL | pre-dose (0 h), 1.5 h |
| | | IL-2/4/5/6/10/13/TNFα/IFN-γ | | | | | (30 min post full |
| | | | | | | | infusion), 24 h and Day 7 |
| | Blood | Hematology | 0.5 | mL | 0.5 | mL | pre-dose (0 h), 5 min |
| | | | | | | | (0.083 h) during |
| | | | | | | | infusion, 24 h and Day 7 |
| | Serum | Clinical Chemistry | 1 | mL | 400 | μL | pre-dose (0 h), 5 min |
| | | including magnesium | | | | | (0.083 h) during |
| | | | | | | | infusion, 24 h and Day 7 |
| | Blood | Coagulation | 1.8 | mL | 1.8 | mL | pre-dose (0 h), 5 min |
| | | | | | | | (0.083 h) during |
| | | | | | | | infusion, 24 h and Day 7 |
| | Urine | Urinalysis including | | | 4 | mL | pre-dose (0 h), and |
| | | Creatinine | | | | | 25~29 h and Day 7 |
| | Urine and | Additional collection | | | 4 | mL | pre-dose (0), 0-6 h, 6-12 |
| | feces* | to sponsor | | | | | h, 12-24 h, 24-48 h |
| | Muscle* | 2 cores per biopsy | | | 2 × (30-50) | mg | Day 2 |
| | | site at biceps brachii | | | | | |
| | Plasma* | PK analysis samples, | 0.5 | mL | 2 × 100 | μL | pre-dose (0 h), 30 min |
| | | sent to sponsor | | | | | (0.5 h) during infusion, |
| | | | | | | | 55 min (0.92 h) during |
| | | | | | | | infusion, 65 min |
| | | | | | | | (1.083 h), 1.5 h, 3 h, 9 h, |
| | | | | | | | 25 h, 39 h, 97 h, and |
| | | | | | | | 169 h (Day 7) |

1. Timer from the start point of the infusion.
2. Pre-dose tests of hematology, clinical chemistry, coagulation and urinalysis are conducted one week before dosing.

Bioanalytical Sample Analysis: Tissues were thawed, weighed, and homogenized (w/v, 1/5) with RIPA buffer spiked with 1× protease inhibitor cocktail (ThermoFisher Scientific, Ref #1860932). The homogeneates centrifuged at 5000 rpm for 5 minutes at 4° C. The supernatants were precipitated with a mixture of H₂O, acetonitrile and MeOH, and centrifuged at 15000 rpm for 15 minutes at 4° C. The supernatants were transferred to an injection plate for LC-MS/MS analysis using Shimadzu UPLC integrated with Triple Quad Sciex 4500 instrument. The dynamic range of the LC-MS/MS assay was 25 to 50,000 ng/g tissue. The details of the LC-MS/MS method are outlined here and in Table 19. Briefly, the UPLC was operated using Waters Acquity UPLC BEH C4, 300 Å, 1.7 um, 2.1×150 mm, buffer A: H₂O, 0.2% FA, buffer B: 95% acetonitrile in H₂O, 0.2% FA, flow rate (0.3 mL/min) and column temperature at 50° C. The 10 min run started with 2% buffer B and ramping up to 35% for 3.5 min followed by 90% for 1 min, staying at 90% gradient for 2.5 min and finally running at 2% gradient for 2 min. The MRM method was established for a duration of 7.5 min according to Table 19 with positive polarity; Turbo Spray ion source; Curtain Gas: 25; Collision Gas: 6; ion spray voltage: 5500; temperature: 500; ion source gas1: 60; ion source gas2: 60. The underlined rows of Table 21 are used for quantifications of intact and corresponding metabolite (AC-PEG12).

TABLE 19
LC-MS/MS Assay

| | Q1 | Charge | Q3 | | | | | |
|---|---|---|---|---|---|---|---|---|
| | Mass | state | Mass | Time | DP | EP | CE | CXP |
| Analyte | (Da) | (m/z) | (Da) | (msec) | (volts) | (volts) | (volts) | (volts) |
| EEV-PMO- | 835.6 | 13 | 112.0 | 50.0 | 80.0 | 8.0 | 80.0 | 10.0 |
| DMD44-1/ | | | | | | | | |
| EEV-PMO- | | | | | | | | |
| DMD44-2 | | | | | | | | |
| EEV-PMO- | 776.0 | 14 | 112.0 | 300.0 | 80.0 | 8.0 | 80.0 | 10.0 |
| DMD44-1/ | | | | | | | | |
| EEV-PMO- | | | | | | | | |
| DMD44-2 | | | | | | | | |
| EEV-PMO- | 724.3 | 15 | 112.0 | 50.0 | 80.0 | 8.0 | 80.0 | 10.0 |
| DMD44-1/ | | | | | | | | |
| EEV-PMO- | | | | | | | | |
| DMD44-2 | | | | | | | | |
| AC-PEG12 | 879.0 | 10 | 112.0 | 50.0 | 80.0 | 8.0 | 80.0 | 10.0 |
| AC-PEG12 | 799.2 | 11 | 112.0 | 50.0 | 80.0 | 8.0 | 80.0 | 10.0 |
| AC-PEG12 | 732.7 | 12 | 112.0 | 300.0 | 80.0 | 8.0 | 80.0 | 10.0 |
| IS | 863.8 | 12 | 112.0 | 100.0 | 80.0 | 8.0 | 80.0 | 10.0 |

Exon Skipping Analysis by RT-PCR: hDMD mice and NHP express full-length human dystrophin mRNA. The delivery of AC can alter the splicing and result in a shortened dystrophin mRNA after exon 44 skipping. The tissues were homogenized using 1 mL of RLT lysis buffer (Qiagen, Cat #79216). The detection of splicing correction process was measured by RT-PCR where extracted RNAs from tissues were first reverse-transcribed into cDNA and further analyzed by one step RT-PCR using the following primer set: forward primer 5′-GCTCAGGTCGGATTGACATT-3′ (SEQ ID NO: 262) and reverse primer 5′-GGGCAACTCTTCCACCAGTA-3′ (SEQ ID NO: 263). The RT-PCR readout of tissues without splicing correction resulted in a 641 bp gene fragment and a new 493 bp gene fragment that showed up after splicing correction. Quantification of the relative intensity of the bands corresponding to skipped and unskipped transcripts were performed to assess AC-induced exon-44-skipping efficacy. The degree (percentage) of splicing correction detected by RT-PCR was calculated using the following equation: % correction=(intensity of 493 bp fragment band)/(intensity of 493 bp fragment band+intensity of 641 bp fragment band).

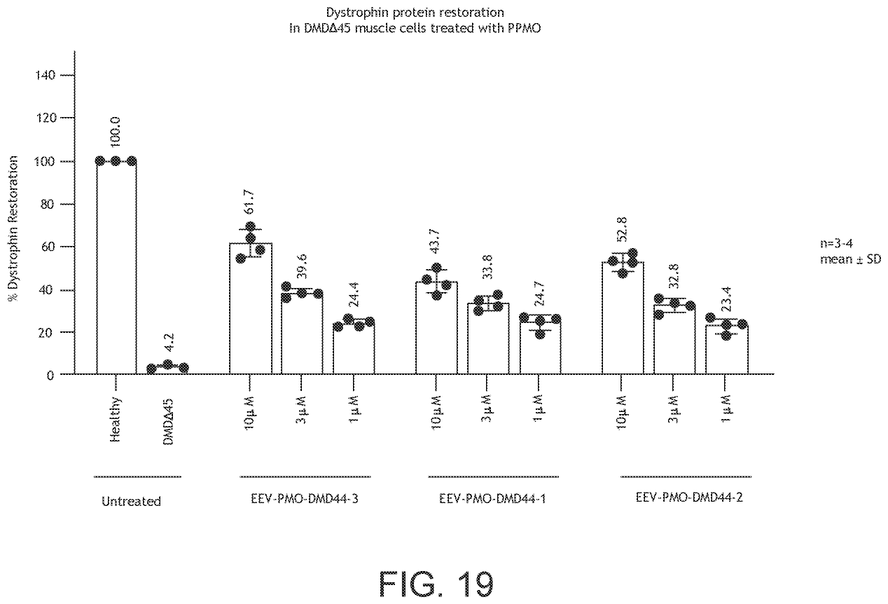

Results: Efficacy of EEV-PMO-DMD44-1,2, and 3 in Patient Myotubes: EEV-PMO-DMD44-1,2, and 3, which each target human dystrophin (DMD) exon 44 were assessed for DMD exon 44 skipping in DMD patient derived muscle cells. Briefly, patient-derived myoblasts harboring an exon 45 deletion (DMDA45) were treated with EEV-PMO-DMD44-1,2, and 3 at 1 μM, 3 μM, and 10 μM for 24 hours in PromoCell Skeletal Muscle Cell Growth Medium supplemented with 2% horse serum and 1% chick embryo extract. After 24 hours, the compound-containing growth medium was replaced with DMEM/2% horse serum and incubated for 5 days to promote myoblast fusion and differentiation into myotubes. Cells were washed and harvested for RNA extraction to assess exon 44 skipping, or in RIPA buffer containing protease inhibitors for protein extraction and Simple Western analysis of dystrophin protein restoration. The results are shown in FIG. 19. Dystrophin levels were normalized to HSP90 and expressed relative to untreated healthy samples. Data are expressed as mean±SD, n=3-4. Untreated DMDA45 patient derived cells express ˜10% spontaneous DMD exon 44 skipping and ˜4% dystrophin protein at baseline. All three compounds resulted in robust exon skipping and dystrophin protein restoration in a dose dependent manner.

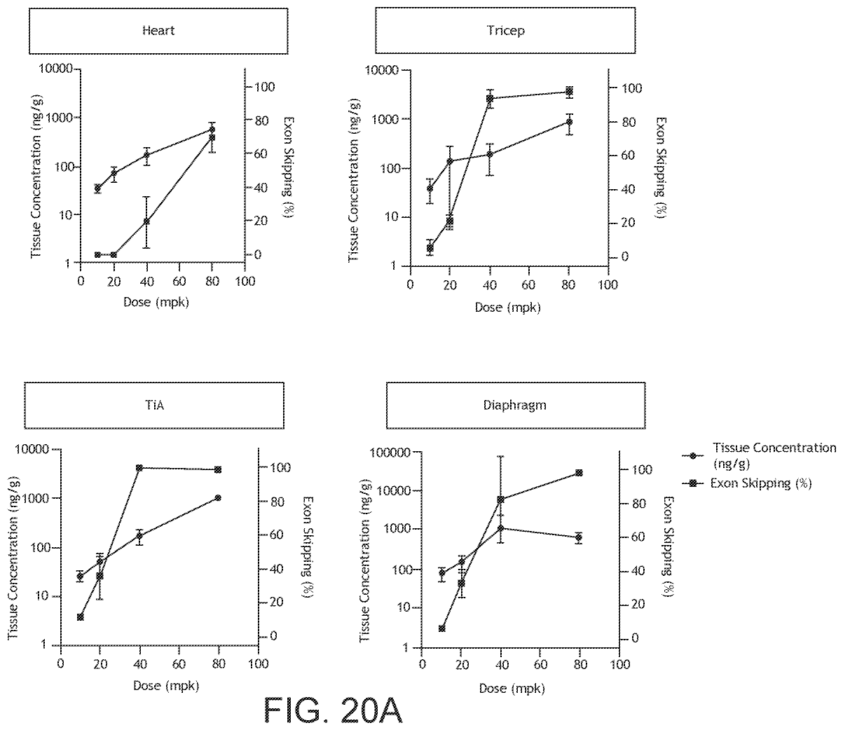

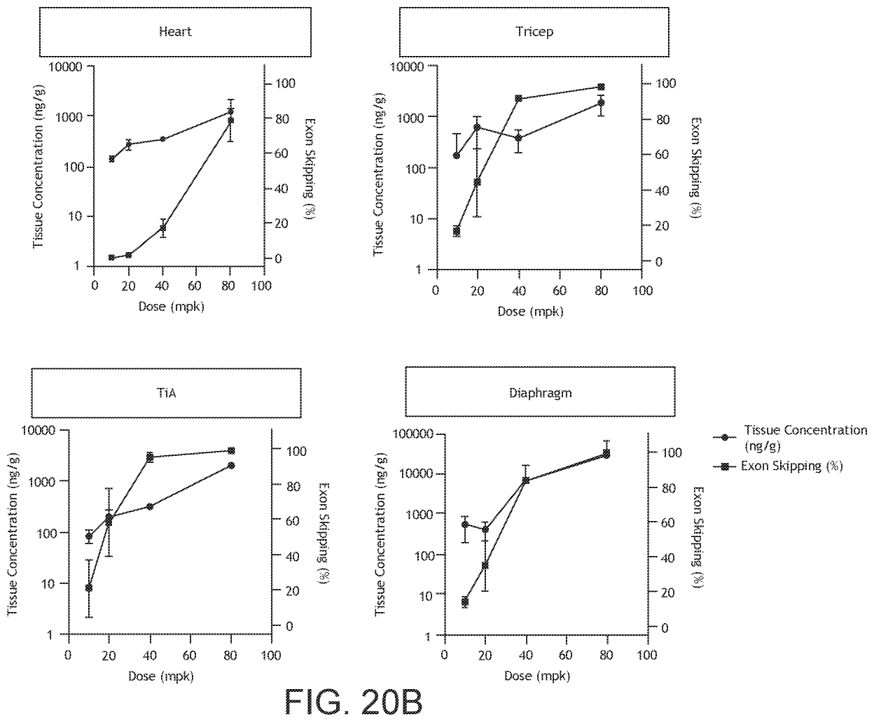

FIG. 20A-B show exon skipping in hDMD mice administered EEV-PMO-DMD44-1 (FIG. 201A) and EEV-PMO-DMD44-2 (FIG. 20B) via IV injection. No severe adverse effect was observed. The mice were all normal post injection, 24 hours post injection and prior to sac date. Clinical chemistries measuring liver and kidney toxicity (Alkaline Phosphatase (ALP), Aspartate transaminase (AST), Alanine Aminotransferase (ALT), Albumin, Blood Urea nitrogen (BUN), Creatinine, Calcium, Phosphorous, Chloride, Potassium, Sodium, BUN/Creatinine, Magnesium) as well as Hemolysis and Lipemia index are evaluated 5 days post IV injection. No significant toxicity was detected by clinical chemistry evaluation in EEV-PMO-DMD44-land EEV-PMO-DMD44-2 treated mice. Tissue concentrations and exon skipping in various muscle groups have been assessed 5-days post 10, 20, 40, and 80 mpk IV dosage. The following exon skipping was achieved for each dose of EEV-PMO-DMD44-1 in heart/Triceps/TiA/Diaphragm tissues, respectively: 10 mpk (0%, 6%, 12%, 6%); 20 mpk (0%, 22, 36%, 33%); 40 mpk (20%, 94%, 99%, 82%); 80 mpk (79%, 97%, 99%, 98%). The following exon skipping was achieved for each dose of EEV-PMO-DMD44-2 in heart/Triceps/TiA/Diaphragm tissues, respectively: 10 mpk (0%, 17%, 22%, 14%); 20 mpk (2%, 44, 58%, 35%); 40 mpk (17%, 92%, 95%, 83%); 80 mpk (79%, 98%, 99%, 99%). Strong dose-dependent accumulation and potent exon skipping was observed for both EEV-PMO-DMD44-1 and EEV-PMO-DMD44-2 in cardiac and skeletal muscles in the transgenic murine model carrying the full-size human DMD gene. At lower doses, 10 and 20 mpk, EEV-PMO-DMD44-2 drug exposure and efficacy were slightly higher than EEV-PMO-DMD44-1. However, this effect was started to diminish at 40 mpk dose, where both compounds resulted in same high level of exon skipping (above 80%) in all skeletal muscles. The corresponding tissue concentrations for EEV-PMO-DMD44-1 was in 100-300 ng/g tissue range while for EEV-PMO-DMD44-2 this range shifted to slightly higher number, 300-500 ng/g tissue concentration. Interestingly, the minimum efficacious dose for both EEV-PMO-DMD44-land EEV-PMO-DMD44-2 in the heart was achieved with 40 mpk corresponding to 170 and 350 ng per gram tissue concentration, respectively.

FIG. 20A and FIG. 20B shows exon skipping and drug concentration in heart, tricep, tibialis anterior and diaphragm tissues of hDMD mice treated with EEV-PMO-DMD44-1 (FIG. 20A) and EEV-PMO-DMD44-2 (FIG. 20B) via IV injection.

EEV-PMO-DMD44-1 was very well tolerated in all doses administered to CD1 mice at 80, 100, 160, 200 and 300 mpk doses. Only transient symptoms were observed which were completely resolved 1 hour post injection. No biomarker abnormalities were observed at 1- and 7-days post injection. EEV-PMO-DMD44-2 was less tolerated. At the highest dose of EEV-PMO-DMD44-2, 300 mpk, one out of three mice died within 1-3 h post injection. At lower dose of EEV-PMO-DMD44-2, 200 mpk, one out of three mice had severe symptoms (non-reactive to stimulation, ears are drawn back, slow respiration, struggles to right self). These symptoms progressively worsened, and they combined with muscle twitches. No symptoms were observed for lower doses at 160 and 80 mpk. Surprisingly, at 100 mpk, one out of three mice showed delayed symptoms 2 hours post injection: but they were completely normal 1- and 7-days post injection.

To further demonstrate the efficacy of exon skipping of cCPP-AC conjugates, NHP were utilized. Specifically, cynomolgus monkeys having intact muscle tissues were administered a 60-minute IV infusion of EEV-PMO-DMD44-1 or EEV-PMO-DMD44-2 at 40 mg/kg which were well-tolerated. More specifically, animals experienced nausea starting 45 minute of the treatment which was significantly resolved approximately 3 hours post treatment and the animal was more alert, less hunched and ate offered produce. Approximately 20 hours post dose the animal was (bright, alert, and responsive such that the animal was phenotypically “normal”) (BAR) and had zero biscuits left in the cage and was observed eating produce.

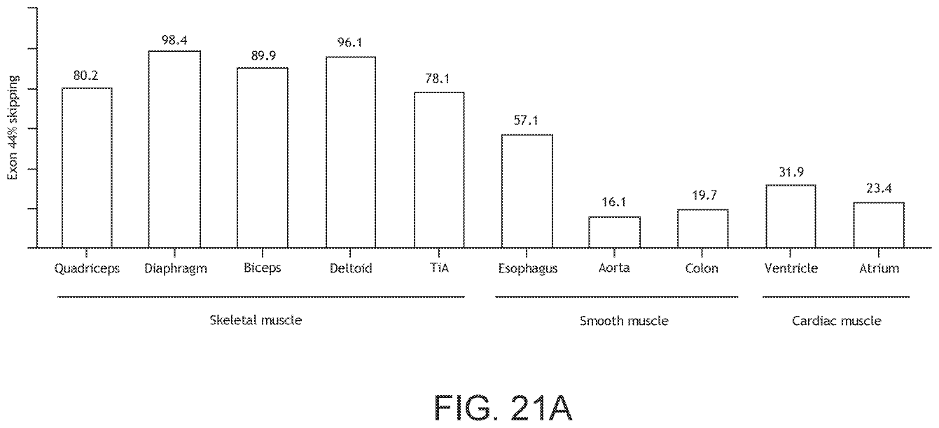

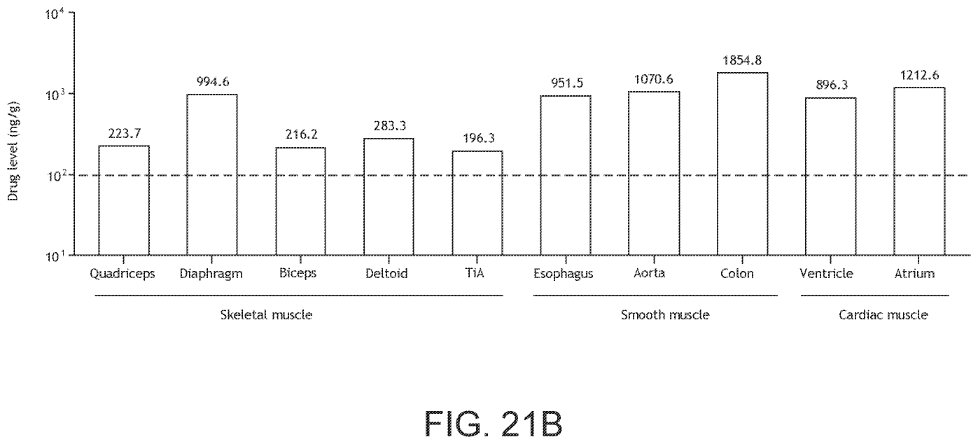

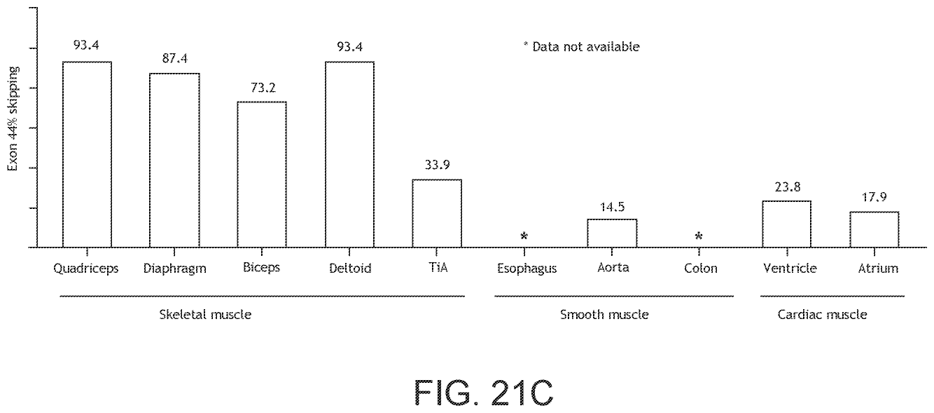

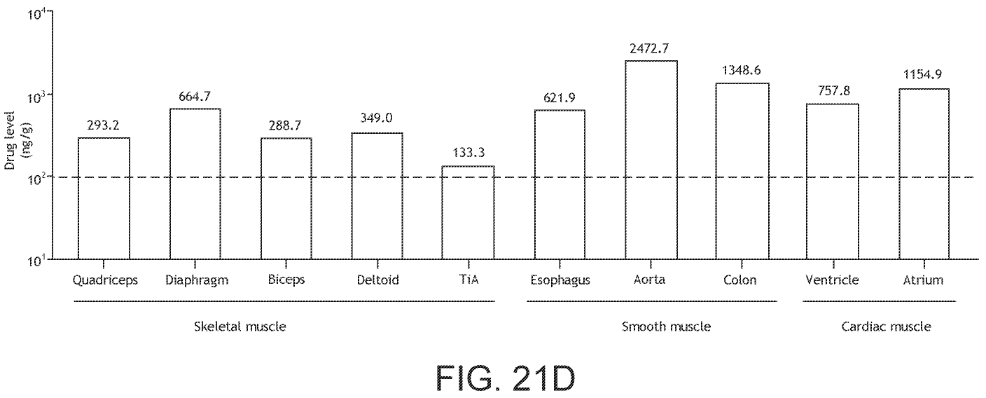

No abnormality in clinical chemistry panel at 2-d and 7-d post injection was observed. Exon 44 skipping percentage in different tissues were analyzed following by the standard protocol. FIGS. 21A-21B depict exon skipping (FIG. 21A) and drug exposure (FIG. 21B) for EEV-PMO-DMD44-1. FIGS. 21C-21D depict exon skipping (FIG. 21C) and drug exposure (FIG. 21D) for EEV-PMO-DMD44-2. Both compounds demonstrated an excellent exon skipping levels across different muscle groups with 40 mpk by IV at 7-d post injection. EEV-PMO-DMD44-1 outperformed in TiA, diaphragm, and less prominently in ventricle and atrium from efficacy standpoint. In all skeletal muscles more than 78% exon skipping achieved with maximum 98.4% in diaphragm. In cardiac tissues, EEV-PMO-DMD44-1 at 40 mpk resulted in 31.9% and 23.4% in ventricle and atrium, respectively. In smooth muscles, esophagus showed highest efficacy of 57.1%. Both EEV-PMO-DMD44-land EEV-PMO-DMD44-2 were distributed at pharmacologically relevant concentrations to various tissues. In some cases, such as ventricle and atrium in cardiac tissues and more prominently in esophagus and colon, the same tissue concentration didn't turn into the same functional delivery. This may indicate that the endosomal escape level in different tissues might be different. Nevertheless, in skeletal muscles, approximately 200 ng per gram tissue concentration correlated to a robust exon skipping of above 80%, while in cardiac tissues, 800-1000 ng per gram tissue concentration, correlated to roughly combined 50% exon skipping in atrium and ventricle. The 50% exon skipping with only single dose, 40 mpk of the cCPP-AC conjugates is very encouraging as the cardiac tissues are more challenging tissue for delivery and one that is crucial for treatment of neuromuscular disorders, such as DMD.

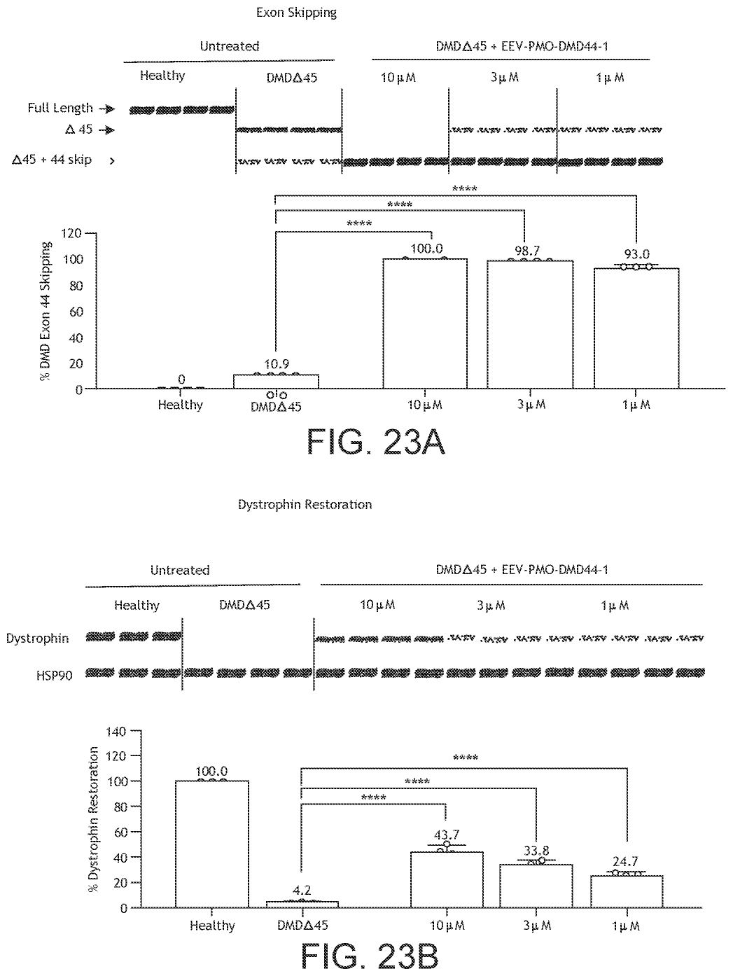

EEV-PMO-DMD44-1 was added to DMD patient-derived muscle cells and administered to hDMD transgenic mice that express full-length human dystrophin gene to test for human sequence-specific PMO for DMD transcript correction. Exon skipping and tissue concentrations in various muscles groups in the mice were assessed 5-day post 10, 20, 40 and 80 mg/kg IV dosage. FIG. 23A shows robust dose-dependent exon skipping and restoration of dystrophin in DMD patient-derived muscle cells treated with EEV-PMO-DMD44-1. Dose-dependent exon 44 skipping and dystrophin protein restoration was observed (up to 100% and 43.7% respectively) in DMD patient-derived muscle cells treated with EEV-PMO-DMD44-1 compared with both untreated patient derived cells and healthy cells. EEV-PMO-DMD44-1 was then studied in the humanized mouse model to assess uptake in tissue and exon skipping potential.

FIG. 23B shows dose-dependent tissue exposure and exon skipping in cardiac and skeletal muscles in a transgenic mouse carrying an integrated copy of the full-length human DMD gene after administering ascending IV doses of EEV-PMO-DMD44-1 at various levels ranging from 10 mg/kg to 80 mg/kg. Exon skipping and tissue exposure were each assessed five days after dosing. Dose dependent levels of tissue exposure of up to 80% and exon skipping up to 100% with translationally relevant doses were observed.

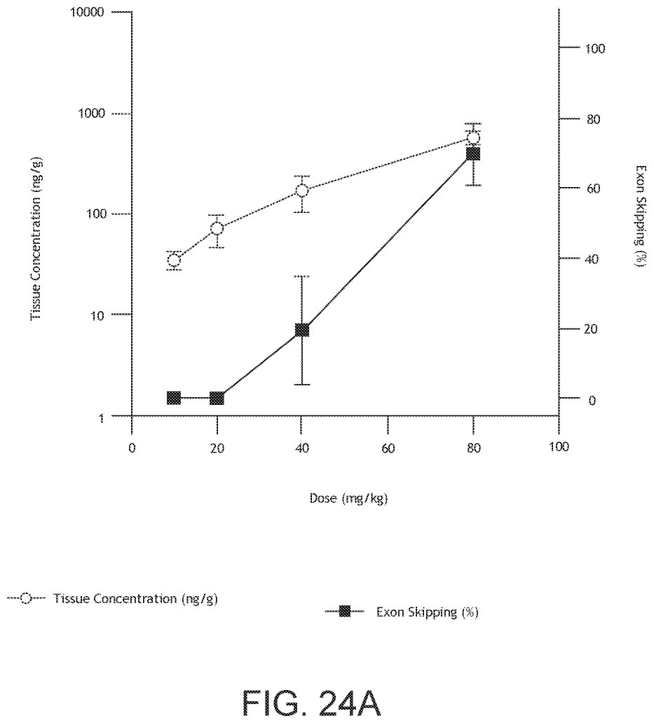

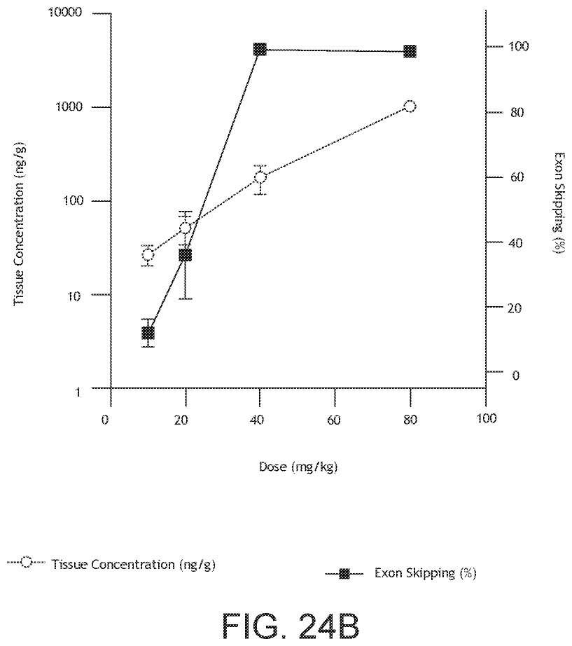

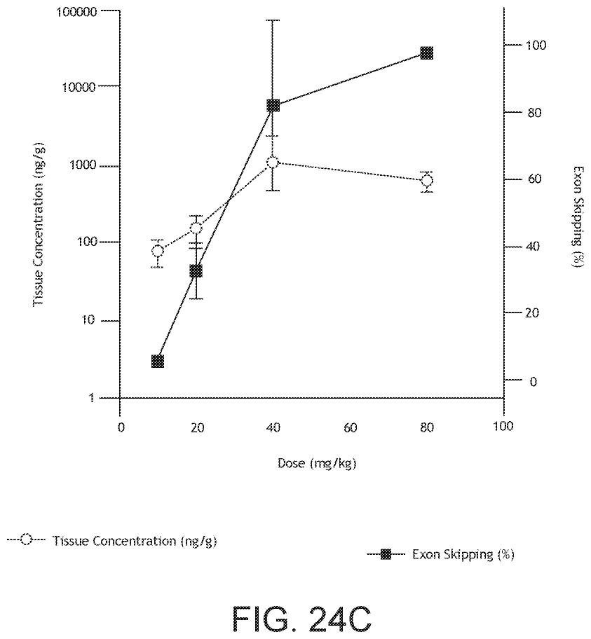

FIGS. 24A-24C depict the tissue concentration and percent of exon skipping for EEV-PMO-DMD44-1 in heart (FIG. 24A), tibialis anterior (FIG. 24B) and diaphragm (FIG. 24C) in a transgenic mouse carrying the full-length human DMD gene.

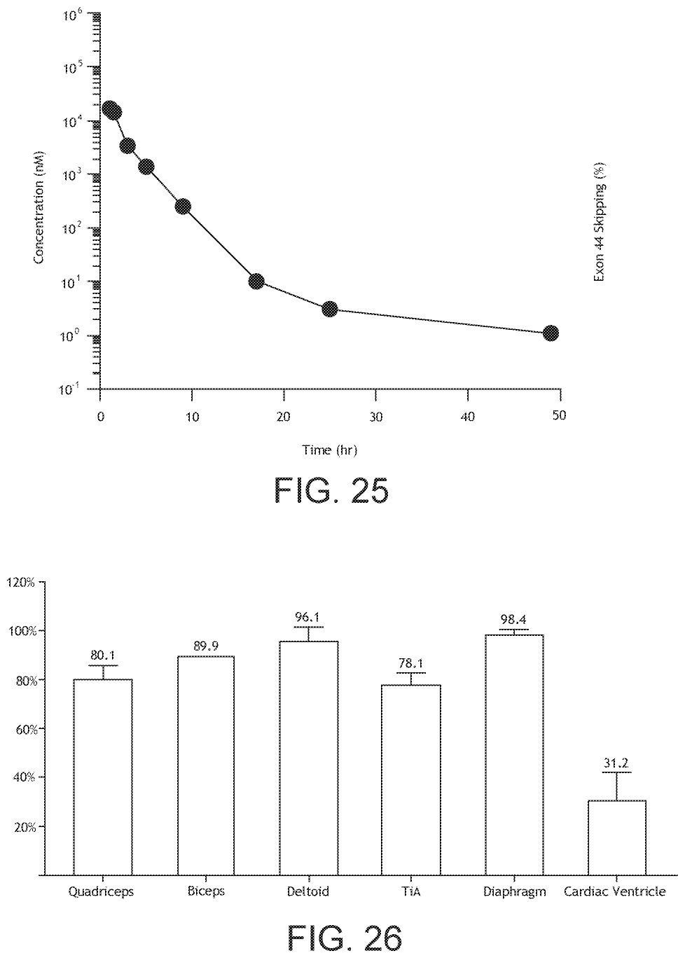

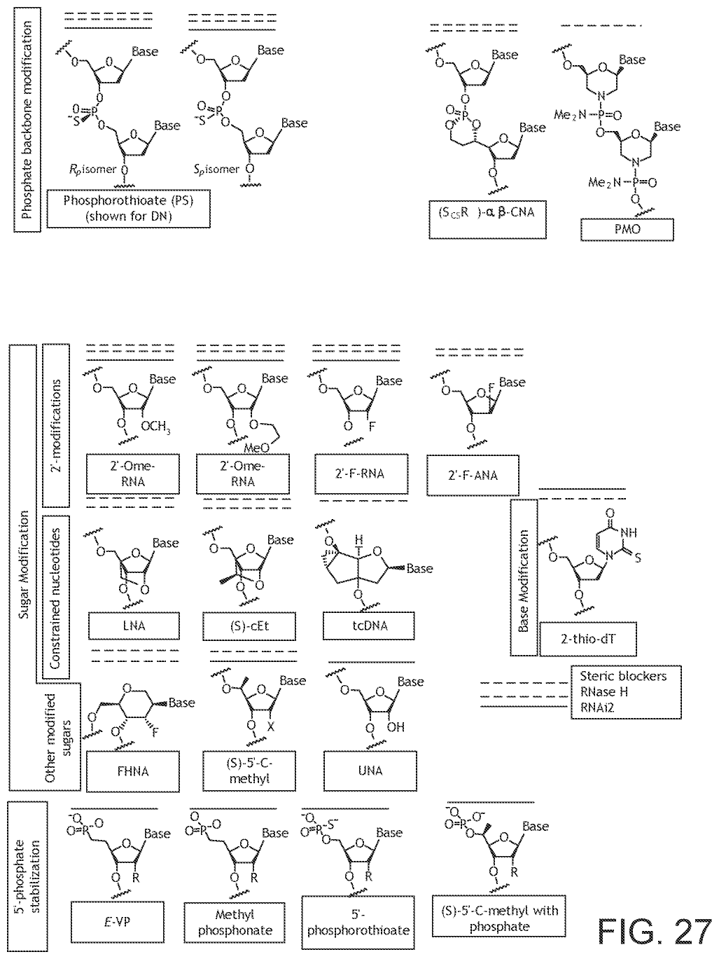

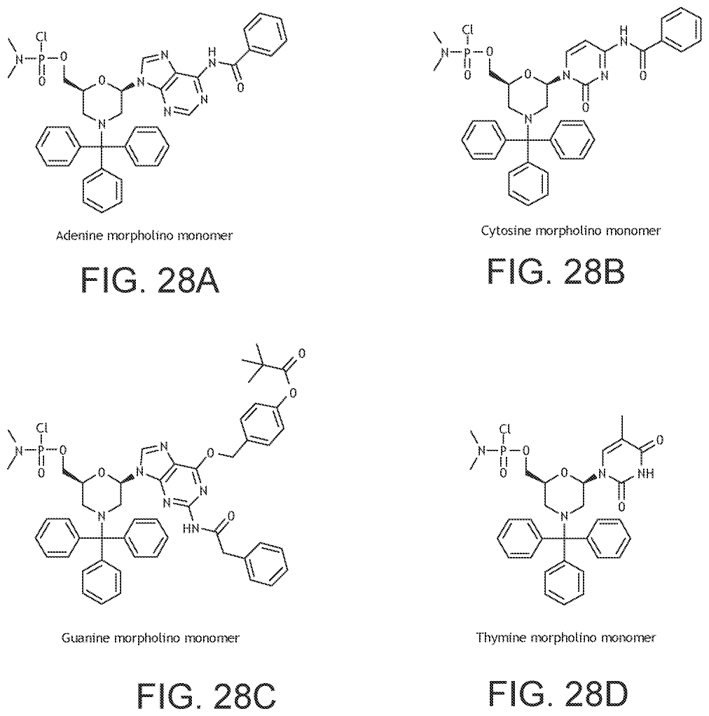

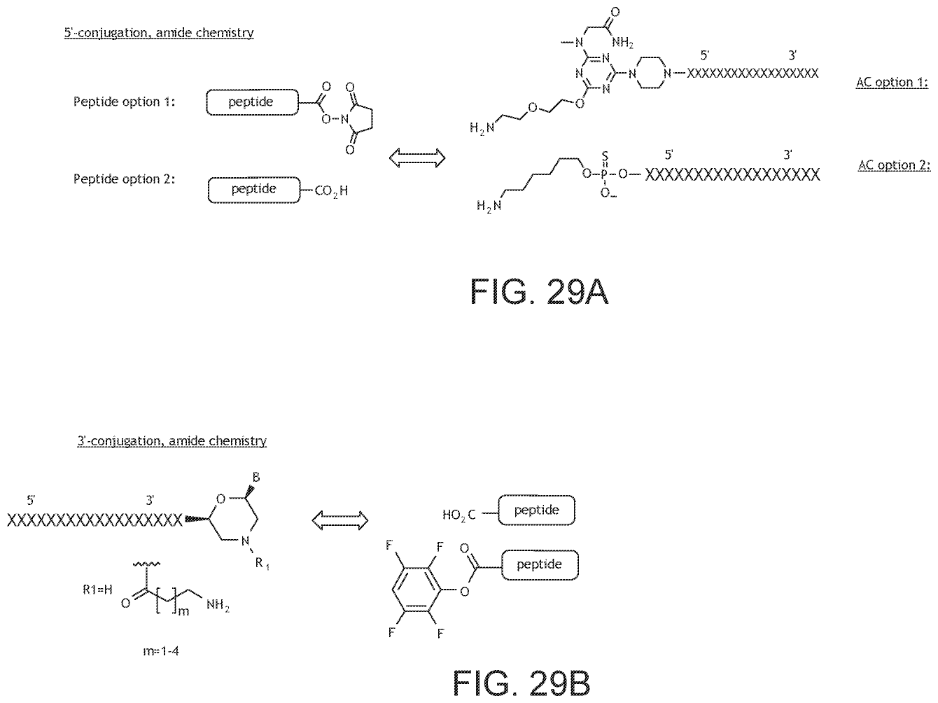

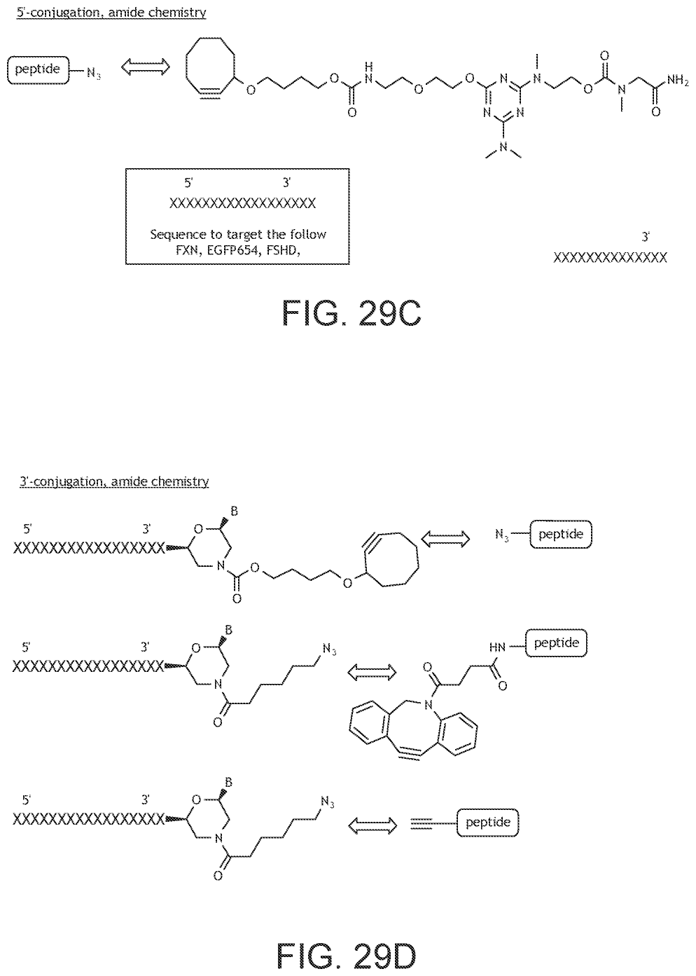

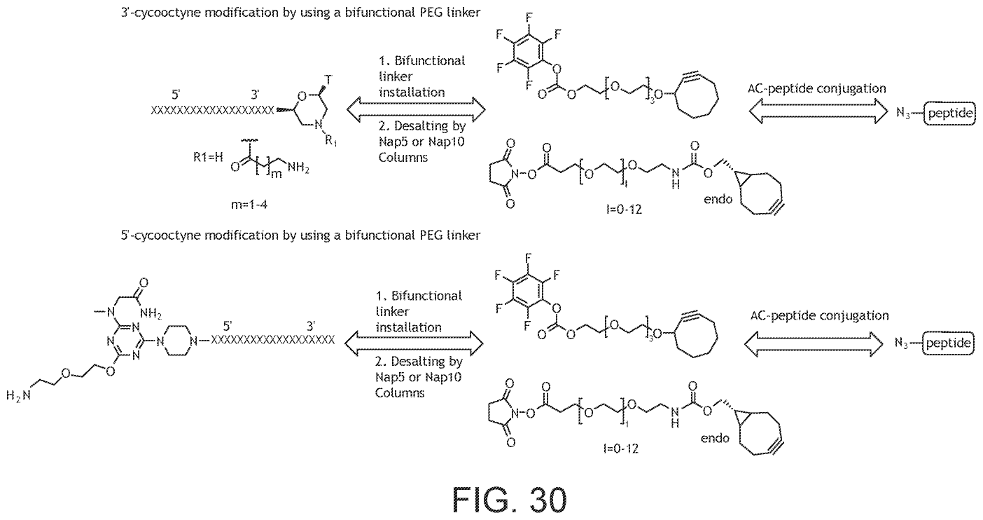

FIG. 25 shows that an extended circulating half-life for EEV-PMO-DMD44-1 was observed in the NHP. An extended circulating half-life for EEV-PMO-DMD44-1 was observed in the plasma of the NHP for up to 50 hours (FIG. 24A). This pharmacokinetic profile suggests an opportunity for intended tissue exposure, target engagement and pharmacodynamic effects.

FIG. 26 shows that a single 30 mg/kg IV dose of EEV-PMO-DMD44-1 resulted in meaningful levels of exon skipping in both skeletal muscles and the heart of the NHP which provides confidence in translational potential. At 7 days post 1 hour IV infusion at 30 mg/kg, robust exon 44 skipping observed across different muscle groups isolated from the EEV-PMO-DMD44-1 treated NHP.

These results represent a robust set of translational data. Exon skipping translates to promising dystrophin production in heart and skeletal muscles. Dystrophin production is sufficient to result in functional improvement. The dystrophin production was durable over 4+ weeks after a single injection.

### Example 7: Use of Cell-Penetrating Peptides Conjugated to Oligonucleotides for Targeting CTG Repeats in Myoblasts from DM1

Compounds Evaluated: The compounds evaluated in this study are shown in Table 21.

TABLE 21
Compounds Evaluated in this Study

| | Sequence | Nucleic Acid | |
|---|---|---|---|
| | | Sequence of AC | |
| Compound | Peptide | (5′ - 3′) | Chemistry |
| EEV- | Ac-PKKKRKV-AEEA-K(cyclo[FfFGRGRQ])- | CAGCAGCAGCAG | PMO |
| PMO- | AEEA-K(N₃)-NH₂ (SEQ ID NOs: 264 and 265) | CAGCAGCAG | |
| DM1- | | (SEQ ID NO: 212) | |
| 1 | | | |
| EEV- | Ac-PKKKRKV-K(cyclo[FfΦRrRrQ])-PEG₁₂-K(N₃)- | CAGCAGCAGCAG | PMO |
| PMO- | NH₂ (SEQ ID NOs: 246 and 151) | CAGCAGCAG | |
| DM1- | | (SEQ ID NO: 212) | |
| 2 | | | |
| EEV- | Ac-PKKKRKV-AEEA-K(cyclo[FGFGRGRQ])- | CAGCAGCAGCAG | PMO |
| PMO- | PEG₁₂-OH (SEQ ID NOs: 264 and 128) | CAGCAGCAG | |
| DM1- | | (SEQ ID NO: 212) | |
| 3 | | | |

Cell culture. Immortalized myoblasts from DM1 (ASA308DM1s) and unaffected individuals (KM1421; AB1190) were obtained from the Institut de Myologie, France, in accordance with French legislation. DM1 patient myoblasts harbor 2600 CTG repeats in the 3′UTR of DMPK. Myoblasts were cultured in a growth medium of Skeletal Muscle Cell Growth Medium (PomoCell), 2% horse serum (Gibco), 1% chick embryo extract (USB), and 0.5 mg/mL penicillin/streptomycin (Gibco). For myogenic differentiation, confluent cultures were switched to differentiation medium of DMEM supplemented with 2% horse serum and cultured for 4 (DM1).

Methods. For DM1, two treatment conditions were assessed, and each condition was run in triplicate. In the first condition, myoblasts were plated at 75-80% confluence, the compounds in Table 14 were serially diluted in growth medium, and cells were bathed for 24 hours with each compound, separately, to allow for free-uptake of the compounds. The media containing the compounds were removed, myoblasts washed with 1×DPBS (Gibco), and differentiated for four days prior to harvest. For the second condition run in parallel, myoblasts were differentiated three days prior to treatment. The compounds were serially diluted in differentiation medium and myotubes were harvested for analysis 24 hours later.

RNA isolation and PCR. Total RNA was isolated with the Qiagen RNeasy Mini Kit according to the manufacturer's instructions. For exon inclusion, 100 ng RNA was reverse transcribed and used for PCR (OneStep RT-PCR Kit, Qiagen). Samples were analyzed by LabChip (PerkinElmer) with the HT DNA High Sensitivity Assay Kit.

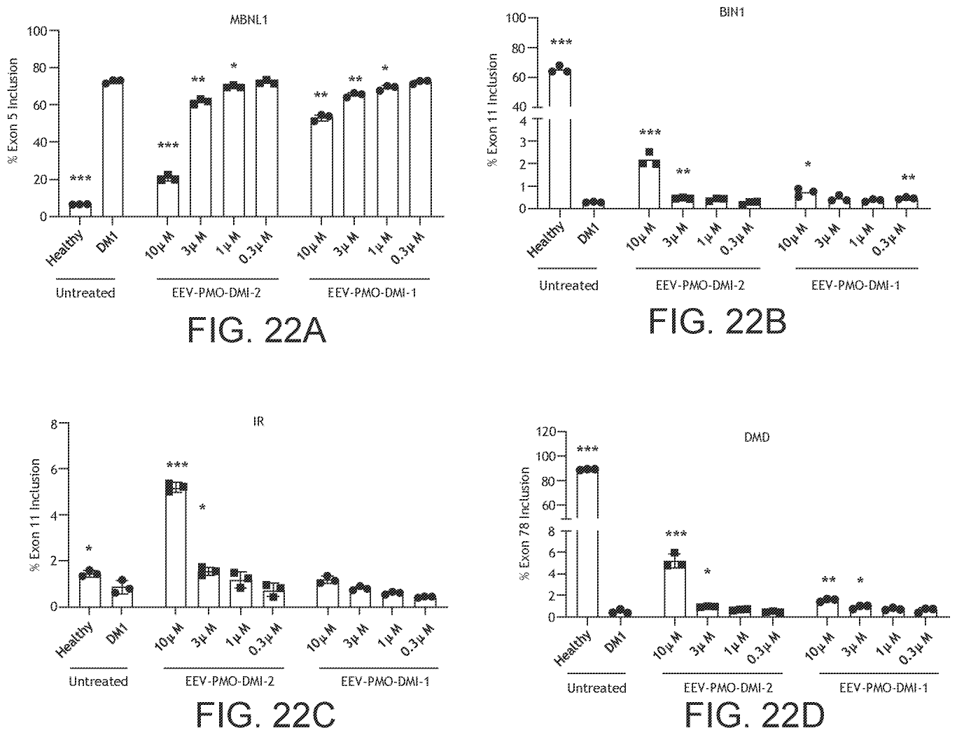

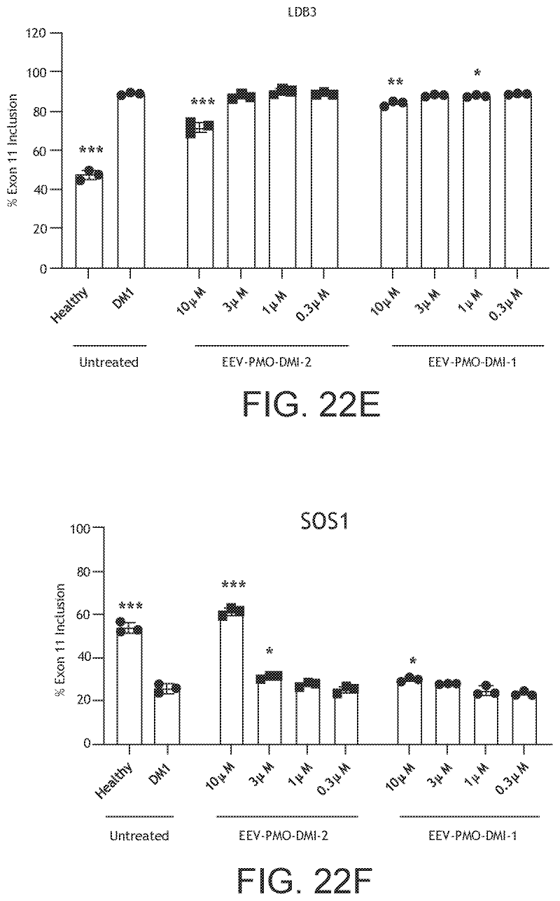

FIGS. 22A-22F demonstrate RNA splicing measurements for Mbnl1 (for exon 5 inclusion; FIG. 22A), Bin1 (for exon 11 inclusion; FIG. 22B), IR (for exon 11 inclusion; FIG. 22C), DMD (for exon 78 inclusion; FIG. 22D), LDB3 (for exon 11 inclusion; FIG. 22E) and Sos1 (for exon inclusion; FIG. 22F). The data generally demonstrates at least partial recovery using the compounds described herein.

### Example 8. Efficacy of Charge-Reduced Cyclic Cell-Penetrating Peptides for Splicing Correction of Exon 23 of DMD in an MDX Mouse Model

The compounds of Table 22 below include additional non-limiting examples of compounds. Compounds were prepared as described in previous examples.

TABLE 22
Sequence

Ac-PKKKRKV-K(cyclo[FfΦR-cit-R-cit-Q])-PEG12-K(N3)(SEQ ID NOs:
246 and 249)
Ac-rr-miniPEG2-Dap[cyclo(FfΦ-Cit-r-Cit-rQ)]-PEG12-OH (SEQ ID NO:
236)
Ac-frr-PEG2-Dap(cyclo(FfΦ-Cit-r-Cit-rQ))-PEG12-OH (SEQ ID NO: 236)
Ac-rfr-PEG2-Dap(cyclo(FfΦ-Cit-r-Cit-rQ))-PEG12-OH (SEQ ID NO: 236)
Ac-rbfbr-PEG2-Dap(cyclo(FfΦ-Cit-r-Cit-rQ))-PEG12-OH(SEQ ID NO:
236)
Ac-rrr-PEG2-Dap(cyclo(FfΦ-Cit-r-Cit-rQ))-PEG12-OH (SEQ ID NO: 236)
Ac-rbr-PEG2-Dap(cyclo(FfΦ-Cit-r-Cit-rQ))-PEG12-OH (SEQ ID NO: 236)
Ac-rbrbr-PEG2-Dap(cyclo(FfΦ-Cit-r-Cit-rQ))-PEG12-OH (SEQ ID NO:
236)
Ac-hh-PEG2-Dap(cyclo(FfΦ-Cit-r-Cit-rQ))-PEG12-OH (SEQ ID NO: 236)
Ac-hbh-PEG2-Dap(cyclo(FfΦ-Cit-r-Cit-rQ))-PEG12-OH (SEQ ID NO:
236)
Ac-hbhbh-PEG2-Dap(cyclo(FfΦ-Cit-r-Cit-rQ))-PEG12-OH (SEQ ID NO:
236)
Ac-rbhbh-PEG2-Dap(cyclo(FfΦ-Cit-r-Cit-rQ))-PEG12-OH (SEQ ID NO:
236)
Ac-hbrbh-PEG2-Dap(cyclo(FfΦ-Cit-r-Cit-rQ))-PEG12-OH (SEQ ID NO:
236)
Ac-rr-Dap(cyclo(FfΦ-Cit-r-Cit-rQ))-b-OH (SEQ ID NO: 236)
Ac-frr-Dap(cyclo(FfΦ-Cit-r-Cit-rQ))-b-OH (SEQ ID NO: 236)
Ac-rfr-Dap(cyclo(FfΦ-Cit-r-Cit-rQ))-b-OH (SEQ ID NO: 236)
Ac-rbfbr-Dap(cyclo(FfΦ-Cit-r-Cit-rQ))-b-OH (SEQ ID NO: 236)
Ac-rrr-Dap(cyclo(FfΦ-Cit-r-Cit-rQ))-b-OH (SEQ ID NO: 236)
Ac-rbr-Dap(cyclo(Ff-Cit-r-Cit-rQ))-b-OH (SEQ ID NO: 236)
Ac-rbrbr-Dap(cyclo(FfΦ-Cit-r-Cit-rQ))-b-OH (SEQ ID NO: 236)
Ac-hh-Dap(cyclo(FfΦ-Cit-r-Cit-rQ))-b-OH (SEQ ID NO: 236)
Ac-hbh-Dap(cyclo(FfΦ-Cit-r-Cit-rQ))-b-OH (SEQ ID NO: 236)
Ac-hbhbh-Dap(cyclo(FfΦ-Cit-r-Cit-rQ))-b-OH (SEQ ID NO: 236)
Ac-rbhbh-Dap(cyclo(FfΦ-Cit-r-Cit-rQ))-b-OH (SEQ ID NO: 236)
Ac-hbrbh-Dap(cyclo(FfΦ-Cit-r-Cit-rQ))-b-OH (SEQ ID NO: 236)
Ac-KKKK-miniPEG2-K(cyclo(Ff-Nal-GrGrQ))-miniPEG2-K(N3)-NH2
(SEQ ID NOs: 64 and 234)
Ac-KGKK-miniPEG2-K(cyclo(Ff-Nal-GrGrQ))-miniPEG2-K(N3)-NH2
(SEQ ID NOs: 70 and 234)
Ac-KKGK-miniPEG2-K(cyclo(Ff-Nal-GrGrQ))-miniPEG2-K(N3)-NH2
(SEQ ID NOs: 71 and 234)
Ac-KKK-miniPEG2-K(cyclo(Ff-Nal-GrGrQ))-miniPEG2-K(N3)-NH2
(SEQ ID NO: 234)
Ac-KK-miniPEG2-K(cyclo(Ff-Nal-GrGrQ))-miniPEG2-K(N3)-NH2 (SEQ
ID NO: 234)
Ac-KGK-miniPEG2-K(cyclo(Ff-Nal-GrGrQ))-miniPEG2-K(N3)-NH2
(SEQ ID NO: 234)
Ac-KBK-miniPEG2-K(cyclo(Ff-Nal-GrGrQ))-miniPEG2-K(N3)-NH2 (SEQ
ID NO: 234)
Ac-KBKBK-miniPEG2-K(cyclo(Ff-Nal-GrGrQ))-miniPEG2-K(N3)-NH2
(SEQ ID NO: 234)
Ac-KR-miniPEG2-K(cyclo(Ff-Nal-GrGrQ))-miniPEG2-K(N3)-NH2(SEQ
ID NO: 234)
Ac-KBR-miniPEG2-K(cyclo(Ff-Nal-GrGrQ))-miniPEG2-K(N3)-NH2 (SEQ
ID NO: 234)
Ac-PKKKRKV-miniPEG2-K(cyclo(Ff-Nal-GrGrQ))-miniPEG2-K(N3)-
NH2 (SEQ ID NOs: 246 and 234)
Ac-PKKKRKV-miniPEG2-K(cyclo(Ff-Nal-GrGrQ))-miniPEG2-K(N3)-
NH2 (SEQ ID NOs: 246 and 234)
Ac-PGKKRKV-miniPEG2-K(cyclo(Ff-Nal-GrGrQ))-miniPEG2-K(N3)-
NH2 (SEQ ID NOs: 104 and 234)
Ac-PKGKRKV-miniPEG2-K(cyclo(Ff-Nal-GrGrQ))-miniPEG2-K(N3)-
NH2 (SEQ ID NOs: 105 and 234)
Ac-PKKGRKV-miniPEG2-K(cyclo(Ff-Nal-GrGrQ))-miniPEG2-K(N3)-
NH2 (SEQ ID NOs: 106 and 234)
Ac-PKKKGKV-miniPEG2-K(cyclo(Ff-Nal-GrGrQ))-miniPEG2-K(N3)-
NH2 (SEQ ID NOs: 107 and 234)
Ac-PKKKRGV-miniPEG2-K(cyclo(Ff-Nal-GrGrQ))-miniPEG2-K(N3)-
NH2 (SEQ ID NOs: 108 and 234)
Ac-PKKKRKG-miniPEG2-K(cyclo(Ff-Nal-GrGrQ))-miniPEG2-K(N3)-
NH2 (SEQ ID NOs: 109 and 234)
Ac-KKKRK-miniPEG2-K(cyclo(Ff-Nal-GrGrQ))-miniPEG2-K(N3)-NH2
(SEQ ID NOs: 76 and 234)
Ac-KKRK-miniPEG2-K(cyclo(Ff-Nal-GrGrQ))-miniPEG2-K(N3)-NH2
(SEQ ID NOs: 65 and 234)
Ac-KRK-miniPEG2-K(cyclo(Ff-Nal-GrGrQ))-miniPEG2-K(N3)-NH2 (SEQ
ID NOs 234)
Ac-KKKK-miniPEG2-K(cyclo(FGFGRGRQ))-miniPEG2-K(N3)-NH2
(SEQ ID NOs: 64 and 128)
Ac-KBKBK-miniPEG2-K(cyclo(FGFGRGRQ))-miniPEG2-K(N3)-NH2
(SEQ ID NOs 128)
Ac-PKKKRKV-miniPEG2-K(cyclo(FGFGRGRQ))-miniPEG2-K(N3)-NH2
(SEQ ID NOs: 103 and 128)
Ac-KGK-miniPEG2-K(cyclo(FGFGRGRQ))-miniPEG2-K(N3)-NH2 (SEQ
ID NO: 128)
Ac-KBK-miniPEG2-K(cyclo(FGFGRGRQ))-miniPEG2-K(N3)-NH2 (SEQ
ID NO: 128)
Ac-KGKK-miniPEG2-K(cyclo(FGFGRGRQ))-miniPEG2-K(N3)-NH2
(SEQ ID NOs: 70 and 128)
Ac-KKKK-miniPEG2-K(cyclo(GfF-GrGrQ))-miniPEG2-K(N3)-NH2 (SEQ
ID NOs: 64 and 233)
Ac-KBKBK-miniPEG2-K(cyclo(GfF-GrGrQ))-miniPEG2-K(N3)-NH2
(SEQ ID NO: 233)
Ac-PKKKRKV-miniPEG2-K(cyclo(GfF-GrGrQ))-miniPEG2-K(N3)-NH2
(SEQ ID NOs: 103 and 233)
Ac-KGK-miniPEG2-K(cyclo(GfF-GrGrQ))-miniPEG2-K(N3)-NH2 (SEQ
ID NO: 233)
Ac-KBK-miniPEG2-K(cyclo(GfF-GrGrQ))-miniPEG2-K(N3)-NH2 (SEQ
ID NO: 233)
Ac-KGKK-miniPEG2-K(cyclo(GfF-GrGrQ))-miniPEG2-K(N3)-NH2 (SEQ
ID NOs: 70 and 233)
Ac-KKKK-miniPEG2-K(cyclo(FfF-GrGrQ))-miniPEG2-K(N3)-NH2 (SEQ
ID NOs: 64 and 235)
Ac-KBKBK-miniPEG2-K(cyclo(FfF-GrGrQ))-miniPEG2-K(N3)-NH2
(SEQ ID NOs: 234)
Ac-PKKKRKV-miniPEG2-K(cyclo(FfF-GrGrQ))-miniPEG2-K(N3)-NH2
(SEQ ID NOs: 103 and 235)
Ac-KGK-miniPEG2-K(cyclo(FfF-GrGrQ))-miniPEG2-K(N3)-NH2 (SEQ
ID NO: 235)
Ac-KBK-miniPEG2-K(cyclo(FfF-GrGrQ))-miniPEG2-K(N3)-NH2 (SEQ
ID NO: 235)
Ac-KGKK-miniPEG2-K(cyclo(FfF-GrGrQ))-miniPEG2-K(N3)-NH2 (SEQ
ID NOs: 70 and 235)
Ac-PKKKRKV-miniPEG2-K(cyclo(FGFGRGRQ))-PEG12-K(N3)-NH2
(SEQ ID NOs: 246 and 128)
Ac-PKKKRKV-miniPEG2-K(cyclo(GfF-GrGrQ))-PEG12-K(N3)-NH2
(SEQ ID NOs: 246 and 233)
Ac-KKKK-miniPEG2-K(cyclo(FGFGRGRQ))-PEG12-K(N3)-NH2 (SEQ
ID NOs: 64 and 128)
Ac-KKKK-miniPEG2-K(cyclo(GfF-GrGrQ))-PEG12-K(N3)-NH2 (SEQ ID
NOs: 64 and 233)
Ac-PKKKRKV-miniPEG2-K(cyclo(FGFGRGRQ))-PEG12-OH (SEQ ID
NOs: 246 and 128)
Ac-PKKKRKV-miniPEG2-K(cyclo(GfF-GrGrQ))-PEG12-OH (SEQ ID
NOs: 246 and 233)
Ac-KKKK-miniPEG2-K(cyclo(FGFGRGRQ))-PEG12-OH (SEQ ID NOs:
64 and 128)
Ac-KKKK-miniPEG2-K(cyclo(GfF-GrGrQ))-PEG12-OH (SEQ ID NOs: 64
and 233)

Non-limiting examples of cCPP chemical structures are shown:

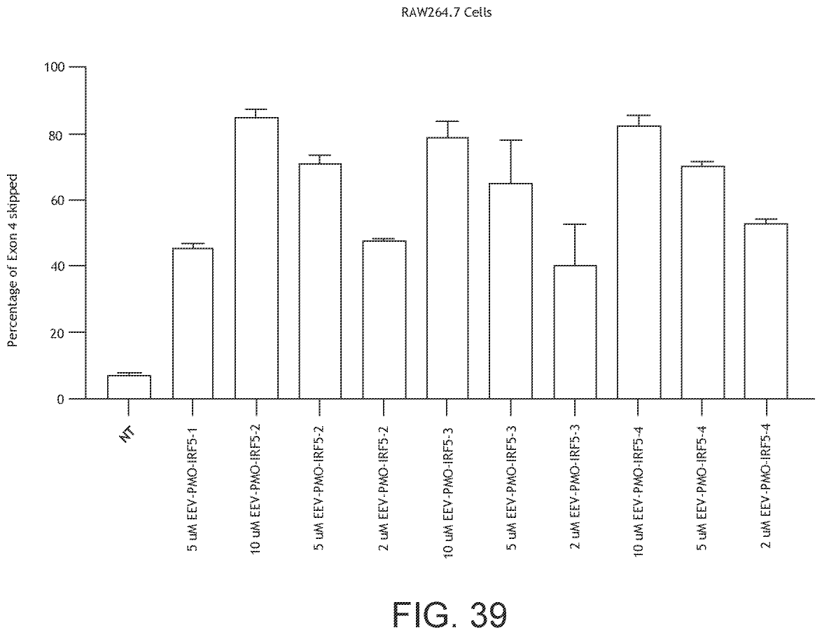

Compounds were tested in the MDX model described in Example 4. Mice were treated with a single intravenous dose at 20 mg/kg on day 1. On day 5 post-injection, animals were sacrificed and the specified tissues were harvested and flash-frozen. RNA was extracted and exon skipping was quantified by RT-PCR as previously described in diaphragm (FIG. 39A), heart (FIG. 39B), tibialis anterior (FIG. 39C) and triceps (FIG. 39D). The results indicated that treatment with EEV-PMO-MDX-4,5,6 and 7 resulted in a lower level of exon skipping in heart tissue as compared to the three the types of muscle tissue examined. Additionally, on day 5 post-injection, dystrophin levels were quantified by Wester blot analysis in diaphragm (FIG. 40A), heart (FIG. 40B) and tibialis anterior (FIG. 40C). The results indicated that treatment with NLS-containing compounds (EEV-PMO-MDX-8, EEV-PMO-MDX-5) also resulted in lower levels dystrophin expression in heart tissue and tibialis anterior tissue as compared to the diaphragm tissue.

TABLE 23
Compounds Evaluated in this Study

| ID | Peptide Sequence | Nucleic Acid Sequence of AC (5′-3′) | Chemistry |
|---|---|---|---|
| EEV- | Ac-PKKKRKV-AEEA- | GGCCAAACCTCGGCTTACCTGAAAT | PMO |
| PMO- | K(cyclo[FGFGRGRQ])- | (SEQ ID NO: 139) | |
| MDX- | AEEA-K(N₃)-NH₂ (SEQ | | |
| 4 | ID NO: 266) | | |
| EEV- | Ac-KBK-AEEA- | GGCCAAACCTCGGCTTACCTGAAAT | PMO |
| PMO- | K(cyclo[FGFGRGRQ])- | (SEQ ID NO: 139) | |
| MDX- | AEEA-K(N₃)-NH₂ (SEQ | | |
| 5 | ID NO: 267) | | |
| EEV- | Ac-PKKKRKV-AEEA- | GGCCAAACCTCGGCTTACCTGAAAT | PMO |
| PMO- | K(cyclo[GfFGrGrQ])- | (SEQ ID NO: 139) | |
| MDX- | AEEA-K(N₃)-NH₂(SEQ | | |
| 6 | ID NO: 268) | | |
| EEV- | Ac-PKKKRKV-AEEA- | GGCCAAACCTCGGCTTACCTGAAAT | PMO |
| PMO- | K(cyclo[FfFGRGRQ])- | | |
| MDX- | AEEA-K(N₃)-NH₂ (SEQ | (SEQ ID NO: 139) | |
| 7 | ID NO: 269) | | |
| EEV- | Ac-KBKBK-AEEA- | GGCCAAACCTCGGCTTACCTGAAAT | PMO |
| PMO- | K(cyclo[FfFGRGRQ])- | (SEQ ID NO: 139) | |
| MDX- | AEEA-K(N₃)-NH₂ (SEQ | | |
| 8 | ID NO: 270) | | |

### Example 9. Knockdown of GYS1 Expression Via Exon Skipping

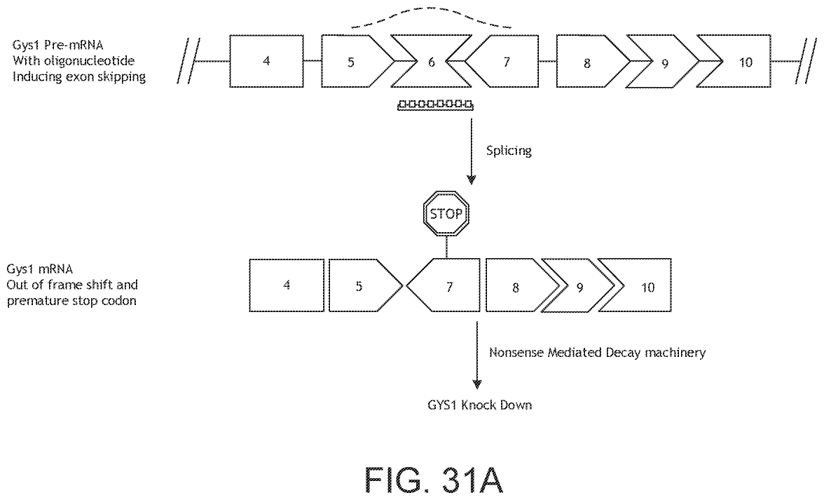

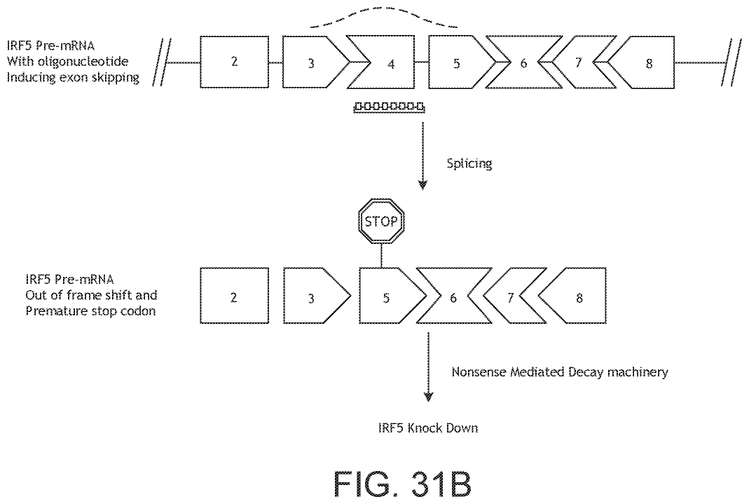

FIG. 31A shows a schematic for knocking down the expression of GYS1 using exon skipping. An EEV-PMO is used to induce exon skipping of exon 6. The EEV used was either Ac—PKKKRKV-PEG₂-K(cyclo[Ff-Na-Cit-r-Cit-rQ])-PEG₁₂-K(N₃)—NH₂(SEQ ID NOs: 246 and 236), or Ac-PKKKRKV-PEG₂-K(cyclo[Ff-Nal-GrGrQ])-PEG₂-K(N₃)—NH₂ (SEQ ID NOs. 246 and 234). Skipping exon 6 shifts the reading frame and incudes the reading a premature stop codon. The GYS1 mRNA is then degraded via nonsense mediated decay machinery thereby preventing expression of a full GYS1 protein.

TABLE 24
Compounds Evaluated in this Study

| | | Nucleic Acid Sequence of AC | |
|---|---|---|---|
| ID | Peptide Sequence | (5′-3′) | Chemistry |
| EEV- | Ac-PKKKRKV-PEG₂- | TCA CTG TCT GGC TCA CAT ACC | PMO |
| PMO- | K(cyclo[Ff-ϕ-Cit-r-Cit- | CAT A (SEQ ID NO: 273) | |
| GYS1- | rQ])-PEG₁₂-K(N₃)-NH₂ | | |
| 1 | (SEQ ID NOs: 103 and | | |
| | 236) | | |
| EEV- | Ac-PKKKRKV-PEG₂- | TCA CTG TCT GGC TCA CAT ACC | PMO |
| PMO- | K(cyclo[Ff-ϕ-GrGrQ])- | CAT A (SEQ ID NO: 273) | |
| GYS-2 | PEG₂-K(N₃)-NH₂ (SEQ | | |
| | ID NOs: 103 and 234). | | |

GYS1/GAA double knockout mice, when compared to the GAA single knockout mice, have exhibited a profound reduction in the amount of glycogen in the heart and skeletal muscles, a significant decrease in lysosomal swelling and autophagic build-up. These cellular-level changes lead to cardiomegaly correction, normalization of glucose metabolism and correction of muscle atrophy. Despite the absence of GAA, the elimination of GYS1 may play an important role in glycogen metabolism.

### Experimental

GAA knockout mice (GAA^{-/-}) were injected with a single IV dose of either 13.5 mg/kg of EEV-PMO-GYS1-1, 27 mg/kg of EEV-PMO-GYS1-1, 27 mg/kg of PMO, or a negative control (vehicle). GYS1 mRNA and protein levels were measured one-week post-injection. Levels of GYS1 were also accessed at one week, two weeks, four weeks, and eight weeks post IV dose of 13.5 mg/kg EEV-PMO-GYS1-2.

### Results

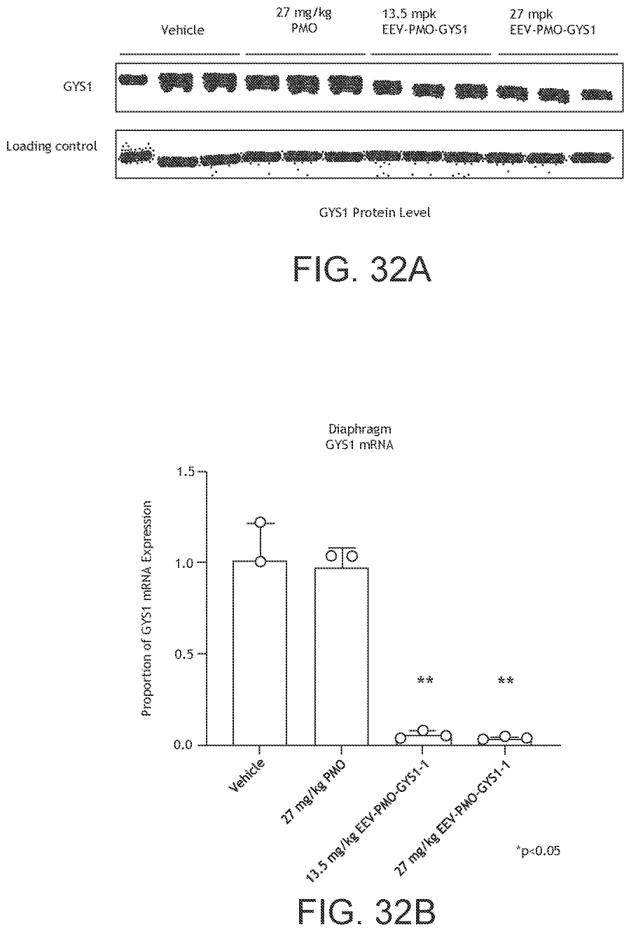

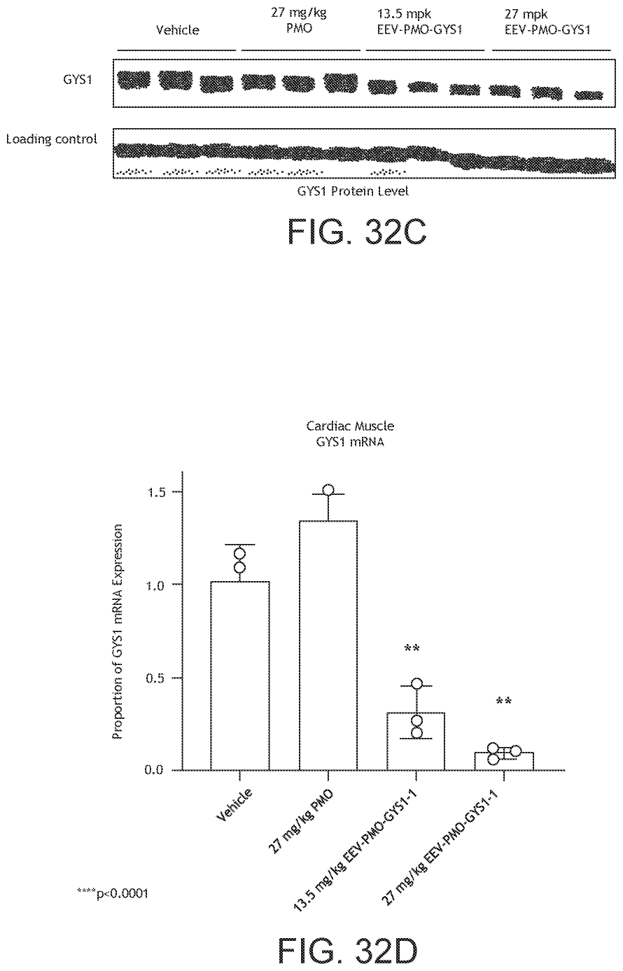

FIGS. 32A-32D show a significant knockdown GYS1 expression in the diaphragm and cardiac muscle in both the EEV-PMO-GYS1-1 and -2 arms, but not in the PMO only arm. This pharmacodynamic result is notable given that this is a single dose experiment administered at very low doses, and it suggests that GYS1 is an addressable target.

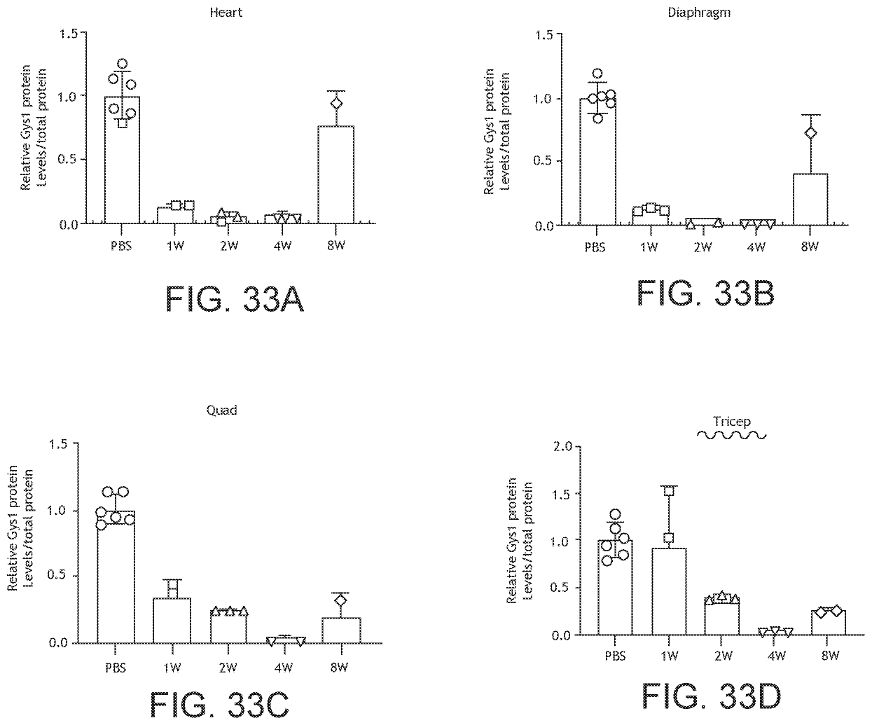

FIGS. 33A-33D show that reduced GYS1 protein levels are sustained for up to eight weeks post injection in the heart, diaphragm, quadriceps, and triceps.

### Example 10. Knockdown of IRF5 Expression Via Exon Skipping

FIG. 31B shows a schematic for knocking down the expression of IRF5 using exon skipping. An EEV-PMO is used to induce exon skipping of exon 4. Skipping exon 4 shifts the reading frame and incudes the reading a premature stop codon. The IRF5 mRNA is then degraded via nonsense mediated decay machinery thereby preventing expression of a full IRF5 protein.

TABLE 25
Compounds Evaluated in this Study

| | | Nucleic Acid Sequence of AC | |
|---|---|---|---|
| ID | Peptide Sequence | (5′-3′) | Chemistry |
| EEV- | Ac-PKKKRKV-AEEA- | AGA ACG TAA TCA TCA GTG GGT | PMO |
| PMO- | K(cyclo[FGFGRGRQ])- | TGG C (SEQ ID NO: 278) | |
| IRF5-1 | PEG₁₂-OH (SEQ ID NO: | | |
| | 274) | | |
| EEV- | Ac-PKKKRKV-AEEA- | AGA ACG TAA TCA TCA GTG GGT | PMO |
| PMO- | K(cyclo[FfϕGrGrQ])- | TGG C (SEQ ID NO: 278) | |
| IRF5-2 | PEG₁₂-OH (SEQ ID NO: | | |
| | 275) | | |
| EEV- | Ac-PKKKRKV-AEEA- | AGA ACG TAA TCA TCA GTG GGT | PMO |
| PMO- | K(cyclo[FGFGRRRQ])- | TGG C (SEQ ID NO: 278) | |
| IRF5-3 | PEG₁₂-OH (SEQ ID NO: | | |
| | 276) | | |
| EEV- | Ac-PKKKRKV-AEEA- | AGA ACG TAA TCA TCA GTG GGT | PMO |
| PMO- | K(cyclo[FGFRRRRQ])- | TGG C (SEQ ID NO: 278) | |
| IRF5-4 | PEG₁₂-OH (SEQ ID NO: | | |
| | 277) | | |

### Experimental

For the in vivo studies, wild type mice were treated with two doses EEV-PMO-IRF5-1 on Days 0 and 3. Samples were collected on Day 7 for qPCR to measure mRNA levels. For the in vitro studies, mouse macrophage cells treated with the EEV-PMO-IRF5-1 or were pre-treated with 2 μM of EEV-PMO-IRF5-1,2,3, or 4 for 4 hours, followed by stimulation with R848, an imidazoquinoline compound that is a specific activator of toll-like receptor (TLR) 7/8, overnight. At 24 hours post treatment, cells were harvested and evaluated by Western Blot.

For each of EEV-PMO-IRF5-1,2,3,4, the PMO was 278 of following sequence 5′-AGA ACG TAA TCA TCA GTG GGT TGG C-3′ (SEQ ID NO: 278).

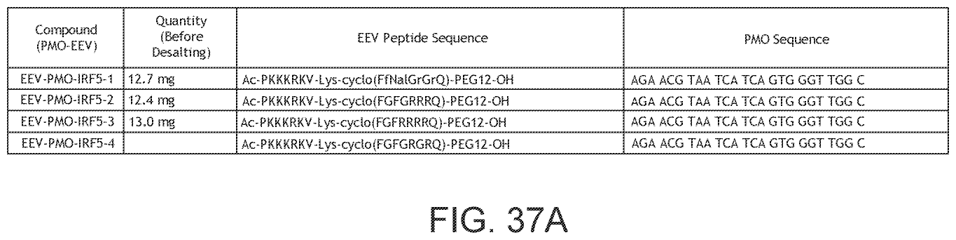

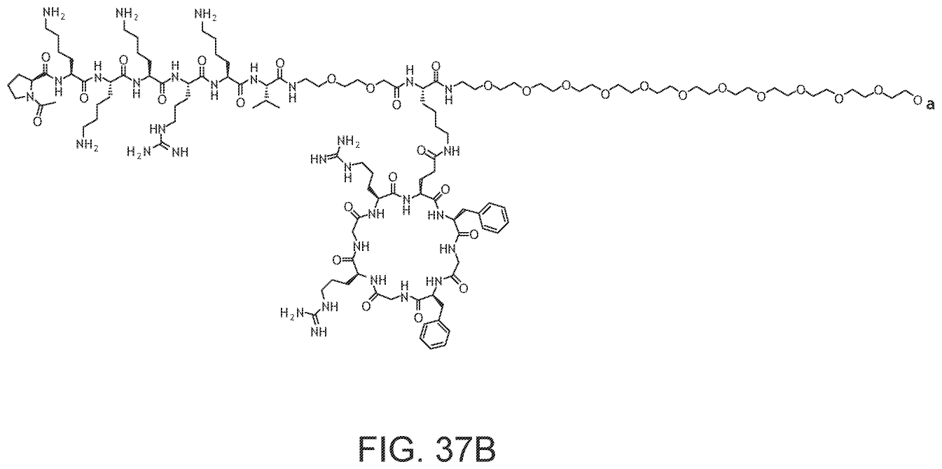

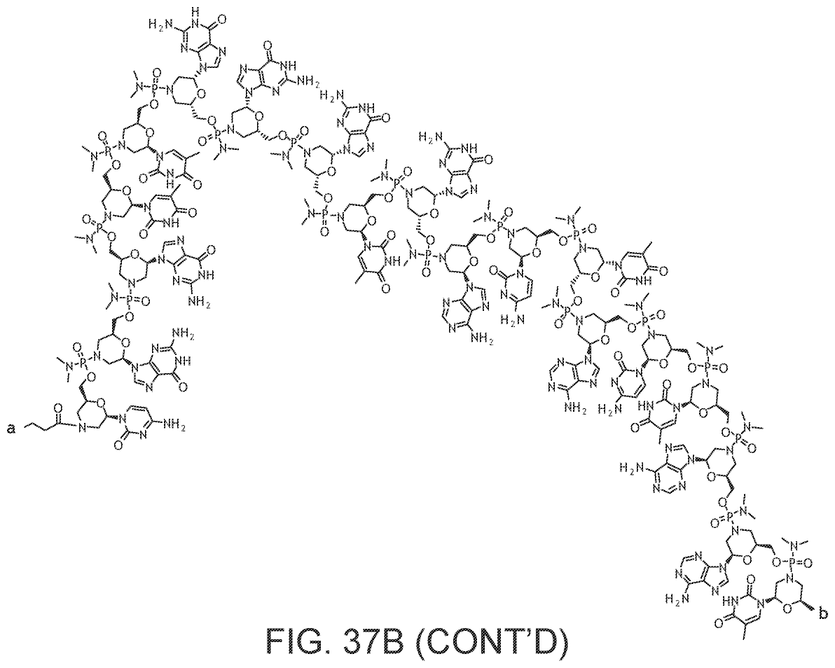

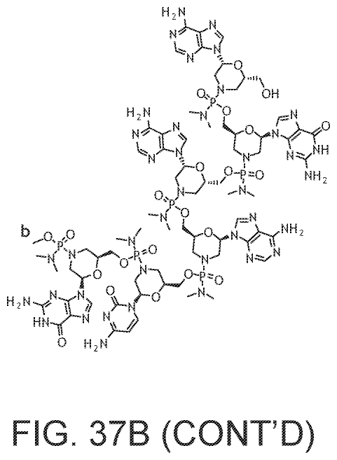

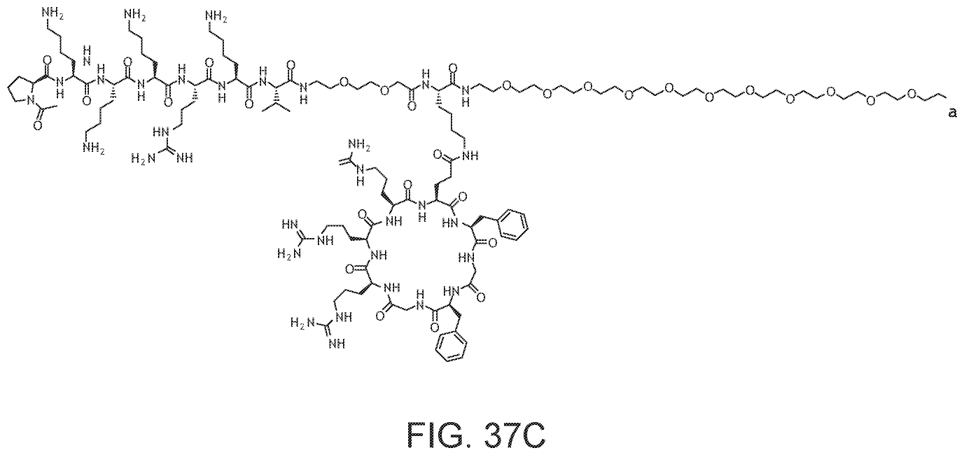

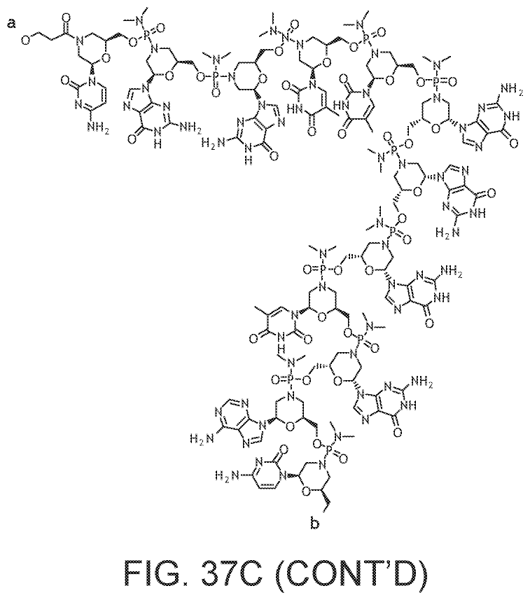

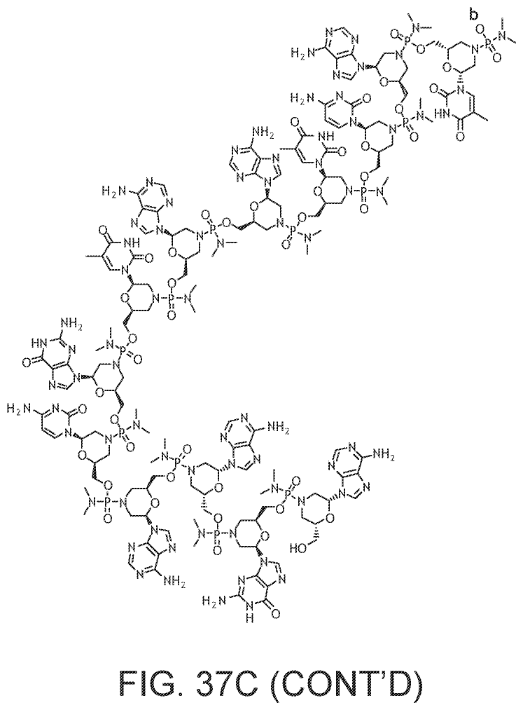

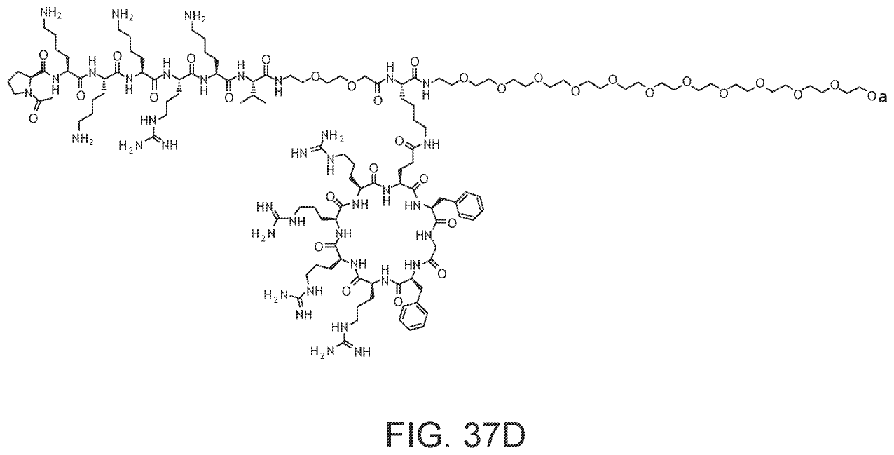

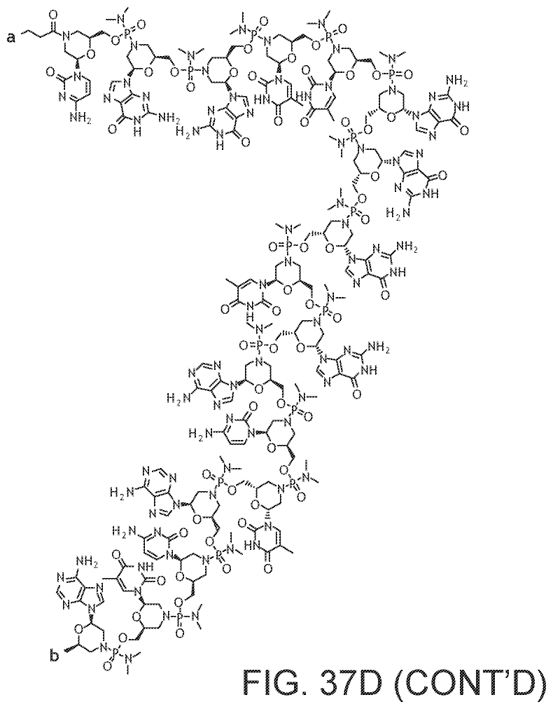

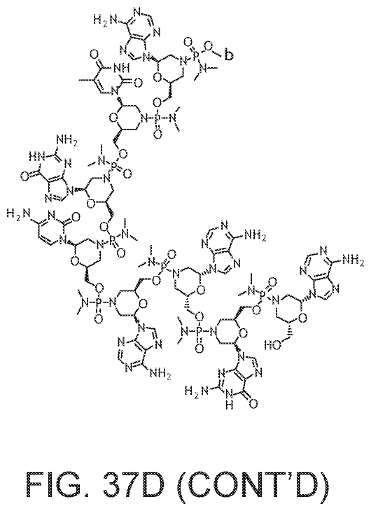

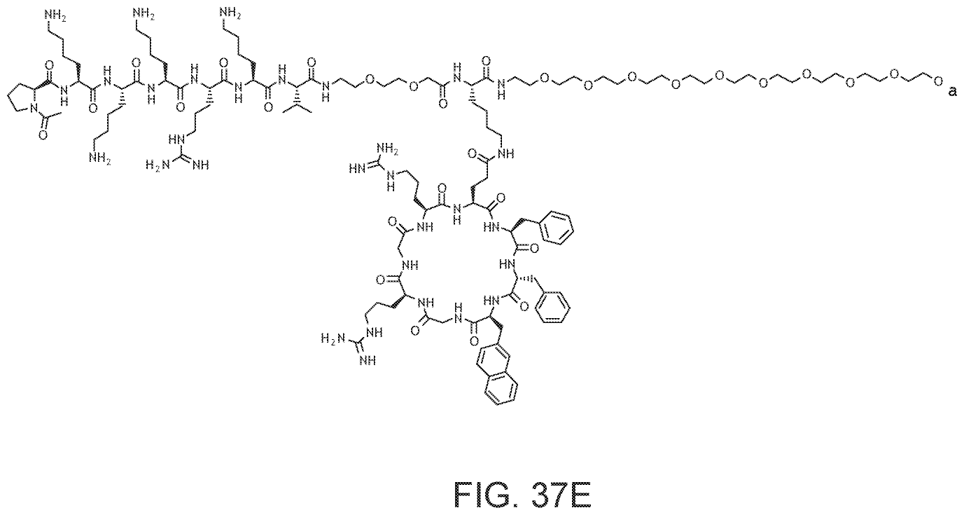

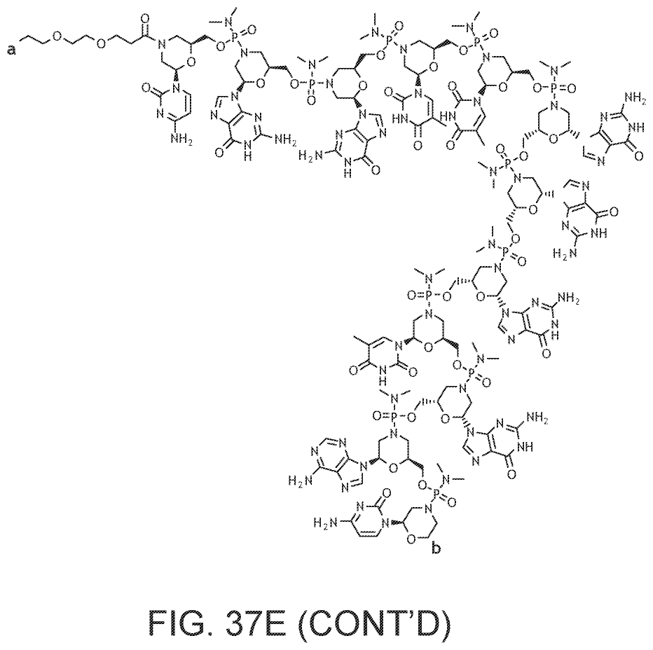

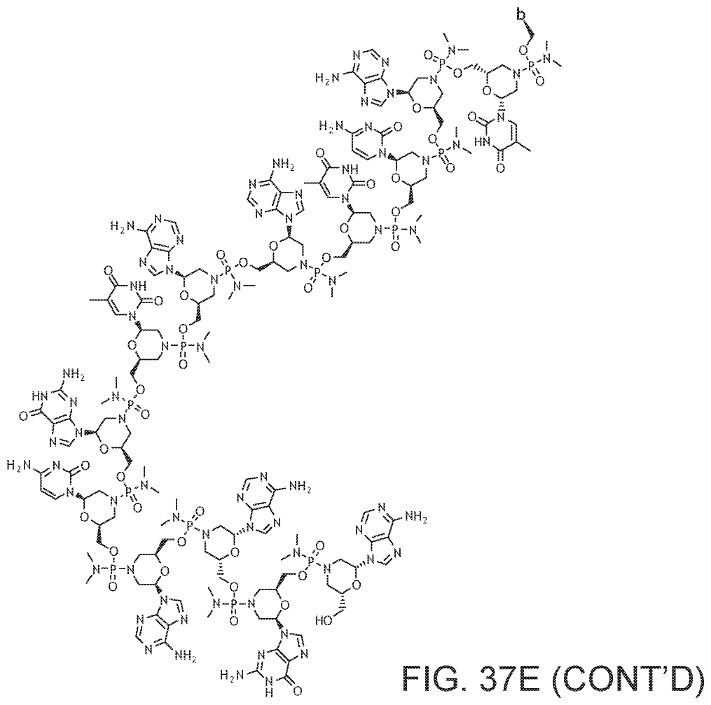

The EEVs for EEV-PMO-IRF5-1,2,3, and 4 are indicated in FIG. 37A-E.

### Results

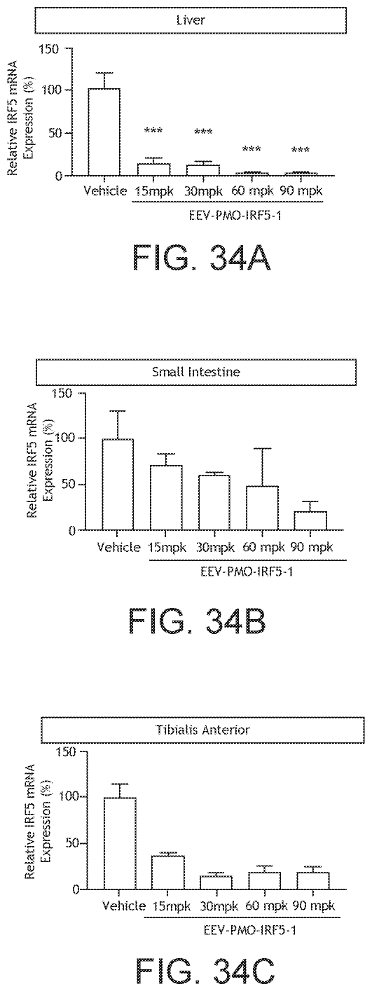

FIGS. 34A-34C show a significant knockdown IRF5 levels the liver (A), small intestine (B), and tibias anterior (C). In all tissues the knockdown was dose dependent.

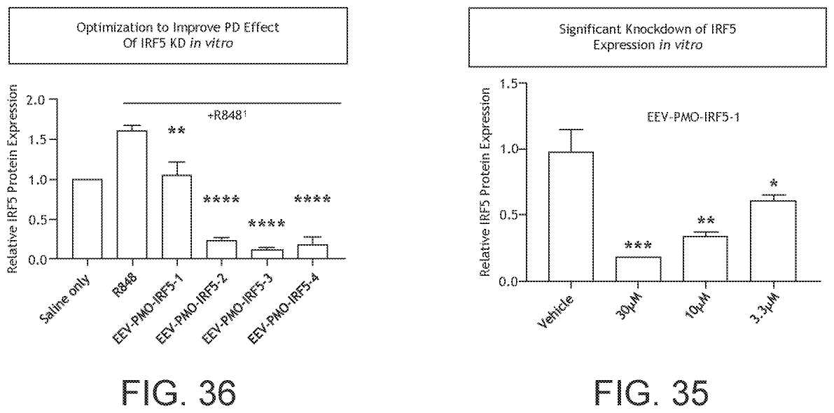

FIG. 35 shows that EEV-PMO-IRF5-2, EEV-PMO-IRF5-3, and EEV-PMO-IRF5-4, had significant improvement in relative potency when compared to EEV-PMO-IRF5-1, as measured by IRF5 protein expression.

FIG. 36 shows that mouse macrophage cells treated with the EEV-PMO-IRF5-1 had a statistically significant reduction of IRF5 protein levels at doses of 30 μM, 10 μM and 3 μM.

### Example 11: Screening EEVs for IRF-5-Targeting PMO In Vitro

RAW 264.7 Monocyte/Macrophage cells were used to evaluate IRF-5 expression and exon skipping after treatment various EEV-PMO constructs shown in FIG. 37A-E.

Briefly, 150K cells/well were seeded in a 24 well plate in 0.5 ml DMEM. After 4 hours, EEV-PMO-IRF5-1,2,3,4 compounds were added to the cells giving a total volume of 500 μL. The cells were then incubated for 24 hours. Following incubation with the EEV-PMO-IRF5-1,2,3,4 compounds, the cells were washed with fresh media then incubated overnight. Following the second incubation, the RNA was harvested and RT-PCR was done using primers that detect exon 5 skipping in the IRF-5 gene.

IRF5 expression levels were determined relative to β-tubulin.

In the IRF-5 expression study, the cells were pre-treated with the EEV-PMO-IRF5-1,2,3,4 compound followed by stimulation with R848 overnight. R484 is a Toll-like receptor agonist and leads to the induction of IRF-5 expression. The total treatment time was 24 hours.

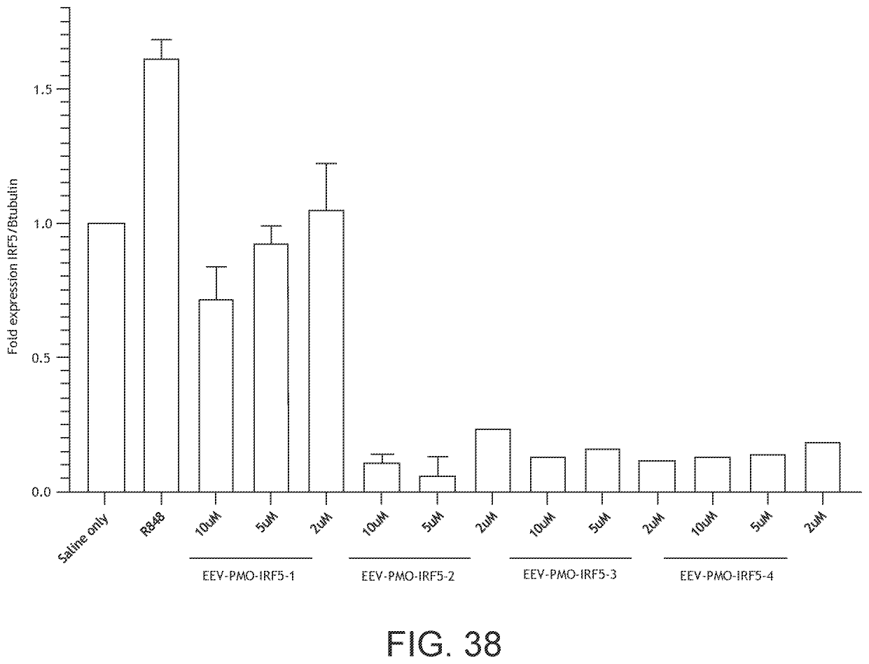

FIGS. 38-39 show the results of the screening study of the present Example.

FIGS. 38 and 40 shows the levels of IRF-5 expression after treatment with the various compounds at various concentrations. R848 significantly increases IRF5 Protein expression in RAW264.7 cells. All EEV-PMO-IRF5-1,2,3,4 treated samples at all the tested concentrations showed a significant reduction in IRF-5 protein expression when compared to cells stimulated with R848. EEV-PMO-IRF5-2,3,4 were on average 5-fold more efficacious than EEV-PMO-IRF5-1 with about 80% IRF-5 protein reduction at concentrations as low as 2 μM when compared to IRF-5 levels in cells stimulation with R848.

FIG. 39 shows the levels of exon skipping after treatment with the various compounds at various concentrations. Compounds EEV-PMO-IRF5-2,3, and 4 exhibited higher exon skipping at 5 μM than EEV-PMO-IRF5-1. No substantial difference in exon skipping was observed between EEV-PMO-IRF5-2, 3, and 4. Knockdown of IRF5 expression via exon skipping

### Example 12: 7 Day Single Dose Range Finding Study for EEV-PMO-DM1-3

The compound evaluated in this study was EEV-PMO-DM1-3, the sequence of which can be found in Example 7, Table 21.

9 week old HSA-LR mice and control FVB mice were employed in a 1 week post single dose range study. The HSA-LR mice were divided into 5 groups. One group was administered saline intravenously and the other groups were administered EEV-PMO-DM1-3 at 15, 30, 60 or 90 mpk of EEV-PMO-DM1-3). Tissues were harvested after 7 days. RT-PCR was used to determine alternative splicing for specific genes (Atp2a1, Clcn1, Nfix, MBNL1). LC-mass was used to determine drug level in Quad, gastro, TA, Triceps, diaphragm, heart, kidney, liver, Brain, plasma. RNA-seq was used to determine the transcription level change between a treated disease model, an untreated disease model and wild-type. Fluorescence imaging was used to determine RNA Foci reduction after treatment with the EEV-oligo compound. Myotonia reduction was recorded 7 days after treatment with EEV-PMO-DM1-3. Q-PCR was used to determine the reduction of mRNA level of actin-HSA after treatment.

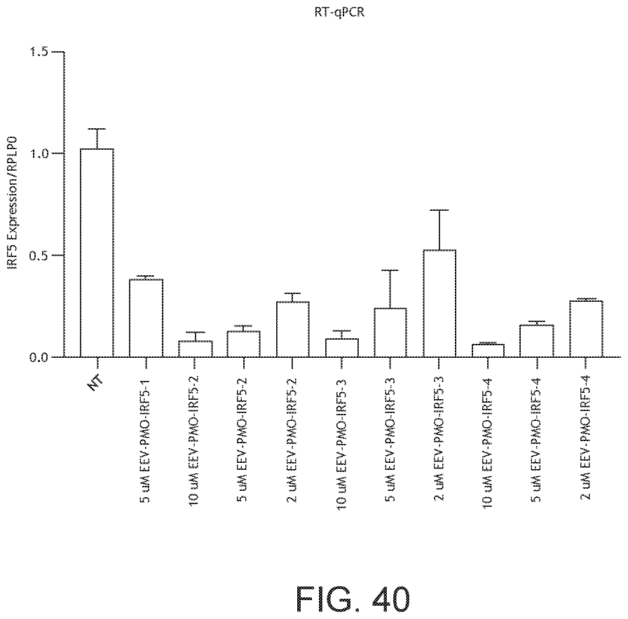

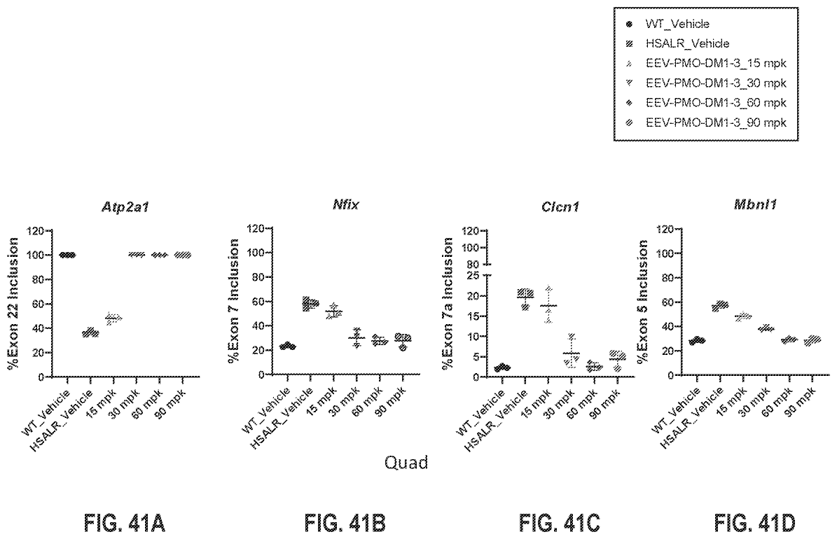

FIG. 41A-D show dose dependent correction of MBNL1 downstream genes in quadriceps 1 week after HSA-LR mice were injected with EEV-PMO-DM1-3; FIG. 41A: Atp2a1, FIG. 41B: Nfix, FIG. 41C: Clcn1, FIG. 41D: Mbnl1.

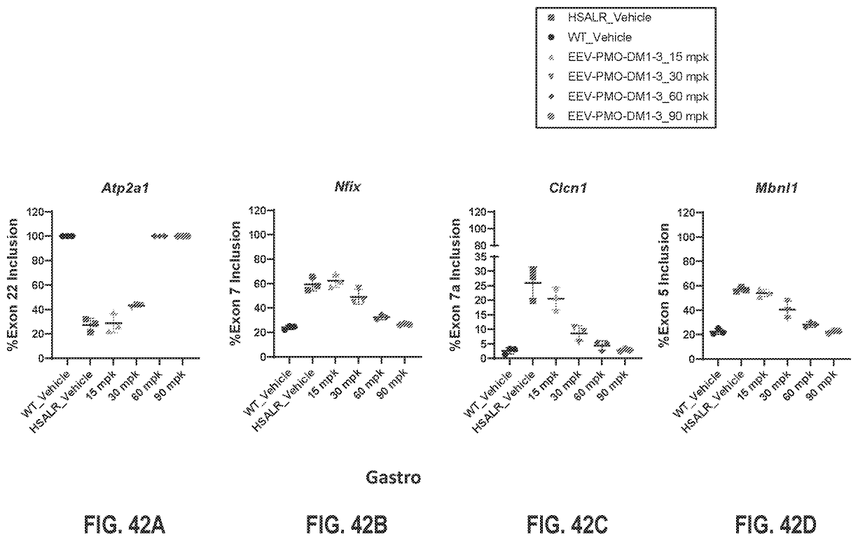

FIG. 42A-D show dose dependent correction of MBNL1 downstream genes in gastrocnemius 1 week after HSA-LR mice were injected with EEV-PMO-DM1-3; FIG. 42A: Atp2a1, FIG. 42B: Nfix, FIG. 42C: Clcn1, FIG. 42D: Mbnl1.

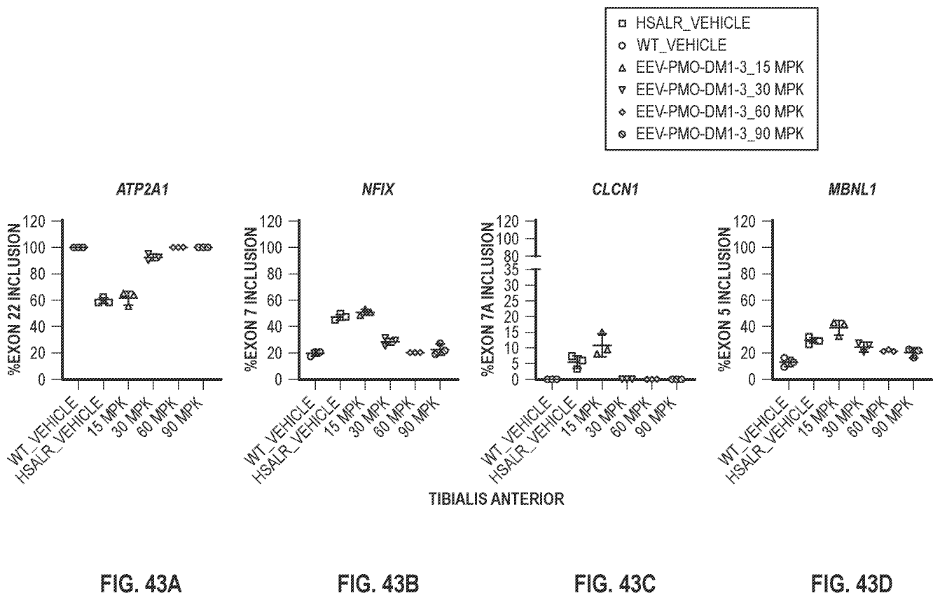

FIG. 43A-D show dose dependent correction of MBNL1 downstream genes in tibialis anterior 1 week after HSA-LR mice were injected with EEV-PMO-DM1-3; FIG. 43A: Atp2a1, FIG. 43B: Nfix, FIG. 43C: Clcn1, FIG. 43D: Mbnl1).

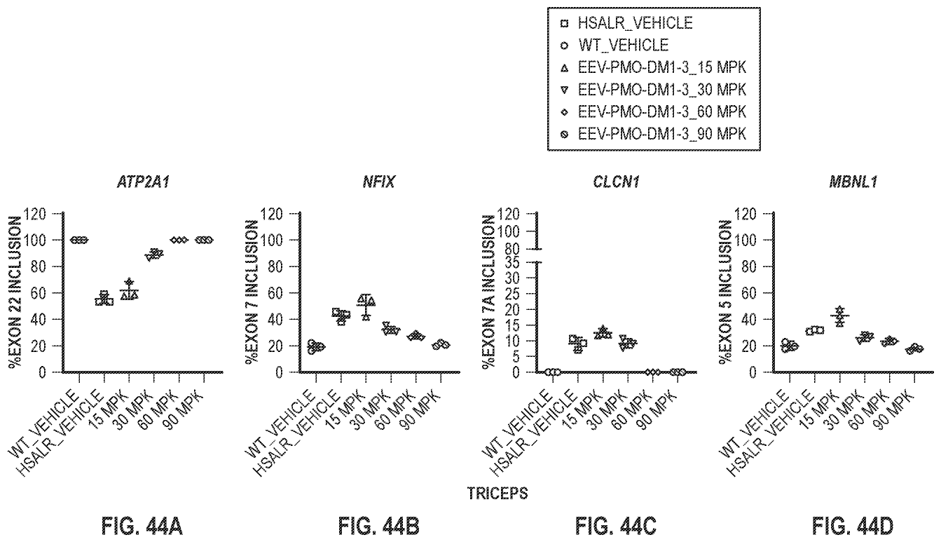

FIG. 44A-D show dose dependent correction of MBNL1 downstream genes in triceps anterior 1 week after HSA-LR mice were injected with EEV-PMO-DM1-3; FIG. 44A: Atp2a1, FIG. 44B: Nfix, FIG. 44C: Clcn1, FIG. 44D: Mbnl1).

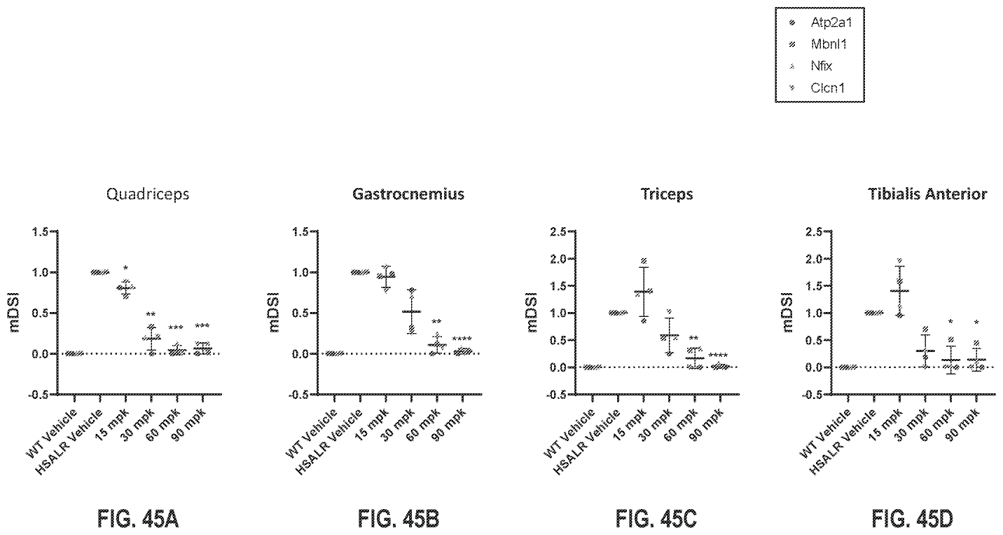

FIG. 45A-D provide an overlay of the data shown in FIGS. 41A-D, 42A-D, 43A-D and 44A-D.

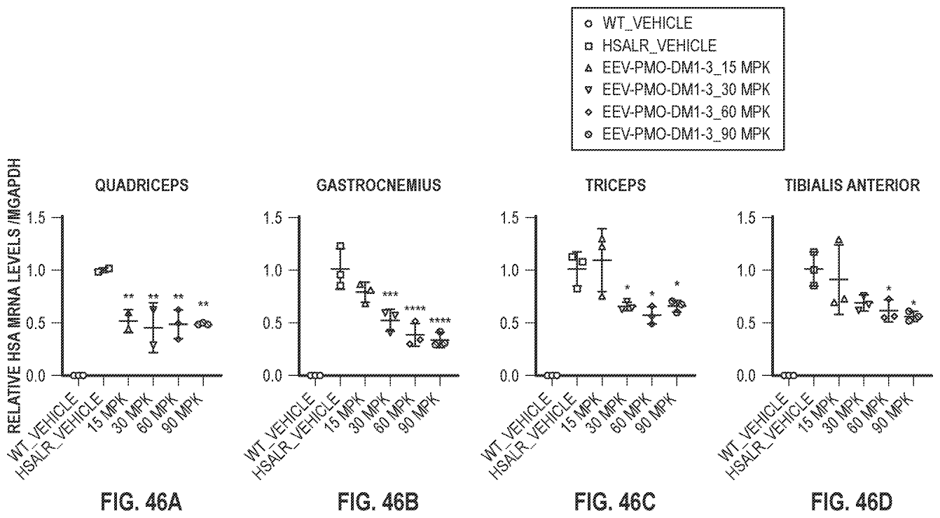

FIG. 46A-D show that administration of EEV-PMO-DM1-3 resulted in ˜50-70% HSA mRNA knockdown in skeletal muscles of HSA-LR mice: FIG. 46A: quadriceps; FIG. 46B: Gastrocnemius; FIG. 46C: Triceps; and FIG. 46D: Tibialis Anterior. Statistical significance is calculated by one-way ANOVA relative to HSALR vehicle treated group (n=3). Dose is based on PMO.

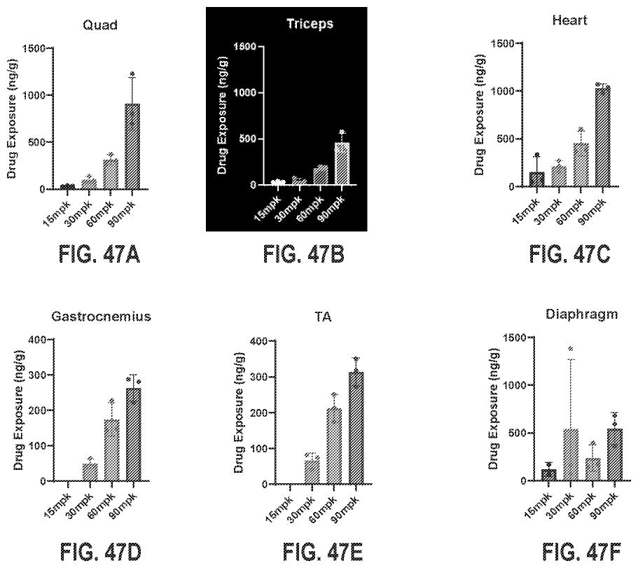

FIG. 47A-F are graphs showing a dose-dependent response for drug levels in various muscle tissues in mice administered EEV-PMO-DM1-3. FIG. 47A: quadriceps; FIG. 47B: triceps; FIG. 47C: heart; FIG. 47D: gastrocnemius; FIG. 47E: tibialis anterior; and FIG. 47F: diaphragm.

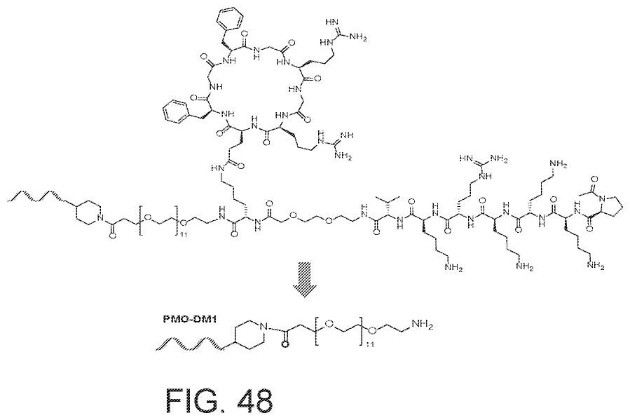

FIG. 48 shows PMO-DM1, the major metabolite detected in vivo.

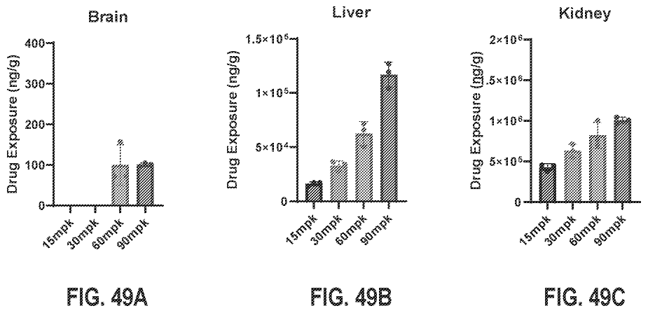

FIG. 49A-C depicts EEV-PMO-DM1-3 exposure in Brain (FIG. 49A), Liver (FIG. 49B) and Kidney (FIG. 49C) after administration at 15, 30, 60 and 90 mpk.

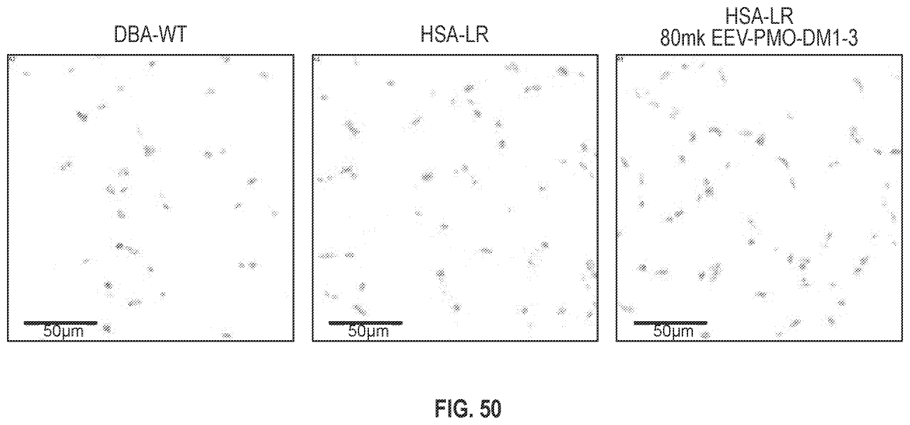

FIG. 50 shows EEV-PMO treatment reduces CUG foci in HSA-LR mouse TA muscle after 1 week.

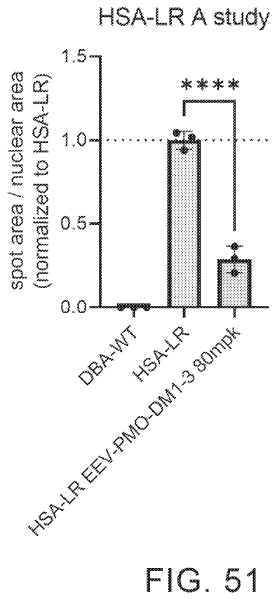

FIG. 51 is a graph showing EEV-PMO treatment reduces CUG foci in HSA-LR mouse TA muscle after 1 week.

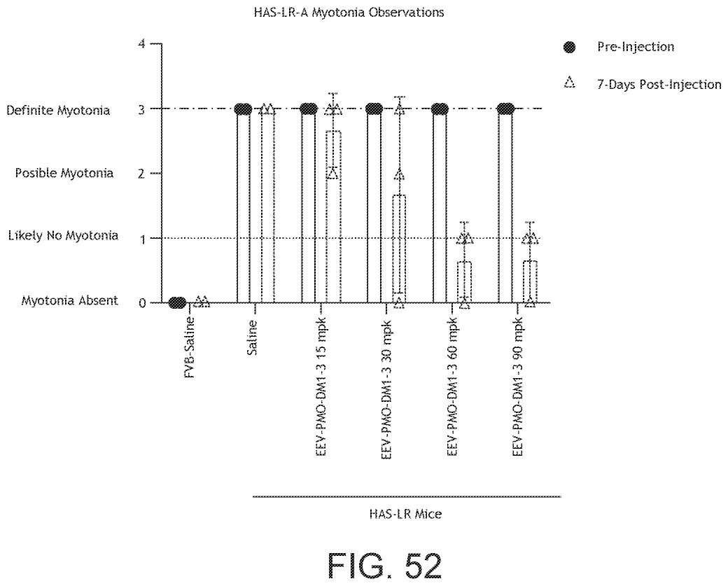

FIG. 52 shows a dose dependent myotonia reduction in HSA-LR mice 7 days after treatment with EEV-PMO-DM1-3 at 15, 30, 60 and 90 mpk.

Myotonia is likely ameliorated 1 week after treatment with EEV-PMO-DM1-3. HSA-LR mice treated with a single dose of 90 mg/kg EEV-PMO-DM1-3 did not exhibit obvious signs of hindlimb myotonia after induction.

### Example 13: 80 Mpk Duration Effect Up to 8 Weeks Using EEV-PMO-DM1-3

The compound evaluated in this study was EEV-PMO-DM1-3, the sequence of which can be found in Example 7, Table 21.

7 week old HSALR mice were administered 80 mpk of EEV-PMO-DM1-3 intravenously and tissues were harvested after 1 week to 8 weeks. RT-PCR was used to determine alternative splicing for specific genes (Atp2a1, Clcn1, Nfix, MBNL1). LC-mass was used to determine drug level in Quad, gastro, TA, Triceps, diaphragm, heart, kidney, liver, Brain, plasma. RNA-seq was used to determine the transcription level change between a treated disease model, an untreated disease model and wild-type. Fluorescence imaging was used to determine RNA Foci reduction after treatment with the EEV-oligo compound. Myotonia reduction was recorded 7 days after treatment with the EEV-oligo compound. Q-PCR was used to determine the reduction of mRNA level of actin-HSA after treatment.

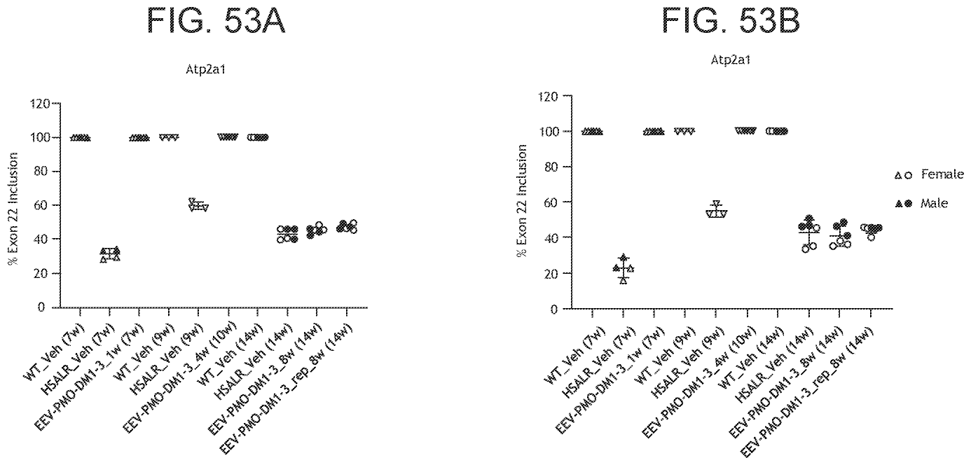

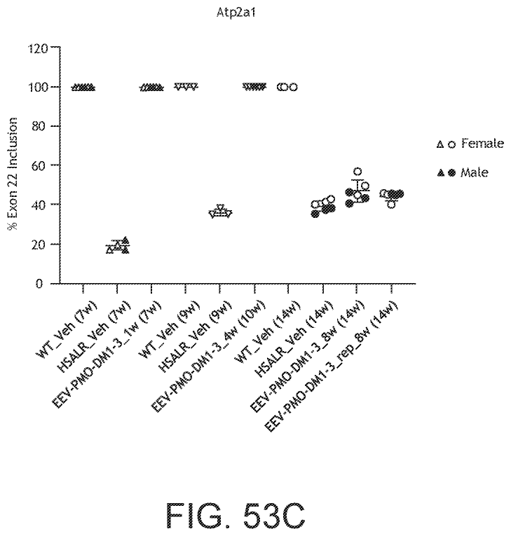

FIG. 53A-C show the duration of effect of 80 mpk EEV-PMO-DM1-3 (60 mpk oligo) on Atp2a1 exon 22 inclusion in HSA-LR mice. Tibialis anterior (FIG. 53A); triceps (FIG. 53B); and quadriceps (FIG. 53C)

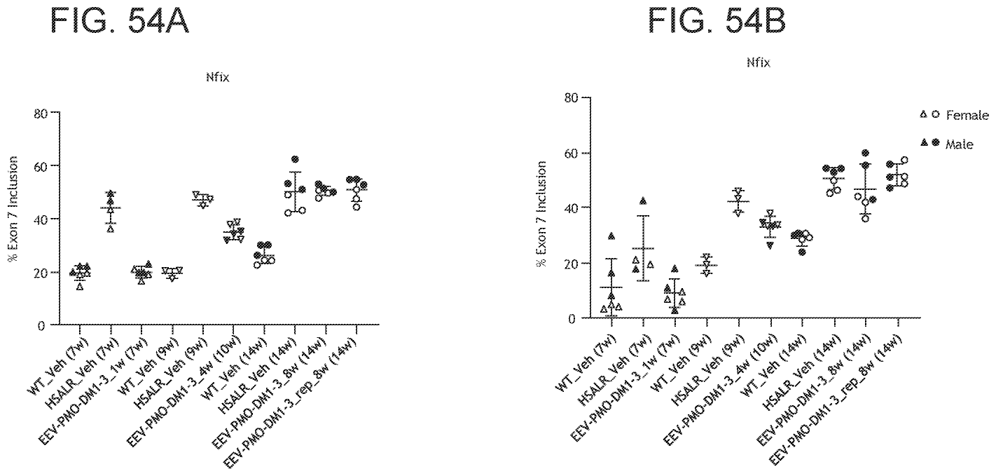

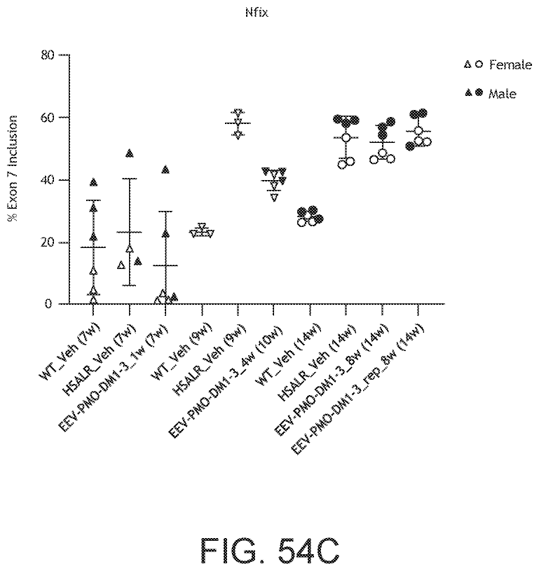

FIG. 54A-C show the duration of effect of 80 mpk EEV-PMO-DM1-3 (60 mpk oligo) on Nfix exon 7 inclusion in HSA-LR mice. Tibialis anterior (FIG. 54A); triceps (FIG. 54B); and quadriceps (FIG. 54C).

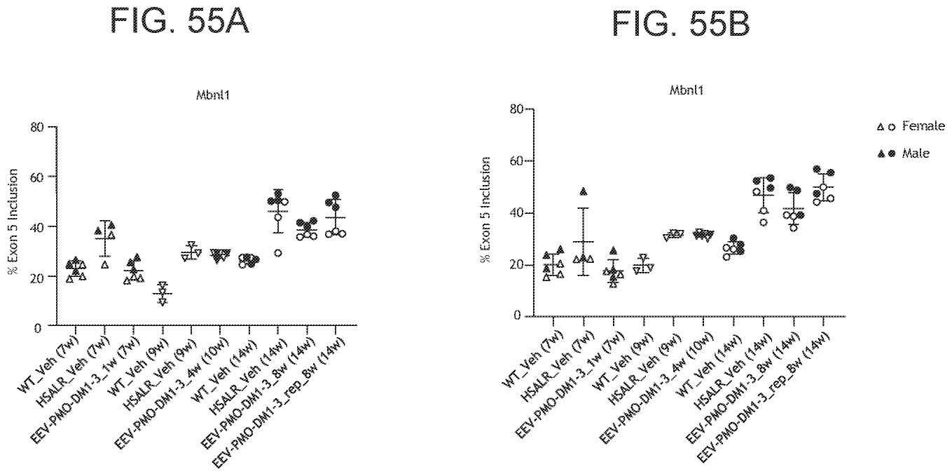

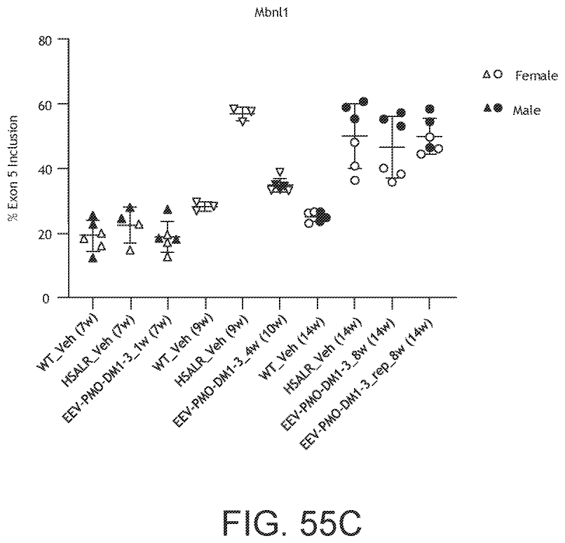

FIG. 55A-C show the duration of effect of 80 mpk EEV-PMO-DM1-3 (60 mpk oligo) on Mbnl1 exon 5 inclusion in HSA-LR mice. Tibialis anterior (FIG. 55A); triceps (FIG. 55B); and quadriceps (FIG. 55C).

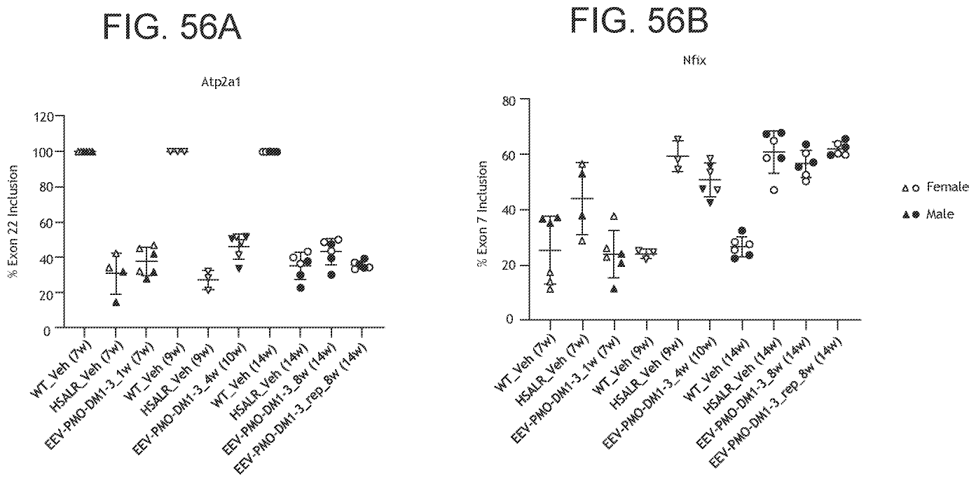

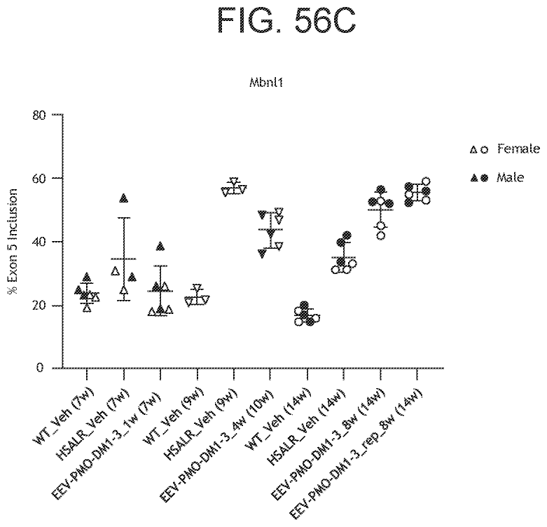

FIG. 56A-C show the duration of effect of 80 mpk EEV-PMO-DM1-3 (60 mpk oligo) on exon 22 inclusion in gastrocnemius of HSA-LR mice. Atp2a1 (FIG. 56A); Nfix (FIG. 56B); and Mbnl1 (FIG. 56C).

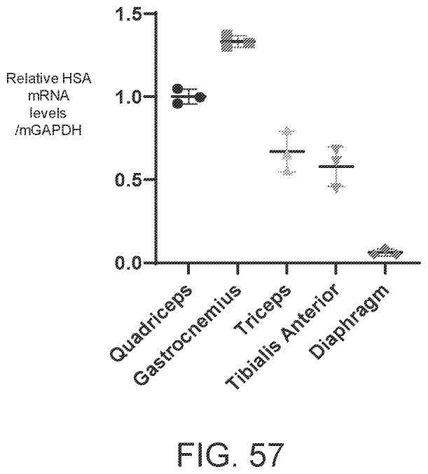

FIG. 57 show the duration of effect of 80 mpk EEV-PMO-DM1-3 (60 mpk oligo) in gastrocnemius, triceps, tibialis anterior and quadricep of HSA-LR mice.

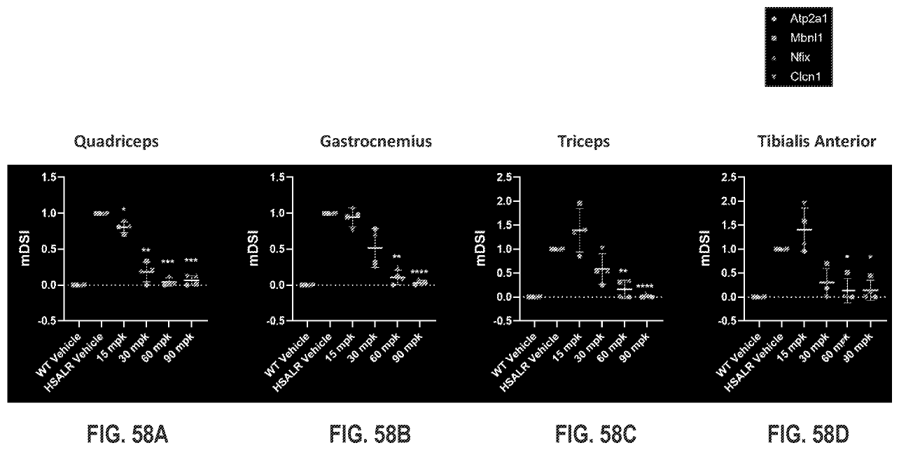

FIG. 58A-D show the duration of effect of 80 mpk EEV-PMO-DM1-3 (60 mpk oligo) in muscle tissue of HSA-LR mice. FIG. 58A: quadriceps, FIG. 58B: gastrocnemius, FIG. 58C: triceps; and FIG. 58D: tibialis anterior.

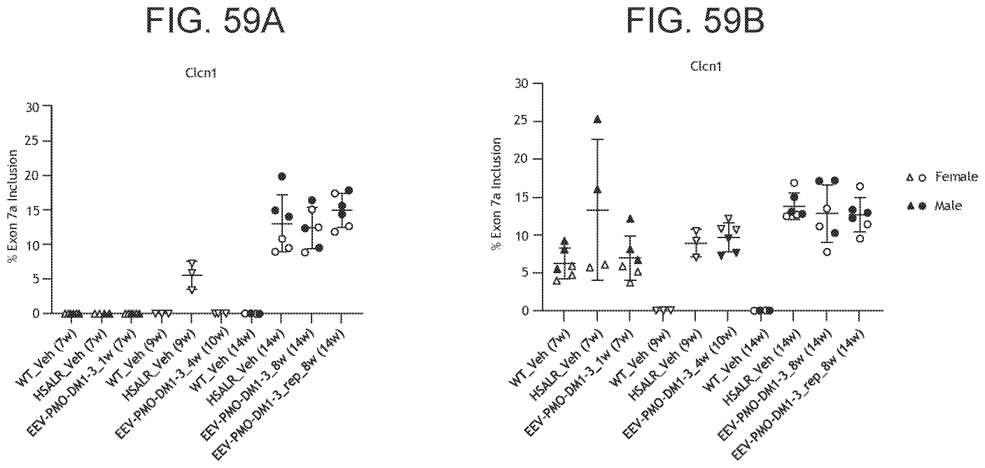

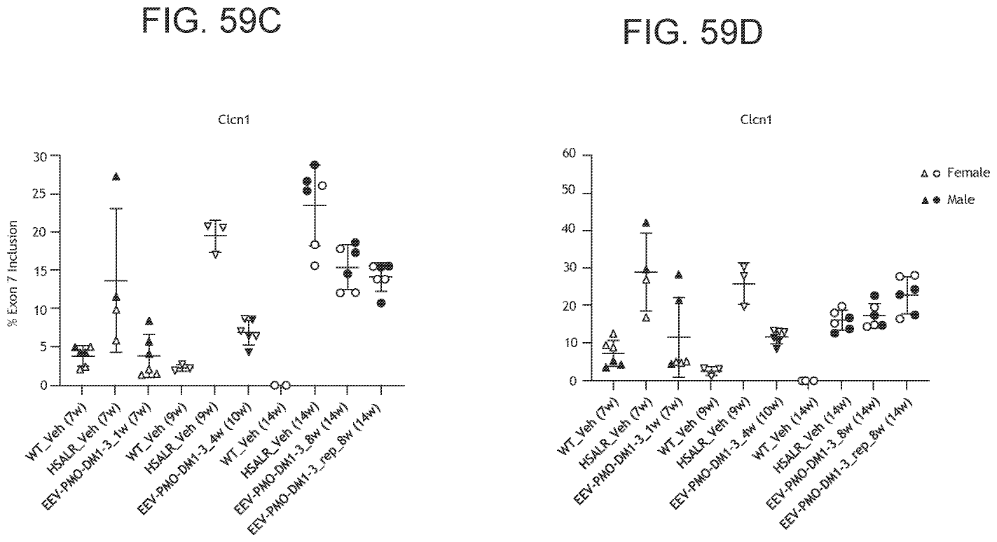

FIG. 59A-D show the duration of effect of 80 mpk EEV-PMO-DM1-3 (60 mpk oligo) on Clcn1 exon 7a inclusion in HSA-LR mice. FIG. 59A: tibialis anterior; FIG. 59B: triceps; FIG. 59C: quadriceps; and FIG. 59D: gastrocnemius.

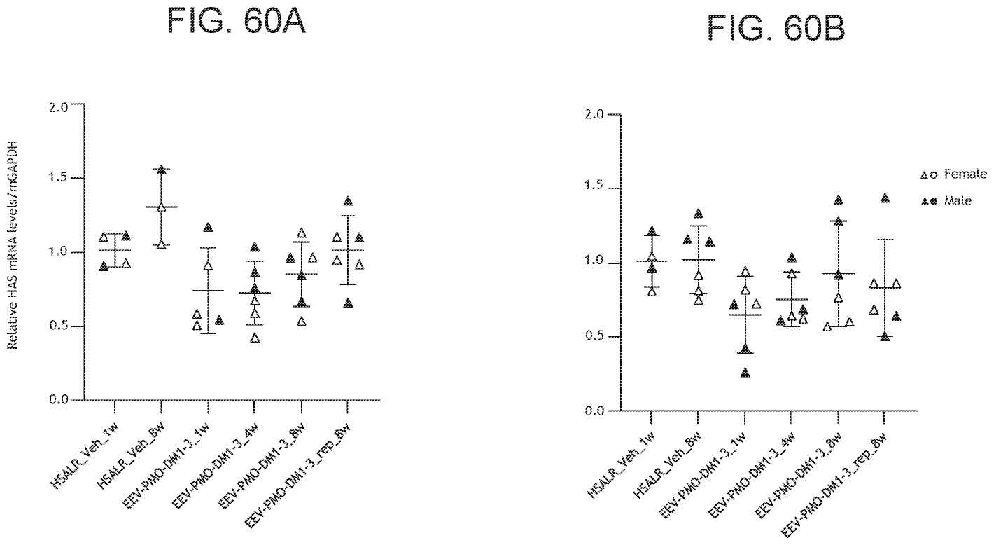

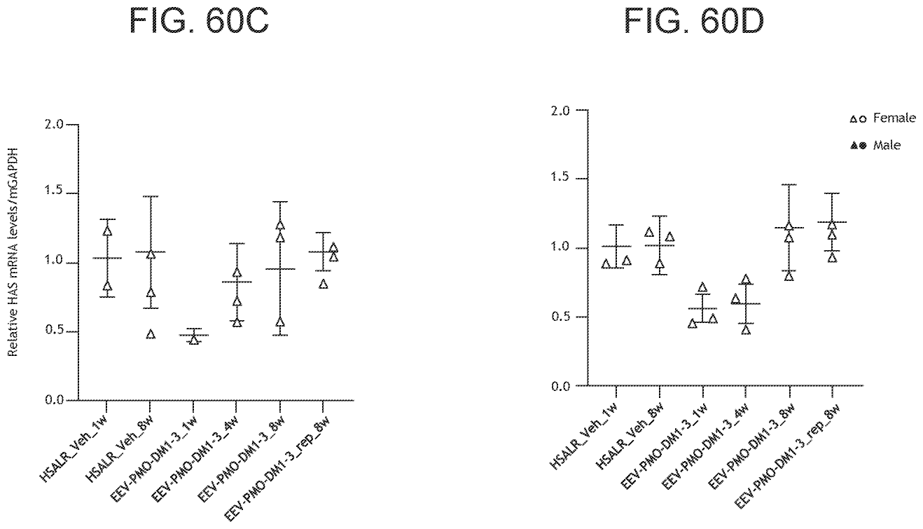

FIG. 60A-D shows EEV-PMO-DM1-3 showed HSA mRNA knockdown trend 1-week and 4-week post-injection. FIG. 60A: tibialis anterior; FIG. 60B: triceps; FIG. 60C: quadriceps; and FIG. 60D: gastrocnemius.

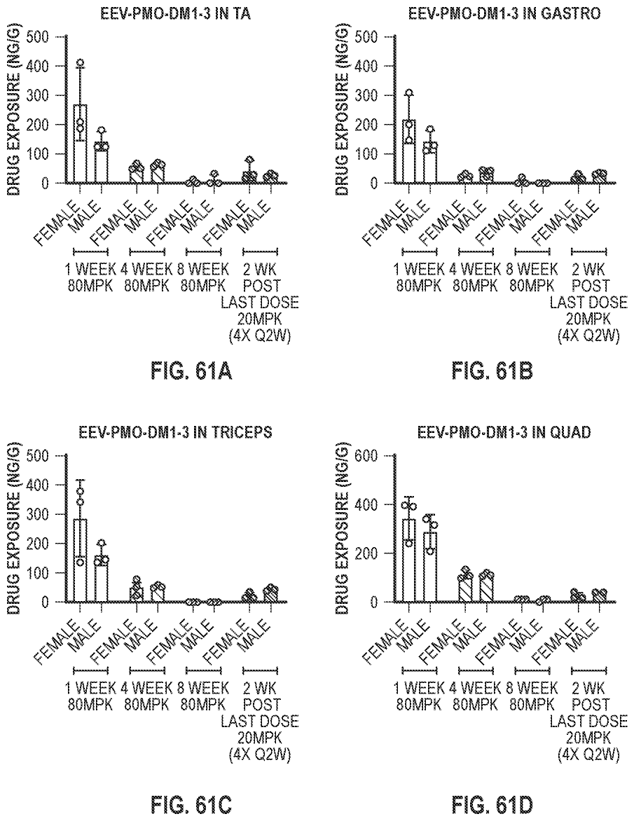

FIG. 61A-D shows a decrease in drug level with 80 mpk EEV-PMO-DM1-3 after 1 week to 4 weeks in muscle tissue. FIG. 61A: tibialis anterior; FIG. 61B: gastrocnemius; FIG. 61C: triceps; and FIG. 61D: gastrocnemius. EEV-PMO-DM1-3 (60 mpk oligo, 80 mpk whole drug) fully correct mis-splicing in gastrocnemius, triceps, tibialis anterior and quadricep post 1 week treatment.

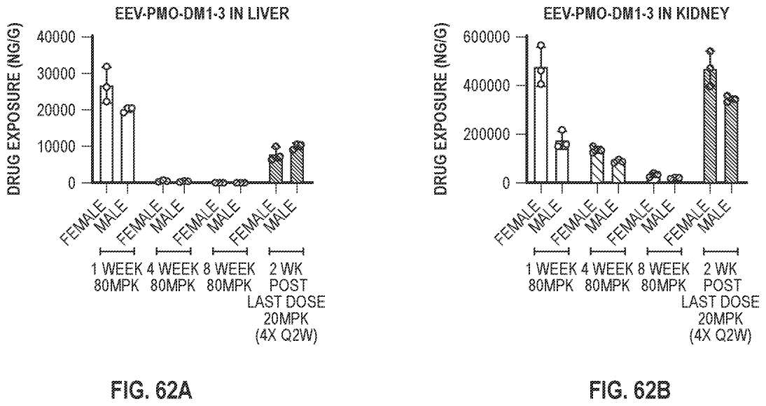

FIG. 62A-B shows a decrease in drug levels was observed with 80 mpk dose of EEV-PMO-DM1-3 after 1 week to 4 week in liver and kidney.

A similar experiment was performed to evaluate intravenous administration of EEV-PMO-DM1-3 for a longer duration and at a higher dose. 8 week old HSALR mice were administered 40, 60, 80 or 120 mpk of EEV-PMO-DM1-3 intravenously and tissues were harvested after 4 to 12 weeks. RT-PCR was used to determine alternative splicing for specific genes (Atp2a1, Clcn1, Nfix, MBNL1). LC-mass was used to determine drug level in Quad, gastro, TA, Triceps, diaphragm, heart, kidney, liver, Brain, plasma. RNA-seq was used to determine the transcription level change between a treated disease model, an untreated disease model and wild-type. Fluorescence imaging was used to determine RNA Foci reduction after treatment with EEV-PMO-DM1-3. Myotonia reduction was recorded 7 days after treatment with EEV-PMO-DM1-3. Q-PCR was used to determine the reduction of mRNA level of actin-HSA after treatment. A similar trend in data was observed (data not shown)

### Example 14: Treatment of Patient Derived DM1 Cells with EEV-PMO-DM1-3

The compound evaluated in this study was EEV-PMO-DM1-3, the sequence of which can be found in Example 7, Table 21.

Patient myoblasts were treated with 30 micromolar of EEV-oligo throughout 4 days of differentiation. Splicing correction was assessed by one-step RT-PCR. HCR-FISH and sequestered MBNL1 protein detection assays were used to detect RNA foci. Results: EEV-PMO-DM1-3 promotes significant biomarker splicing correction and a reduction in nuclear foci in DM1 patient-derived muscle cells.

Patient myoblasts harbor 2600 CTG repeats within the DMPK 3′UTR. Free uptake of 30 μM EEV-PMO-DM1-3 was throughout 4 days of differentiation. Splicing correction was assessed by one-step RT-PCR and Labchip analysis. Plotted mean+SD; n=4 HCR-FISH assay for CUG foci detection in place.

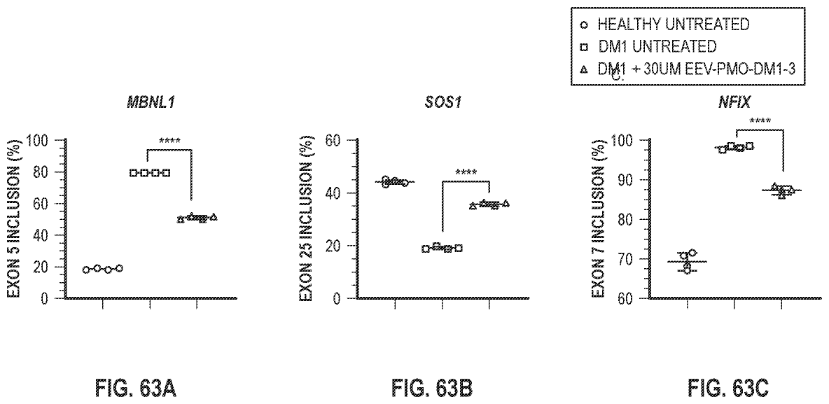

FIG. 63A-C shows that EEV-PMO-DM1-3 promotes significant biomarker splicing correction in DM1 patient-derived muscle cells.

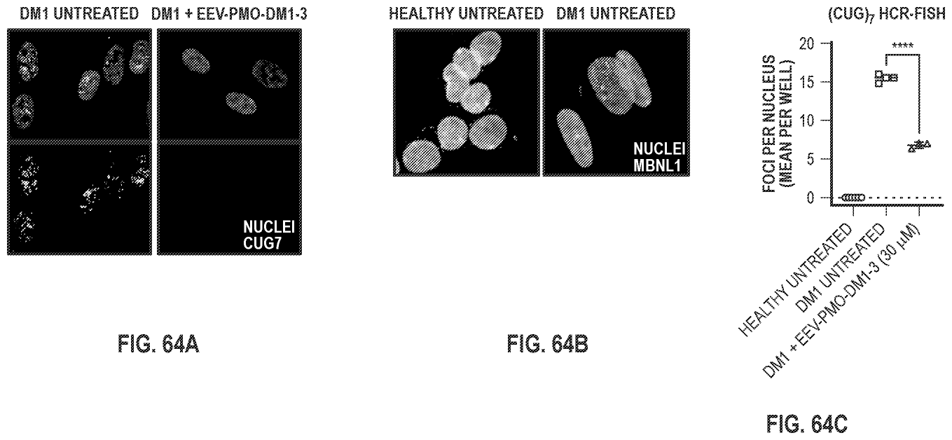

FIG. 64A-C shows that EEV-PMO-DM1-3 reduces nuclear foci in DM1 patient-derived muscle cells.

### Example 15: Cytotoxicity Screening of EEV-PMO-DM1-3 in Renal Cells

The compound evaluated in this study was PMO-DM1 or EEV-PMO-DM1-3, the sequences of which can be found in Example 7, Table 21.

Human Primary Renal Proximal Tubular Epithelial Cells (RPTECs) were exposed to varying concentrations (1:2 serial dilution in saline with a final dilution factor of 4× from about 6 to about 800 micromolar) of PMO-DM1 and EEV-PMO-DM1-3 for 24 hours and screened for viability. Melittin was used as a positive control at 16.6 uM.

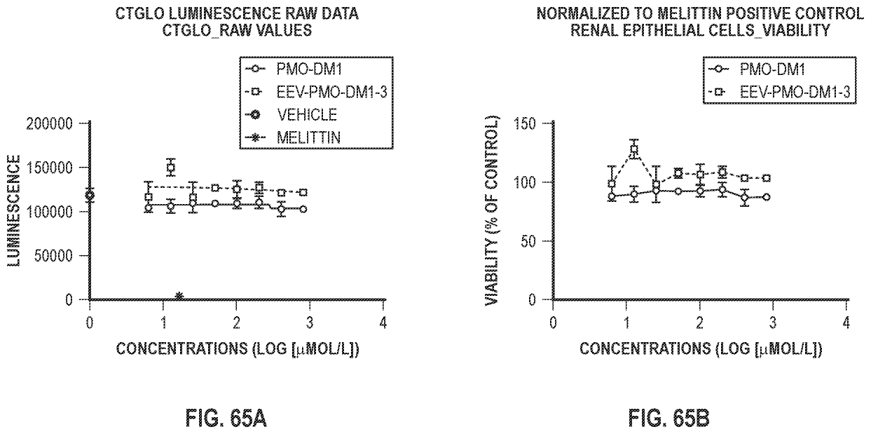

FIG. 65A-B show that PMO-DM1 or its conjugated EEV-PMO-DM1-3 did not show any toxicity even with the highest concentration of 817 uM or 797 uM, respectively.

### Example 16. Use of Cell-Penetrating Peptide Coupled to an Oligonucleotide and Nuclear Localization Sequence for Splicing Correction of Exon 23 of DMD in an MDX Mouse Model

Purpose. This study employs an MDX mouse model, a model of DMD, to study the effect of compositions comprising an AC, a CPP, and a nuclear localization sequence (NLS, or modulatory peptide, MP) on dystrophin expression and muscle fiber damage.

Preparation and design of CPP-PMO targeting murine DMD exon 23. Design of antisense compounds (AC) that target DMD exon 23 are shown below in Table 26. Design of CPP-NLS-PMO constructs (ENTR-0164, ENTR-0165, ENTR-0201) are shown below in Table B2.

TABLE 26
Compounds that target DMD

| Oligo# | Design | Target |
|---|---|---|
| ENTR-0013 | 5′-GGC CAA ACC TCG GCT TAC CTG AAA T-3′ (all | Murine DMD |
| | PMO monomers) (SEQ ID NO: 139) | exon 23 |
| ENTR-0017 | 5′-GGC CAA ACC TCG GCT TAC CTG AAA T-3′-C3-NH2 | Murine DMD |
| | (all PMO monomers) (SEQ ID NO: 139) | exon 23 |
| ENTR-0066 | Cyclooctyne-5′-GGC CAA ACC TCG GCT TAC CTG AAA | Murine DMD |
| | T-3′ (all PMO monomers) (SEQ ID NO: 139) | exon 23 |
| ENTR-0068 | NH2-5′-C3-GGCCAAACCTCGGCTTACCTGAAAT-3′- | Murine DMD |
| | C3-NH2 (all PMO monomers) (SEQ ID NO: 139) | exon 23 |
| ENTR-0149 | 5′-GGC CAA ACC TCG GCT TAC CTG AAA T-3′-C4- | Murine DMD |
| | cyclooctyne (all PMO monomers) (SEQ ID NO: 139) | exon 23 |

TABLE 27
CPP-NLS-PMO Compounds that target DMD

| Oligo# | Design | Peptide Fusion |
|---|---|---|
| ENTR- | 5′-GGC CAA ACC TCG GCT TAC CTG | NLS: PKKKRKV |
| 0164 | AAA T-3′-C3NH-PEG4-BCN+K(N3)- | (SEQ ID NO: 103) |
| | miniPEG-NLS-ss-bA-bA-CPP12 (all PMO | |
| | monomers) (SEQ ID NO: 139) | |
| ENTR- | 5′-GGC CAA ACC TCG GCT TAC CTG | NLS: PKKKRKV |
| 0165 | AAA T-3′-C3NH-PEG4-BCN+Ac-NLS- | (SEQ ID NO: 103) |
| | K(CPP12)-PEG12-K(N3) (all PMO | |
| | monomers) (SEQ ID NO: 139) | |
| ENTR- | 5′-GGC CAA ACC TCG GCT TAC CTG | NLS: PKKKRKV |
| 0201 | AAA T-3′-click-K-PEG12-K(CPP12)-NLS- | (SEQ ID NO: 103) |
| | Ac (all PMO monomers) (SEQ ID NO: 139) | |

For 3′ or 5′ conjugation via click reaction (ENTR-0164, ENTR-0165, ENTR-0201), a solution of peptide-azide in nuclease-free water (1 mM) was added to the PMO-3′-cyclooctyne or cyclooctyne-5′-PMO solids. The mixture was vortexed to dissolve the peptide-PMO conjugate, centrifuged to settle the solution, and incubated at room temperature for 8-12 hours for completion as confirmed by LCMS (Q-TOF). For purifications, crude mixtures were diluted with DMSO, loaded onto a C18 reverse-phase column (150 mm*21.2 mm), flow rate of 20 mL/min and purified by an appropriate gradient using water with 0.05% TFA and acetonitrile as solvents. Desired fractions were pooled, pH of the solution was adjusted to 5-6 by 1M NaOH and the solution underwent the lyophilization process, affording white lyophilized powder. For in vitro and in vivo formulations, the conjugates were reconstituted in appropriate amount of PBS or Saline for the desired concentration (2-10 mg/mL). Concentration of the non LSR labeled conjugates were measured by preparing 10, 20, and 50-fold dilutions in formulated buffer and reading the absorbance at 260 nm or 280 nm using a nanodrop. Once the linear range of dilution achieved, the absorbance was measured in triplicates and concentration was calculated using the average absorbance and ε₂₆₀ or ε₂₈₀. ε₂₈₀ for conjugates were calculated by the following formula: ε₂₈₀=138993+(n*3550); n=number of CPP. The diluted samples were analyzed by LCMS (Q-TOF) for the conjugate identity confirmation. Table below summarizes the calculated MW and experimental MWs. All experimental MWs reasonably matched the calculated average MW with expected ±6 Da assay variation.

| | | Experimental | |
|---|---|---|---|
| Name | Calculated MW | Average MW | Purity |
| ENTR-0013 | 8413.1 | 8410.02 | 99 |
| ENTR-0017 | 8487 | 8484 | 95 |
| ENTR-0066 | 9001.7 | 9003.8 | 95 |
| ENTR-0068 | 8751.07 | 8748.02 | 85 |
| ENTR-0149 | 8635.07 | 8635.8 | 82 |
| ENTR-0164 | 11838.19 | 11836.3 | 97 |
| ENTR-0165 | 11944.31 | 11942.3 | 99 |
| ENTR-0201 | 11669.5 | 11669.5 | >99 |

The structures of examples of compounds comprising antisense oligonucleotides (underlined; also shown in ENTR-0013) that target murine DMD and CPPs are shown below.

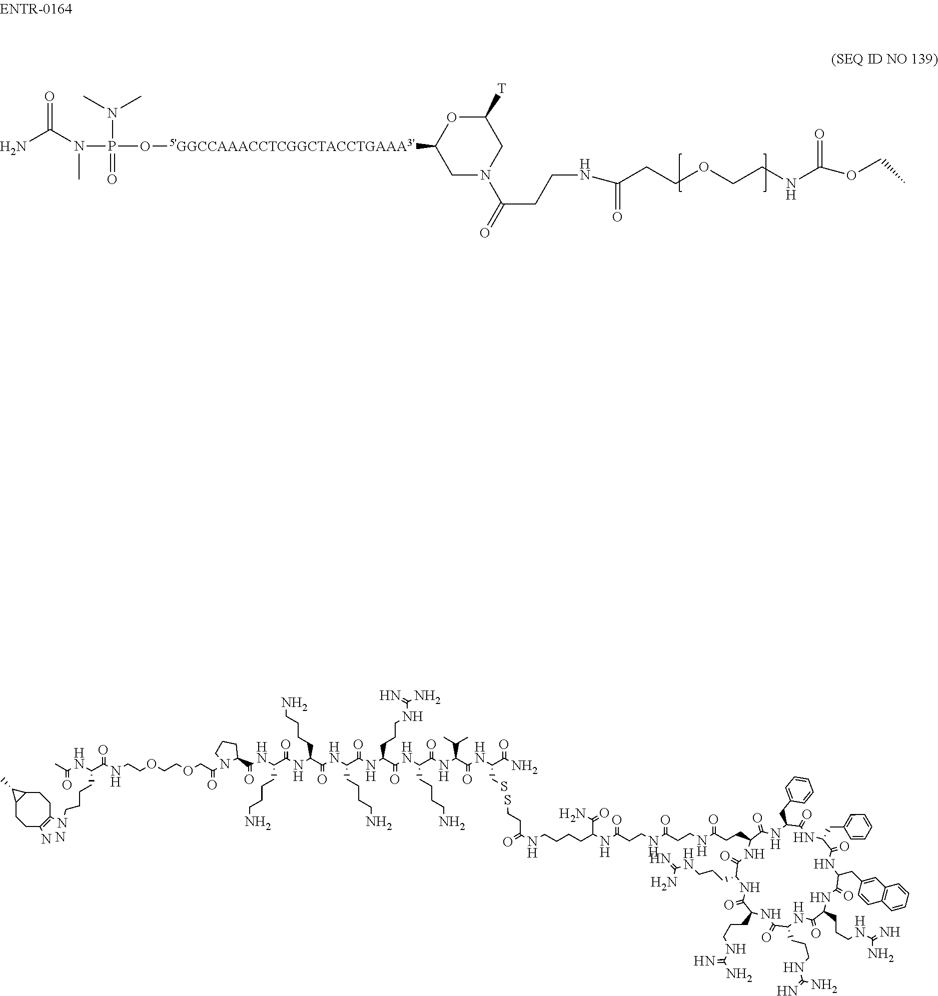

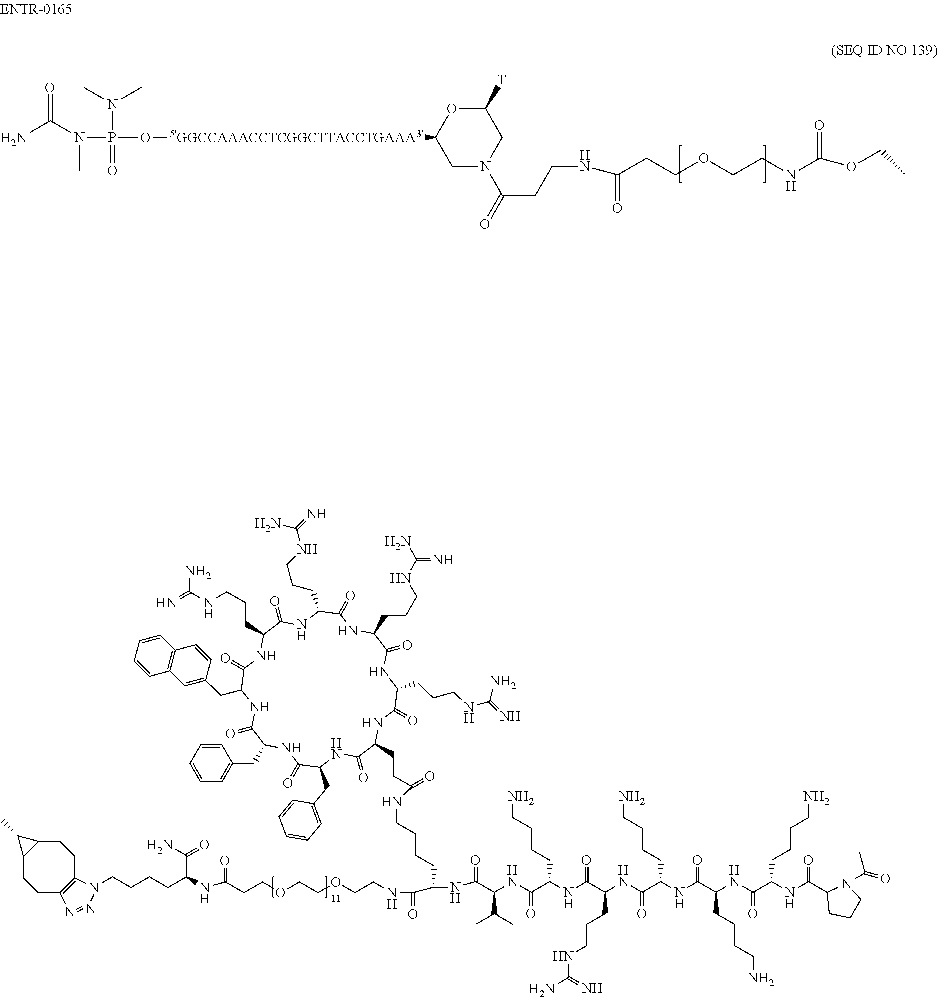

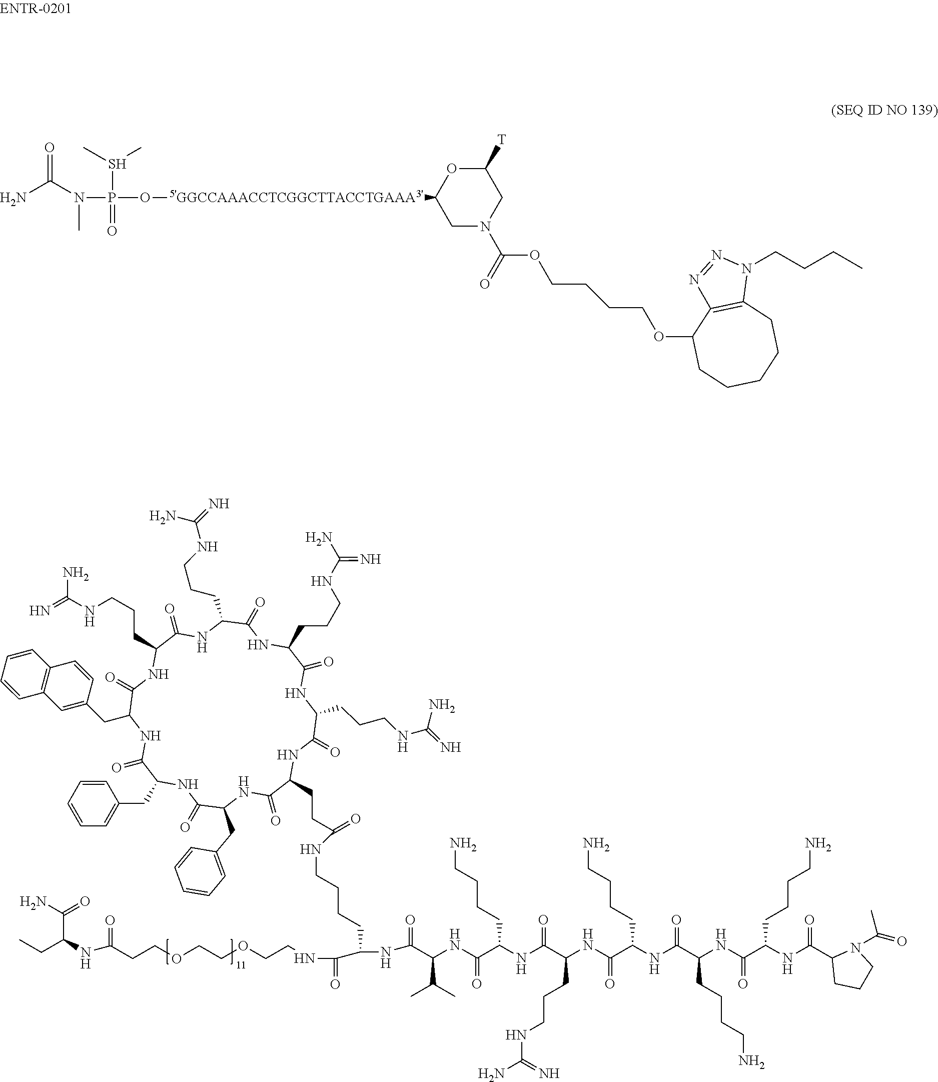

Study design. Compositions comprising an AC having a sequence of 5′-GGCCAAACCTCGGCTTACCTGAAAT-3′ (SEQ ID NO: 139), a cCPP12 (amino acid sequence is FfΦRrRr (SEQ ID NO: 279)), and a nuclear localization sequence PKKKRKV (SEQ ID NO: 103) (referred to herein as “ENTR-201”) are applied to MDX mice to evaluate the ability of the compositions to skip exon 23 and thus treat DMD. A control composition lacks cCPP12 and a nuclear localization sequence. The sequence of the AC of the control composition is 5′-GGC CAA ACC TCG GCT TAC CTG AAA T-3′ (SEQ ID NO: 139). The ENTR-201 composition is administered to the mice intravenously (IV) at a dose of 10 mg/kg once per week for four weeks or at a dose of 20 mpk once. The control composition is administered to mice intravenously (IV) at a dose of 20 mpk. Total RNA is extracted from tissue samples and analyzed by RT-PCR and protein is extracted from tissue sample and analyzed by Western Blot to visualize the efficiency of splicing correction and to detect dystrophin products. The percentage of exon 23 corrected products is evaluated. The dystrophin protein level is evaluated with respect to alpha-actinin (loading control) as well as in comparison to dystrophin expression in wild-type mice. Serum levels of creatine kinase, which is increased in DMD patients as a result of muscle fiber damage, were also evaluated by a commercially available kit purchased from Sigma Chemicals.

Evaluations of peptide fusions to the CPP12-PMO construct. To further increase the functional delivery of PMO, we also explored various peptides fusions for the CPP-PMO constructs. For one example, T9 peptide (SKTFNTHPQSTP (SEQ ID NO: 220)) (Y. Seow et al./Peptides 31 (2010) 1873-1877) and muscle specific peptide (MSP peptide, ASSLNIA (SEQ ID NO: 280) (Gao et al. Molecular Therapy (2014) 22, 7: 1333-1341) which have been demonstrated to enhanced muscle targeting were also fused to CPP12 construct as in ENTR-0119 and ENTR-0163 respectively. Neither of these peptide fusions improved the activity in MDX mice.

Notably, CPP12 with a Nuclear Localization Sequence (PKKKRKV (SEQ ID NO: 103)) outperformed CPP12 alone significantly (e.g. ENTR-164, ENTR-0165, and ENTR-0201, Table B2). One week after a single intravenous dose at 20 mpk, ENTR-164 yielded 59.5±2.2%, 29.1 f 0.8%, and 61.0 f 13.7% exon 23 skipping in Quad, heart, and diaphragm respectively. One week after a single intravenous dose at 20 mpk, ENTR-165 yielded 38.5±2.5%, 30.5±17.0%, and 30.2±2.8% exon 23 skipping in Quad, heart, and diaphragm respectively. One week after a single intravenous dose at 20 mpk, ENTR-201 yielded 73.5 f 8.4%, 60.5 t 17.2%, and 79.0±6.8% exon 23 skipping in Quad, heart, and diaphragm respectively. The preparation of these NLS fusions is described in detail above. The structure of ENTR-164, ENTR-165 are shown above. Consistent with the exon skipping data, by ENTR-0164 and ENTR-0165 also significantly corrected the expression of dystrophin protein levels as analyzed by Western Blot. By comparing to the level in respective tissues from wild type (C57BL/10) and using the actinin as loading control, percentage of dystrophin protein correction is further quantified to that of wild type. Instead of using the modified PMO with 3′ amide bond formation, we also tested the incorporation of cyclooctyne on solid support by modifying the morpholino amino group with a bifunctional linker comprised of a cyclooctyne moiety for click reaction to a CPP-azide and a PFP easter to form a carbamate with PMO and thus produced the precursor which can be used for synthesis of ENTR-201. Similar to that of ENTR-165, ENTR-201 also demonstrated high exon skipping activity across all the muscle groups.

Activity of ENTR-201 in MDX mice. The table below outlines the injection, sample collection and bioanalysis to study the duration of effects of ENTR-201 after single IV injection.

| | | | Dosage | SAC time | | |
|---|---|---|---|---|---|---|
| Group | N | Treatment | (mpk) | (p.i.) | | Endpoints |
| 1 | 3 | PBS | 0 | 1 | week | Exon skipping |
| 2 | 3 | ENTR-201 | 20 | 1 | week | (RT-PCR) |
| 3 | 3 | ENTR-201 | 20 | 2 | weeks | Dystrophin expression |
| 4 | 3 | ENTR-201 | 20 | 4 | weeks | (Western blot and |

immunohistochemistry)

After a single dose of ENTR-201 at 20 mg/kg on day 1, animals were sacrificed at 1 week, 2 weeks and 4 weeks post injection. Vehicle (PBS) only was used as a negative control. Heart, diaphragm, quadriceps and transverse abdominis were collected for RT-PCR to detect dystrophin exon 23 skipped product, and Western blot analysis to detect dystrophin protein expression (relative to alpha-actinin). Samples from 4 weeks post single IV injection of ENTR-201 at 20 mpk or PBS were also analyzed by immunohistochemistry staining to detect expression and distribution of dystrophin in various muscle tissues.

Treatment of mice with single dose 20 mg/kg ENTR-0201 resulted in splicing correction of dystrophin in the heart, diaphragm, quadriceps and transverse abdominis (TrA). ENTR-0201 delivers significant enhancements in exon skipping efficiency up to four weeks post single IV injection. The corresponding dystrophin protein levels were analyzed by Western Blot. Restored dystrophin protein sustained up to four weeks after single IV injection at 20 mpk in the heart, diaphragm, quadriceps and transverse abdominis (TrA). The level of protein correction is consistent with the RNA analysis. The tissue samples from the last injection were also analyzed by immunohistochemistry, which show that all the skeletal muscle fibers immunostained positive for dystrophin protein as visualized by brown color staining. The intensity of dystrophin expression was significant in the heart muscle tissue reaching near normal levels. Widespread uniform expression of dystrophin protein over multiple tissue sections within each of muscle group analyzed.

Activity of ENTR-201 in MDX mice after repeated dosage. The table below outlines the injection, sample collection and bioanalysis to study the activity of ENTR-201 after repeated dosage.

| | | | Dosage | Dosage | |
|---|---|---|---|---|---|
| Group | N | Treatment | (mpk) | Frequency | Endpoints |
| 1 | 3 | PBS | 0 | QW × 4 | Exon skipping |
| 2 | 3 | ENTR-013 | 20 | QW × 4 | (RT-PCR) |
| 3 | 3 | ENTR-201 | 10 | QW × 4 | Creatine Kinase |
| | | | | | Dystrophin Expression |
| | | | | | (Western blot and |
| | | | | | immunohistochemistry) |

Mice were treated with 10 mg/kg of ENTR-201 once every week for 4 weeks. ENTR-013 at 20 mg/kg (PMO only) and vehicle (PBS) only were used as control groups. All animals were sacrificed at 1 week post the last injection. Heart, diaphragm, quadriceps and transverse abdominis were collected for RT-PCR to detect dystrophin exon skipped products, Western blot analysis and immunohistochemistry staining to detect dystrophin protein expression to detect expression and distribution of dystrophin in various tissues. Serum creatine kinase level was quantified as a muscle functional biomarker.

Treatment of MDX mice with 10 mg/kg ENTR-201 once per week for four weeks also resulted in significant splicing correction of dystrophin mRNA and dystrophin protein levels in various muscle tissues (heart, diaphragm, quadricep, and transverse abdominis (TrA)). In comparison to treatment with 20 mpk PMO, treatment with ENTR-201 at 10 mg/kg results in a higher amount of both splicing correction and dystrophin protein in all four muscle tissues. Notably, the mRNA correction and dystrophin protein expression in the heart are only observed in mpk ENTR-201 treated MDX mice, not in 20 mg/kg PMO treated MDX mice. The findings from IHC study was also consistent with RT-PCR and WB analysis. Treatment with 10 mpk ENTR-201 once per week for four weeks also normalized the serum creatine kinase level, which is a muscle damage biomarker, suggesting that Oligo 201 treatment reduces muscle fiber damage in a DMD mouse model. PMO (ENTR-0013) treatment alone in contract did not significantly reduce the elevated serum CK level. Serum samples were collected one week after the last injection from repeated dosing study. Analysis of CK levels was performed using commercially available CK measurement kit (Millipore Sigma chemicals, MAK116) as per instructions from the manufacturer. Quantification of dystrophin protein showed nearly 40% cells are positive for dystrophin in cardiac tissue compared to 5% or less in vehicle treated or PMO alone treated cardia tissues.

Treatment of mice with 20 mg/kg Oligo 201 one time per week resulted in splicing correction of dystrophin in the heart and in the diaphragm. Treatment of mice with 10 mg/kg Oligo 201 or 5 mg/kg Oligo 201 four times per week also resulted in splicing correction of dystrophin in the heart and in the diaphragm. Treatment with Oligo 201 at 10 mg/kg results in a higher amount of splicing correction than treatment with 5 mg/kg in the heart and diaphragm. Treatment with 5 mg/kg or 10 mg/kg Oligo 201 four times per week also resulted in a decrease in creatine kinase expression in comparison to control, suggesting that Oligo 201 treatment reduces muscle fiber damage in patients with DMD.

### Example 17. Tissue Modulation in Muscle Using a Cell-Penetrating Peptide Coupled to an Oligonucleotide and Nuclear Localization Sequence for Splicing Correction of Exon 23 of DMD in an MDX Mouse Model

The compounds of Table 28 below include additional non-limiting examples of NLS-containing compounds. Compounds were prepared as described in previous examples.

TABLE 28

| Sequence | Entrada ID |
|---|---|
| Ac-PKKKRKV-K(cyclo[FfϕR-cit-R-cit-Q])-PEG12-K(N3) (SEQ ID NO: | ETRD-965 |
| 281) | |
| Ac-rr-miniPEG2-Dap[cyclo(Ffϕ-Cit-r-Cit-rQ)]-PEG12-OH (SEQ ID NO: | ETRD-997 |
| 236) | |
| Ac-frr-PEG2-Dap(cyclo(Ffϕ-Cit-r-Cit-rQ))-PEG12-OH (SEQ ID NO: 236) | ETRD-998 |
| Ac-rfr-PEG2-Dap(cyclo(Ffϕ-Cit-r-Cit-rQ))-PEG12-OH (SEQ ID NO: 236) | ETRD-999 |
| Ac-rbfbr-PEG2-Dap(cyclo(Ffϕ-Cit-r-Cit-rQ))-PEG12-OH (SEQ ID NO: 239) | ETRD-1000 |
| Ac-rrr-PEG2-Dap(cyclo(Ffϕ-Cit-r-Cit-rQ))-PEG12-OH (SEQ ID NO: 236) | ETRD-1001 |
| Ac-rbr-PEG2-Dap(cyclo(Ffϕ-Cit-r-Cit-rQ))-PEG12-OH (SEQ ID NO: 236) | ETRD-1002 |
| Ac-rbrbr-PEG2-Dap(cyclo(Ffϕ-Cit-r-Cit-rQ))-PEG12-OH (SEQ ID NO: | ETRD-1003 |
| 236) | |
| Ac-hh-PEG2-Dap(cyclo(Ffϕ-Cit-r-Cit-rQ))-PEG12-OH (SEQ ID NO: 236) | ETRD-1004 |
| Ac-hbh-PEG2-Dap(cyclo(Ffϕ-Cit-r-Cit-rQ))-PEG12-OH (SEQ ID NO: | ETRD-1005 |
| 236) | |
| Ac-hbhbh-PEG2-Dap(cyclo(Ffϕ-Cit-r-Cit-rQ))-PEG12-OH (SEQ ID NO: | ETRD-1006 |
| 236) | |
| Ac-rbhbh-PEG2-Dap(cyclo(Ffϕ-Cit-r-Cit-rQ))-PEG12-OH (SEQ ID NO: | ETRD-1007 |
| 236) | |
| Ac-hbrbh-PEG2-Dap(cyclo(Ffϕ-Cit-r-Cit-rQ))-PEG12-OH (SEQ ID NO: | ETRD-1008 |
| 236) | |
| Ac-rr-Dap(cyclo(Ffϕ-Cit-r-Cit-rQ))-b-OH (SEQ ID NO: 236) | ETRD-1009 |
| Ac-fir-Dap(cyclo(Ffϕ-Cit-r-Cit-rQ))-b-OH (SEQ ID NO: 236) | ETRD-1010 |
| Ac-rfr-Dap(cyclo(Ffϕ-Cit-r-Cit-rQ))-b-OH (SEQ ID NO: 236) | ETRD-1011 |
| Ac-rbfbr-Dap(cyclo(Ffϕ-Cit-r-Cit-rQ))-b-OH (SEQ ID NO: 236) | ETRD-1012 |
| Ac-rrr-Dap(cyclo(Ffϕ-Cit-r-Cit-rQ))-b-OH (SEQ ID NO: 236) | ETRD-1013 |
| Ac-rbr-Dap(cyclo(Ffϕ-Cit-r-Cit-rQ))-b-OH (SEQ ID NO: 236) | ETRD-1014 |
| Ac-rbrbr-Dap(cyclo(Ffϕ-Cit-r-Cit-rQ))-b-OH (SEQ ID NO: 236) | ETRD-1015 |
| Ac-hh-Dap(cyclo(Ffϕ-Cit-r-Cit-rQ))-b-OH (SEQ ID NO: 236) | ETRD-1016 |
| Ac-hbh-Dap(cyclo(Ffϕ-Cit-r-Cit-rQ))-b-OH (SEQ ID NO: 236) | ETRD-1017 |
| Ac-hbhbh-Dap(cyclo(Ffϕ-Cit-r-Cit-rQ))-b-OH (SEQ ID NO: 236) | ETRD-1018 |
| Ac-rbhbh-Dap(cyclo(Ffϕ-Cit-r-Cit-rQ))-b-OH (SEQ ID NO: 236) | ETRD-1019 |
| Ac-hbrbh-Dap(cyclo(Ffϕ-Cit-r-Cit-rQ))-b-OH (SEQ ID NO: 236) | ETRD-1020 |
| Ac-KKKK-miniPEG2-K(cyclo(Ff-Nal-GrGrQ))-miniPEG2-K(N3)-NH2 | ETRD-1045 |
| (SEQ ID NOs: 64 and 234) | |
| Ac-KGKK-miniPEG2-K(cyclo(Ff-Nal-GrGrQ))-miniPEG2-K(N3)-NH2 | ETRD-1046 |
| (SEQ ID NOs: 70 and 234) | |
| Ac-KKGK-miniPEG2-K(cyclo(Ff-Nal-GrGrQ))-miniPEG2-K(N3)-NH2 | ETRD-1047 |
| (SEQ ID NOs: 71 and 234) | |
| Ac-KKK-miniPEG2-K(cyclo(Ff-Nal-GrGrQ))-miniPEG2-K(N3)-NH2 (SEQ | ETRD-1048 |
| ID NO: 234) | |
| Ac-KK-miniPEG2-K(cyclo(Ff-Nal-GrGrQ))-miniPEG2-K(N3)-NH2 (SEQ | ETRD-1049 |
| ID NO: 234) | |
| Ac-KGK-miniPEG2-K(cyclo(Ff-Nal-GrGrQ))-miniPEG2-K(N3)-NH2 | ETRD-1050 |
| (SEQ ID NO: 234) | |
| Ac-KBK-miniPEG2-K(cyclo(Ff-Nal-GrGrQ))-miniPEG2-K(N3)-NH2 (SEQ | ETRD-1051 |
| ID NO: 234) | |
| Ac-KBKBK-miniPEG2-K(cyclo(Ff-Nal-GrGrQ))-miniPEG2-K(N3)-NH2 | ETRD-1052 |
| (SEQ ID NO: 234) | |
| Ac-KR-miniPEG2-K(cyclo(Ff-Nal-GrGrQ))-miniPEG2-K(N3)-NH2 (SEQ | ETRD-1053 |
| ID NO: 234) | |
| Ac-KBR-miniPEG2-K(cyclo(Ff-Nal-GrGrQ))-miniPEG2-K(N3)-NH2 (SEQ | ETRD-1054 |
| ID NO: 234) | |
| Ac-PKKKRKV-miniPEG2-K(cyclo(Ff-Nal-GrGrQ))-miniPEG2-K(N3)- | ETRD-1055 |
| NH2 (SEQ ID NOs: 103 an d234) | |
| Ac-PKKKRKV-miniPEG2-K(cyclo(Ff-Nal-GrGrQ))-miniPEG2-K(N3)- | ETRD-1055 |
| NH2 (SEQ ID NOs: 103 and 234) | |
| Ac-PGKKRKV-miniPEG2-K(cyclo(Ff-Nal-GrGrQ))-miniPEG2-K(N3)- | ETRD-1056 |
| NH2 (SEQ ID NOs: 283 and 234) | |
| Ac-PKGKRKV-miniPEG2-K(cyclo(Ff-Nal-GrGrQ))-miniPEG2-K(N3)- | ETRD-1057 |
| NH2 (SEQ ID NOs: 105 and 234) | |
| Ac-PKKGRKV-miniPEG2-K(cyclo(Ff-Nal-GrGrQ))-miniPEG2-K(N3)- | ETRD-1058 |
| NH2 (SEQ ID NOs: 106 and 234) | |
| Ac-PKKKGKV-miniPEG2-K(cyclo(Ff-Nal-GrGrQ))-miniPEG2-K(N3)- | ETRD-1059 |
| NH2 (SEQ ID NOs: 107 and 234) | |
| Ac-PKKKRGV-miniPEG2-K(cyclo(Ff-Nal-GrGrQ))-miniPEG2-K(N3)- | ETRD-1060 |
| NH2 (SEQ ID NOs: 108 and 234) | |
| Ac-PKKKRKG-miniPEG2-K(cyclo(Ff-Nal-GrGrQ))-miniPEG2-K(N3)- | ETRD-1061 |
| NH2 (SEQ ID NOs: 109 and 234) | |
| Ac-KKKRK-miniPEG2-K(cyclo(Ff-Nal-GrGrQ))-miniPEG2-K(N3)-NH2 | ETRD-1062 |
| (SEQ ID NOs: 76 and 234) | |
| Ac-KKRK-miniPEG2-K(cyclo(Ff-Nal-GrGrQ))-miniPEG2-K(N3)-NH2 | ETRD-1063 |
| (SEQ ID NOs: 65 and 234) | |
| Ac-KRK-miniPEG2-K(cyclo(Ff-Nal-GrGrQ))-miniPEG2-K(N3)-NH2 (SEQ | ETRD-1064 |
| ID NO: 64) | |
| Ac-KKKK-miniPEG2-K(cyclo(FGFGRGRQ))-miniPEG2-K(N3)-NH2 | ETRD-1092 |
| (SEQ ID NOs: 64 and 128) | |
| Ac-KBKBK-miniPEG2-K(cyclo(FGFGRGRQ))-miniPEG2-K(N3)-NH2 | ETRD-1093 |
| (SEQ ID NO: 128) | |
| Ac-PKKKRKV-miniPEG2-K(cyclo(FGFGRGRQ))-miniPEG2-K(N3)-NH2 | ETRD-1094 |
| (SEQ ID NOs: 246 and 128) | |
| Ac-KGK-miniPEG2-K(cyclo(FGFGRGRQ))-miniPEG2-K(N3)-NH2 (SEQ | ETRD-1095 |
| ID NO: 128) | |
| Ac-KBK-miniPEG2-K(cyclo(FGFGRGRQ))-miniPEG2-K(N3)-NH2 (SEQ | ETRD-1096 |
| ID NO: 128) | |
| Ac-KGKK-miniPEG2-K(cyclo(FGFGRGRQ))-miniPEG2-K(N3)-NH2 | ETRD-1097 |
| (SEQ ID NOs: 70 and 128) | |
| Ac-KKKK-miniPEG2-K(cyclo(GfF-GrGrQ))-miniPEG2-K(N3)-NH2 (SEQ | ETRD-1098 |
| ID NOs: 64 and 128) | |
| Ac-KBKBKminiPEG2-K(cyclo(GfF-GrGrQ))-miniPEG2-K(N3)-NH2 | ETRD-1099 |
| (SEQ ID NOs: 233) | |
| Ac-PKKKRKV-miniPEG2-K(cyclo(GfF-GrGrQ))-miniPEG2-K(N3)-NH2 | ETRD-1100 |
| (SEQ ID NOs: 246 and 233) | |
| Ac-KGK-miniPEG2-K(cyclo(GfF-GrGrQ))-miniPEG2-K(N3)-NH2 (SEQ | ETRD-1101 |
| ID NO: 233) | |
| Ac-KBK-miniPEG2-K(cyclo(GfF-GrGrQ))-miniPEG2-K(N3)-NH2 (SEQ | ETRD-1102 |
| ID NO: 233) | |
| Ac-KGKK-miniPEG2-K(cyclo(GfF-GrGrQ))-miniPEG2-K(N3)-NH2 (SEQ | ETRD-1103 |
| ID NOs: 70 and 233) | |
| Ac-KKKK-miniPEG2-K(cyclo(FfF-GrGrQ))-miniPEG2-K(N3)-NH2 (SEQ | ETRD-1104 |
| ID NOs: 64 and 235) | |
| Ac-KBKBK-miniPEG2-K(cyclo(FfF-GrGrQ))-miniPEG2-K(N3)-NH2 | ETRD-1105 |
| (SEQ ID NO: 235) | |
| Ac-PKKKRKV-miniPEG2-K(cyclo(FfF-GrGrQ))-miniPEG2-K(N3)-NH2 | ETRD-1106 |
| (SEQ ID NOs: 246 and 235) | |
| Ac-KGK-miniPEG2-K(cyclo(FfF-GrGrQ))-miniPEG2-K(N3)-NH2 (SEQ | ETRD-1107 |
| ID NO: 235) | |
| Ac-KBK-miniPEG2-K(cyclo(FfF-GrGrQ))-miniPEG2-K(N3)-NH2 (SEQ | ETRD-1108 |
| ID NO: 235) | |
| Ac-KGKK-miniPEG2-K(cyclo(FfF-GrGrQ))-miniPEG2-K(N3)-NH2 (SEQ | ETRD-1109 |
| ID NOs: 70 and 235) | |
| Ac-PKKKRKV-miniPEG2-K(cyclo(FGFGRGRQ))-PEG12-K(N3)-NH2 | ETRD-1131 |
| (SEQ ID NOs: 246 and 128) | |
| Ac-PKKKRKV-miniPEG2-K(cyclo(GfF-GrGrQ))-PEG12-K(N3)-NH2 | ETRD-1132 |
| (SEQ ID NOs: 246 and 233) | |
| Ac-KKKK-miniPEG2-K(cyclo(FGFGRGRQ))-PEG12-K(N3)-NH2 (SEQ | ETRD-1133 |
| ID NOs: 64 and 233) | |
| Ac-KKKK-miniPEG2-K(cyclo(GfF-GrGrQ))-PEG12-K(N3)-NH2 (SEQ ID | ETRD-1134 |
| NOs: 64 and 233) | |
| Ac-PKKKRKV-miniPEG2-K(cyclo(FGFGRGRQ))-PEG12-OH (SEQ ID | ETRD-1135 |
| NOs: 246 and 128) | |
| Ac-PKKKRKV-miniPEG2-K(cyclo(GfF-GrGrQ))-PEG12-OH (SEQ ID | ETRD-1136 |
| NOs: 246 and 233) | |
| Ac-KKKK-miniPEG2-K(cyclo(FGFGRGRQ))-PEG12-OH (SEQ ID NOs: | ETRD-1137 |
| 64 and 128) | |
| Ac-KKKK-miniPEG2-K(cyclo(GfF-GrGrQ))-PEG12-OH (SEQ ID NOs: 64 | ETRD-1138 |
| and 233) | |

Non-limiting examples of CCP-AC chemical structures that further comprise a modulatory peptide (MP; or NLS) are shown:

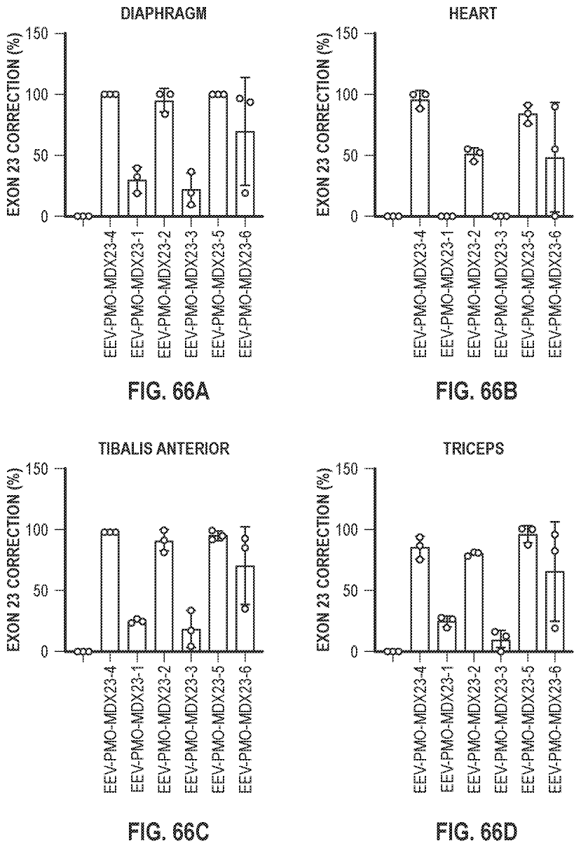

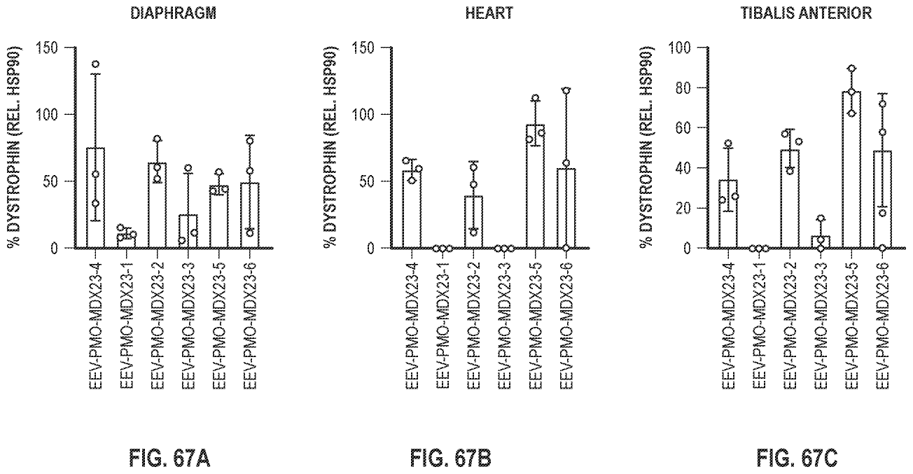

Exemplary compounds were tested in the MDX model described in Example 16. Mice were treated with a single intravenous dose at 20 mg/kg on day 1. On day 5 post-injection, animals were sacrificed and the specified tissues were harvested and flash-frozen. RNA was extracted and exon skipping was quantified by RT-PCR as previously described in diaphragm (FIG. 66A), heart (FIG. 66B), tibialis anterior (FIG. 66C) and triceps (FIG. 66D). The results indicated that treatment with NLS-containing compounds (ENTR-1491093, ENTR-1491094, ENTR-1491096) resulted in a lower level of exon skipping in heart tissue as compared to the three the types of muscle tissue examined. Additionally, on day 5 post-injection, dystrophin levels were quantified by Wester blot analysis in diaphragm (FIG. 67A), heart (FIG. 67B) and tibialis anterior (FIG. 67C). The results indicated that treatment with NLS-containing compounds (ENTR-1491093, ENTR-1491096) also resulted in lower levels dystrophin expression in heart tissue and tibialis anterior tissue as compared to the diaphragm tissue.

### NUMBERED EMBODIMENTS

Embodiment 1 relates to a cyclic peptide of Formula (A):

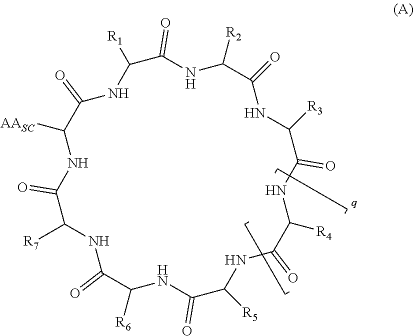

or a protonated form or salt thereof,
wherein:
R₁, R₂, and R₃ are each independently H or an aromatic or heteroaromatic side chain of an amino acid;
   at least one of R₁, R₂, and Rₐ is an aromatic or heteroaromatic side chain of an amino acid;
   R₄, R₅, R₆, R₇ are independently H or an amino acid side chain;
   at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine: AA_{SC} is an amino acid side chain; and
   q is 1, 2, 3 or 4;
   wherein the cyclic peptide of the Formula (A) is not FfΦRrRrE.

Embodiment 2 relates to the cyclic peptide of Embodiment 1, wherein the cyclic peptide is of Formula (I):

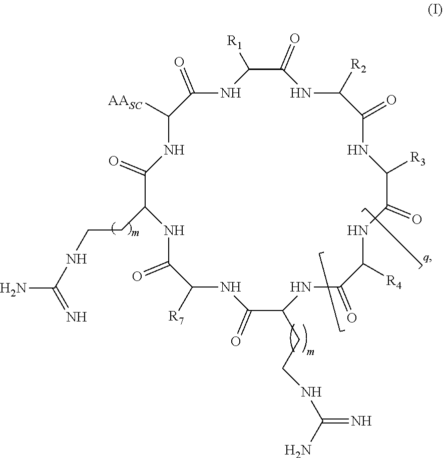

or a protonated form or salt thereof,
wherein each m is independently an integer from 0-3.

Embodiment 3 relates to the cyclic peptide of Embodiment for 2, wherein R₁, R₂, and R₃ are independently H or a side chain comprising an aryl group.

Embodiment 4 relates to the cyclic peptide of any one of the preceding Embodiments, wherein the side chain comprising an aryl group is a side chain of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, or 3-(9-anthryl)-alanine.

Embodiment 5 relates to the cyclic peptide of any one of the preceding Embodiments, wherein the side chain comprising an aryl group is a side chain of phenylalanine.

Embodiment 6 relates to the cyclic peptide of any one of the preceding Embodiments, wherein two of R₁, R₂, and R₃ are a side chain of phenylalanine.

Embodiment 7 relates to the cyclic peptide of any one of the preceding Embodiments, wherein two of R₁, R₂, R₃, and R₄ are H.

Embodiment 8 relates to the cyclic peptide of any one of the preceding Embodiments, wherein the cyclic peptide is of Formula (I-1),

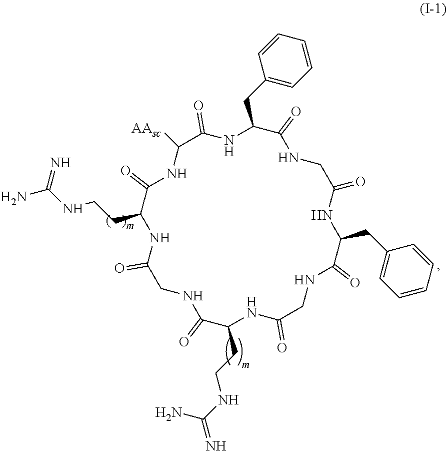

or a protonated form or salt thereof.

Embodiment 9 relates to the cyclic peptide of any one of the preceding Embodiments, wherein the cyclic peptide is of Formula (I-2):

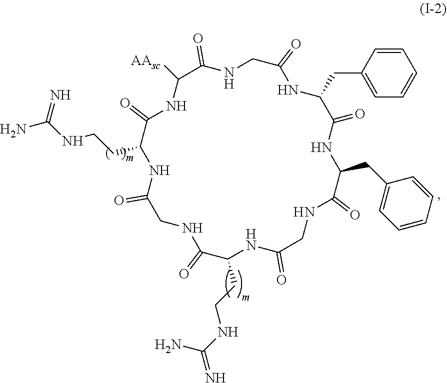

or a protonated form or salt thereof.

Embodiment 10 relates to the cyclic peptide of any one of the preceding Embodiments, wherein the cyclic peptide is of Formula (I-3):

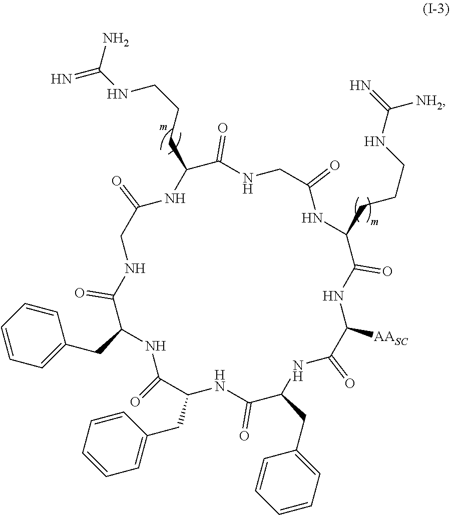

or a protonated form or salt thereof.

Embodiment 11 relates to the cyclic peptide of any one of the preceding Embodiments, wherein the cyclic peptide is of Formula (I4):

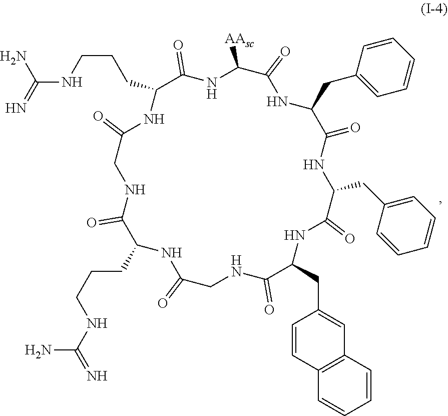

or a protonated form or salt thereof.

Embodiment 12 relates to the cyclic peptide of any one of the preceding Embodiments, wherein the cyclic peptide is of Formula (I-5):

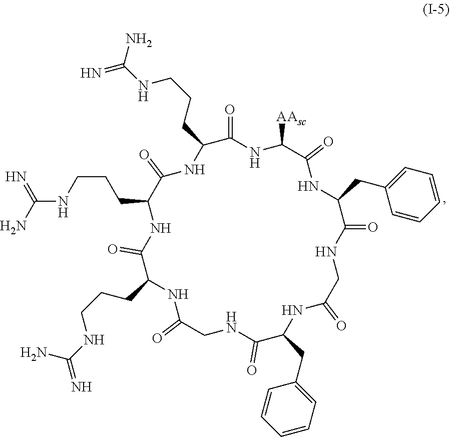

or a protonated form or salt thereof.

Embodiment 13 relates to the cyclic peptide of any one of the preceding Embodiments, wherein the cyclic peptide is of Formula (I-6):

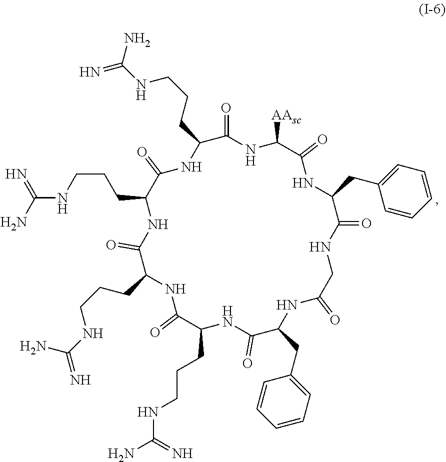

or a protonated form or salt thereof.

Embodiment 14 relates to a cyclic peptide of Formula (II):

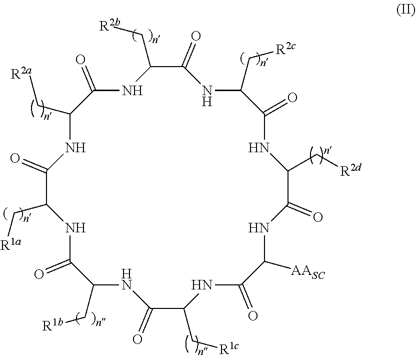

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is

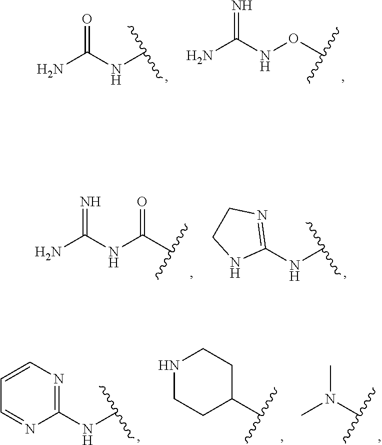

or a protonated form or salt thereof;
at least one of R^{2a}, R^{2b}, R^{2c} and R^{2d} is guanidine or a protonated form or salt thereof;
each n″ is independently an integer from 0 to 5;
each n′ is independently an integer from 0 to 3; and
if n′ is 0 then R^{2a}, R^{2b}, R^{2c} and R^{2d} is absent.

Embodiment 15 relates to the cyclic peptide of Embodiment 14, wherein the cyclic peptide is of Formula (II-1):

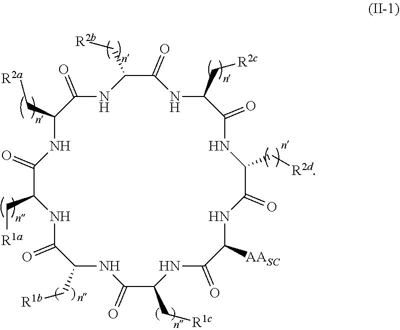

Embodiment 16 relates to the cyclic peptide of Embodiment 14 or 15, wherein R^{1a}, R^{1b}, and R^{1c} are each independently selected from the group consisting of phenyl, naphthyl, and anthracenyl.

Embodiment 17 relates to the cyclic peptide of Embodiment 14 or 15, wherein the cyclic peptide is of Formula (IIa):

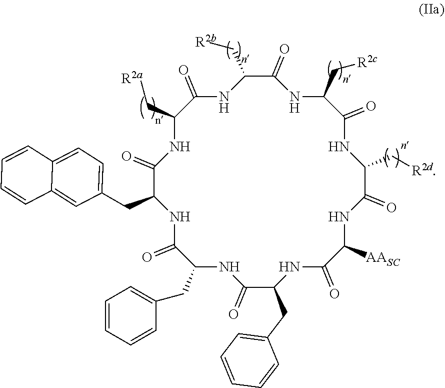

Embodiment 18 relates to the cyclic peptide of any one of Embodiments 14-17, wherein at least one of R^{2a}, R^{2b}, R^{2c} and R^{2d} is

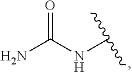 [SMILES: *NC(N)=O]

and the remaining R^{2a}, R^{2b}, R^{2c} and R^{2d} are guanidine, or a protonated form or salt thereof.

Embodiment 19 relates to the cyclic peptide of any one of Embodiments 14-18, wherein at least two R^{2a}, R^{2b}, R^{2c} and R^{2d} are

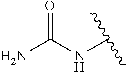 [SMILES: *NC(N)=O]

and the remaining R^{2a}, R^{2b}, R^{2c} and R^{2d} are guanidine, or a protonated form or salt thereof.

Embodiment 20 relates to the cyclic peptide of any one of Embodiments 14-19, wherein the cyclic tide is of Formula (IIb):

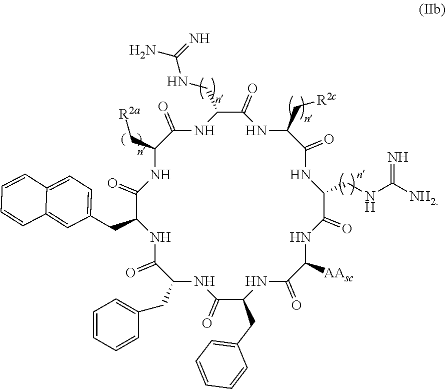

Embodiment 21 relates to the cyclic peptide of any one of Embodiments 14-20, wherein R^{2a} and R^{2c} are each

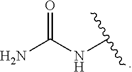 [SMILES: *NC(N)=O]

Embodiment 22 relates to the cyclic peptide of any one of Embodiments 14-21, wherein the cyclic peptide is of Formula (IIc);

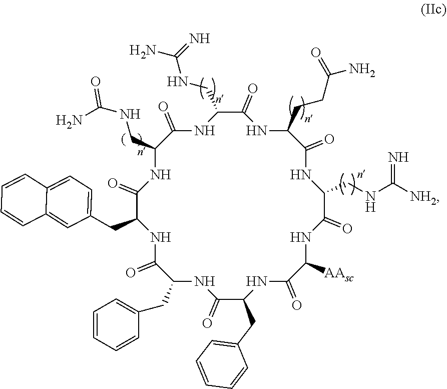

or a protonated form or salt thereof.

Embodiment 23 relates to the cyclic peptide of any one of the preceding Embodiments, wherein AA_{SC} is a side chain of an asparagine residue, aspartic acid residue, glutamic acid residue, homoglutamic acid residue, or homoglutamate residue.

Embodiment 24 relates to the cyclic peptide of any one of the preceding Embodiments, wherein AA_{SC} is a side chain of a glutamic acid residue.

Embodiment 25 relates to the cyclic peptide of any one of the preceding Embodiments, wherein AA_{SC} is:

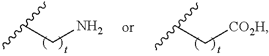

wherein t is an integer from 0 to 5.

Embodiment 26 relates to the cyclic peptide of any one of the preceding Embodiments, having the structure:

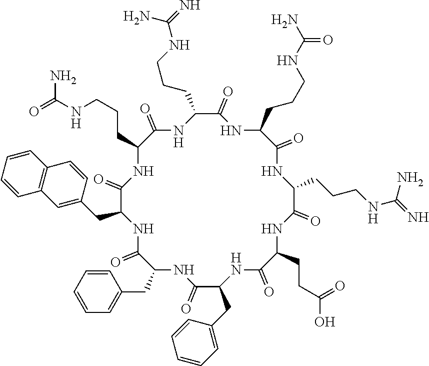 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

or a protonated form or salt thereof.

Embodiment 27 relates to the cyclic peptide of any one of the preceding Embodiments, having the structure:

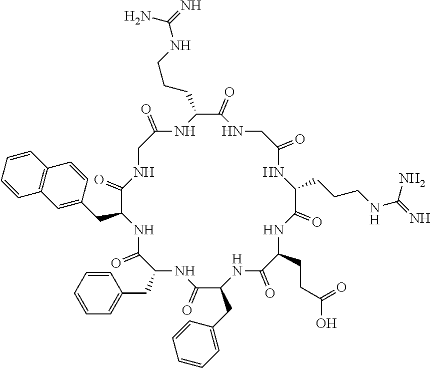 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]

or a protonated form or salt thereof.

Embodiment 28 relates to the cyclic peptide of any one of the preceding Embodiments, wherein at least one atom on the AA_{SC} is replaced by a cargo moiety or at least one lone pair forms a bond to a cargo moiety.

Embodiment 29 relates to the cyclic peptide of any one of the preceding Embodiments, wherein the AA_{SC} is conjugated to a linker.

Embodiment 30 relates to the cyclic peptide of any one of the preceding Embodiments, wherein in the conjugated form the AA_{SC} is a side chain of an asparagine residue, glutamine residue, or homoglutamine residue.

Embodiment 31 relates to the cyclic peptide of any one of the preceding Embodiments, wherein in the conjugated form the AA_{SC} is a side chain of a glutamine residue.

Embodiment 32 relates to the cyclic peptide of any one of the preceding Embodiments, wherein a cargo moiety is conjugated to AA_{SC} by a linker.

Embodiment 33 relates to the cyclic peptide of any one of the preceding Embodiments, wherein the linker comprises a —(OCH₂CH₂)_{z′}— subunit, wherein z′ is an integer from 1 to 23.

Embodiment 34 relates to the cyclic peptide of any one of the preceding Embodiments, wherein the linker comprises:
(i) a —(OCH₂CH₂)_{z}— subunit, wherein z′ is an integer from 1 to 23;
(ii) one or more amino acid residues, such as a residue of glycine, β-alanine, 4-aminobutyric acid, 5-aminopentoic acid or 6-aminohexanoic acid, or combinations thereof; or (iii) combinations of (i) and (ii).

Embodiment 35 relates to the cyclic peptide of any one of the preceding Embodiments, wherein the linker comprises:
(i) a —(OCH₂CH₂)_{z}— subunit, wherein z is an integer from 2 to 20;
(ii) one or more residues of glycine, β-alanine, 4-aminobutyric acid, 5-aminopentoic acid 6-aminohexanoic acid, or combinations thereof; or
(iii) combinations of (i and (ii).)

Embodiment 36 relates to the cyclic peptide of any one of the preceding Embodiments, wherein the linker comprises a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heteroaryl, cycloalkyl, or cycloalkenyl.

Embodiment 37 relates to the cyclic peptide of any one of the preceding Embodiments, wherein the linker has the structure.

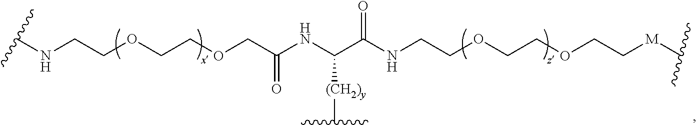

wherein:
x′ is an integer from 1-23; y is an integer from 1-5; z′ is an integer from 1-23; * is the point of attachment to the AA_{SC}, and AA_{SC} is a side chain of an amino acid residue of the cyclic peptide; and M is a bonding group.

Embodiment 38 relates to the cyclic peptide of any one of the preceding Embodiments, wherein the linker has the structure:

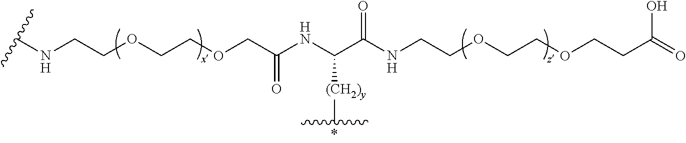

Embodiment 39 relates to the cyclic peptide of any one of the preceding Embodiments, wherein z′ is 11.

Embodiment 40 relates to the cyclic peptide of any one of the preceding Embodiments, wherein x′ is 1.

Embodiment 41 relates to an endosomal escape vehicle (EEV) comprising a cyclic peptide of any one of claims 29-31 and 33-40, and an exocyclic peptide conjugated to the linker at the amino end of the linker.

Embodiment 42 relates to the EEV of Embodiment 41, wherein the cyclic peptide is of Formula (B):

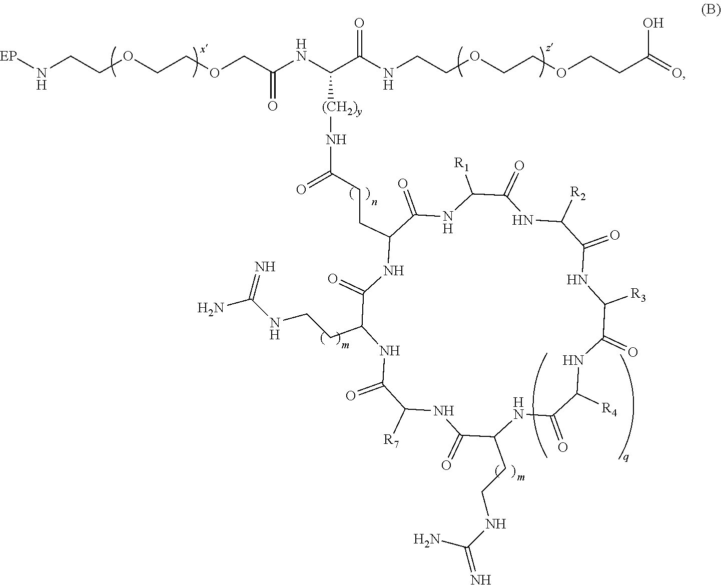

or a protonated form or salt thereof, wherein:
R₁, R₂, and R₃ are each independently H or an aromatic or heteroaromatic side chain of an amino acid;
R₄ and R₇ are independently H or an amino acid side chain;
EP is an exocyclic peptide;
each m is independently an integer from 0-3;
n is an integer from 0-2;
x′ is an integer from 1-20;
y is an integer from 1-5;
q is 1-4; and
z′ is an integer from 1-23.

Embodiment 43 relates to the EEV of Embodiment 41 or 42, wherein the cyclic peptide is of Formula (B-1)-(B-4):

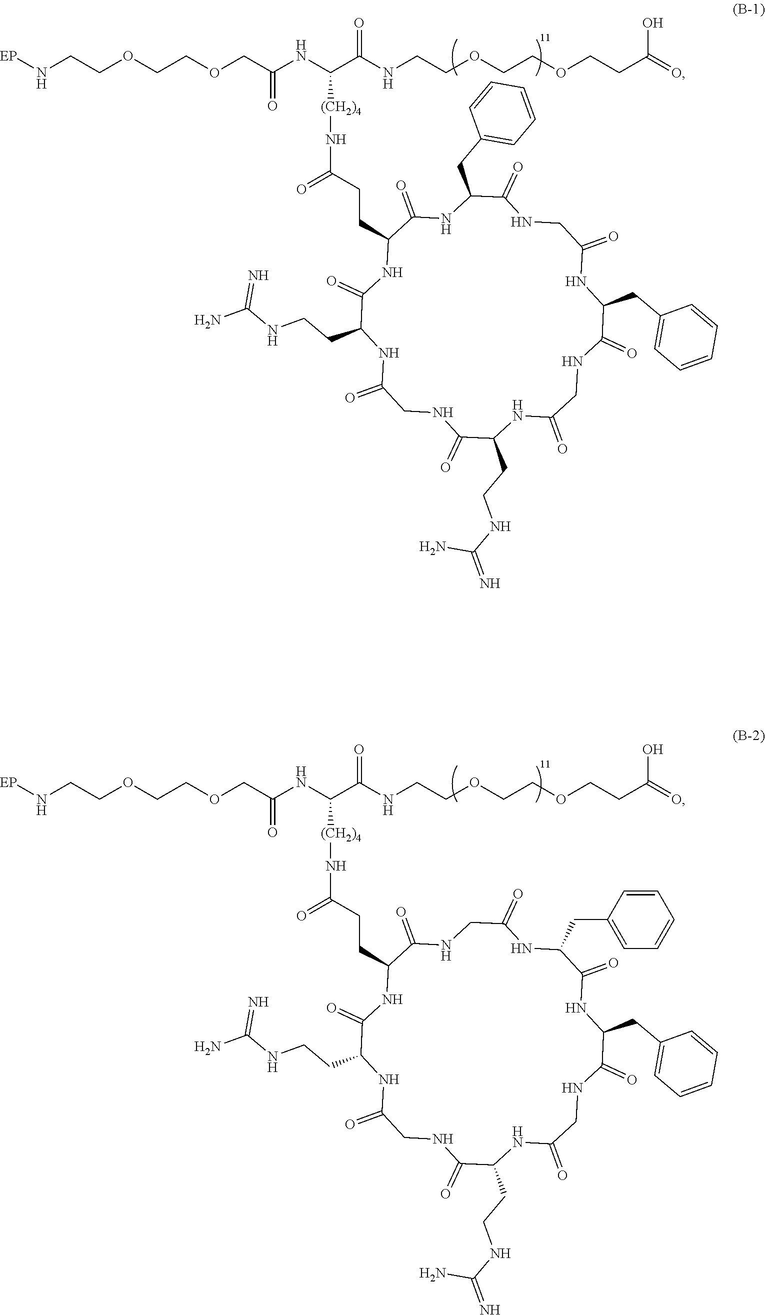

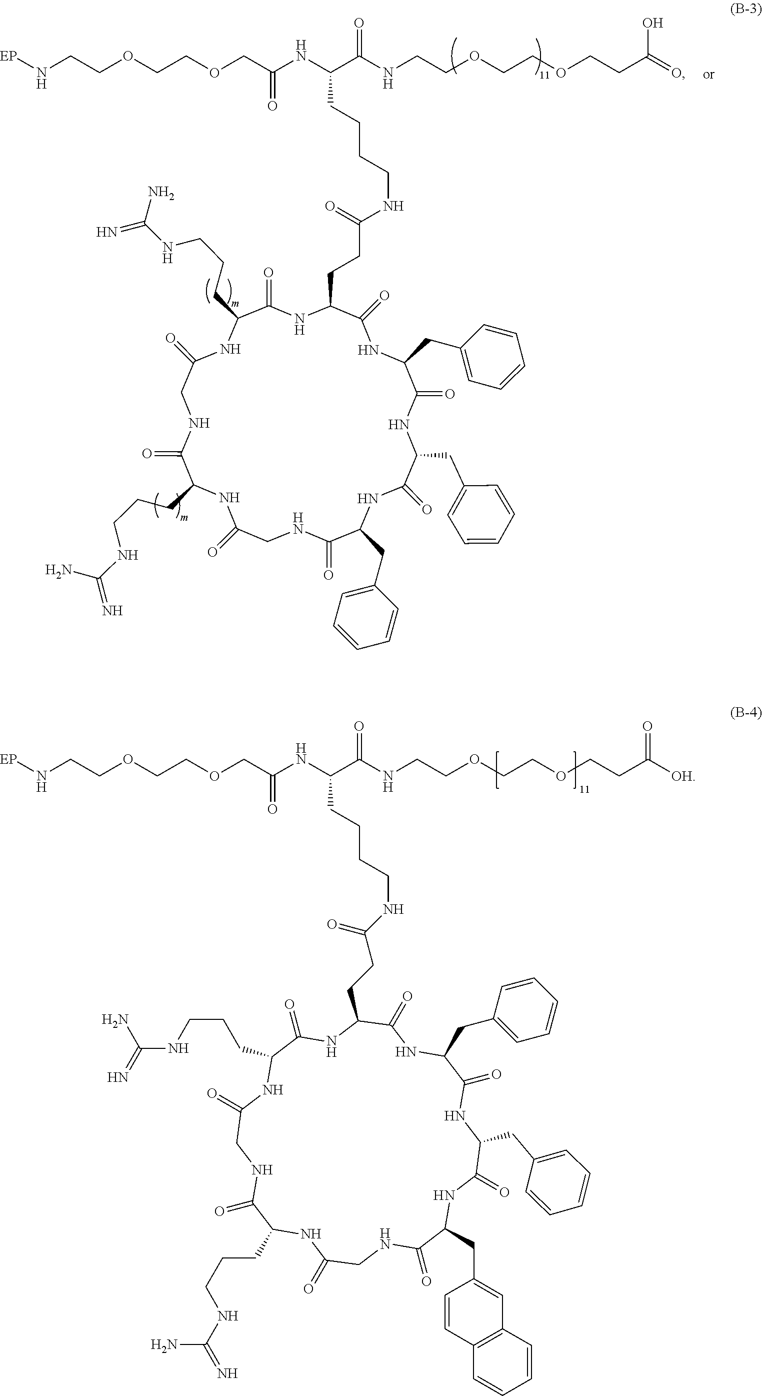

Embodiment 44 relates to the EEV of any one of Embodiments 41-43, wherein the exocyclic peptide comprises from 2 to 10 amino acid residues.

Embodiment 45 relates to the EEV of any one of Embodiments 41-44, wherein the exocyclic peptide comprises from 4 to 8 amino acid residues.

Embodiment 46 relates to the EEV of any one of Embodiments 41-45, wherein the exocyclic peptide comprises 1 or 2 amino acid residues comprising a side chain comprising a guanidine group, or a protonated form or salt thereof.

Embodiment 47 relates to the EEV of any one of Embodiments 41-46, wherein the exocyclic peptide comprises 2, 3, or 4 lysine residues.

Embodiment 48 relates to the EEV of any one of Embodiments 41-47, wherein the amino group on the side chain of each lysine residue is substituted with a trifluoroacetyl (—COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group.

Embodiment 49 relates to the EEV of any one of Embodiments 41-48, wherein the exocyclic peptide comprises at least 2 amino acid residues with a hydrophobic side chain.

Embodiment 50 relates to the EEV of any one of Embodiments 41-49, wherein the amino acid residue with a hydrophobic side chain is selected from valine, proline, alanine, leucine, isoleucine, and methionine.

Embodiment 51 relates to the EEV of any one of Embodiments 41-50, wherein the exocyclic peptide comprises one of the following sequences: KK, KR, RR, HH, HK, HR, RH, KKK, KGK, KBK, KBR, KRK, KRR, RKK, RRR, KKH, KHK, HKK, HRR, HRH, HHR, HBH, HHH, HHHH (SEQ ID NO: 58), KHKK (SEQ ID NO: 59), KKHK (SEQ ID NO: 60), KKKH (SEQ ID NO: 61), KHKH (SEQ ID NO: 62), HKHK (SEQ ID NO: 63), KKKK (SEQ ID NO: 64), KKRK (SEQ ID NO: 65), KRKK (SEQ ID NO: 66), KRRK (SEQ ID NO: 67), RKKR (SEQ ID NO: 68), RRRR (SEQ ID NO: 69), KGKK (SEQ ID NO: 70), KKGK (SEQ ID NO: 71), HBHBH, HBKBH, RRRRR (SEQ ID NO: 74), KKKKK (SEQ ID NO: 75), KKKRK (SEQ ID NO: 76), RKKKK (SEQ ID NO: 77), KRKKK (SEQ ID NO: 78), KKRKK (SEQ ID NO: 79), KKKKR (SEQ ID NO: 80), KBKBK, RKKKKG (SEQ ID NO: 82), KRKKKG (SEQ ID NO: 83), KKRKKG (SEQ ID NO: 84), KKKKRG (SEQ ID NO: 85), RKKKKB (SEQ ID NO: 86), KRKKKB (SEQ ID NO: 87), KKRKKB (SEQ ID NO: 88), KKKKRB (SEQ ID NO: 89), KKKRKV (SEQ ID NO: 90), RRRRRR (SEQ ID NO: 91), HHHHHH (SEQ ID NO: 92), RHRHRH (SEQ ID NO: 93), HRHRHR (SEQ ID NO: 94), KRKRKR (SEQ ID NO: 95), RKRKRK (SEQ ID NO: 96), RBRBRB, KBKBKB, PKKKRKV (SEQ ID NO: 103), PGKKRKV (SEQ ID NO: 104), PKGKRKV (SEQ ID NO: 105), PKKGRKV (SEQ ID NO: 106), PKKKGKV (SEQ ID NO: 107), PKKKRGV (SEQ ID NO: 108) or PKKKRKG (SEQ ID NO: 109).

Embodiment 52 relates to the EEV of any one of Embodiments 41-50, wherein the exocyclic peptide comprises one of the following sequences: PKKKRKV (SEQ ID NO: 103), RR, RRR, RHR, RBR, RBRBR, RBHBR, or HBRBH, wherein B is beta-alanine.

Embodiment 53 relates to the EEV of any one of Embodiments 41-50, wherein the exocyclic peptide comprises one of the following sequences: KK, KR, RR, KKK, KGK, KBK, KBR, KRK, KRR, RKK, RRR, KKKK (SEQ ID NO: 64), KKRK (SEQ ID NO: 65), KRKK (SEQ ID NO: 66), KRRK (SEQ ID NO: 67), RKKR (SEQ ID NO: 68), RRRR (SEQ ID NO: 69), KGKK (SEQ ID NO: 70), KKGK (SEQ ID NO: 71), KKKKK (SEQ ID NO: 75), KKKRK (SEQ ID NO: 76), KBKBK, KKKRKV (SEQ ID NO: 90), PKKKRKV (SEQ ID NO: 103), PGKKRKV (SEQ ID NO: 104), PKGKRKV (SEQ ID NO: 105), PKKGRKV (SEQ ID NO: 106), PKKKGKV (SEQ ID NO: 107), PKKKRGV (SEQ ID NO: 108) or PKKKRKG (SEQ ID NO: 109).

Embodiment 54 relates to the EEV of any one of Embodiments 41-50, wherein the exocyclic peptide comprises one of the following sequences: PKKKRKV (SEQ ID NO: 103), RR, RRR, RHR, RBR, RBRBR, RBHBR, or HBRBH, wherein B is beta-alanine.

Embodiment 55 relates to the EEV of any one of Embodiments 41-50, wherein the exocyclic peptide comprises: PKKKRKV (SEQ ID NO: 103).

Embodiment 56 relates to the EEV of any one of Embodiments 41-50, wherein the exocyclic peptide comprises one of the following sequences: Ac-PKKKRKV (SEQ ID NO: 103).

Embodiment 57 relates to the EEV of any one of Embodiments 41-50, wherein the exocyclic peptide comprises one of the following sequences: NLSKRPAAIKKAGQAKKKK (SEQ ID NO: 253), PAAKRVKLD (SEQ ID NO: 112), RQRRNELKRSF (SEQ ID NO: 113), RMRKFKNKGKDTAELRRRRVEVSVELR (SEQ ID NO: 114), KAKKDEQILKRRNV (SEQ ID NO: 115), VSRKRPRP (SEQ ID NO: 116), PPKKARED (SEQ ID NO: 117), PQPKKKPL (SEQ ID NO: 118), SALIKKKKKMAP (SEQ ID NO: 119), DRLRR (SEQ ID NO: 120), PKQKKRK (SEQ ID NO: 121), RKLKKKIKKL (SEQ ID NO: 122), REKKKFLKRR (SEQ ID NO: 123), KRKGDEVDGVDEVAKKKSKK (SEQ ID NO: 124) or RKCLQAGMNLEARKTKK (SEQ ID NO: 125).

Embodiment 58 relates to the EEV of any one of Embodiments 41-50, wherein the exocyclic peptide comprises one of the following sequences: NLSKRPAAIKKAGQAKKKK, PAAKRVKLD (SEQ ID NO: 112), RQRRNELKRSF (SEQ ID NO: 113), RMRKFKNKGKDTAELRRRRVEVSVELR (SEQ ID NO: 114), KAKKDEQILKRRNV (SEQ ID NO: 115), VSRKRPRP (SEQ ID NO: 116), PPKKARED (SEQ ID NO: 117), PQPKKKPL (SEQ ID NO: 118), SALIKKKKKMAP (SEQ ID NO: 119), DRLRR (SEQ ID NO: 120), PKQKKRK (SEQ ID NO: 121), RKLKKKIKKL (SEQ ID NO. 122), REKKKFLKRR (SEQ ID NO: 123), KRKGDEVDGVDEVAKKKSKK (SEQ ID NO: 124) or RKCLQAGMNLEARKTKK (SEQ ID NO: 125).

Embodiment 59 relates to a compound comprising an EEV of any one of Embodiments 41-58 conjugated to a cargo moiety, wherein the —OH of the terminal carboxylic acid group of the EEV is replaced by the cargo moiety.

Embodiment 60 relates to a compound of Embodiment 59, wherein the cargo moiety. is a small molecule, peptide, oligonucleotide, protein, antibody or derivatives thereof.

Embodiment 61 relates to a compound of Embodiment 59 or 60, wherein the compound is of Formula (C):

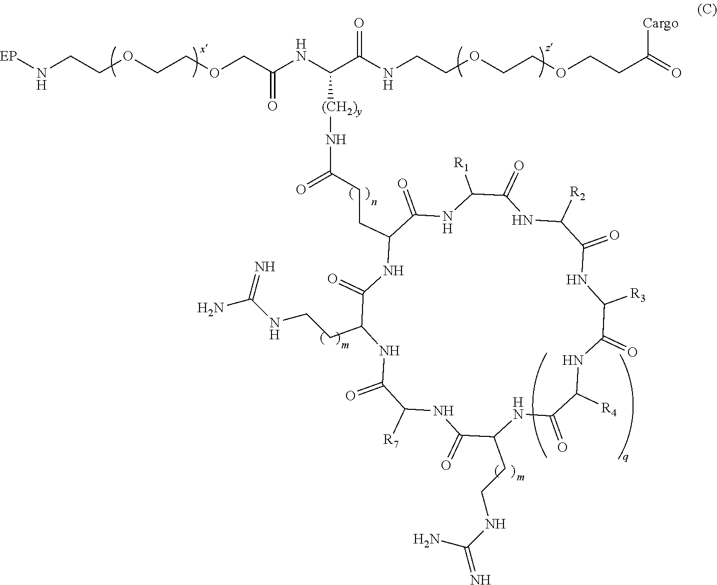

or a protonated form or salt thereof,
wherein:
R₁, R₂, and R₃ are each independently H or a side chain comprising an aryl or heteroaryl group, wherein at least one of R₁, R₂, and R₃ is a side chain comprising an aryl or heteroaryl group;
   R₄ and R₇ are independently H or an amino acid side chain;
EP is an exocyclic peptide;
each m is independently an integer from 0-3;
   n is an integer from 0-2;
   x′ is an integer from 1-23;
   y is an integer from 1-5;
   q is an integer from 1-4; and
z′ is an integer from 1-23.

Embodiment 62 relates to a compound of Embodiment 61, wherein R₁, R₂, and R₃ is H or a side chain comprising an aryl group.

Embodiment 63 relates to a compound of Embodiment 61 or 62, wherein the side chain comprising an aryl group is a side chain of phenylalanine.

Embodiment 64 relates to a compound of any one of Embodiments 61-63, wherein two of R₁, R₂, and R₃ are a side chain of phenylalanine.

Embodiment 65 relates to a compound of any one of Embodiments 61-64, wherein two of R₁, R₂, R₃, and R₄ are H.

Embodiment 66 relates to a compound of any one of Embodiments 61-65, wherein z′ is 11.

Embodiment 67 relates to a compound of any one of Embodiments 61-66, wherein x′ is 1.

Embodiment 68 relates to a compound of any one of Embodiments 61-67, wherein the EP comprises from 2 to 10 amino acid residues.

Embodiment 69 relates to a compound of any one of Embodiments 61-68, wherein the EP comprises from 4 to 8 amino acid residues.

Embodiment 70 relates to a compound of any one of Embodiments 61-69, wherein the EP comprises 1 or 2 amino acid residues comprising a side chain comprising a guanidine group, or a protonated form or salt thereof.

Embodiment 71 relates to a compound of any one of Embodiments 61-70, wherein the EP comprises at least 1 lysine residue.

Embodiment 72 relates to a compound of any one of Embodiments 61-71, wherein the EP comprises 2, 3, or 4 lysine residues.

Embodiment 73 relates to a compound of any one of Embodiments 61-72, wherein the EP comprises at least 2 amino acids with a hydrophobic side chain.

Embodiment 74 relates to a compound of any one of Embodiments 61-73, wherein the amino acid residue with a hydrophobic side chain is selected from valine, proline, alanine, leucine, isoleucine, and methionine residues.

Embodiment 75 relates to a compound of any one of Embodiments 61-c4, wherein the EP comprises one of the following sequences: PKKKRKV (SEQ ID NO: 103); KR, RR, KKK, KGK; KBK, KBR; KRK, KRR; RKK; RRR; KKKK (SEQ ID NO: 64); KKRK (SEQ ID NO: 65); KRKK (SEQ ID NO: 66); KRRK (SEQ ID NO: 67); RKKR (SEQ ID NO: 68); RRRR (SEQ ID NO: 69); KGKK (SEQ ID NO: 70); KKGK (SEQ ID NO: 71); KKKKK (SEQ ID NO: 75); KKKRK (SEQ ID NO: 76); KBKBK, KKKRKV (SEQ ID NO: 90); PGKKRKV (SEQ ID NO: 104); PKGKRKV (SEQ ID NO: 105); PKKGRKV (SEQ ID NO: 106); PKKKGKV (SEQ ID NO: 107); PKKKRGV (SEQ ID NO: 108); or PKKKRKG (SEQ ID NO: 109).

Embodiment 76 relates to a compound of any one of Embodiments 62-75, wherein the EP has the structure: Ac-PKKKRKV (SEQ ID NO: 103).

Embodiment 77 relates to a compound of any one of Embodiments 61-76, wherein the EEV is conjugated to a cargo moiety comprising a therapeutic moiety selected from an oligonucleotide, a peptide and a small molecule.

Embodiment 78 relates to a compound of any one of Embodiments 61-77, comprising the structure of Formula (C-1), (C-2), (C-3), or (C-4):

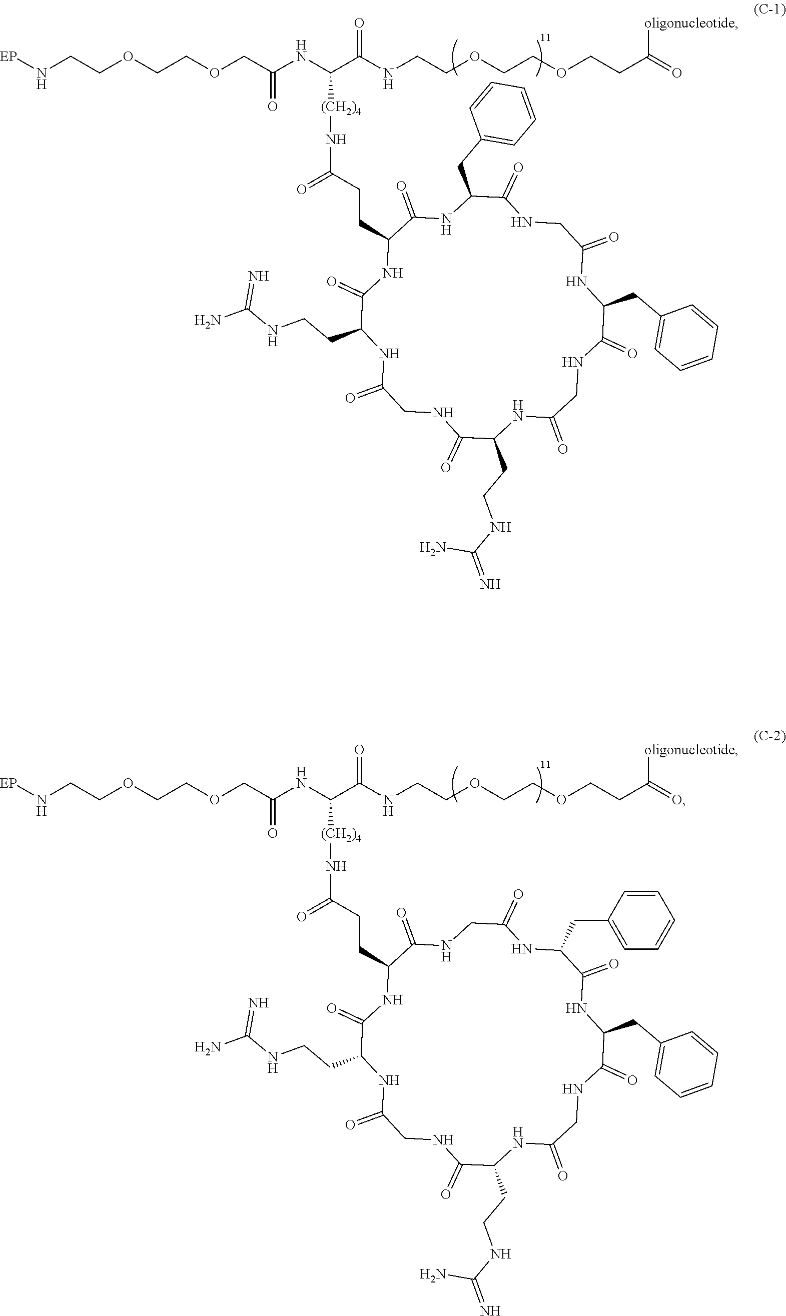

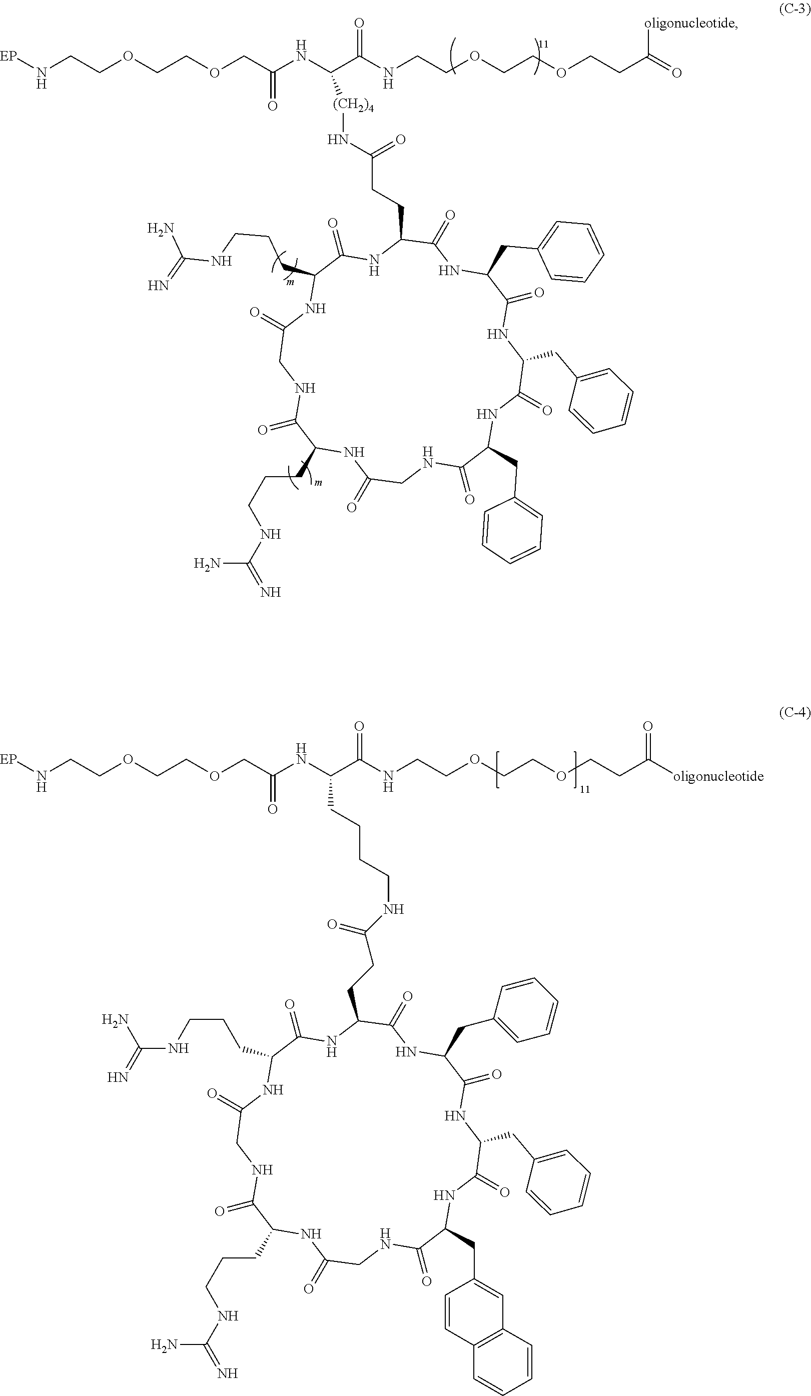

or a protonated form or salt thereof.

Embodiment 79 relates to a compound of formula:

(SEQ ID NO: 274)
Ac-PKKKRKVAEEAK(cyclo[FGFGRGRQ])-PEG₁₂-OH
or
(SEQ ID NO: 268)
Ac-PKKKRKVAEEAK(cyclo[GfFGrGrQ])-PEG₁₂-OH.

Embodiment 80 relates to a cargo, a linker of and a cyclic peptide of formula:

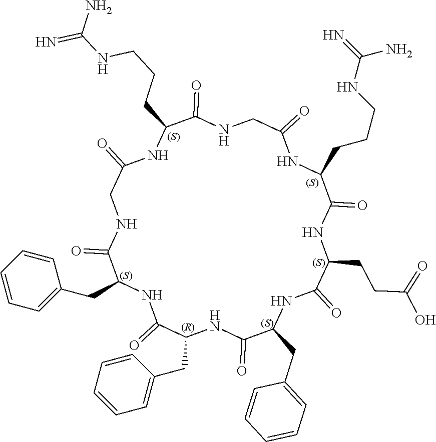

Embodiment 81 relates to an EEV of formula:
Ac-PKKKRKV-miniPEG2-K(cyclo(FfFGRGRQ)-miniPEG2-K(N₃) (SEQ ID NOs: 246 and 235).
Embodiment 82 relates to an EEV of formula:

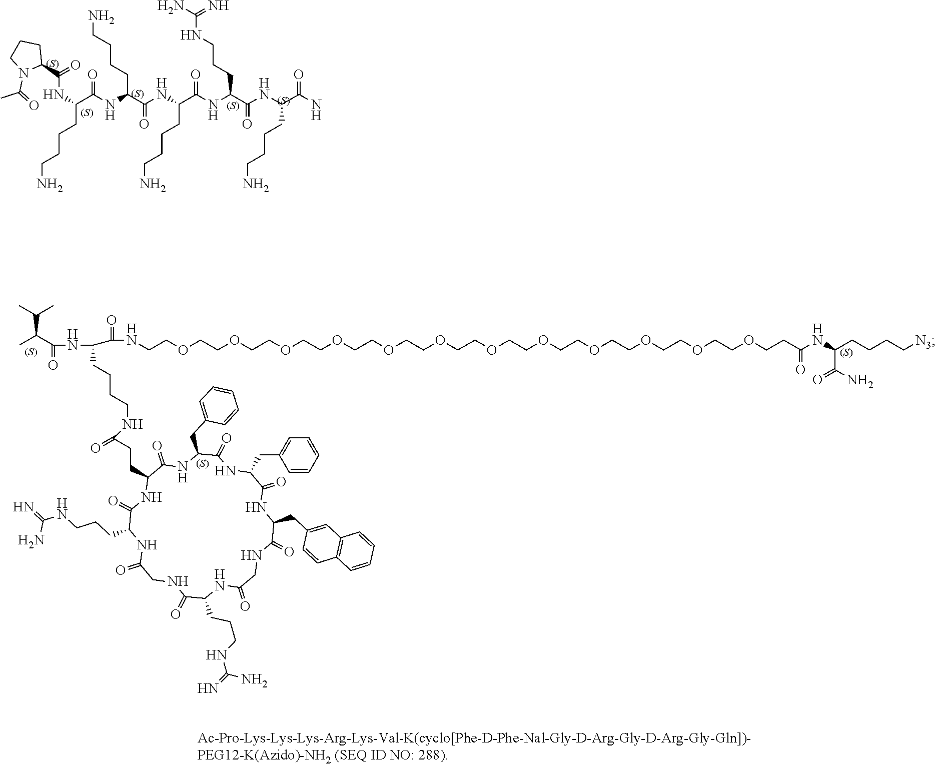

Embodiment 83 relates to an EEV of formula:

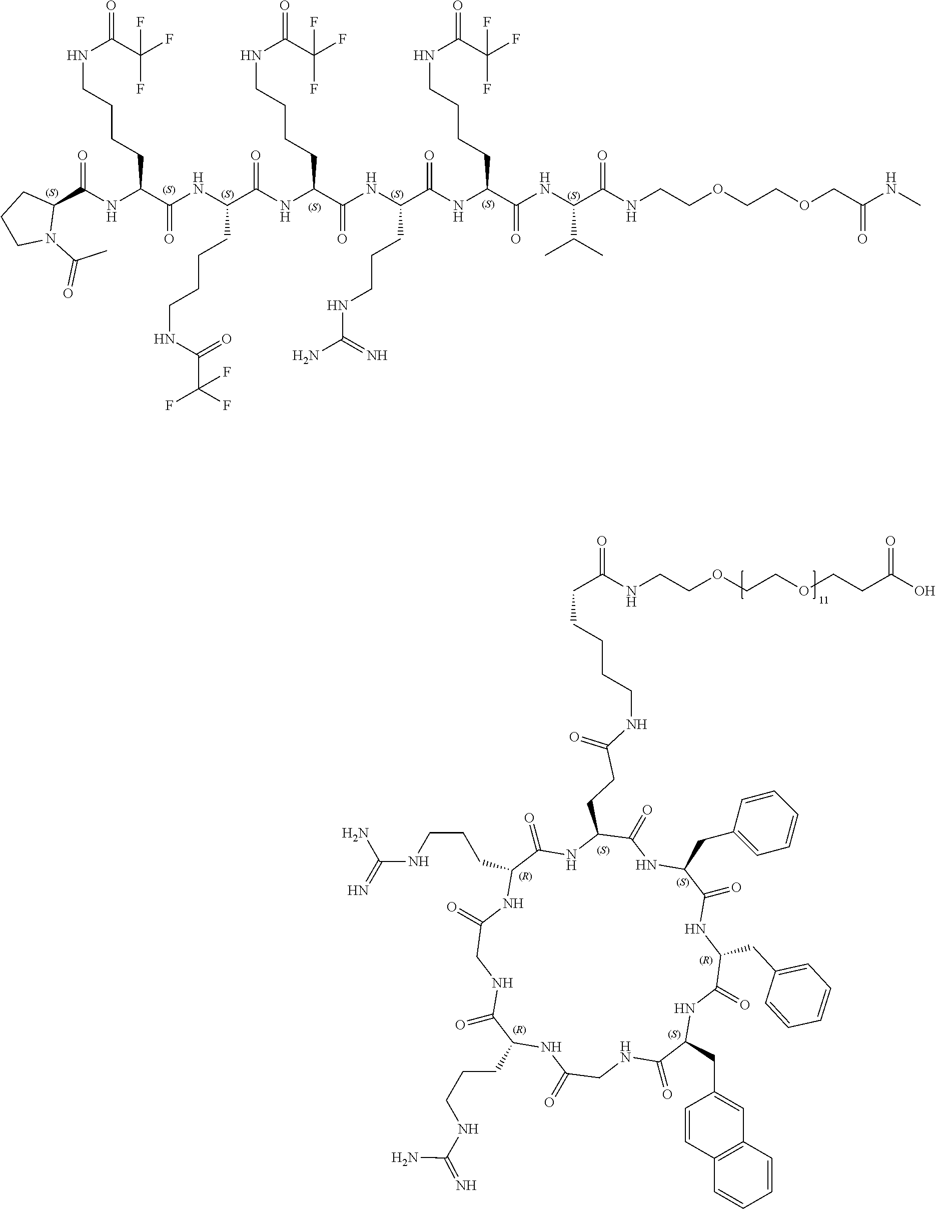

Embodiment 84 relates EEV of formula: Ac-P-K(Tfa)-K(Tfa)-K(Tfa)-R-K(Tfa)-V-miniPEG-K(cyclo(Ff-Nal-GrGrQ)-PEG12-OH (SEQ ID NO: 272).

Embodiment 85 relates to an EEV of formula:

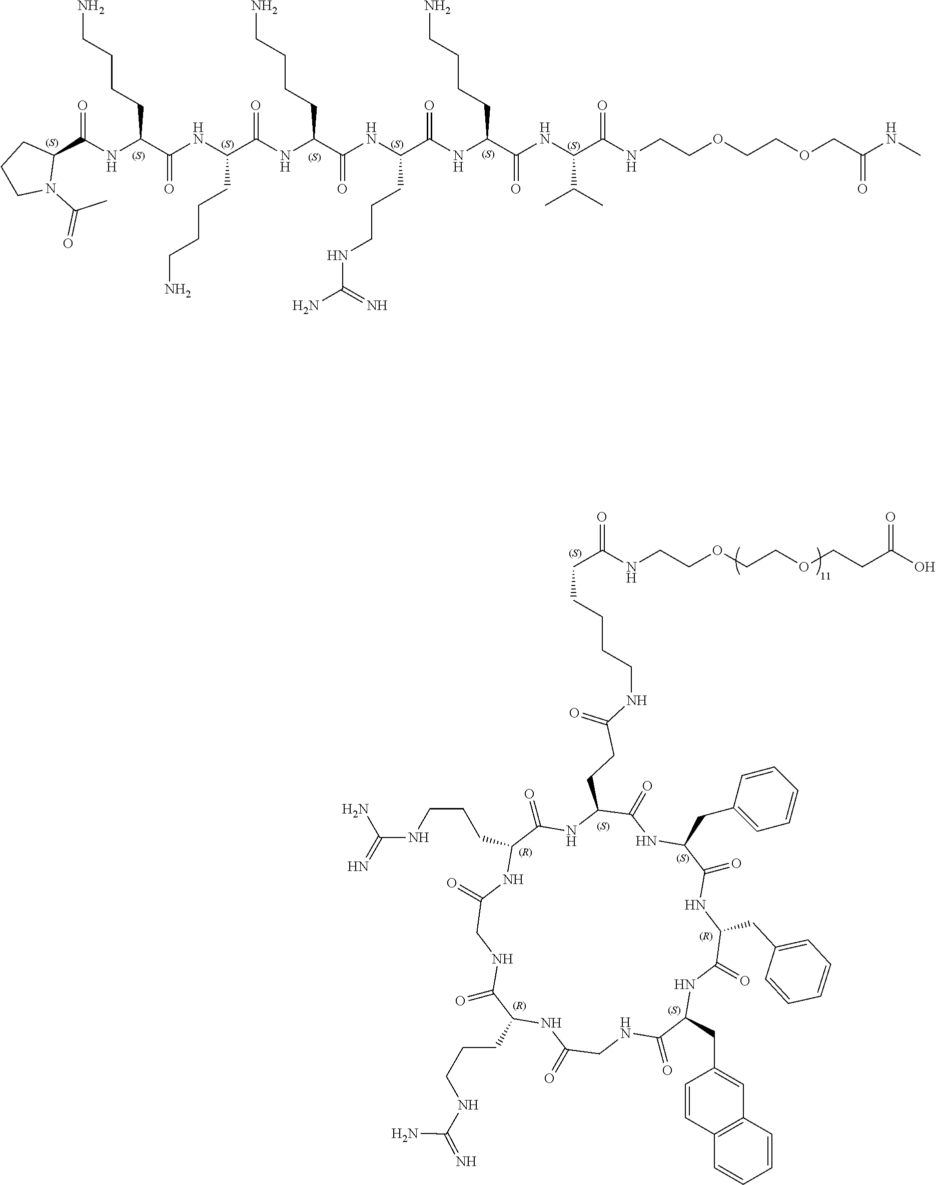

Embodiment 86 relates to an EEV of formula: Ac-P-K-K-K-R-K-V-miniPEG-K(cyclo(Ff-Nal-GrGrQ)-PEG12-OH (SEQ ID NO: 272).

Embodiment 87 relates to an EEV of formula:

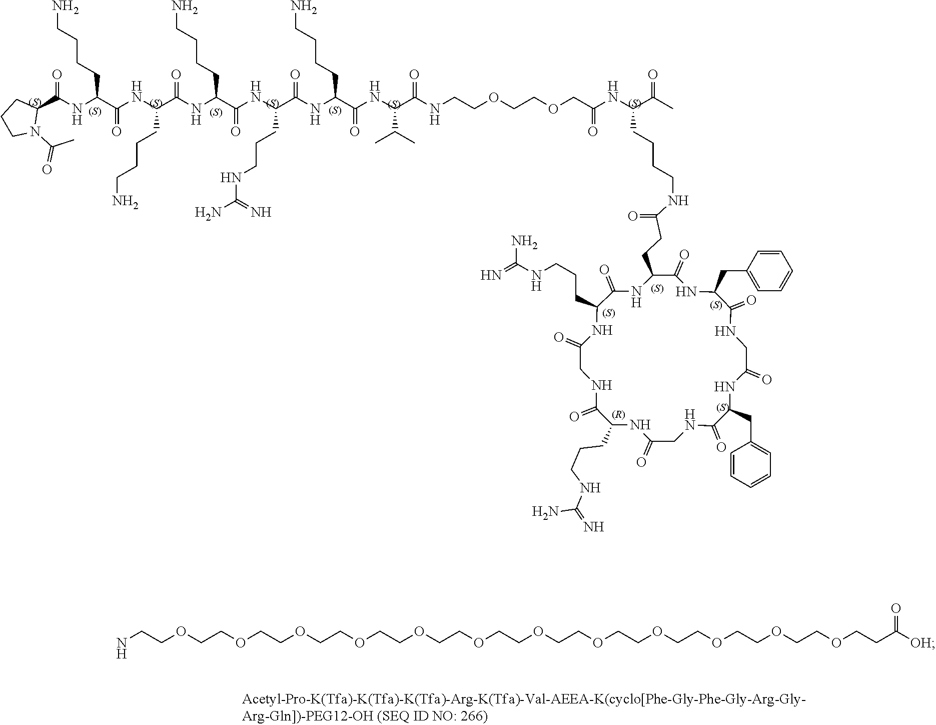

Embodiment 88 relates to an EEV of formula:

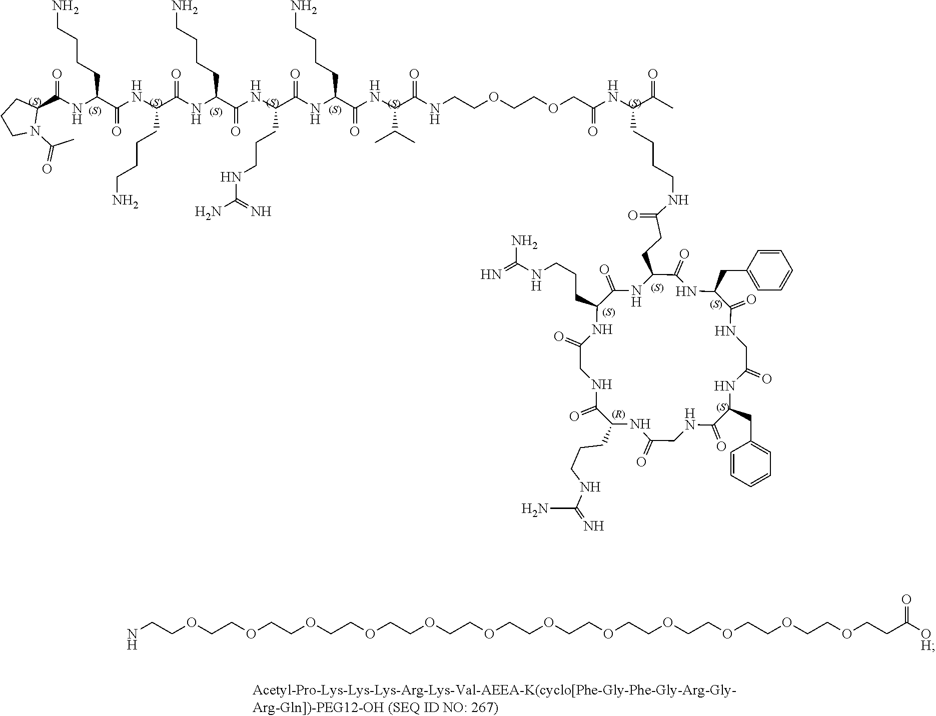

Embodiment 89 relates to an EEV of formula:

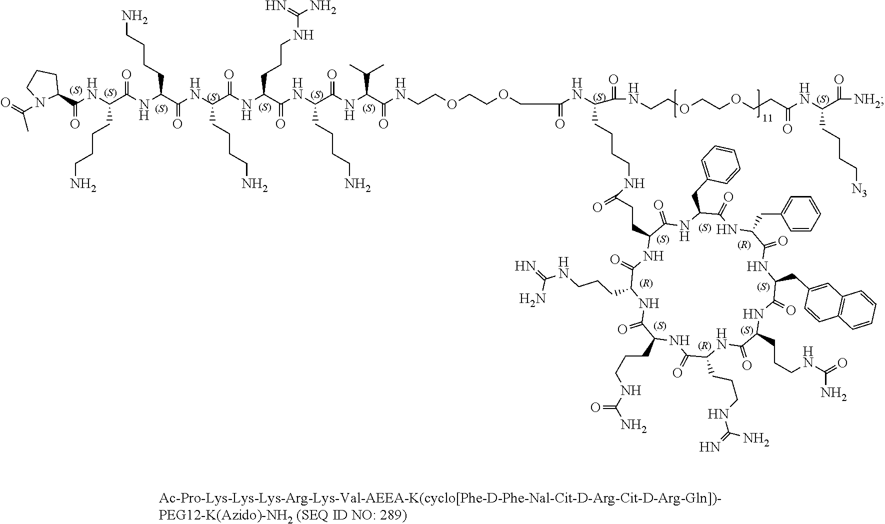

Embodiment 90 relates to an EEV of formula:

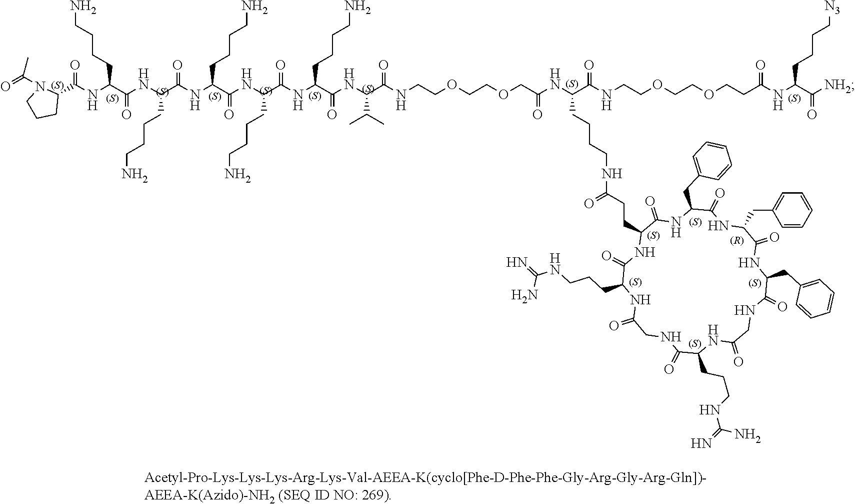

Embodiment 91 relates to an EEV of formula:

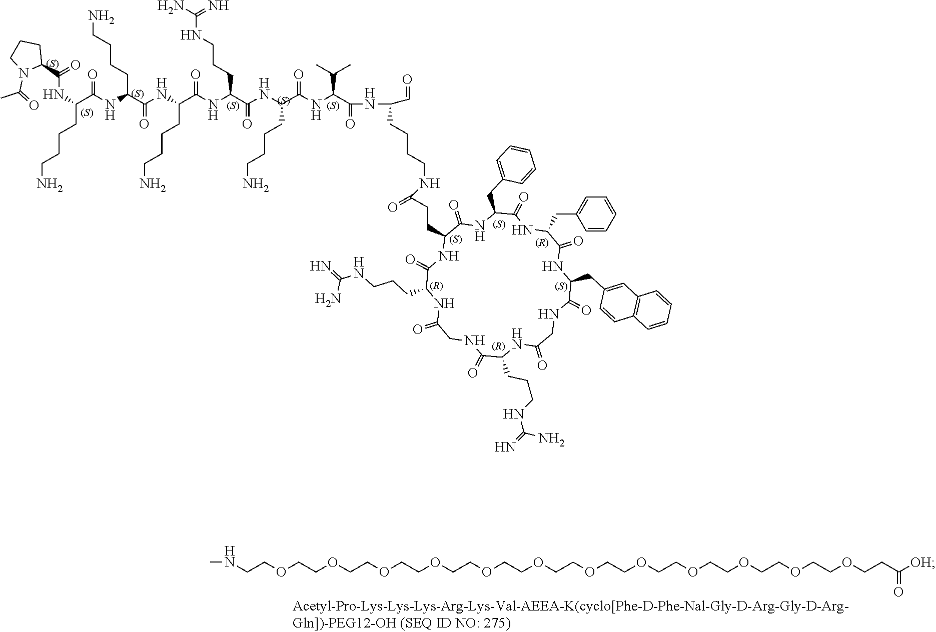

Embodiment 92 relates to an EEV of formula:

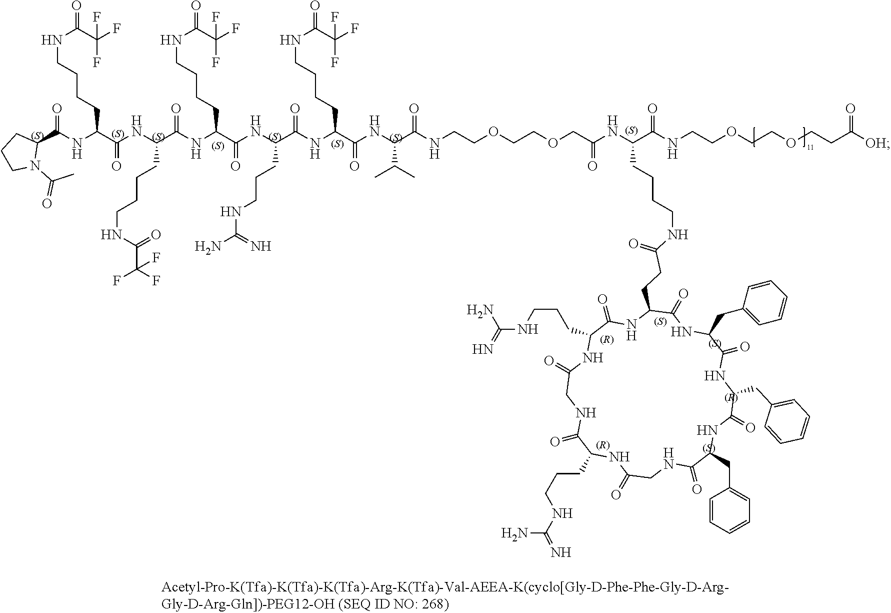

Embodiment 93 relates to an EEV of formula:

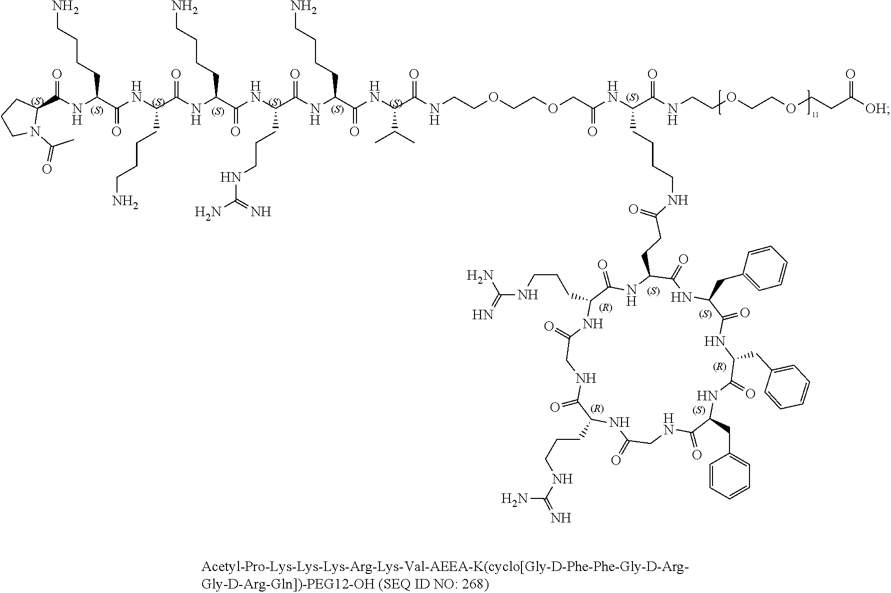

Embodiment 94 relates to an EEV of formula:

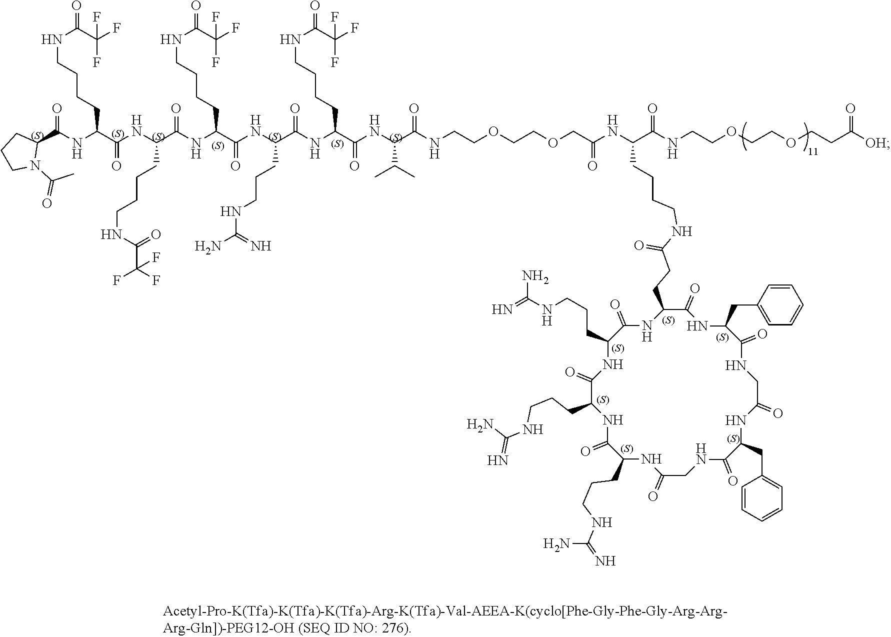

Embodiment 95 relates to an EEV of formula:

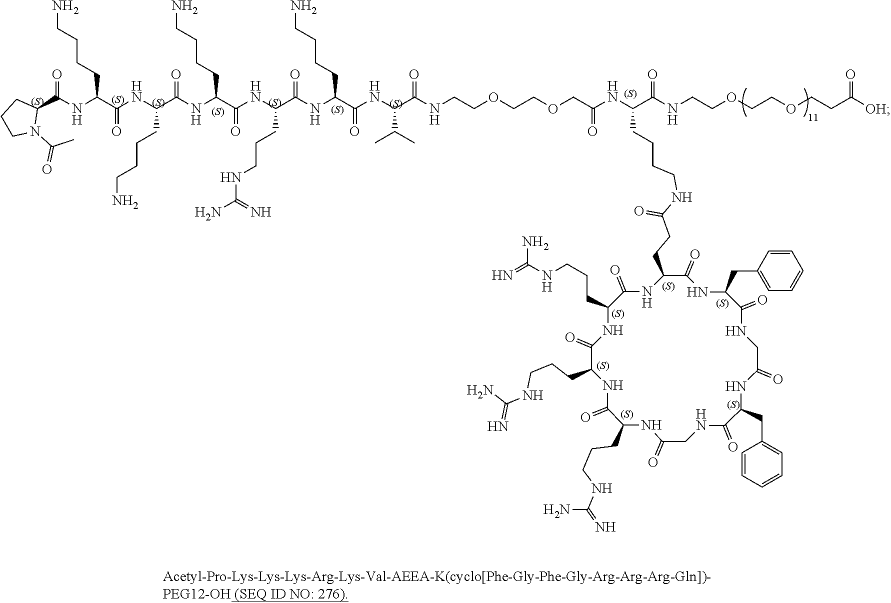

Embodiment 96 relates to an EEV of formula:

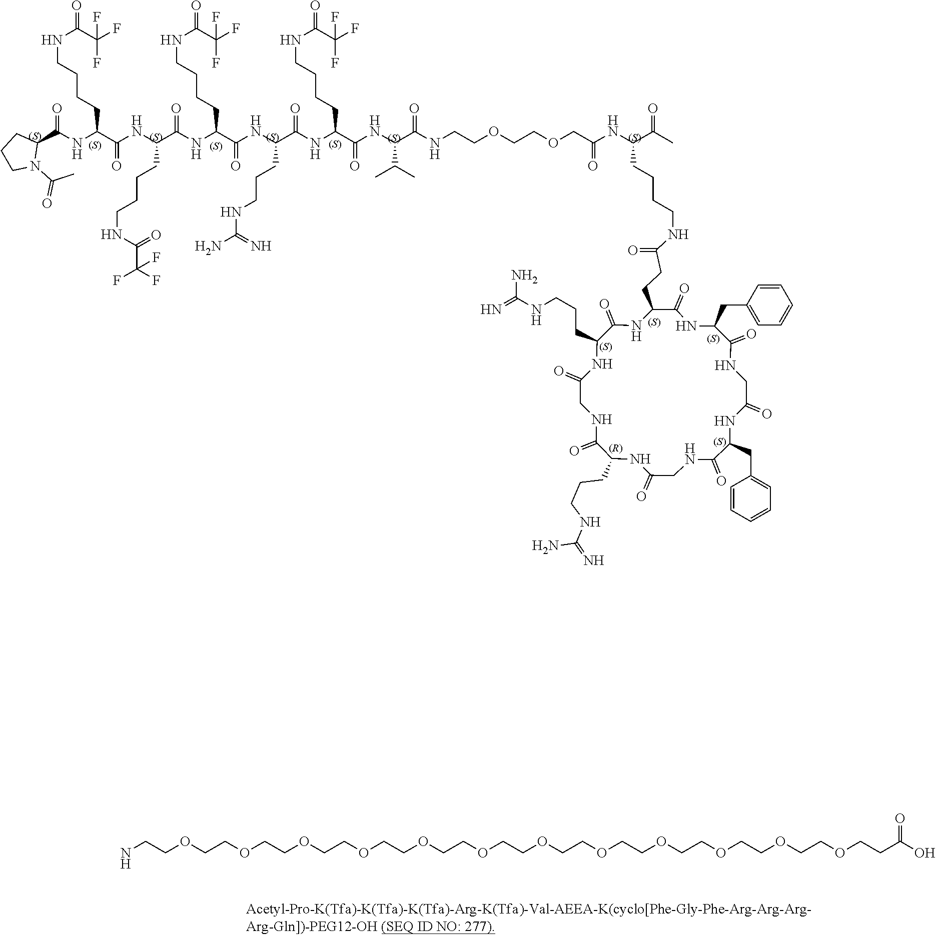

Embodiment 97 relates to an EEV of formula:

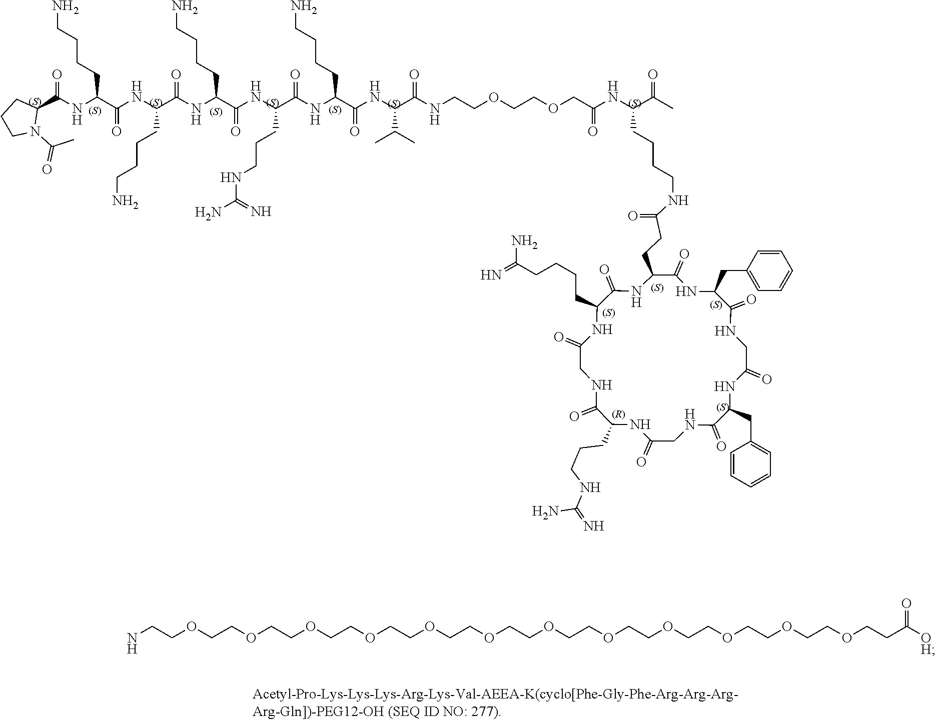

Embodiment 98 relates to an EEV selected from:

(SEQ ID NO: 236)
Ac-rr-miniPEG2-Dap[cyclo(Ffϕ-Cit-r-Cit-rQ)]-PEG12-OH
(SEQ ID NO: 239)
Ac-frr-PEG2-Dap(cyclo(Ffϕ-Cit-r-Cit-rQ))-PEG12-OH
(SEQ ID NO: 239)
Ac-rfr-PEG2-Dap(cyclo(Ffϕ-Cit-r-Cit-rQ))-PEG12-OH
(SEQ ID NO: 239)
Ac-rbfbr-PEG2-Dap(cyclo(Ffϕ-Cit-r-Cit-rQ))-PEG12-OH
(SEQ ID NO: 239)
Ac-rrr-PEG2-Dap(cyclo(Ffϕ-Cit-r-Cit-rQ))-PEG12-OH
(SEQ ID NO: 239)
Ac-rbr-PEG2-Dap(cyclo(Ffϕ-Cit-r-Cit-rQ))-PEG12-OH
(SEQ ID NO: 239)
Ac-rbrbr-PEG2-Dap(cyclo(Ffϕ-Cit-r-Cit-rQ))-PEG12-OH
(SEQ ID NO: 239)
Ac-hh-PEG2-Dap(cyclo(Ffϕ-Cit-r-Cit-rQ))-PEG12-OH
(SEQ ID NO: 239)
Ac-hbh-PEG2-Dap(cyclo(Ffϕ-Cit-r-Cit-rQ))-PEG12-OH
(SEQ ID NO: 239)
Ac-hbhbh-PEG2-Dap(cyclo(Ffϕ-Cit-r-Cit-rQ))-PEG12-OH
(SEQ ID NO: 239)
Ac-rbhbh-PEG2-Dap(cyclo(Ffϕ-Cit-r-Cit-rQ))-PEG12-OH
(SEQ ID NO: 239)
Ac-hbrbh-PEG2-Dap(cyclo(Ffϕ-Cit-r-Cit-rQ))-PEG12-OH
(SEQ ID NO: 239)
Ac-rr-Dap(cyclo(Ffϕ-Cit-r-Cit-rQ))-b-OH
(SEQ ID NO: 239)
Ac-frr-Dap(cyclo(Ffϕ-Cit-r-Cit-rQ))-b-OH
(SEQ ID NO: 239)
Ac-rfr-Dap(cyclo(Ffϕ-Cit-r-Cit-rQ))-b-OH
(SEQ ID NO: 239)
Ac-rbfbr-Dap(cyclo(Ffϕ-Cit-r-Cit-rQ))-b-OH
(SEQ ID NO: 239)
Ac-rrr-Dap(cyclo(Ffϕ-Cit-r-Cit-rQ))-b-OH
(SEQ ID NO: 239)
Ac-rbr-Dap(cyclo(Ffϕ-Cit-r-Cit-rQ))-b-OH
(SEQ ID NO: 239)
Ac-rbrbr-Dap(cyclo(Ffϕ-Cit-r-Cit-rQ))-b-OH
(SEQ ID NO: 239)
Ac-hh-Dap(cyclo(Ffϕ-Cit-r-Cit-rQ))-b-OH
(SEQ ID NO: 239)
Ac-hbh-Dap(cyclo(Ffϕ-Cit-r-Cit-rQ))-b-OH
(SEQ ID NO: 239)
Ac-hbhbh-Dap(cyclo(Ffϕ-Cit-r-Cit-rQ))-b-OH
(SEQ ID NO: 239)
Ac-rbhbh-Dap(cyclo(Ffϕ-Cit-r-Cit-rQ))-b-OH
(SEQ ID NO: 239)
Ac-hbrbh-Dap(cyclo(Ffϕ-Cit-r-Cit-rQ))-b-OH
(SEQ ID NOs: 64 and 234)
Ac-KKKK-miniPEG2-K(cyclo(Ff-Nal-GrGrQ))-miniPEG2-K(N3)-NH2
(SEQ ID NOs: 70 and 234)
Ac-KGKK-miniPEG2-K(cyclo(Ff-Nal-GrGrQ))-miniPEG2-K(N3)-NH2
(SEQ ID NOs: 71 and 234)
Ac-KKGK-miniPEG2-K(cyclo(Ff-Nal-GrGrQ))-miniPEG2-K(N3)-NH2
(SEQ ID NO: 234)
Ac-KKK-miniPEG2-K(cyclo(Ff-Nal-GrGrQ))-miniPEG2-K(N3)-NH2
(SEQ ID NO: 234)
Ac-KK-miniPEG2-K(cyclo(Ff-Nal-GrGrQ))-miniPEG2-K(N3)-NH2
(SEQ ID NO: 234)
Ac-KGK-miniPEG2-K(cyclo(Ff-Nal-GrGrQ))-miniPEG2-K(N3)-NH2
(SEQ ID NO: 234)
Ac-KBK-miniPEG2-K(cyclo(Ff-Nal-GrGrQ))-miniPEG2-K(N3)-NH2
(SEQ ID NO: 234)
Ac-KBKBK-miniPEG2-K(cyclo(Ff-Nal-GrGrQ))-miniPEG2-K(N3)-NH2
(SEQ ID NO: 234)
Ac-KR-miniPEG2-K(cyclo(Ff-Nal-GrGrQ))-miniPEG2-K(N3)-NH2
(SEQ ID NO: 234)
Ac-KBR-miniPEG2-K(cyclo(Ff-Nal-GrGrQ))-miniPEG2-K(N3)-NH2
(SEQ ID NOs: 103and 234)
Ac-PKKKRKV-miniPEG2-K(cyclo(Ff-Nal-GrGrQ))-miniPEG2-K(N3)-NH2
(SEQ ID NOs: 103 and 234)
Ac-PKKKRKV-miniPEG2-K(cyclo(Ff-Nal-GrGrQ))-miniPEG2-K(N3)-NH2
(SEQ ID NOs: 104 and 234)
Ac-PGKKRKV-miniPEG2-K(cyclo(Ff-Nal-GrGrQ))-miniPEG2-K(N3)-NH2
(SEQ ID NOs: 105 and 234)
Ac-PKGKRKV-miniPEG2-K(cyclo(Ff-Nal-GrGrQ))-miniPEG2-K(N3)-NH2
(SEQ ID NOs: 106 and 234)
Ac-PKKGRKV-miniPEG2-K(cyclo(Ff-Nal-GrGrQ))-miniPEG2-K(N3)-NH2
(SEQ ID NOs: 107 and 234)
Ac-PKKKGKV-miniPEG2-K(cyclo(Ff-Nal-GrGrQ))-miniPEG2-K(N3)-NH2
(SEQ ID NOs: 103 and 234)
Ac-PKKKRGV-miniPEG2-K(cyclo(Ff-Nal-GrGrQ))-miniPEG2-K(N3)-NH2
(SEQ ID NOs: 108 and 234)
Ac-PKKKRKG-miniPEG2-K(cyclo(Ff-Nal-GrGrQ))-miniPEG2-K(N3)-NH2
(SEQ ID NOs: 76 and 234)
Ac-KKKRK-miniPEG2-K(cyclo(Ff-Nal-GrGrQ))-miniPEG2-K(N3)-NH2
(SEQ ID NOs: 65 and 234)
Ac-KKRK-miniPEG2-K(cyclo(Ff-Nal-GrGrQ))-miniPEG2-K(N3)-NH2
and
(SEQ ID NO: 234)
Ac-KRK-miniPEG2-K(cyclo(Ff-Nal-GrGrQ))-miniPEG2-K(N3)-NH2,

Embodiment 99 relates to an EEV selected from:

(SEQ ID NO: 272)
Ac-PKKKRKV-Lys(cyclo[FfϕGrGrQ])-PEG₁₂-K(Ns)-NH₂
(SEQ ID NOs: 103 and 234)
Ac-PKKKRKV-miniPEG₂-Lys(cyclo[FfϕGrGrQ])-miniPEG₂-K(N₃)-NH₂
(SEQ ID NOs: 103 and 128)
Ac-PKKKRKV-miniPEG₂-Lys(cyclo[FGFGRGRQ])-miniPEG₂-K(N₃)-NH₂
(SEQ ID NO: 128)
Ac-KR-PEG₂-K(cyclo[FGFGRGRQ])-PEG₂-K(N₃)-NH₂
(SEQ ID NOs: 103 and 128)
Ac-PKKKGKV-PEG₂-K(cyclo[FGFGRGRQ])-PEG₂-K(N₃)-NH₂
(SEQ ID NOs: 109 and 128)
Ac-PKKKRKG-PEG₂-K(cyclo[FGFGRGRQ])-PEG₂-K(N₃)-NH₂
(SEQ ID NOs: 76 and 128)
Ac-KKKRK-PEG₂-K(cyclo[FGFGRGRQ])-PEG₂-K(N₃)-NH₂
(SEQ ID NOs: 103 and 234)
Ac-PKKKRKV-miniPEG₂-K(cyclo[FFϕGRGRQ])-miniPEG₂-K(N₃)-NH₂
(SEQ ID NOS: 103 and 234)
Ac-PKKKRKV-miniPEG₂-K(cyclo[BhFfϕGrGrQ])-miniPEG₂-K(N₃)-NH₂
and
(SEQ ID NOs: 103 and 251)
Ac-PKKKRKV-miniPEG₂-K(cyclo[FfϕSrSrQ])-miniPEG₂-K(N₃)-NH₂.

Embodiment 100 relates to an An EEV selected from.

(SEQ ID NOs: 103 and 233)
Ac-PKKKRKV-miniPEG₂-K(cyclo(GfFGrGrQ])-PEG₁₂-OH
(SEQ ID NOs: 103 and 132)
Ac-PKKKRKV-miniPEG₂-K(cyclo[FGFKRKRQ])-PEG₁₂-OH
(SEQ ID NOs: 103 and 133)
Ac-PKKKRKV-miniPEG₂-K(cyclo[FGFRGRGQ])-PEG₁₂-OH
(SEQ ID NOs: 103 and 134)
Ac-PKKKRKV-miniPEG₂-K(cyclo[FGFGRGRGRQ])-PEG₁₂-OH
(SEQ ID NOs: 103 and 130)
Ac-PKKKRKV-miniPEG₂-K(cyclo[FGFGRrRQ])-PEG₁₂-OH
(SEQ ID NOs: 103 and 130)
Ac-PKKKRKV-miniPEG₂-K(cyclo[FGFGRRRQ])-PEG₁₂-OH
and
(SEQ ID NOs: 103 and 129)
Ac-PKKKRKV-miniPEG₂-K(cyclo[FGFRRRRQ])-PEG₁₂-OH.

(SEQ ID NOs: 290 and 128)
Ac-KKKRKG-miniPEG₂-K(cyclo[FGFGRGRQ])-PEG₁₂-OH
(SEQ ID NOs: 76 and 128)
Ac-KKKRK-miniPEG₂-K(cyclo[FGFGRGRQ])-PEG₁₂-OH
(SEQ ID NOs: 79 and 128)
Ac-KKRKK-PEG₄-K(cyclo[FGFGRGRQ])-PEG₁₂-OH
(SEQ ID NOs: 78 and 128)
Ac-KRKKK-PEG₄-K(cyclo[FGFGRGRQ])-PEG₁₂-OH
(SEQ ID NOs: 80 and 128)
Ac-KKKKR-PEG₄-K(cyclo[FGFGRGRQ])-PEG₁₂-OH
(SEQ ID NOs: 77 and 128)
Ac-RKKKK-PEG₄-K(cyclo[FGFGRGRQ])-PEG₁₂-OH
and
(SEQ ID NOs: 76 and 128)
Ac-KKKRK-PEG₄-K(cyclo[FGFGRGRQ])-PEG₁₂-OH.

Embodiment 102 relates to an EEV selected from:

(SEQ ID NOs: 103 and 128)
Ac-PKKKRKV-PEG₂-K(cyclo[FGFGRGRQ])-PEG₂-K(N₃)-NH₂
(SEQ ID NOs: 103 and 128)
Ac-PKKKRKV-PEG₂-K(cyclo[FGFGRGRQ])-PEG₁₂-OH
(SEQ ID NOs: 103 and 233)
Ac-PKKKRKV-PEG₂-K(cyclo[GfFGrGrQ])-PEG₂-K(N₃)-NH₂
and
(SEQ ID NOs: 103 and 233)
Ac-PKKKRKV-PEG₂-K(cyclo[GfFGrGrQ])-PEG₁₂-OH.

Embodiment 103 relates to a cargo, wherein the cargo is a protein and can EEV selected from:

(SEQ ID NOs: 103 and 234)
Ac-PKKKRKV-PEG₂-K(cyclo[Ff-Nal-GrGrQ])-PEG₁₂-OH
(SEQ ID NOs: 103 and 236)
Ac-PKKKRKV-PEG₂-K(cyclo[Ff-Nal-Cit-r-Cit-rQ])-
PEG₁₂-OH
(SEQ ID NOs: 103 and 235)
Ac-PKKKRKV-PEG₂-K(cyclo[FfF-GRGRQ])-PEG₁₂-OH
(SEQ ID NOs: 103 and 128)
Ac-PKKKRKV-PEG₂-K(cyclo[FGFGRGRQ])-PEG₁₂-OH
(SEQ ID NOs: 103 and 233)
Ac-PKKKRKV-PEG₂-K(cyclo[GfFGrGrQ])-PEG₁₂-OH
(SEQ ID NOs: 103 and 128)
Ac-PKKKRKV-PEG₂-K(cyclo[FGFGRRRQ])-PEG₁₂-OH
(SEQ ID NOs: 103 and 129)
Ac-PKKKRKV-PEG₂-K(cyclo[FGFRRRRQ])-PEG₁₂-OH
(SEQ ID NO: 234)
Ac-rr-PEG₂-K(cyclo[Ff-Nal-GrGrQ])-PEG₁₂-OH
(SEQ ID NO: 236)
Ac-rr-PEG₂-K(cyclo[Ff-Nal-Cit-r-Cit-rQ])-PEG₁₂-OH
(SEQ ID NO: 235)
Ac-rr-PEG₂-K(cyclo[FfF-GRGRQ])-PEG₁₂-OH
(SEQ ID NO: 128)
Ac-rr-PEG₂-K(cyclo[FGFGRGRQ])-PEG₁₂-OH
(SEQ ID NO: 233)
Ac-rr-PEG₂-K(cyclo[GfFGrGrQ])-PEG₁₂-OH
(SEQ ID NO: 130)
Ac-rr-PEG₂-K(cyclo[FGFGRRRQ])-PEG₁₂-OH
(SEQ ID NO: 129)
Ac-rr-PEG₂-K(cyclo[FGFRRRRQ])-PEG₁₂-OH
(SEQ ID NO: 234)
Ac-rrr-PEG₂-K(cyclo[Ff-Nal-GrGrQ])-PEG₁₂-OH
(SEQ ID NO: 236)
Ac-rrr-PEG₂-K(cyclo[Ff-Nal-Cit-r-Cit-rQ])-PEG₁₂-
OH
(SEQ ID NO: 235)
Ac-rrr-PEG₂-K(cyclo[FfF-GRGRQ])-PEG₁₂-OH
(SEQ ID NO: 128)
Ac-rrr-PEG₂-K(cyclo[FGFGRGRQ])-PEG₁₂-OH
(SEQ ID NO: 233)
Ac-rrr-PEG₂-K(cyclo[GfFGrGrQ])-PEG₁₂-OH
(SEQ ID NO: 130)
Ac-rrr-PEG₂-K(cyclo[FGFGRRRQ])-PEG₁₂-OH
(SEQ ID NO: 129)
Ac-rrr-PEG₂-K(cyclo[FGFRRRRQ])-PEG₁₂-OH
(SEQ ID NO: 234)
Ac-rhr-PEG₂-K(cyclo[Ff-Nal-GrGrQ])-PEG₁₂-OH
(SEQ ID NO: 236)
Ac-rhr-PEG₂-K(cyclo[Ff-Nal-Cit-r-Cit-rQ])-PEG₁₂-
OH
(SEQ ID NO: 235)
Ac-rhr-PEG₂-K(cyclo[FfF-GRGRQ])-PEG₁₂-OH
(SEQ ID NO: 128)
Ac-rhr-PEG₂-K(cyclo[FGFGRGRQ])-PEG₁₂-OH
(SEQ ID NO: 233)
Ac-rhr-PEG₂-K(cyclo[GfFGrGrQ])-PEG₁₂-OH
(SEQ ID NO: 130)
Ac-rhr-PEG₂-K(cyclo[FGFGRRRQ])-PEG₁₂-OH
(SEQ ID NO: 131)
Ac-rhr-PEG₂-K(cyclo[FGFRRRRQ])-PEG₁₂-OH
(SEQ ID NO: 234)
Ac-rbr-PEG₂-K(cyclo[Ff-Nal-GrGrQ])-PEG₁₂-OH
(SEQ ID NO: 236)
Ac-rbr-PEG₂-K(cyclo[Ff-Nal-Cit-r-Cit-rQ])-PEG₁₂-
OH
(SEQ ID NO: 235)
Ac-rbr-PEG₂-K(cyclo[FfF-GRGRQ])-PEG₁₂-OH
(SEQ ID NO: 128)
Ac-rbr-PEG₂-K(cyclo[FGFGRGRQ])-PEG₁₂-OH
(SEQ ID NO: 233)
Ac-rbr-PEG₂-K(cyclo[GfFGrGrQ])-PEG₁₂-OH
(SEQ ID NO: 130)
Ac-rbr-PEG₂-K(cyclo[FGFGRRRQ])-PEG₁₂-OH
(SEQ ID NO: 131)
Ac-rbr-PEG₂-K(cyclo[FGFRRRRQ])-PEG₁₂-OH
(SEQ ID NO: 234)
Ac-rbrbr-PEG₂-K(cyclo[Ff-Nal-GrGrQ])-PEG₁₂-OH
(SEQ ID NO: 236)
Ac-rbrbr-PEG₂-K(cyclo[Ff-Nal-Cit-r-Cit-rQ])-PEG₁₂-
OH
(SEQ ID NO: 235)
Ac-rbrbr-PEG₂-K(cyclo[FfF-GRGRQ])-PEG₁₂-OH
(SEQ ID NO: 128)
Ac-rbrbr-PEG₂-K(cyclo[FGFGRGRQ])-PEG₁₂-OH
(SEQ ID NO: 233)
Ac-rbrbr-PBG₂-K(cyclo[GfFGrGrQ])-PEG₁₂-OH
(SEQ ID NO: 130)
Ac-rbrbr-PEG₂-K(cyclo[FGFGRRRQ])-PEG₁₂-OH
(SEQ ID NO: 131)
Ac-rbrbr-PEG₂-K(cyclo[FGFRRRRQ])-PEG₁₂-OH
(SEQ ID NO: 234)
Ac-rbhbr-PEG₂-K(cyclo[Ff-Nal-GrGrQ])-PEG₁₂-OH
(SEQ ID NO: 236)
Ac-rbhbr-PEG₂-K(cyclo[Ff-Nal-Cit-r-Cit-rQ])-PEG₁₂-
OH
(SEQ ID NO: 235)
Ac-rbhbr-PEG₂-K(cyclo[FfF-GRGRQ])-PEG₁₂-OH
(SEQ ID NO: 128)
Ac-rbhbr-PEG₂-K(cyclo[FGFGRGRQ])-PEG₁₂-OH
(SEQ ID NO: 233)
Ac-rbhbr-PEG₂-K(cyclo[GfFGrGrQ])-PEG₁₂-OH
(SEQ ID NO: 130)
Ac-rbhbr-PEG₂-K(cyclo[FGFGRRRQ])-PEG₁₂-OH
(SEQ ID NO: 131)
Ac-rbhbr-PEG₂-K(cyclo[FGFRRRRQ])-PEG₁₂-OH
(SEQ ID NO: 234)
Ac-hbrbh-PEG₂-K(cyclo[Ff-Nal-GrGrQ])-PEG₁₂-OH
(SEQ ID NO: 236)
Ac-hbrbh-PEG₂-K(cyclo[Ff-Nal-Cit-r-Cit-rQ])-PEG₁₂-
OH
(SEQ ID NO: 235)
Ac-hbrbh-PEG₂-K(cyclo[FfF-GRGRQ])-PEG₁₂-OH
(SEQ ID NO: 128)
Ac-hbrbh-PEG₂-K(cyclo[FGFGRGRQ])-PEG₁₂-OH
(SEQ ID NO: 233)
Ac-hbrbh-PEG₂-K(cyclo[GfFGrGrQ])-PEG₁₂-OH
(SEQ ID NO: 130)
Ac-hbrbh-PEG₂-K(cyclo[FGFGRRRQ])-PEG₁₂-OH
(SEQ ID NO: 131)
Ac-hbrbh-PEG₂-K(cyclo[FGFRRRRQ])-PEG₁₂-OH

What is claimed is:

1. A compound comprising:
(a) a cyclic peptide of formula: 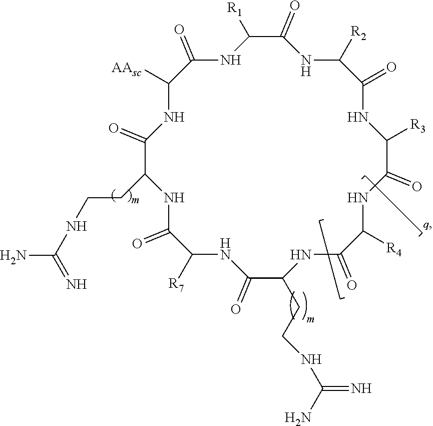
or a protonated form thereof,
wherein
R₁, R₂, and R₃ are each independently H or a side chain comprising an aryl or heteroaryl group;
at least two of R₁, R₂, and R₃ are a side chain of phenylalanine;
AA_{SC} is an amino acid side chain;
R₄ and R₇ are independently H or an amino acid side chain;
each m is independently an integer from 0-3; and
q is an integer from 1-4;
(b) an exocyclic peptide comprising from 2 to 10 amino acid residues, wherein 1 or 2 amino acid residues comprise a side chain of a guanidine group, or a protonated form thereof or 2, 3, or 4 lysine residues; and
(c) a linker comprising:
(i) a —(OCH₂CH₂)_{z}— subunit, wherein z′ is an integer from 1 to 23;
(ii) one or more amino acid residues, such as a residue of glycine, β-alanine, 4-aminobutyric acid, 5-aminopentoic acid or 6-aminohexanoic acid, or combinations thereof; or
(iii) combinations of (i) and (ii).

2. The compound of claim 1, wherein the linker is of formula: 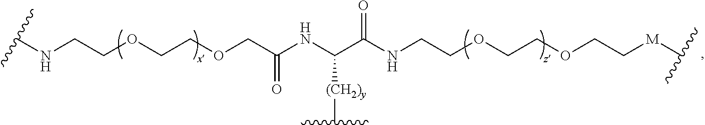
wherein
x′ is an integer from 1-23;
y is an integer from 1-5;
z′ is an integer from 1-23;
* is the point of attachment to the AA_{SC}, and
M is a bonding group.

3. The compound of claim 2, wherein M comprises

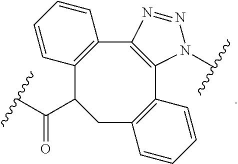 [SMILES: *C(=O)C1Cc2ccccc2-c2c(nnn2*)-c2ccccc21]

4. The compound of claim 2, wherein M comprises 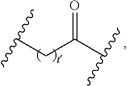
wherein t′ is an integer from 0 to 10.

5. The compound of claim 2, wherein the linker has the formula:

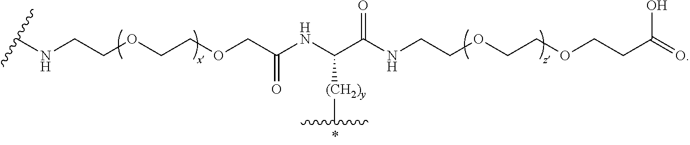

6. The compound of claim 2 wherein:
z′ is 11;
x′ is 1; or
z′ is 11 and x′ is 1.

7. The compound of claim 1, wherein two of R₁, R₂, R₃, and R₄ are H.

8. The compound of claim 1, wherein q is 1.

9. The compound of claim 1, conjugated to a therapeutic moiety selected from an oligonucleotide, a peptide and a small molecule.

10. The compound of claim 1 wherein:
(i) the exocyclic peptide comprises 1 or 2 amino acid residues comprising a side chain comprising a guanidine group, or a protonated form thereof; and/or
(ii) the exocyclic peptide comprises 2, 3, or 4 lysine residues; and/or
(iii) the exocyclic peptide comprises at least 2 amino acid residues with a hydrophobic side chain, for example wherein the hydrophobic side chain is selected from valine, proline, alanine, leucine, isoleucine, and methionine.

11. The compound of claim 1, wherein:
(i) the exocyclic peptide comprises one of the following sequences: KK, KR, RR, HH, HK, HR, RH, KKK, KGK, KBK, KBR, KRK, KRR, RKK, RRR, KKH, KHK, HKK, HRR, HRH, HHR, HBH, HHH, HHHH, KHKK, KKHK, KKKH, KHKH, HKHK, KKKK, KKRK, KRKK, KRRK, RKKR, RRRR, KGKK, KKGK, HBHBH, HBKBH, RRRRR, KKKKK, KKKRK, RKKKK, KRKKK, KKRKK, KKKKR, KBKBK, RKKKKG, KRKKKG, KKRKKG, KKKKRG, RKKKKB, KRKKKB, KKRKKB, KKKKRB, KKKRKV, RRRRRR, HHHHHH, RHRHRH, HRHRHR, KRKRKR, RKRKRK, RBRBRB, KBKBKB, PKKKRKV, PGKKRKV, PKGKRKV, PKKGRKV, PKKKGKV, PKKKRGV or PKKKRKG; or
(ii) the exocyclic peptide comprises one of the following sequences: RHR, RBR, RBRBR, RBHBR, or HBRBH, wherein B is beta-alanine.

12. The compound of claim 1, wherein the EP has the structure: Ac-PKKKRKV.

13. The compound of claim 1, wherein the cyclic peptide comprises: 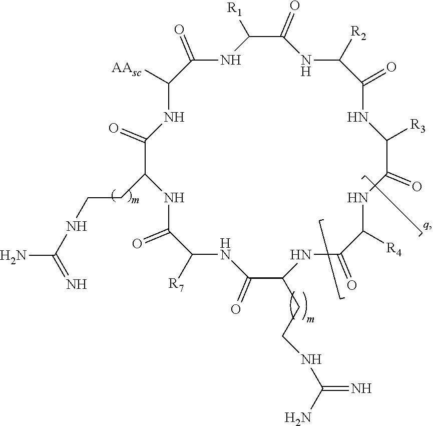
or a protonated form thereof,
wherein,
one of R₁, R₂, and R₃ is H;
two of R₁, R₂, and R₃ are CH₂Ph;
R₄ and R₆ are independently H or an amino acid side chain;
AA_{sc} is an amino acid side chain;
q is 1, 2, 3 or 4; and
each m is independently an integer 0, 1, 2, or 3.

14. The compound of claim 1, wherein the cyclic peptide comprises: 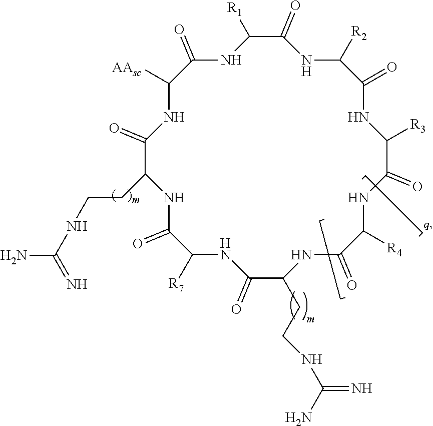
or a protonated form thereof,
wherein,
R₁, R₂, and R₃ are —CH₂Ph;
R₄ and R₆ are independently H or an amino acid side chain;
AA_{sc} is an amino acid side chain;
q is 1, 2, 3 or 4; and
each m is independently an integer 0, 1, 2, or 3.

15. The compound of claim 1, wherein the cyclic peptide comprises: 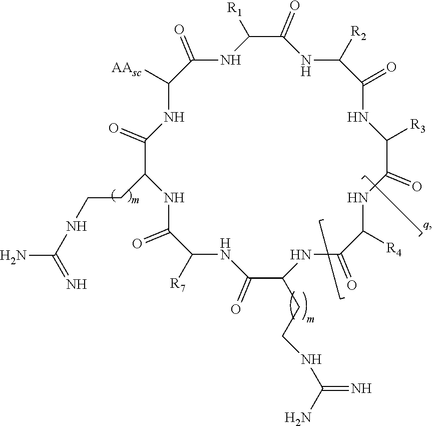
or a protonated form thereof,
wherein,
R₁, R₂, and R₃ are each independently a side chain comprising an aryl or heteroaryl group;
at least two of R₁, R₂, and R₃ are a side chain of phenylalanine;
AA_{SC} is an amino acid side chain;
R₄ and R₇ are independently H or an amino acid side chain of serine or citrulline
q is 1, 2, 3 or 4; and
each m is independently an integer 0, 1, 2, or 3.

16. The compound of claim 1 wherein the cyclic peptide has a structure selected from:
Formula (I-1), 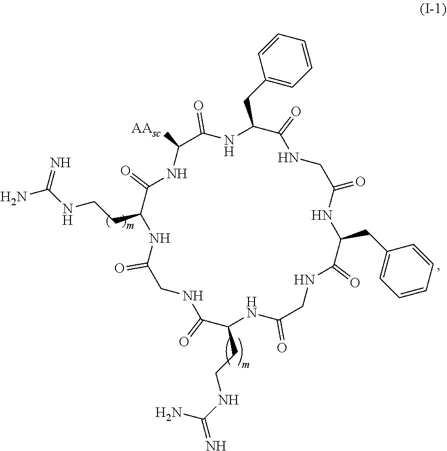
or a protonated form thereof;
Formula (I-2): 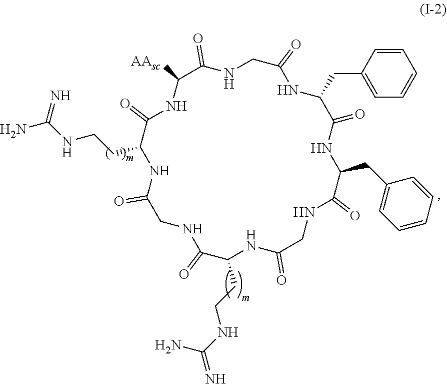
or a protonated form thereof;
Formula (I-3): 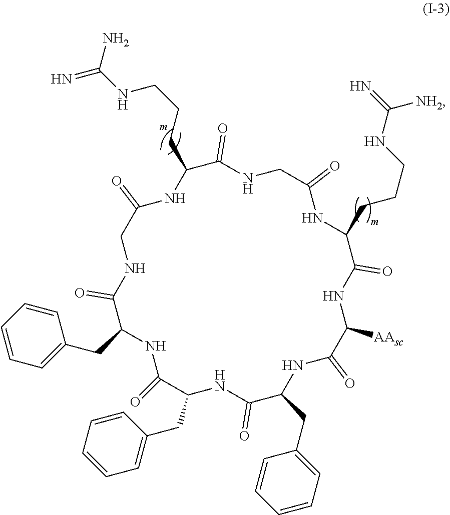
or a protonated form thereof;
Formula (I-4) 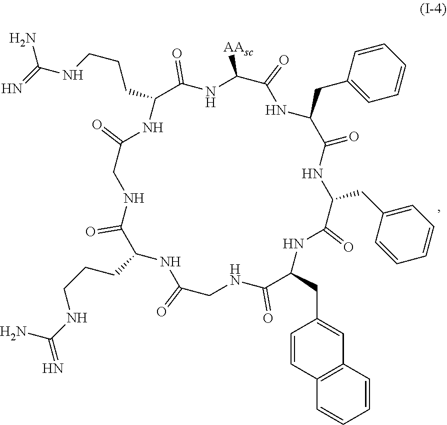
or a protonated form thereof;
Formula (I-5): 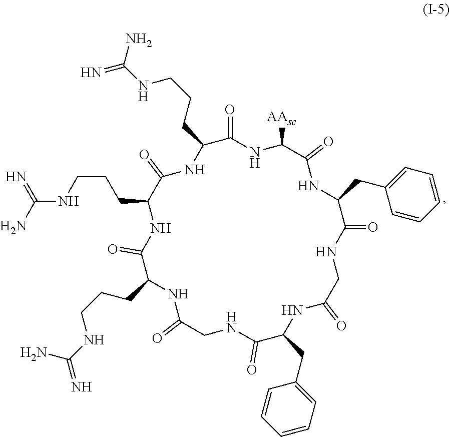
or a protonated form thereof; and
Formula (I-6): 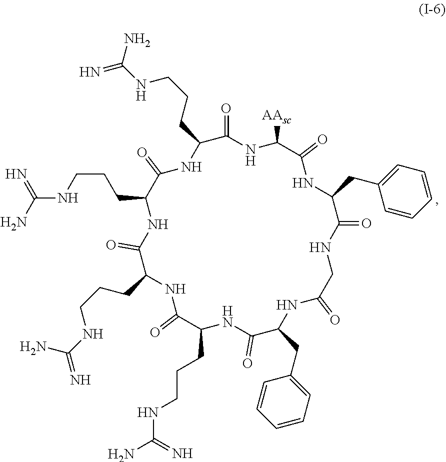
a protonated form thereof;
wherein each m is independently an integer from 0-3.

17. The compound of claim 1, wherein the cyclic peptide is selected from Ff-Nal-GrGrQ; FfFGRGRQ; FGFGRGRQ; GfFGrGrQ; FfFGRGRQ; FGFGRRRQ and FGFRRRRQ.

18. The compound of claim 1 having a formula selected from:
Ac-PKKKRKV-K(cyclo[Ff-Nal-GrGrQ])-PEG₁₂-K(N₃)-NH₂;
Ac-PKKKRKV-PEG₂-K(cyclo[FGFGRGRQ])-PEG₂-K(N₃)-NH₂;
Ac-PKKKRKV-PEG₂-K(cyclo[GfFGrGrQ])-PEG₂-K(N₃)-NH₂;
Ac-PKKKRKV-PEG₂-K(FfFGRGRQ)-miniPEG₂-K(N₃)-NH₂;
Ac-PKKKRKV-PEG₂-K(cyclo(Ff-Nal-GrGrQ)-PEG₁₂-OH;
Ac-PKKKRKV-PEG₂-K(cyclo[FGFGRGRQ])-PEG₁₂-OH;
Ac-PKKKRKV-PEG₂-K(cyclo[GfFGrGrQ])-PEG₁₂-OH;
Ac-PKKKRKV-K(cyclo[Ff-Nal-GrGrQ])-PEG₁₂-K(N3)-NH₂;
Ac-PKKKRKV-K(cyclo[Ff-Nal-GrGrQ])-PEG₂-K(N3)-NH₂;
Ac-PKKKRKV-PEG₂-K(cyclo[Ff-Nal-GrGrQ])-PEG₂-K(N₃)-
NH₂;
Ac-PKKKRKV-PEG₂-K(cyclo(Ff-Nal-GrGrQ)-PEG₁₂-OH;
Ac-PKKKRKV-PEG₂-K(cyclo(Ff-Nal-GrGrQ)-PEG₂-OH;
Ac-PKKKRKV-K(cyclo[FGFGRGRQ])-PEG₁₂-K(N₃)-NH₂;
Ac-PKKKRKV-K(cyclo[FGFGRGRQ])-PEG₂-K(N₃)-NH₂;
Ac-PKKKRKV-PEG₂-K(cyclo[FGFGRGRQ])-PEG₂-K(N₃)-NH₂;
Ac-PKKKRKV-PEG₂-K(cyclo(FGFGRGRQ)-PEG₁₂-OH;
Ac-PKKKRKV-PEG₂-K(cyclo(FGFGRGRQ)-PEG₂-OH;
Ac-PKKKRKV-K(cyclo[GfFGrGrQ])-PEG₁₂-K(N₃)-NH₂;
Ac-PKKKRKV-K(cyclo[GfFGrGrQ])-PEG₂-K(N₃)-NH₂;
Ac-PKKKRKV-PEG₂-K(cyclo[GfFGrGrQ])-PEG₂-K(N₃)-NH₂,
Ac-PKKKRKV-PEG₂-K(cyclo(GfFGrGrQ PEG₁₂-OH;
Ac-PKKKRKV-PEG₂-K(cyclo(GfFGrGrQ)-PEG₂-OH;
Ac-PKKKRKV-K(cyclo[FfFGRGRQ])-PEG₁₂-K(N₃)-NH₂;
Ac-PKKKRKV-K(cyclo[FfFGRGRQ])-PEG₂-K(N₃)-NH₂;
Ac-PKKKRKV-PEG₂-K(cyclo[FfFGRGRQ])-PEG₂-K(N₃)-NH₂;
Ac-PKKKRKV-PEG₂-K(cyclo(FfFGRGRQ)-PEG₁₂-OH;
Ac-PKKKRKV-PEG₂-K(cyclo(FfFGRGRQ)-PEG₂-OH;
Ac-PKKKRKV-PEG₂-K(cyclo[FGFGRRRQ])-PEG₁₂-OH;
Ac-PKKKRKV-K(cyclo[FGFGRRRQ])-PEG₁₂-K(N₃)-NH₂;
Ac-PKKKRKV-K(cyclo[FGFGRRRQ])-PEG₂-K(N₃)-NH₂;
Ac-PKKKRKV-PEG₂-K(cyclo[FGFGRGRQ])-PEG₂-K(N3)-NH₂;
Ac-PKKKRKV-PEG₂-K(cyclo(FGFGRRRQ)-PEG₁₂-OH;
and
Ac-PKKKRKV-PEG₂-K(cyclo(FGFGRRRQ)-PEG₂-OH.

19. A compound comprising:
(a) a cyclic peptide of claim 13;
(b) a linker of formula: 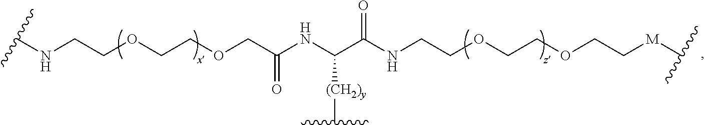
wherein
x′ is an integer from 1-23;
y is an integer from 1-5;
z′ is an integer from 1-23;
* is the point of attachment to the AA_{SC}, and
M is a bonding group; and
(c) an exocyclic peptide comprising from 2 to 10 amino acid residues wherein at least one of the amino acid residues is positively charged.

20. The compound of claim 1, having a formula selected from:
Ac-PKKKRKV-PEG₂-K(cyclo[Ff-Nal-Cit-r-Cit-r-Q])-PEG₁₂-K(N₃)-NH₂;
Ac-PKKKRKV-K(cyclo[Ffϕ-R-r-Cit-rQ])-PEG₁₂-K(N₃)-NH₂;
Ac-PKKKRKV-K(cyclo[Ffϕ-Cit-r-R-rQ])-PEG₁₂-K(N₃)-NH₂;
Ac-PKKKRKV-K(cyclo(FfϕDR-cit-R-cit-Q))-PEG₁₂-K(N₃)-NH₂;
Ac-PKKKRKV-PEG₂-K(cyclo[Ffϕ-Cit-r-Cit-rQ])-PEG₂-k(N₃)-NH₂;
Ac-PKKKRKV-PEG₂-K(cyclo[Ffϕ-Cit-r-Cit-rQ])-PEG₄-k(N₃)-NH₂;
Ac-PKKKRKV-K(cyclo[Ffϕ-Cit-r-Cit-rQ])-PEG₁₂-k(N₃)-NH₂;
Ac-PKKKRKV-PEG₂-K(cyclo[Ffϕ-Cit-r-Cit-r-Q])-PEG₁₂-k(N₃)-NH₂;
and
Ac-PKKKRKV-PEG₂-K(cyclo[Ffϕ-Cit-r-Cit-r-Q]-PEG₁₂-K(N₃)-NH₂.

21. A method of treating a disease or pathology in a subject in need thereof comprising administering to the subject an effective amount of the compound of claim 1.


SEQUENCE LISTING (290 sequences)

SEQ ID NO: 1 - protein, 5 aa, synthetic construct
  REGION 1..5: note = A synthetic sequence
RRRRR

SEQ ID NO: 2 - protein, 5 aa, synthetic construct
  REGION 1..5: note = A synthetic sequence
AAAAA

SEQ ID NO: 3 - protein, 4 aa, synthetic construct
  REGION 1..4: note = A synthetic sequence
FFFF

SEQ ID NO: 4 - protein, 5 aa, synthetic construct
  MOD_RES 3: note = phosphocoumaryl amino propionic acid
DEXLI

SEQ ID NO: 5 - protein, 7 aa, synthetic construct
  REGION 1..7: note = A synthetic sequence
AAAAAAA

SEQ ID NO: 6 - protein, 5 aa, synthetic construct
  REGION 1..5: note = A synthetic sequence
RARAR

SEQ ID NO: 7 - protein, 5 aa, synthetic construct
  REGION 1..5: note = A synthetic sequence
DADAD

SEQ ID NO: 8-10 - skipped (3 entries)

SEQ ID NO: 11 - protein, 6 aa, synthetic construct
  MOD_RES 2: note = L-homoproline
  MOD_RES 4: note = L-4-(phosphonodifluoromethyl)phenyalanine
PXGXYR

SEQ ID NO: 12 - skipped

SEQ ID NO: 13 - protein, 6 aa, synthetic construct
  MOD_RES 4: note = L-4-(phosphonodifluoromethyl)phenylalanine
IHIXIR

SEQ ID NO: 14-16 - skipped (3 entries)

SEQ ID NO: 17 - protein, 6 aa, synthetic construct
  MOD_RES 2: note = L-phenylglycine
  MOD_RES 4: note = L-4-(phosphonodifluoromethyl)phenylalanine
YXAXGR

SEQ ID NO: 18 - protein, 6 aa, synthetic construct
  MOD_RES 4: note = L-4-(phosphonodifluoromethyl)phenylalanine
  MOD_RES 5: note = D-Alanine
AHIXXR

SEQ ID NO: 19-20 - skipped (2 entries)

SEQ ID NO: 21 - protein, 6 aa, synthetic construct
  MOD_RES 4: note = L-4-(phosphonodifluoromethyl)phenylalanine
SPGXHR

SEQ ID NO: 22 - protein, 6 aa, synthetic construct
  MOD_RES 1: note = L-homoproline
  MOD_RES 4: note = L-4-(phosphonodifluoromethyl)phenylalanine
XYIXHR

SEQ ID NO: 23 - protein, 6 aa, synthetic construct
  MOD_RES 2: note = D-Valine
  MOD_RES 4: note = L-4-(phosphonodifluoromethyl)phenylalanine
SXPXHR

SEQ ID NO: 24 - protein, 6 aa, synthetic construct
  MOD_RES 4: note = L-4-(phosphonodifluoromethyl)phenylalanine
  MOD_RES 5: note = D-Asn
AIPXXR

SEQ ID NO: 25 - protein, 6 aa, synthetic construct
  MOD_RES 1: note = L-4-fluorophenylalanine
  MOD_RES 4: note = L-4-(phosphonodifluoromethyl)phenylalanine
XSIXQR

SEQ ID NO: 26-40 - skipped (15 entries)

SEQ ID NO: 41 - protein, 6 aa, synthetic construct
  MOD_RES 3: note = D-Thr
  MOD_RES 4: note = L-4-(phosphonodifluoromethyl)phenylalanine
SIXXYR

SEQ ID NO: 42-44 - skipped (3 entries)

SEQ ID NO: 45 - protein, 6 aa, synthetic construct
  MOD_RES 3: note = D-Asn
  MOD_RES 4: note = L-4-(phosphonodifluoromethyl)phenylalanine
AWXXAR

SEQ ID NO: 46-48 - skipped (3 entries)

SEQ ID NO: 49 - protein, 6 aa, synthetic construct
  MOD_RES 4: note = L-4-(phosphonodifluoromethyl)phenylalanine
PAHXGR

SEQ ID NO: 50 - protein, 6 aa, synthetic construct
  MOD_RES 4: note = L-4-(phosphonodifluoromethyl)phenylalanine
AYHXIR

SEQ ID NO: 51-57 - skipped (7 entries)

SEQ ID NO: 58 - protein, 4 aa, synthetic construct
  REGION 1..4: note = A synthetic sequence
HHHH

SEQ ID NO: 59 - protein, 4 aa, synthetic construct
  REGION 1..4: note = A synthetic sequence
KHKK

SEQ ID NO: 60 - protein, 4 aa, synthetic construct
  REGION 1..4: note = A synthetic sequence
KKHK

SEQ ID NO: 61 - protein, 4 aa, synthetic construct
  REGION 1..4: note = A synthetic sequence
KKKH

SEQ ID NO: 62 - protein, 4 aa, synthetic construct
  REGION 1..4: note = A synthetic sequence
KHKH

SEQ ID NO: 63 - protein, 4 aa, synthetic construct
  REGION 1..4: note = A synthetic sequence
HKHK

SEQ ID NO: 64 - protein, 4 aa, synthetic construct
  REGION 1..4: note = A synthetic sequence
KKKK

SEQ ID NO: 65 - protein, 4 aa, synthetic construct
  REGION 1..4: note = A synthetic sequence
KKRK

SEQ ID NO: 66 - protein, 4 aa, synthetic construct
  REGION 1..4: note = A synthetic sequence
KRKK

SEQ ID NO: 67 - protein, 4 aa, synthetic construct
  REGION 1..4: note = A synthetic sequence
KRRK

SEQ ID NO: 68 - protein, 4 aa, synthetic construct
  REGION 1..4: note = A synthetic sequence
RKKR

SEQ ID NO: 69 - protein, 4 aa, synthetic construct
  REGION 1..4: note = A synthetic sequence
RRRR

SEQ ID NO: 70 - protein, 4 aa, synthetic construct
  REGION 1..4: note = A synthetic sequence
KGKK

SEQ ID NO: 71 - protein, 4 aa, synthetic construct
  REGION 1..4: note = A synthetic sequence
KKGK

SEQ ID NO: 72-73 - skipped (2 entries)

SEQ ID NO: 74 - protein, 5 aa, synthetic construct
  REGION 1..5: note = A synthetic sequence
RRRRR

SEQ ID NO: 75 - protein, 5 aa, synthetic construct
  REGION 1..5: note = A synthetic sequence
KKKKK

SEQ ID NO: 76 - protein, 5 aa, synthetic construct
  REGION 1..5: note = A synthetic sequence
KKKRK

SEQ ID NO: 77 - protein, 5 aa, synthetic construct
  REGION 1..5: note = A synthetic sequence
RKKKK

SEQ ID NO: 78 - protein, 5 aa, synthetic construct
  REGION 1..5: note = A synthetic sequence
KRKKK

SEQ ID NO: 79 - protein, 5 aa, synthetic construct
  REGION 1..5: note = A synthetic sequence
KKRKK

SEQ ID NO: 80 - protein, 5 aa, synthetic construct
  REGION 1..5: note = A synthetic sequence
KKKKR

SEQ ID NO: 81 - skipped

SEQ ID NO: 82 - protein, 6 aa, synthetic construct
  REGION 1..6: note = A synthetic sequence
RKKKKG

SEQ ID NO: 83 - protein, 6 aa, synthetic construct
  REGION 1..6: note = A synthetic sequence
KRKKKG

SEQ ID NO: 84 - protein, 6 aa, synthetic construct
  REGION 1..6: note = A synthetic sequence
KKRKKG

SEQ ID NO: 85 - protein, 6 aa, synthetic construct
  REGION 1..6: note = A synthetic sequence
KKKKRG

SEQ ID NO: 86 - protein, 6 aa, synthetic construct
  MOD_RES 6: note = beta-alanine
RKKKKX

SEQ ID NO: 87 - protein, 6 aa, synthetic construct
  MOD_RES 6: note = beta-alanine
KRKKKX

SEQ ID NO: 88 - protein, 6 aa, synthetic construct
  MOD_RES 6: note = beta-alanine
KKRKKX

SEQ ID NO: 89 - protein, 6 aa, synthetic construct
  MOD_RES 6: note = beta-alanine
KKKKRX

SEQ ID NO: 90 - protein, 6 aa, synthetic construct
  REGION 1..6: note = A synthetic sequence
KKKRKV

SEQ ID NO: 91 - protein, 6 aa, synthetic construct
  REGION 1..6: note = A synthetic sequence
RRRRRR

SEQ ID NO: 92 - protein, 6 aa, synthetic construct
  REGION 1..6: note = A synthetic sequence
HHHHHH

SEQ ID NO: 93 - protein, 6 aa, synthetic construct
  REGION 1..6: note = A synthetic sequence
RHRHRH

SEQ ID NO: 94 - protein, 6 aa, synthetic construct
  REGION 1..6: note = A synthetic sequence
HRHRHR

SEQ ID NO: 95 - protein, 6 aa, synthetic construct
  REGION 1..6: note = A synthetic sequence
KRKRKR

SEQ ID NO: 96 - protein, 6 aa, synthetic construct
  REGION 1..6: note = A synthetic sequence
RKRKRK

SEQ ID NO: 97-101 - skipped (5 entries)

SEQ ID NO: 102 - protein, 5 aa, synthetic construct
  REGION 1..5: note = A synthetic sequence
KKKRK

SEQ ID NO: 103 - protein, 7 aa, synthetic construct
  REGION 1..7: note = A synthetic sequence
PKKKRKV

SEQ ID NO: 104 - protein, 7 aa, synthetic construct
  REGION 1..7: note = A synthetic sequence
PGKKRKV

SEQ ID NO: 105 - protein, 7 aa, synthetic construct
  REGION 1..7: note = A synthetic sequence
PKGKRKV

SEQ ID NO: 106 - protein, 7 aa, synthetic construct
  REGION 1..7: note = A synthetic sequence
PKKGRKV

SEQ ID NO: 107 - protein, 7 aa, synthetic construct
  REGION 1..7: note = A synthetic sequence
PKKKGKV

SEQ ID NO: 108 - protein, 7 aa, synthetic construct
  REGION 1..7: note = A synthetic sequence
PKKKRGV

SEQ ID NO: 109 - protein, 7 aa, synthetic construct
  REGION 1..7: note = A synthetic sequence
PKKKRKG

SEQ ID NO: 110 - protein, 7 aa, synthetic construct
  MOD_RES 5: note = citrulline
PKKKXKV

SEQ ID NO: 111 - protein, 19 aa, synthetic construct
  REGION 1..19: note = A synthetic sequence
NLSKRPAAIKKAGQAKKKK

SEQ ID NO: 112 - protein, 9 aa, synthetic construct
  REGION 1..9: note = A synthetic sequence
PAAKRVKLD

SEQ ID NO: 113 - protein, 11 aa, synthetic construct
  REGION 1..11: note = A synthetic sequence
RQRRNELKRSF

SEQ ID NO: 114 - protein, 27 aa, synthetic construct
  REGION 1..27: note = A synthetic sequence
RMRKFKNKGKDTAELRRRRVEVSVELR

SEQ ID NO: 115 - protein, 14 aa, synthetic construct
  REGION 1..14: note = A synthetic sequence
KAKKDEQILKRRNV

SEQ ID NO: 116 - protein, 8 aa, synthetic construct
  REGION 1..8: note = A synthetic sequence
VSRKRPRP

SEQ ID NO: 117 - protein, 8 aa, synthetic construct
  REGION 1..8: note = A synthetic sequence
PPKKARED

SEQ ID NO: 118 - protein, 8 aa, synthetic construct
  REGION 1..8: note = A synthetic sequence
PQPKKKPL

SEQ ID NO: 119 - protein, 12 aa, synthetic construct
  REGION 1..12: note = A synthetic sequence
SALIKKKKKMAP

SEQ ID NO: 120 - protein, 5 aa, synthetic construct
  REGION 1..5: note = A synthetic sequence
DRLRR

SEQ ID NO: 121 - protein, 7 aa, synthetic construct
  REGION 1..7: note = A synthetic sequence
PKQKKRK

SEQ ID NO: 122 - protein, 10 aa, synthetic construct
  REGION 1..10: note = A synthetic sequence
RKLKKKIKKL

SEQ ID NO: 123 - protein, 10 aa, synthetic construct
  REGION 1..10: note = A synthetic sequence
REKKKFLKRR

SEQ ID NO: 124 - protein, 20 aa, synthetic construct
  REGION 1..20: note = A synthetic sequence
KRKGDEVDGVDEVAKKKSKK

SEQ ID NO: 125 - protein, 17 aa, synthetic construct
  REGION 1..17: note = A synthetic sequence
RKCLQAGMNLEARKTKK

SEQ ID NO: 126 - protein, 7 aa, synthetic construct
  REGION 1..7: note = A synthetic sequence
FGFGRGR

SEQ ID NO: 127 - skipped

SEQ ID NO: 128 - protein, 8 aa, synthetic construct
  REGION 1..8: note = A synthetic sequence
FGFGRGRQ

SEQ ID NO: 129 - protein, 8 aa, synthetic construct
  REGION 1..8: note = A synthetic sequence
FGFRRRRQ

SEQ ID NO: 130 - protein, 8 aa, synthetic construct
  REGION 1..8: note = A synthetic sequence
FGFGRRRQ

SEQ ID NO: 131 - protein, 7 aa, synthetic construct
  MOD_RES 2: note = 3-(2-naphthyl)-alanine
FXRRRRQ

SEQ ID NO: 132 - protein, 8 aa, synthetic construct
  REGION 1..8: note = A synthetic sequence
FGFKRKRQ

SEQ ID NO: 133 - protein, 8 aa, synthetic construct
  REGION 1..8: note = A synthetic sequence
FGFRGRGQ

SEQ ID NO: 134 - protein, 10 aa, synthetic construct
  REGION 1..10: note = A synthetic sequence
FGFGRGRGRQ

SEQ ID NO: 135 - protein, 8 aa, synthetic construct
  REGION 1..8: note = A synthetic sequence
FGFGRRRQ

SEQ ID NO: 136 - protein, 8 aa, synthetic construct
  REGION 1..8: note = A synthetic sequence
FGFRRRRQ

SEQ ID NO: 137 - protein, 8 aa, synthetic construct
  REGION 1..8: note = A synthetic sequence
FGFGRRRQ

SEQ ID NO: 138 - DNA, 15 bp, synthetic construct
  misc_feature 1..15: note = A synthetic sequence
  source 1..15: mol_type = other DNA
taacgttgagggcat

SEQ ID NO: 139 - DNA, 25 bp, synthetic construct
  misc_feature 1..25: note = A synthetic sequence
  source 1..25: mol_type = other DNA
ggccaaacctcggcttacctgaaat

SEQ ID NO: 140 - DNA, 21 bp, synthetic construct
  misc_feature 1..21: note = A synthetic sequence
  source 1..21: mol_type = other DNA
cagcagcagcagcagcagcag

SEQ ID NO: 141 - protein, 6 aa, synthetic construct
  MOD_RES 2: note = L-naphthylalanine
FXRRRQ

SEQ ID NO: 142 - protein, 6 aa, synthetic construct
  MOD_RES 2: note = L-naphthylalanine
FXRRRC

SEQ ID NO: 143 - protein, 5 aa, synthetic construct
  MOD_RES 2: note = L-naphthylalanine
FXRRR

SEQ ID NO: 144 - protein, 6 aa, synthetic construct
  MOD_RES 4: note = L-naphthylalanine
RRRXFQ

SEQ ID NO: 145 - protein, 6 aa, synthetic construct
  MOD_RES 5: note = L-naphthylalanine
RRRRXF

SEQ ID NO: 146 - protein, 6 aa, synthetic construct
  MOD_RES 2: note = L-naphthylalanine
FXRRRR

SEQ ID NO: 147 - protein, 7 aa, synthetic construct
  MOD_RES 5: note = L-naphthylalanine
RRFRXRQ

SEQ ID NO: 148 - protein, 7 aa, synthetic construct
  MOD_RES 6: note = L-naphthylalanine
FRRRRXQ

SEQ ID NO: 149 - protein, 7 aa, synthetic construct
  MOD_RES 3: note = L-naphthylalanine
RRXFRRQ

SEQ ID NO: 150 - protein, 8 aa, synthetic construct
  REGION 1..8: note = A synthetic sequence
CRRRRFWQ

SEQ ID NO: 151 - protein, 8 aa, synthetic construct
  MOD_RES 3: note = L-naphthylalanine
FFXRRRRQ

SEQ ID NO: 152 - protein, 8 aa, synthetic construct
  MOD_RES 6: note = L-naphthylalanine
RFRFRXRQ

SEQ ID NO: 153 - protein, 7 aa, synthetic construct
  REGION 1..7: note = A synthetic sequence
RRRRFWQ

SEQ ID NO: 154 - protein, 8 aa, synthetic construct
  REGION 1..8: note = A synthetic sequence
CRRRRFWQ

SEQ ID NO: 155 - protein, 8 aa, synthetic construct
  MOD_RES 2: note = L-naphthylalanine
FXRRRRQK

SEQ ID NO: 156 - protein, 8 aa, synthetic construct
  MOD_RES 2: note = L-naphthylalanine
FXRRRRQC

SEQ ID NO: 157 - protein, 8 aa, synthetic construct
  MOD_RES 2: note = L-naphthylalanine
FXRRRRRQ

SEQ ID NO: 158 - protein, 9 aa, synthetic construct
  MOD_RES 5: note = L-naphthylalanine
  MOD_RES 8: note = L-norleucine
RRRRXFDXC

SEQ ID NO: 159 - protein, 5 aa, synthetic construct
  MOD_RES 2: note = L-naphthylalanine
FXRRR

SEQ ID NO: 160 - protein, 5 aa, synthetic construct
  REGION 1..5: note = A synthetic sequence
FWRRR

SEQ ID NO: 161 - protein, 5 aa, synthetic construct
  MOD_RES 4: note = L-naphthylalanine
RRRXF

SEQ ID NO: 162 - protein, 5 aa, synthetic construct
  REGION 1..5: note = A synthetic sequence
RRRWF

SEQ ID NO: 163-168 - skipped (6 entries)

SEQ ID NO: 169 - DNA, 19 bp, synthetic construct
  misc_feature 1..19: note = A synthetic sequence
  source 1..19: mol_type = other DNA
gtaactgatttggtacttc

SEQ ID NO: 170 - DNA, 14 bp, synthetic construct
  misc_feature 1..14: note = A synthetic sequence
  source 1..14: mol_type = other DNA
ctgtatttggtact

SEQ ID NO: 171 - DNA, 18 bp, synthetic construct
  misc_feature 1..18: note = A synthetic sequence
  source 1..18: mol_type = other DNA
ttcttcttcttcttcttc

SEQ ID NO: 172 - DNA, 20 bp, synthetic construct
  misc_feature 1..20: note = A synthetic sequence
  source 1..20: mol_type = other DNA
cttcttcttcttcttcttct

SEQ ID NO: 173 - DNA, 20 bp, synthetic construct
  misc_feature 1..20: note = A synthetic sequence
  source 1..20: mol_type = other DNA
tcttcttcttcttcttcttc

SEQ ID NO: 174 - DNA, 13 bp, synthetic construct
  misc_feature 1..13: note = A synthetic sequence
  source 1..13: mol_type = other DNA
gaagaagaagaag

SEQ ID NO: 175 - RNA, 18 nt, synthetic construct
  misc_feature 1..18: note = A synthetic sequence
  source 1..18: mol_type = other RNA
cststcttcttcttcttc

SEQ ID NO: 176 - DNA, 14 bp, synthetic construct
  misc_feature 1..14: note = A synthetic sequence
  source 1..14: mol_type = other DNA
gaagaagaagaaga

SEQ ID NO: 177 - RNA, 14 nt, synthetic construct
  misc_feature 1..14: note = A synthetic sequence
  source 1..14: mol_type = other RNA
tcttcttcttcttc

SEQ ID NO: 178 - DNA, 14 bp, synthetic construct
  misc_feature 1..14: note = A synthetic sequence
  source 1..14: mol_type = other DNA
gaagaagaagaaga

SEQ ID NO: 179 - RNA, 14 nt, synthetic construct
  misc_feature 1..14: note = A synthetic sequence
  source 1..14: mol_type = other RNA
tcttcttcttcttc

SEQ ID NO: 180 - DNA, 21 bp, synthetic construct
  misc_feature 1..21: note = A synthetic sequence
  source 1..21: mol_type = other DNA
gaagaagaagaagaagaagtt

SEQ ID NO: 181 - RNA, 21 nt, synthetic construct
  source 1..21: mol_type = other RNA
  variation 20: note = T
  variation 21: note = T
cttcttcttcttcttcttcnn

SEQ ID NO: 182 - RNA, 21 nt, synthetic construct
  source 1..21: mol_type = other RNA
  variation 20: note = T
  variation 21: note = T
gctataccagcgtcgtcatnn

SEQ ID NO: 183 - DNA, 21 bp, synthetic construct
  misc_feature 1..21: note = A synthetic sequence
  source 1..21: mol_type = other DNA
atgacgacgctggtatagctt

SEQ ID NO: 184 - RNA, 21 nt, synthetic construct
  source 1..21: mol_type = other RNA
  variation 20: note = T
  variation 21: note = T
gagtgtctatttgatgaatnn

SEQ ID NO: 185 - RNA, 21 nt, synthetic construct
  source 1..21: mol_type = other RNA
  variation 20: note = T
  variation 21: note = T
attcatcaaatagacactcnn

SEQ ID NO: 186 - RNA, 17 nt, synthetic construct
  misc_feature 3: note = A synthetic sequence, n = T
  source 1..17: mol_type = other RNA
  variation 3: note = T
ccncaaaagcaggaata

SEQ ID NO: 187 - DNA, 15 bp, synthetic construct
  misc_feature 1..15: note = A synthetic sequence
  source 1..15: mol_type = other DNA
ccgggtctgccgccc

SEQ ID NO: 188 - DNA, 17 bp, synthetic construct
  misc_feature 1..17: note = A synthetic sequence
  source 1..17: mol_type = other DNA
cctcaaaagcaggaata

SEQ ID NO: 189 - DNA, 15 bp, synthetic construct
  misc_feature 1..15: note = A synthetic sequence
  source 1..15: mol_type = other DNA
ccgggtctgccgccc

SEQ ID NO: 190 - DNA, 25 bp, synthetic construct
  misc_feature 1..25: note = A synthetic sequence
  source 1..25: mol_type = other DNA
ccaactgtcctcaaaagcaggaata

SEQ ID NO: 191 - DNA, 25 bp, synthetic construct
  misc_feature 1..25: note = A synthetic sequence
  source 1..25: mol_type = other DNA
ccgggtctgccgcccgctccgccct

SEQ ID NO: 192 - DNA, 25 bp, synthetic construct
  misc_feature 1..25: note = A synthetic sequence
  source 1..25: mol_type = other DNA
cttcttcttcttcttcttcttcttc

SEQ ID NO: 193 - DNA, 18 bp, synthetic construct
  misc_feature 1..18: note = A synthetic sequence
  source 1..18: mol_type = other DNA
cttcttcttcttcttctt

SEQ ID NO: 194 - DNA, 19 bp, synthetic construct
  misc_feature 1..19: note = A synthetic sequence
  source 1..19: mol_type = other DNA
tcttcttcttcttcttctt

SEQ ID NO: 195 - DNA, 20 bp, synthetic construct
  misc_feature 1..20: note = A synthetic sequence
  source 1..20: mol_type = other DNA
cttcttcttcttcttcttct

SEQ ID NO: 196 - DNA, 20 bp, synthetic construct
  misc_feature 1..20: note = A synthetic sequence
  source 1..20: mol_type = other DNA
tcttcttcttcttcttcttc

SEQ ID NO: 197 - DNA, 18 bp, synthetic construct
  misc_feature 1..18: note = A synthetic sequence
  source 1..18: mol_type = other DNA
cttcttcttcttcttctt

SEQ ID NO: 198 - DNA, 20 bp, synthetic construct
  misc_feature 1..20: note = A synthetic sequence
  source 1..20: mol_type = other DNA
acagacaataaataccgagg

SEQ ID NO: 199 - DNA, 20 bp, synthetic construct
  misc_feature 1..20: note = A synthetic sequence
  source 1..20: mol_type = other DNA
acagacaataaataccgagg

SEQ ID NO: 200 - DNA, 20 bp, synthetic construct
  misc_feature 1..20: note = A synthetic sequence
  source 1..20: mol_type = other DNA
acagacaataaataccgagg

SEQ ID NO: 201 - DNA, 16 bp, synthetic construct
  misc_feature 1..16: note = A synthetic sequence
  source 1..16: mol_type = other DNA
acaataaataccgagg

SEQ ID NO: 202 - DNA, 16 bp, synthetic construct
  misc_feature 1..16: note = A synthetic sequence
  source 1..16: mol_type = other DNA
acaataaataccgagg

SEQ ID NO: 203 - DNA, 20 bp, synthetic construct
  misc_feature 1..20: note = A synthetic sequence
  source 1..20: mol_type = other DNA
cggagcggttgtgaactggc

SEQ ID NO: 204 - DNA, 20 bp, synthetic construct
  misc_feature 1..20: note = A synthetic sequence
  source 1..20: mol_type = other DNA
cggagcggtgtgaactggca

SEQ ID NO: 205 - DNA, 20 bp, synthetic construct
  misc_feature 1..20: note = A synthetic sequence
  source 1..20: mol_type = other DNA
acagacaataaataccgagg

SEQ ID NO: 206 - DNA, 20 bp, synthetic construct
  misc_feature 1..20: note = A synthetic sequence
  source 1..20: mol_type = other DNA
acagacaataaataccgagg

SEQ ID NO: 207 - DNA, 20 bp, synthetic construct
  misc_feature 1..20: note = A synthetic sequence
  source 1..20: mol_type = other DNA
acagacaataaataccgagg

SEQ ID NO: 208 - DNA, 20 bp, synthetic construct
  misc_feature 1..20: note = A synthetic sequence
  source 1..20: mol_type = other DNA
acagacaataaataccgagg

SEQ ID NO: 209 - DNA, 20 bp, synthetic construct
  misc_feature 1..20: note = A synthetic sequence
  source 1..20: mol_type = other DNA
acagacaataaataccgagg

SEQ ID NO: 210 - DNA, 20 bp, synthetic construct
  misc_feature 1..20: note = A synthetic sequence
  source 1..20: mol_type = other DNA
acagacaataaataccgagg

SEQ ID NO: 211 - DNA, 20 bp, synthetic construct
  misc_feature 1..20: note = A synthetic sequence
  source 1..20: mol_type = other DNA
acagacaataaataccgagg

SEQ ID NO: 212 - DNA, 21 bp, synthetic construct
  misc_feature 1..21: note = A synthetic sequence
  source 1..21: mol_type = other DNA
cagcagcagcagcagcagcag

SEQ ID NO: 213 - DNA, 21 bp, synthetic construct
  misc_feature 1..21: note = A synthetic sequence
  source 1..21: mol_type = other DNA
cagcagcagcagcagcagcag

SEQ ID NO: 214 - DNA, 20 bp, synthetic construct
  misc_feature 1..20: note = A synthetic sequence
  source 1..20: mol_type = other DNA
acagacaataaataccgagg

SEQ ID NO: 215 - DNA, 20 bp, synthetic construct
  misc_feature 1..20: note = A synthetic sequence
  source 1..20: mol_type = other DNA
cggagcggttgtgaactggc

SEQ ID NO: 216 - DNA, 18 bp, synthetic construct
  misc_feature 1..18: note = A synthetic sequence
  source 1..18: mol_type = other DNA
ccataataaagggctcag

SEQ ID NO: 217 - DNA, 20 bp, synthetic construct
  misc_feature 1..20: note = A synthetic sequence
  source 1..20: mol_type = other DNA
cagctggagtttatcttttg

SEQ ID NO: 218 - DNA, 18 bp, synthetic construct
  misc_feature 1..18: note = A synthetic sequence
  source 1..18: mol_type = other DNA
gctattaccttaacccag

SEQ ID NO: 219 - DNA, 18 bp, synthetic construct
  misc_feature 1..18: note = A synthetic sequence
  source 1..18: mol_type = other DNA
gctattaccttaacccag

SEQ ID NO: 220 - protein, 12 aa, synthetic construct
  REGION 1..12: note = A synthetic sequence
SKTFNTHPQSTP

SEQ ID NO: 221 - DNA, 21 bp, synthetic construct
  misc_feature 1..21: note = A synthetic sequence
  source 1..21: mol_type = other DNA
gtaactgtatttggtacttcc

SEQ ID NO: 222 - DNA, 15 bp, synthetic construct
  misc_feature 1..15: note = A synthetic sequence
  source 1..15: mol_type = other DNA
ctgtatttggtactt

SEQ ID NO: 223 - DNA, 18 bp, synthetic construct
  misc_feature 1..18: note = A synthetic sequence
  source 1..18: mol_type = other DNA
ctcagacatgccgctgct

SEQ ID NO: 224 - DNA, 22 bp, synthetic construct
  misc_feature 1..22: note = A synthetic sequence
  source 1..22: mol_type = other DNA
ttgagctggatggttctctccg

SEQ ID NO: 225 - DNA, 21 bp, synthetic construct
  misc_feature 1..21: note = A synthetic sequence
  source 1..21: mol_type = other DNA
gtaactgtatttggtacttcc

SEQ ID NO: 226 - DNA, 12 bp, synthetic construct
  source 1..12: mol_type = other DNA
atgcatgcatgc

SEQ ID NO: 227 - DNA, 26 bp, synthetic construct
  source 1..26: mol_type = other DNA
cgtactgatcattgtgcagctgcagc

SEQ ID NO: 228 - protein, 8 aa, synthetic construct
PKKKRKVK

SEQ ID NO: 229 - DNA, 25 bp, synthetic construct
  source 1..25: mol_type = other DNA
agaacgtaatcatcagtgggttggc

SEQ ID NO: 230 - protein, 7 aa, synthetic construct
GFFGRGR

SEQ ID NO: 231 - protein, 7 aa, synthetic construct
  MOD_RES 3: note = 3-(2-naphthyl)-alanine
FFXGRGR

SEQ ID NO: 232 - protein, 7 aa, synthetic construct
FFFGRGR

SEQ ID NO: 233 - protein, 8 aa, synthetic construct
GFFGRGRQ

SEQ ID NO: 234 - protein, 8 aa, synthetic construct
  MOD_RES 3: note = 3-(2-naphthyl)-alanine
FFXGRGRQ

SEQ ID NO: 235 - protein, 8 aa, synthetic construct
FFFGRGRQ

SEQ ID NO: 236 - protein, 8 aa, synthetic construct
  MOD_RES 3: note = 3-(2-naphthyl)-alanine
  MOD_RES 4: note = citrulline
  MOD_RES 6: note = citrulline
FFXXRXRQ

SEQ ID NO: 237-240 - skipped (4 entries)

SEQ ID NO: 241 - protein, 8 aa, synthetic construct
  MOD_RES 3: note = 3-(2-naphthyl)-alanine
FFXRGRGQ

SEQ ID NO: 242 - protein, 8 aa, synthetic construct
  MOD_RES 3: note = 3-(2-naphthyl)-alanine
  MOD_RES 4: note = 4-guanidino-L-phenylalanine
  MOD_RES 6: note = 4-guanidino-L-phenylalanine
FFXXRXRQ

SEQ ID NO: 243 - protein, 8 aa, synthetic construct
  MOD_RES 3: note = 3-(2-naphthyl)-alanine
  MOD_RES 4: note = 3-(4-piperidinyl)-L-alanine
  MOD_RES 6: note = 3-(4-piperidinyl)-L-alanine
FFXXRXRQ

SEQ ID NO: 244 - protein, 8 aa, synthetic construct
  MOD_RES 3: note = 3-(2-naphthyl)-alanine
  MOD_RES 4: note = N-dimethyl-L-lysine
  MOD_RES 6: note = N-dimethyl-L-lysine
FFXXRXRQ

SEQ ID NO: 245 - protein, 7 aa, synthetic construct
  MOD_RES 2: note = 3-(2-naphthyl)-alanine
  MOD_RES 4: note = citrulline
  MOD_RES 6: note = citrulline
FXRXRXQ

SEQ ID NO: 246 - protein, 8 aa, synthetic construct
PKKKRKVK

SEQ ID NO: 247 - protein, 8 aa, synthetic construct
  MOD_RES 3: note = 3-(2-naphthyl)-alanine
  MOD_RES 6: note = citrulline
FFXRRXRQ

SEQ ID NO: 248 - protein, 8 aa, synthetic construct
  MOD_RES 3: note = 3-(2-naphthyl)-alanine
  MOD_RES 4: note = citrulline
FFXXRRRQ

SEQ ID NO: 249 - protein, 8 aa, synthetic construct
  MOD_RES 3: note = 3-(2-naphthyl)-alanine
  MOD_RES 5: note = citrulline
  MOD_RES 7: note = citrulline
FFXRXRXQ

SEQ ID NO: 250 - protein, 12 aa, synthetic construct
PKKKRKVAEEAK

SEQ ID NO: 251 - protein, 8 aa, synthetic construct
  MOD_RES 3: note = 3-(2-naphthyl)-alanine
FFXSRSRQ

SEQ ID NO: 252 - protein, 8 aa, synthetic construct
FGFRRRRQ

SEQ ID NO: 253 - protein, 19 aa, synthetic construct
NLSKRPAAIKKAGQAKKKK

SEQ ID NO: 254 - protein, 8 aa, synthetic construct
  MOD_RES 3: note = 3-(2-naphthyl)-alanine
  MOD_RES 4: note = L-2-amino-3-guanidinopropionic
  MOD_RES 6: note = L-2-amino-3-guanidinopropionic
FFXXRXRQ

SEQ ID NO: 255 - protein, 8 aa, synthetic construct
  MOD_RES 3: note = 3-(2-naphthyl)-alanine
  MOD_RES 4: note = L-homoarginine
  MOD_RES 6: note = L-homoarginine
FFXXRXRQ

SEQ ID NO: 256 - protein, 7 aa, synthetic construct
  MOD_RES 5: note = citrulline
PKKKXKV

SEQ ID NO: 257 - DNA, 22 bp, synthetic construct
  source 1..22: mol_type = other DNA
cagaattctgccaattgctgag

SEQ ID NO: 258 - DNA, 20 bp, synthetic construct
  source 1..20: mol_type = other DNA
ttcttcagcttgtgtcatcc

SEQ ID NO: 259 - DNA, 24 bp, synthetic construct
  source 1..24: mol_type = other DNA
cccagtctaccaccctatcagagc

SEQ ID NO: 260 - DNA, 20 bp, synthetic construct
  source 1..20: mol_type = other DNA
cctgcctttaaggcttcctt

SEQ ID NO: 261 - DNA, 25 bp, synthetic construct
  source 1..25: mol_type = other DNA
aaacgccgccatttctcaacagatc

SEQ ID NO: 262 - DNA, 20 bp, synthetic construct
  source 1..20: mol_type = other DNA
gctcaggtcggattgacatt

SEQ ID NO: 263 - DNA, 20 bp, synthetic construct
  source 1..20: mol_type = other DNA
gggcaactcttccaccagta

SEQ ID NO: 264 - protein, 12 aa, synthetic construct
PKKKRKVAEEAK

SEQ ID NO: 265 - protein, 13 aa, synthetic construct
FFFGRGRQAEEAK

SEQ ID NO: 266 - protein, 24 aa, synthetic construct
PKKKRKVAEEAKFGFGRGRQAEEA

SEQ ID NO: 267 - protein, 20 aa, synthetic construct
KBKAEEAKFGFGRGRQAEEA

SEQ ID NO: 268 - protein, 24 aa, synthetic construct
PKKKRKVAEEAKGFFGRGRQAEEA

SEQ ID NO: 269 - protein, 24 aa, synthetic construct
PKKKRKVAEEAKFFFGRGRQAEEA

SEQ ID NO: 270 - protein, 22 aa, synthetic construct
KBKBKAEEAKFFFGRGRQAEEA

SEQ ID NO: 271-272 - skipped (2 entries)

SEQ ID NO: 273 - DNA, 25 bp, synthetic construct
  source 1..25: mol_type = other DNA
tcactgtctggctcacatacccata

SEQ ID NO: 274 - protein, 20 aa, synthetic construct
PKKKRKVAEEAKFGFGRGRQ

SEQ ID NO: 275 - protein, 20 aa, synthetic construct
  MOD_RES 15: note = 3-(2-naphthyl)-alanine
PKKKRKVAEEAKFFXGRGRQ

SEQ ID NO: 276 - protein, 20 aa, synthetic construct
PKKKRKVAEEAKFGFGRRRQ

SEQ ID NO: 277 - protein, 20 aa, synthetic construct
PKKKRKVAEEAKFGFRRRRQ

SEQ ID NO: 278 - DNA, 25 bp, synthetic construct
  source 1..25: mol_type = other DNA
agaacgtaatcatcagtgggttggc

SEQ ID NO: 279 - protein, 7 aa, synthetic construct
  MOD_RES 3: note = 3-(2-naphthyl)-alanine
FFXRRRR

SEQ ID NO: 280 - protein, 7 aa, synthetic construct
ASSLNIA

SEQ ID NO: 281 - protein, 16 aa, synthetic construct
  MOD_RES 11: note = 3-(2-naphthyl)-alanine
  MOD_RES 13: note = citrulline
  MOD_RES 15: note = citrulline
PKKKRKVKFFXRXRXQ

SEQ ID NO: 282 - skipped

SEQ ID NO: 283 - protein, 8 aa, synthetic construct
PGKKKRKV

SEQ ID NO: 284-287 - skipped (4 entries)

SEQ ID NO: 288 - protein, 17 aa, synthetic construct
  MOD_RES 11: note = 3-(2-naphthyl)-alanine
PKKKRKVKPPXGRGRGQ

SEQ ID NO: 289 - protein, 20 aa, synthetic construct
  MOD_RES 15: note = 3-(2-naphthyl)-alanine
  MOD_RES 16: note = citrulline
  MOD_RES 18: note = citrulline
PKKKRKVAEEAKFFXXRXRQ

SEQ ID NO: 290 - protein, 6 aa, synthetic construct
KKKRKG